(19) United States
(12) Patent Application Publication
(10) Pub. No.: US 2026/0007666 A1
(43) Pub. Date: Jan. 8, 2026

(54) INHALABLE IMATINIB FORMULATION

(71) Applicant: INAYA THERAPEUTICS, INC., Los Gatos, CA (US)

(72) Inventors: Grace E. COLON, Los Gatos, CA (US); Michael Laird HURREY, San Ramon, CA (US); Carlos SCHULER, Kensington, CA (US); Brian WIEST, Castro Valley, CA (US)

(21) Appl. No.: 18/992,238

(22) PCT Filed: Jul. 13, 2023
(86) PCT No.: PCT/US2023/027645

§ 371 Date: Jan. 8, 2025

Related U.S. Application Data
(60) Provisional application No. 63/388,785, filed on Jul. 13, 2022.

(51) Int. Cl. A61K 31/506; A61K 9/00; A61K 9/08; A61K 9/10; A61K 47/02; A61K 47/40

(52) U.S. Cl. CPC A61K 31/506; A61K 9/0078; A61K 9/08; A61K 9/10; A61K 47/02; A61K 47/40

(57) ABSTRACT

Pharmaceutical compositions comprising imatinib or a derivative thereof for treatment of a pulmonary disease via inhalation. Methods of treating a pulmonary disease include administering by inhalation an effective amount of imatinib or a derivative thereof to a patient in need thereof. In aspects, the pharmaceutical composition provided herein comprises an aqueous solution or suspension of imatinib or a derivative thereof that is formulated for inhalational administration.

### CROSS-REFERENCE

This application claims the benefit of U.S. Provisional Application No. 63/388,785, filed Jul. 13, 2022, which is incorporated herein by reference in its entirety.

### BACKGROUND

Imatinib is a small molecule kinase inhibitor that can inhibit the Bcr-Abl tyrosine kinase, the constitutive abnormal tyrosine kinase created by the Philadelphia chromosome abnormality in chronic myeloid leukemia (CML). It can be used to treat certain types of cancer. It is currently marketed by Novartis as Gleevec (USA) or Glivec (Europe/Australia) as its mesylate salt, imatinib mesylate (INN). It can inhibit proliferation and induce apoptosis in Bcr-Abl positive cell lines as well as fresh leukemic cells from Philadelphia chromosome positive chronic myeloid leukemia. Imatinib can also inhibit the receptor tyrosine kinases for platelet derived growth factor (PDGF) and stem cell factor (SCF). There are also reports that injection (subcutaneous or intraperitoneal) or oral delivery of imatinib mesylate can have therapeutic effects on pulmonary arterial hypertension in animal models and patients enrolled in clinical trials.

### SUMMARY

Provided herein, in some aspects, is a method of treating a subject in need thereof, comprising administering to said subject a pharmaceutical composition, wherein said administering comprises delivering said pharmaceutical composition via inhalation, wherein said pharmaceutical composition comprises an aqueous solution or suspension that comprises imatinib or a derivative thereof and cyclodextrin, wherein said pharmaceutical composition provides a pharmacokinetic profile when measured in a human clinical trial in which human subjects receive said administering of a single dose of said pharmaceutical composition, and wherein said pharmacokinetic profile is characterized by: (a) a maximum measured plasma concentration of said imatinib or derivative thereof (Cₘₐₓ) ranging from about 50 ng/mL to about 1900 ng/mL; (b) an area under the plasma concentration of said imatinib or derivative thereof-time curve from 0 to 24 hour (AUC₀₋₂₄) ranging from about 1 μg*h/mL to about 24 μg*h/mL; (c) an area under the plasma concentration of said imatinib or derivative thereof-time curve from 0 to infinity (AUC_{0-∞}) ranging from about 1 μg*h/mL to about 38 μg*h/mL; or (d) any combination of (a)-(c).

In another aspect, provided herein is a method of treating a subject in need thereof, comprising administering to said subject in need thereof a pharmaceutical composition,
wherein said administering comprises delivering said pharmaceutical composition via inhalation,
wherein said pharmaceutical composition comprises an aqueous solution or suspension that comprises imatinib or a derivative thereof and cyclodextrin,
wherein said pharmaceutical composition provides a pharmacokinetic profile when measured in an animal study in which mammalian animal subjects receive a single dose of said pharmaceutical composition or a diluted version thereof via inhalational administration or oral administration, and
wherein said pharmacokinetic profile is characterized by:
(i) a ratio of maximum measured lung concentration of said imatinib or derivative thereof post said inhalational administration (C_{max,inhalation,lung}) to maximum plasma concentration of said imatinib or derivative thereof said inhalational administration (C_{max,inhalation,plasma}) ranging from about 20 to about 1000; or
(ii) a ratio of maximum measured lung concentration of said imatinib or derivative thereof post said inhalational administration (C_{max,inhalation,lung}) to maximum measured lung concentration of said imatinib or derivative thereof post said oral administration (C_{max,po,lung}) ranging from about 20 to about 1000; or
(iii) a ratio of area under lung concentration of said imatinib or derivative thereof-time curve from 0 to 24 hours post said inhalational administration (AUC_{inhalation,lung,0-24}) to area under plasma concentration of said imatinib or derivative thereof-time curve from 0 to 24 hours post said inhalational administration (AUC_{inhalation,plasma,0-24}) ranging from about 10 to about 1000; or
(iv) a ratio of area under lung concentration of said imatinib or derivative thereof-time curve from 0 to 24 hours post said inhalational administration (AUC_{inhalation,lung,0-24}) to area under lung concentration of said imatinib or derivative thereof-time curve from 0 to 24 hours post said oral administration (AUC_{po,lung,0-24}) ranging from about 10 to about 1000; or
(v) a therapeutic advantage (Rd) of inhalational administration compared to oral administration ranging from about 2 to about 200, wherein Rd is calculated according to the following equation:

$$Rd = \frac{( {AUC}_{inhalation , lung , 0 - 24} / {AUC}_{inhalation , plasma , 0 - 24} )}{( {AUC}_{po , lung , 0 - 24} / {AUC}_{po , plasma , 0 - 24} )} ,$$

wherein AUC_{po,plasma,0-24} is area under plasma concentration of said imatinib or derivative thereof-time curve from 0 to 24 hours post said oral administration; or
(vi) any combination of (i)-(v).

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure may be obtained by reference to the following detailed description that sets forth illustrative embodiments, in which the principles of the disclosure are utilized, and the accompanying drawings of which:

FIG. 1 shows a graph demonstrating the maximum concentration of imatinib free base (mg/mL) as a function of pH.

FIG. 2 shows a graph demonstrating the maximum concentration of imatinib free base (mg/mL) as a function of percent hydroxypropyl β cyclodextrin (HPβCD or “HPBCD” in the figure) (w/v) at a pH of 5 and 7.5.

FIG. 3A shows a graph demonstrating the maximum concentration of imatinib free base (mg/mL) as a function of percent hydroxypropyl β cyclodextrin (HPβCD or “HPBCD” in the figure) (w/v) at a pH of 5 and 7.5 and percent sulfobutylether β cyclodextrin (SBEβCD or “SBEBCD” in the figure) (w/v) at a pH of 5.

FIG. 3B shows pictures of exemplary suspension and solutions of about 30 mg/mL imatinib free base in an aqueous solution of 30% SBEβCD and 50 mM phosphate buffer at different pH levels.

FIG. 4A is a plot depicting lung tissue concentration of imatinib post IT (freebase suspension) or IV (mesylate solution) administration over time.

FIG. 4B is a plot depicting plasma concentration of imatinib post IT (freebase suspension) or IV (mesylate solution) administration over time.

FIG. 4C is a plot depicting lung tissue concentration of imatinib vs. plasma concentration of imatinib over time post IT administration of the imatinib free base suspension.

FIG. 4D is a plot depicting lung tissue concentration of imatinib vs. plasma concentration of imatinib over time post IV administration of the imatinib mesylate solution.

FIG. 4E is a plot depicting lung tissue concentration of imatinib vs. plasma concentration of imatinib over time post IT administration of the imatinib free base suspension plotted on a log scale.

FIG. 4F is a plot on a log scale depicting lung tissue concentration of imatinib vs. plasma concentration of imatinib over time post IV administration of the imatinib mesylate solution.

### DETAILED DESCRIPTION

In some aspects, the present disclosure provides compositions (e.g., pharmaceutical compositions), methods (e.g., methods of treatment, methods of making the compositions), kits, and systems that relate to an aqueous solution or suspension of imatinib. The aqueous solution or suspension of imatinib disclosed herein can be used as an inhalable formulation, e.g., via aerosolization by a nebulizer, for use in human patients. In some embodiments, the pharmaceutical compositions and methods of treatment provided herein are advantageous in offering fast, efficient, and safe therapeutic solution to treating pulmonary conditions. In some embodiments, the present disclosure relates to inhalational administration of a pharmaceutical composition in an aqueous solution or suspension form that comprises imatinib and a solubility enhancer.

In some embodiments, the pharmaceutical composition (or formulation) provided herein enables delivery of more pharmaceutically active ingredient, e.g., imatinib, to the subject, in a single dose, or in multiples doses over a period of time. In some embodiments, the pharmaceutical composition or formulation disclosed herein has at least one solubility enhancer. In some embodiments, the solubility enhancers comprise cyclodextrin, pH buffer, lipids, fatty acids, co-solvents, or organic solvents. In some cases, the pharmaceutical compositions described herein comprises one or more solubility enhancers to increase solubility of imatinib or a derivative thereof, or a pharmaceutically acceptable salt thereof. As used herein, the term “solubility” with respect to a designated solute in a solution can refer to the maximum amount of the solute that can be dissolved in a unit amount of a designated solvent in the solution. The term “solubility” when used with reference to imatinib or a derivative thereof (e.g., imatinib free base) that is dissolved in an aqueous solution can refer to the maximum amount of imatinib or derivative thereof that can be dissolved in a unit amount of water present in the aqueous solution. In some embodiments, solubility enhancers in the aqueous solution or suspension provided herein decreases the number of inhalations required to deliver a given dose. A given dose as formulated in a pharmaceutical composition described herein can be delivered at a higher speed, for instance, as compared to a comparable formulation that does not have the solubility enhancer and has a lower concentration of imatinib. Shorter inhalation duration can improve subject compliance, which can further increase the delivery efficiency of the drug.

In some embodiments, the pharmaceutical composition or formulation provided herein reduces adverse coughing of the subject during inhalation, has improved organoleptic properties, and improves overall patient experience during inhalation. In some embodiments, the improved overall inhalation experience results in better compliance with the full inhalation program. In some embodiments, more effective drug delivery is achieved when the subject has better inhalation compliance, and thus more drug is delivered. Some aqueous solutions of imatinib mesylate or other salts of imatinib can have poor organoleptic properties, which can induce, for instance, severe respiratory irritation, adverse sensations in mouth and throat when inhaled by a human subject, and/or coughing of sufficient severity to preclude continuous deep lung (e.g., peripheral lung) inhalation. In some cases, solutions of imatinib mesylate or certain other salts of imatinib are not inhalable, e.g., because they are not organoleptically tolerable to human subjects to enable continuous deep lung inhalation of the nebulized aerosol. In contrast, formulations according to some embodiments of the present disclosure can have improved organoleptic properties and suitable for deep lung inhalation of their nebulized aerosols. In some cases, formulations provided herein, for instance, aqueous solutions made of imatinib freebase, are not irritating or minimally irritating to mouth and throat when being inhaled in the form of a nebulized aerosol. For instance, the subject may not experience any adverse or severely adverse sensational irritation when inhaling nebulized aerosol of some formulations provided herein. Some formulations provided herein may not induce a cough reflex or strong coughing in the subject inhaling the formulations. A subject inhaling some formulations provided herein may report some formulations provided herein as tolerable and can continuously conduct deep lung inhalation of them for a desirable period of time.

Without being bound by a certain theory, the mesylate salt form of imatinib may contribute to the adverse organoleptic properties of the nebulized aerosol of its aqueous solution. In some embodiments, the formulations provided herein circumvent the problem associated with the adverse organoleptic properties by use of imatinib freebase or other salt forms of imatinib. The absence of mesylate form of imatinib can contribute to the improved organoleptic properties of certain formulations provided herein.

In one aspect of the present disclosure, provided herein is a unit dose of a pharmaceutical composition provided herein. In some embodiments, the unit dose comprises about 20 mg to about 500 mg of imatinib free base. In another aspect, provided herein are kits comprising the pharmaceutical composition or the unit dose provided herein and instructions for use of the pharmaceutical composition for treatment of a pulmonary disease.

### Composition

In some aspects, provided herein are compositions (e.g., pharmaceutical compositions or formulations) that comprise an aqueous solution or suspension of imatinib or a derivative thereof. In some cases, the composition comprises an aqueous solution or suspension that comprises: imatinib or a derivative thereof, a solubility enhancer, and a pH buffer. In some embodiments, the aqueous solution or suspension has a concentration of imatinib of from 20 to 500 mg/mL. In some cases, the aqueous solution or suspension has a viscosity of at most 10 centipoise. In some cases, the aqueous solution or suspension has a pH of 3 to 8. In some cases, the aqueous solution or suspension has a concentration of imatinib or derivative thereof of from 20 to 500 mg/m, has a viscosity of at most 10 centipoise and has a pH of 3 to 8.

In some cases, provided herein is a composition that comprises an aqueous solution or suspension that comprises imatinib or a derivative thereof, and cyclodextrin. In some cases, the aqueous solution or suspension has the cyclodextrin at a concentration of from about 1% (w/v) to about 80% (w/v). In some cases, the cyclodextrin is anionic cyclodextrin.

In some cases, a composition provided herein has from 20 mg/mL to 400 mg/mL, from 20 mg/mL to 300 mg/mL, from 20 mg/mL to 200 mg/mL, from 100 mg/mL to 500 mg/mL, from 200 mg/mL to 500 mg/mL, from 300 mg/mL to 500 mg/mL, from 400 mg/mL to 500 mg/mL, from 100 mg/mL to 400 mg/mL, from 100 mg/mL to 300 mg/mL, from 100 mg/mL to 200 mg/mL, from 200 mg/mL to 400 mg/mL, from 200 mg/mL to 300 mg/mL, from 20 to 100, from 20 mg/mL to 80 mg/mL, from 20 mg/mL to 60 mg/mL, from 20 mg/mL to 40 mg/mL, from 20 mg/mL to 30 mg/mL, from 30 mg/mL to 40 mg/mL, from 40 mg/mL to 60 mg/mL, from 40 mg/mL to 80 mg/mL, from 40 mg/mL to 100 mg/mL, from 40 mg/mL to 120 mg/mL, from 40 mg/mL to 150 mg/mL, from 60 mg/mL to 80 mg/mL, from 60 mg/mL to 100 mg/mL, from 60 mg/mL to 120 mg/mL, or from 60 mg/mL to 150 mg/mL imatinib or derivative thereof.

In some cases, a composition provided herein has a viscosity of at most 10 centipoise, such as at most 9.5 centipoise, at most 9.0 centipoise, at most 8.5 centipoise, at most 8.0 centipoise, at most 7.6 centipoise, at most 7.4 centipoise, at most 7.2 centipoise, at most 7.0 centipoise, at most 6.8 centipoise, at most 6.6 centipoise, at most 6.4 centipoise, at most 6.2 centipoise, at most 6.0 centipoise, at most 5.8 centipoise, at most 5.6 centipoise, at most 5.4 centipoise, at most 5.2 centipoise, at most 5.0 centipoise, at most 4.8 centipoise, at most 4.6 centipoise, at most 4.4 centipoise, at most 4.2 centipoise, at most 4.0 centipoise, at most 3.8 centipoise, at most 3.6 centipoise, at most 3.4 centipoise, at most 3.2 centipoise, at most 3.0 centipoise, at most 2.8 centipoise, at most 2.6 centipoise, at most 2.4 centipoise, at most 2.2 centipoise, at most 2.0 centipoise, at most 1.8 centipoise, at most 1.6 centipoise, at most 1.4 centipoise, at most 1.2 centipoise, at most 1.0 centipoise, at most 0.8 centipoise, at most 0.6 centipoise, at most 0.4 centipoise, or at most 0.2 centipoise. For instance, the composition can have a viscosity of about 0.1 centipoise, 0.2 centipoise, 0.3 centipoise, 0.4 centipoise, 0.5 centipoise, 0.6 centipoise, 0.7 centipoise, 0.8 centipoise, 0.9 centipoise, 1.0 centipoise, 1.1 centipoise, 1.2 centipoise, 1.3 centipoise, 1.4 centipoise, 1.5 centipoise, 1.6 centipoise, 1.7 centipoise, 1.8 centipoise, 1.9 centipoise, 2.0 centipoise, 2.1 centipoise, 2.2 centipoise, 2.3 centipoise, 2.4 centipoise, 2.5 centipoise, 2.6 centipoise, 2.8 centipoise, 3.0 centipoise, 3.2 centipoise, 3.5 centipoise, 3.8 centipoise, 4.0 centipoise, 4.2 centipoise, 4.5 centipoise, 4.8 centipoise, 5.0 centipoise, 5.5 centipoise, 6.0 centipoise, 6.5 centipoise, 7.0 centipoise, 7.5 centipoise, 8.0 centipoise, or 8.5 centipoise.

In some cases, a composition provided herein has a pH of 3 to 7, such as from 3 to 6, from 4 to 6, from 4.5 to 5.5, from 5 to 6, from 4 to 7, from 5 to 7, or from 6 to 7. For instance, the composition can have a pH of about 4.5, about 4.7, about 4.8, about 4.9, about 5.0, about 5.1, about 5.2, about 5.4, about 5.5, or about 5.6. In some cases, the composition comprises an aqueous solution of imatinib or derivative thereof, and has a pH of 3 to 7.

In some cases, a composition provided herein has a pH of 7 to 8, such as about 7.0, about 7.2, about 7.4, about 7.6, about 7.8, or about 8.0. In some cases, the composition comprises an aqueous suspension of imatinib or derivative thereof, and has a pH of 3 to 8.

In some cases, a composition provided herein does not comprise imatinib mesylate. In some cases, a composition provided herein has a substantially low amount of imatinib mesylate, for instance, less than 1 mg/mL, less than 0.5 mg/mL, less than 0.4 mg/mL, less than 0.3 mg/mL, less than 0.2 mg/mL, less than 0.1 mg/mL, less than 0.075 mg/mL, less than 0.05 mg/mL, less than 0.025 mg/mL, less than 0.01 mg/mL, less than 0.0075 mg/mL, less than 0.005 mg/mL, less than 0.0025 mg/mL, less than 0.001 mg/mL, less than 0.00075 mg/mL, less than 0.0005 mg/mL, less than 0.00025 mg/mL, or less than 0.0001 mg/mL imatinib mesylate, or less. In some cases, the composition has less than less than 0.2%, less than 0.15%, less than 0.1%, less than 0.075%, less than 0.05%, less than 0.025%, less than 0.02%, less than 0.015%, less than 0.01%, less than 0.0075%, less than 0.005%, less than 0.0025%, less than 0.002%, less than 0.0015%, or less than 0.001% imatinib mesylate, or even less in total amount of imatinib contained in the composition.

In some cases, the composition provided herein comprises an aqueous solution of imatinib or a derivative thereof. The term “solution,” as used herein, can refer to a homogenous mixture of one or more solutes dissolved in a solvent. The term “solvent” can refer to the substance in which a solute dissolves to produce the homogeneous mixture, and the term “solute” can refer to the substance that dissolves in a solvent to produce the homogeneous mixture. As used herein, the term “aqueous solution” can refer to a solution in which one of the one or more solvents is water.

In some cases, the composition provided herein is an aqueous suspension of imatinib or a derivative thereof. The term “suspension,” as used herein, can refer to a heterogenous mixture in which at least some of the solute particles do not dissolve, and remain as solid particles distributed throughout the bulk of the solvent. A suspension disclosed herein can have some of the solute (e.g., imatinib or a derivative thereof) dissolved in the solvent (e.g., water) while the remainder suspended in water. The term “aqueous suspension,” as used herein, can refer to a suspension in which one of the one or more solvents is water. A suspension provided herein may be used for therapeutic treatment and may be prepared (e.g., mixing the components and suspending the undissolved imatinib or derivative thereof in the water-based solution) immediately prior to the therapeutic use.

### Imatinib

In some aspects, a pharmaceutical composition disclosed herein comprises imatinib or a derivative thereof. In some aspects of the present disclosure, the pharmaceutical composition comprises a pharmaceutical agent for treating pulmonary diseases. In some embodiments, the pharmaceutical agent for treating pulmonary diseases is imatinib or a pharmaceutically acceptable salt thereof. In some embodiments, the pharmaceutically active ingredient in the composition (e.g., pharmaceutical composition or formulation) is imatinib free base. In some embodiments, the pharmaceutically active ingredient, e.g., the pharmaceutically acceptable salt of imatinib, comprises imatinib mesylate. In some embodiments, the pharmaceutical agent for treating pulmonary diseases is an imatinib derivative (e.g., Nilotinib, Sorafenib, Dasatinib) or a pharmaceutically acceptable salt thereof. Exemplary imatinib derivatives can include those described in Skobridis K et al. ChemMedChem. 2010 January; 5(1):130-9, A. Mortlock et al. Comprehensive Medicinal Chemistry II, Volume 7, 2007, Pages 183-220, and Musumeci F et al. Expert Opin Ther Pat. 2015; 25(12):1411-21, all of which are incorporated herein in its entirety.

The chemical structure of imatinib is shown in Compound I.

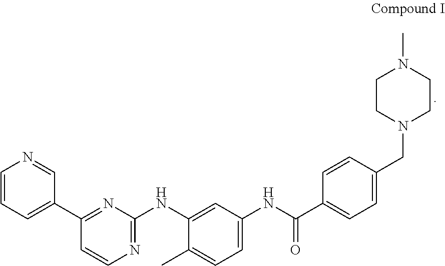 [SMILES: Cc1ccc(NC(=O)c2ccc(CN3CCN(C)CC3)cc2)cc1Nc1nccc(-c2cccnc2)n1]

The disclosure provides the use of pharmaceutically-acceptable salts of any therapeutic compound described herein. Pharmaceutically-acceptable salts include, for example, acid-addition salts and base-addition salts. The acid that is added to the compound to form an acid-addition salt can be an organic acid or an inorganic acid. A base that is added to the compound to form a base-addition salt can be an organic base or an inorganic base. In some embodiments, a pharmaceutically-acceptable salt is a metal salt. In some embodiments, a pharmaceutically-acceptable salt is an ammonium salt.

Metal salts can arise from the addition of an inorganic base to a compound of the disclosure. The inorganic base consists of a metal cation paired with a basic counterion, such as, for example, hydroxide, carbonate, bicarbonate, or phosphate. The metal can be an alkali metal, alkaline earth metal, transition metal, or main group metal. In some embodiments, the metal is lithium, sodium, potassium, cesium, cerium, magnesium, manganese, iron, calcium, strontium, cobalt, titanium, aluminum, copper, cadmium, or zinc.

In some embodiments, a metal salt is a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, or a zinc salt.

Ammonium salts can arise from the addition of ammonia or an organic amine to a compound of the present disclosure. In some embodiments, the organic amine is triethyl amine, diisopropyl amine, ethanol amine, diethanol amine, triethanol amine, morpholine, N-methylmorpholine, piperidine, N-methylpiperidine, N-ethylpiperidine, dibenzylamine, piperazine, pyridine, pyrazole, imidazole, or pyrazine.

In some embodiments, an ammonium salt is a triethyl amine salt, a trimethyl amine salt, a diisopropyl amine salt, an ethanol amine salt, a diethanol amine salt, a triethanol amine salt, a morpholine salt, an N-methylmorpholine salt, a piperidine salt, an N-methylpiperidine salt, an N-ethylpiperidine salt, a dibenzylamine salt, a piperazine salt, a pyridine salt, a pyrazole salt, a pyridazine salt, a pyrimidine salt, an imidazole salt, or a pyrazine salt.

Acid addition salts can arise from the addition of an acid to a compound of the present disclosure. In some embodiments, the acid is organic. In some embodiments, the acid is inorganic. In some embodiments, the acid is hydrochloric acid, hydrobromic acid, hydroiodic acid, nitric acid, nitrous acid, sulfuric acid, sulfurous acid, a phosphoric acid, isonicotinic acid, lactic acid, salicylic acid, tartaric acid, ascorbic acid, gentisic acid, gluconic acid, glucuronic acid, saccharic acid, formic acid, benzoic acid, glutamic acid, pantothenic acid, acetic acid, trifluoroacetic acid, mandelic acid, cinnamic acid, aspartic acid, stearic acid, palmitic acid, glycolic acid, propionic acid, butyric acid, fumaric acid, succinic acid, methanesulfonic acid, ethanesulfonic acid, benzenesulfonic acid, p-toluenesulfonic acid, citric acid, oxalic acid, or maleic acid.

In some embodiments, the salt is a hydrochloride salt, a hydrobromide salt, a hydroiodide salt, a nitrate salt, a nitrite salt, a sulfate salt, a sulfite salt, a phosphate salt, isonicotinate salt, a lactate salt, a salicylate salt, a tartrate salt, an ascorbate salt, a gentisate salt, a gluconate salt, a glucuronate salt, a saccharate salt, a formate salt, a benzoate salt, a glutamate salt, a pantothenate salt, an acetate salt, a trifluoroacetate salt, a mandelate salt, a cinnamate salt, an aspartate salt, a stearate salt, a palmitate salt, a glycolate salt, a propionate salt, a butyrate salt, a fumarate salt, a succinate salt, a methanesulfonate salt, an ethanesulfonate salt, a benzenesulfonate salt, a p-toluenesulfonate salt, a citrate salt, an oxalate salt, or a maleate salt.

In some embodiments, the salt is a salt prepared by reaction of a compound described herein with a mineral acid, organic acid, or inorganic base, such salts including acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate, metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate, undeconate, and xylenesulfonate.

In some embodiments, the salt is a salt formed by reacting a free base form of a compound disclosed herein with a pharmaceutically acceptable inorganic or organic acid, including, but not limited to, inorganic acids such as hydrochloric acid, hydrobromic acid, sulfuric acid, nitric acid, phosphoric acid, metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid.

In some embodiments, the salt is a salt formed by reacting a free base form of a compound disclosed herein with a suitable base, such as the hydroxide, carbonate, bicarbonate, or sulfate of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts, and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine, and the like. It should be understood that the compounds described herein also include the quaternization of any basic nitrogen-containing groups they contain. In some embodiments, water or oil-soluble or dispersible products are obtained by such quaternization.

In some embodiments, the pharmaceutically acceptable salt of imatinib is an acetate salt, formate salt, citrate salt, phosphate salt, maleate salt, fumarate salt, tartrate salt, malonate salt, lactic salt, or succinate salt.

### Solubility Enhancer

In some aspects of the present disclosure, a solubility enhancer described herein provides the pharmaceutical agent for treating pulmonary diseases, e.g., imatinib or a derivative thereof, or a pharmaceutically acceptable salt thereof, increased solubility in an aqueous solution. In some aspects of the present disclosure, the solubility enhancer is a cyclodextrin. In some aspects of the present disclosure, the solubility enhancer is a lipid or a fatty acid. In some aspects of the present disclosure, the solubility enhancer is a co-solvent. In some aspects of the present disclosure, the solubility enhancer is an organic acid or generally recognized as safe (GRAS) excipient acid. In some aspects of the present disclosure, the solubility enhancer is a surfactant, such as Tween, sodium lauryl sulfate (SLS), or dipalmitoylphosphatidylcholine (DPPC).

A lipid or fatty acid in a composition (e.g., pharmaceutical composition or formulation) provided herein can be, for example, polyethoxylated castor oil, phospholipids, glycolipids, ganglioside GM1, sphingomyelin, phosphatidic acid, cardiolipin; a lipid bearing polymer chain such as polyethylene glycol (PEG, PEG300, PEG400), propylene glycol (PG), chitin, hyaluronic acid, and polyvinylpyrrolidone; lipids bearing sulfonated monosaccharides, a lipid-bearing sulfonated disaccharide, a lipid bearing sulfonated polysaccharide; a fatty acid such as palmitic acid, stearic acid, and oleic acid; cholesterol, cholesterol esters, and cholesterol hemisuccinate. In some aspects of the present disclosure, the lipid is polymeric. In some aspects of the present disclosure, the polymeric lipid is polyvinylpyrrolidone (PVP). In some aspects of the present disclosure, the polymeric lipid is polyethylene glycol (PEG). In some aspects of the present disclosure, the lipid is a sulfonated polysaccharide. In some aspects of the present disclosure, the lipid is a fatty acid. In some aspects of the present disclosure, the fatty acid is steric or oleic acid. In some aspects of the present disclosure, the fatty acid a phospholipid. In some embodiments, the phospholipid is lecirhin or 1,2-dipalmitoyl-sn-glycerol-3-phosphocholine (DPPC).

The co-solvent in a composition (e.g., pharmaceutical composition or formulation) provided herein can be, for example, ethylene glycol or ethanol. The organic acid in a composition (e.g., pharmaceutical composition or formulation) provided herein can be, for example, acetic acid, acid modified starch, aconitic acid, adipic acid, hexanedioic acid, L-ascorbic acid, benzoic acid, caprylic acid, octanoic acid, cholic acid, citric acid, desoxycholic acid, erythorbic acid (D-isoascorbic acid), formic acid, L-glutamic acid, L-glutamic acid hydrochloride, glycocholic acid, hydrochloric acid, iron naphthenate, iron tallate, D(−)-lactic acid, lactic acid, L(+)-lactic acid, linoleic acid, malic acid, L-malic acid, niacin (nicotinic acid), oleic acid, pectin, pectinic acid, phosphoric acid, L(+)-potassium acid tartrate, propionic acid, acid hydrolyzed proteins, sodium acid pyrophosphate, acidic sodium aluminum phosphate, sorbic acid, stearic acid, succinic acid, sulfamic acid, sulfuric acid, tannic acid, L(+)-tartaric acid, taurocholic acid, or thiodipropionic acid. The GRAS excipient acid in a composition (e.g., pharmaceutical composition or formulation) provided herein can be, for example, acetic acid, formic acid, citrate, tartrate, maleate, fumarate, tartrate, malonate, lactic, or succinate.

### Cyclodextrin

In some aspects of the present disclosure, a cyclodextrin is used as a solubility enhancer of imatinib or a derivative thereof. In some aspects of the present disclosure, a cyclodextrin is used as a solubility enhancer of imatinib free base. In some aspects of the present disclosure, a cyclodextrin is used as a solubility enhancer of a salt of imatinib. Cyclodextrins are cyclic carbohydrates derived from starch. The unmodified cyclodextrins differ by the number of glucopyranose units joined together in the cylindrical structure. The parent cyclodextrins contain 6, 7, or 8 glucopyranose units and are referred to as α-, β-, and γ-cyclodextrin respectively. Each cyclodextrin subunit can have secondary hydroxyl groups at the 2 and 3 positions and a primary hydroxyl group at the 6-position. The cyclodextrins can be pictured as hollow truncated cones with hydrophilic exterior surfaces and hydrophobic interior cavities. In aqueous solutions, these hydrophobic cavities can sequester hydrophobic organic compounds that can fit all or part of their structure into these cavities. This process, known as inclusion complexation, can result in increased apparent aqueous solubility and stability for the complexed drug.

Cyclodextrins suitable for use in a composition (e.g., a pharmaceutical composition) provided herein can include, but not limited to, α-cyclodextrin (αCD), β-cyclodextrin (βCD), γ-cyclodextrin (γCD), derivatized α-cyclodextrins, derivatized β-cyclodextrins, and derivatized γ-cyclodextrins. Non-limiting examples of cyclodextrin that can be used in the subject composition (e.g., pharmaceutical composition or formulation) include α-cyclodextrin, β-cyclodextrin, γ-cyclodextrin, hydroxypropyl-β-cyclodextrin (HPβCD), hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin dimaltosyl-β-cyclodextrin, methyl-β-cyclodextrin (MβCD), succinyl-α-cyclodextrin (SαCD), succinyl-R-cyclodextrin (SβCD), succinyl-γ-cyclodextrin (SγCD), 6A-amino-6A-deoxy-N-(3-hydroxypropyl)-β-cyclodextrin, sulfobutylether-α-cyclodextrin (SBEαCD) sulfobutylether-β-cyclodextrin (SBEβCD), sulfobutylether-γ-cyclodextrin, sulfoalkylether-β-cyclodextrins, sulfoalkylether-γ-cyclodextrins, carboxymethyl-α-cyclodextrin (CMαCD), carboxymethyl-β-cyclodextrin (CMβCD), carboxymethyl-γ-cyclodextrin (CM-γCD), 2-carboxyethyl-α-cyclodextrin (CEαCD), 2-carboxyethyl-β-cyclodextrin (CEβCD), 2-carboxyethyl-γ-cyclodextrin (CEγCD), phosphate-α-cyclodextrin (PαCD), phosphate-β-cyclodextrin (PβCD), and phosphate-γ-cyclodextrin (PγCD). In some embodiments, the composition (e.g., the pharmaceutical composition) comprises hydroxypropyl-β-cyclodextrin (HPβCD). In some embodiments, the composition (e.g., the pharmaceutical composition) comprises more than one species of cyclodextrins, such as, 2, 3, 4, 5, 6, 7, 8, 9, 10 or more different species of cyclodextrins. In some embodiments, the composition (e.g., the pharmaceutical composition) comprises HPβCD and one or more other cyclodextrins, such as, 1, 2, 3, 4, 5, 6, 7, 8, 9, 10 or more other different species of cyclodextrins. In some cases, the cyclodextrin is selected from the group consisting of: α-cyclodextrin, 3-cyclodextrin, γ-cyclodextrin, derivatized α-cyclodextrins, derivatized 3-cyclodextrins, and derivatized γ-cyclodextrins. In some cases, the cyclodextrin is selected from the group consisting of: α-cyclodextrin, 3-cyclodextrin, γ-cyclodextrin, hydroxypropyl-3-cyclodextrin, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin dimaltosyl-β-cyclodextrin, succinyl-β-cyclodextrin, 6A-amino-6A-deoxy-N-(3-hydroxypropyl)-β-cyclodextrin, sulfobutylether-β-cyclodextrin, sulfobutylether-γ-cyclodextrin, sulfoalkylether-β-cyclodextrins, and sulfoalkylether-γ-cyclodextrins.

In some cases, a salt of cyclodextrin is used to prepare the composition disclosed herein. For instance, a metal salt of cyclodextrin can be used, such as, for example, a sodium salt, a calcium salt, a magnesium salt, an iron salt, a chromium salt, a copper salt, or a zinc salt. Alternatively or additionally, an amino acid salt of cyclodextrin can be used, such as, for example, a lysine, an arginine, or a histidine salts. In some cases, the aqueous solution or suspension disclosed herein comprises a salt of a cyclodextrin, for instance, a salt of an anionic cyclodextrin, e.g., a salt of sulfobutylether-β-cyclodextrin. In some cases, the aqueous solution or suspension disclosed herein comprises sulfobutylether-β-cyclodextrin sodium.

In some embodiments, the concentration of the cyclodextrin contributes to the viscosity of the solution, which can reduce the nebulization efficiency (or rate) of the solution. For instance, in some cases, the higher the concentration of the cyclodextrin is, the higher viscosity of the solution is. In some cases, the concentration of the cyclodextrin in the composition is controlled so that the viscosity of the solution is not higher than a reference value, such as about 0.1 centipoise (cP), 0.2 cP, 0.3 cP, 0.4 cP, 0.5 cP, 0.6 cP, 0.7 cP, 0.8 cP, 0.9 cP, 1.0 cP, 1.1 cP, 1.2 cP, 1.3 cP, 1.4 cP, 1.5 cP, 1.6 cP, 1.7 cP, 1.8 cP, 1.9 cP, 2.0 cP, 2.1 cP, 2.2 cP, 2.3 cP, 2.4 cP, 2.5 cP, 2.6 cP, 2.7 cP, 2.8 cP, 2.9 cP, 3.0 cP, 3.2 cP, 3.4 cP, 3.5 cP, 3.6 cP, 3.8 cP, 4.0 cP, 4.2 cP, 4.4 cP, 4.6 cP, 4.8 cP, 5.0 cP, 5.2 cP, 5.4 cP, 5.6 cP, 5.8 cP, 6.0 cP, 6.2 cP, 6.4 cP, 6.6 cP, 6.8 cP, 7.0 cP, 7.2 cP, 7.4 cP, 7.6 cP, 7.8 cP, 8.0 cP, 8.2 cP, 8.4 cP, 8.6 cP, 8.8 cP, 9.0 cP, 9.2 cP, 9.4 cP, 9.6 cP, 9.8 cP, or 10.0 cP. In some cases, the concentration of the cyclodextrin in the composition (e.g., pharmaceutical composition or formulation) is at most about 2%, 5%, 8%, 10%, 12%, 15%, 18%, 20%, 20.5%, 21%, 21.5%, 22%, 22.5%, 23%, 23.5%, 24%, 24.5%, 25%, 25.5%, 26%, 26.5%, 27%, 27.5%, 28%, 28.5%, 29%, 29.5%, 30%, 31%, 32%, 33%, 34%, 35%, 38%, 40%, 45%, 50%, 55%, 60%, 65%, 70%, 75%, or 80% (w/v) of the solution.

Without wishing to be bound to a certain theory, imatinib or a derivative thereof (e.g., imatinib free base) in acidic conditions can be protonated, leading to an increase in the solubility of imatinib free base. Without wishing to be bound to a certain theory, cyclodextrin can increase the solubility of imatinib or a derivative thereof (e.g., imatinib free base) by stabilizing the positively charged piperazine ring in acidic conditions. Without wishing to be bound to a certain theory, cyclodextrins that are anionic can increase the stabilization of the positively charged imatinib or a derivative thereof (e.g., positively charged imatinib free base) and increase solubility. In some embodiments, cyclodextrins that are anionic are in the compositions and methods disclosed herein, which can be, for example, succinyl-α-cyclodextrin (SαCD), succinyl-β-cyclodextrin (SβCD), succinyl-γ-cyclodextrin (SγCD), sulfobutylether-α-cyclodextrin (SBEαCD) sulfobutylether-β-cyclodextrin (SBEβCD), sulfobutylether-γ-cyclodextrin (SBEγCD), carboxymethyl-α-cyclodextrin (CMαCD), carboxymethyl-R-cyclodextrin (CMβCD), carboxymethyl-γ-cyclodextrin (CMγCD), 2-carboxyethyl-α-cyclodextrin (CEαCD), 2-carboxyethyl-β-cyclodextrin (CEβCD), 2-carboxyethyl-γ-cyclodextrin (CEγCD), phosphate-α-cyclodextrin (PαCD), phosphate-β-cyclodextrin (PβCD), and γ-cyclodextrin (PγCD).

In some aspects of the present disclosure, cyclodextrin, e.g., SBEβCD, is used as a solubility enhancer of imatinib or a derivative thereof. In some aspects of the present disclosure, cyclodextrin, e.g., SBEβCD, is used as a solubility enhancer of imatinib free base. In some aspects of the present disclosure, cyclodextrin, e.g., SBEβCD, is used as a solubility enhancer of a salt of imatinib. In some cases, the concentration of the cyclodextrin, e.g., SBEβCD, in the composition (e.g., the pharmaceutical composition) is at most about 2%, 5%, 8%, 10%, 12%, 15%, 18%, 20%, 20.5%, 21%, 21.5%, 22%, 22.5%, 23%, 23.5%, 24%, 24.5%, 25%, 25.5%, 26%, 26.5%, 27%, 27.5%, 28%, 28.5%, 29%, 29.5%, 30%, 31%, 32%, 33%, 34%, 35%, 38%, 40%, 45%, 50%, 55%, 60%, 65%, 70%, 75%, or 80% (w/v) of the solution at a pH from about 3 to about 8. In some cases, the concentration of the cyclodextrin, e.g., SBEβCD, in the composition (e.g., the pharmaceutical composition) is at most about 20% (w/v) of the solution at a pH from about 3 to about 8. In some embodiments, the concentration of cyclodextrin, e.g., SBEβCD, in the composition is at about 2% to about 80% the solution at a pH of about 5. In some embodiments, the concentration of cyclodextrin, e.g., SBEβCD, in the composition is at about 2% to about 60% the solution at a pH of about 5. In some embodiments, the concentration of cyclodextrin, e.g., SBEβCD, in the composition is at about 2% to about 50% the solution at a pH of about 5. In some embodiments, the concentration of cyclodextrin, e.g., SBEβCD, in the composition is at about 5% to about 45% the solution at a pH of about 5. In some embodiments, the concentration of cyclodextrin, e.g., SBEβCD, in the composition is at about 10% to about 20% the solution at a pH of about 4. In some embodiments, the concentration of cyclodextrin, e.g., SBEβCD, in the composition is at about 2% to about 80% the solution at a pH of about 4. In some embodiments, the concentration of cyclodextrin, e.g., SBEβCD, in the composition is at about 2% to about 60% the solution at a pH of about 4. In some embodiments, the concentration of cyclodextrin, e.g., SBEβCD, in the composition is at about 2% to about 50% the solution at a pH of about 4. In some embodiments, the concentration of cyclodextrin, e.g., SBEβCD, in the composition is at about 5% to about 45% the solution at a pH of about 4. In some embodiments, the concentration of cyclodextrin, e.g., SBEβCD, in the composition is at about 10% to about 20% the solution at a pH of about 4. In some embodiments, the concentration of cyclodextrin, e.g., SBEβCD, in the composition (e.g., pharmaceutical composition or formulation) is at about 2% to about 80% the solution at a pH of about 3. In some embodiments, the concentration of cyclodextrin, e.g., SBEβCD, in the composition (e.g., pharmaceutical composition or formulation) is at about 2% to about 60% the solution at a pH of about 3. In some embodiments, the concentration of cyclodextrin, e.g., SBEβCD, in the composition is at about 2% to about 50% the solution at a pH of about 3. In some embodiments, the concentration of cyclodextrin, e.g., SBEβCD, in the composition is at about 5% to about 45% the solution at a pH of about 3. In some embodiments, the concentration of cyclodextrin, e.g., SBEβCD, in the composition is at about 10% to about 20% the solution at a pH of about 3. In some embodiments, the concentration of cyclodextrin, e.g., SBEβCD, in the composition is at about 2% to about 80% the solution at a pH of about 6. In some embodiments, the concentration of cyclodextrin, e.g., SBEβCD, in the composition is at about 2% to about 60% the solution at a pH of about 6. In some embodiments, the concentration of cyclodextrin, e.g., SBEβCD, in the composition is at about 2% to about 50% the solution at a pH of about 6. In some embodiments, the concentration of cyclodextrin, e.g., SBEβCD, in the composition is at about 5% to about 45% the solution at a pH of about 6. In some embodiments, the concentration of cyclodextrin, e.g., SBEβCD, in the composition is at about 10% to about 20% the solution at a pH of about 6.

In some cases, the concentration of the cyclodextrin, e.g., SBEβCD, in the composition (e.g., the pharmaceutical composition) is at most about 2%, 5%, 8%, 10%, 12%, 15%, 18%, 20%, 20.5%, 21%, 21.5%, 22%, 22.5%, 23%, 23.5%, 24%, 24.5%, 25%, 25.5%, 26%, 26.5%, 27%, 27.5%, 28%, 28.5%, 29%, 29.5%, 30%, 31%, 32%, 33%, 34%, 35%, 38%, 40%, 45%, 50%, 55%, 60%, 65%, 70%, 75%, or 80% (w/v) of the solution or suspension at a pH from about 3 to about 8 and the concentration of imatinib dissolved is about 0.0001 mg/mL to about 500 mg/mL. In some cases, the concentration of the cyclodextrin, e.g., SBEβCD, in the composition is at most about 2% to about 80% (w/v) of the solution at a pH at about 5 and the concentration of imatinib dissolved is about 1 mg/mL to about 200 mg/mL.

In some cases, the formulation disclosed herein, e.g. a formulation containing imatinib free base and cyclodextrin e.g., SBEβCD, at an acidic pH range can lead to low systemic absorption of imatinib and a high lung residence time for imatinib. In some cases, the formulation disclosed herein, e.g. a formulation containing imatinib free base and cyclodextrin e.g., SBEβCD, at an acidic pH range can lead to highly preferential retention of the pharmaceutically active ingredient, e.g., imatinib or derivative thereof, in the lungs as compared to in plasma. Without wishing to be bound to a certain theory, minimizing systemic exposure can lead to decreasing systemic side effects. In some cases, an acidic solution of imatinib or derivative thereof, e.g. containing imatinib free base and cyclodextrin (e.g., SBEβCD), when administered to the lungs results in precipitation of imatinib or its salt upon coming in contact with the lung lining fluid.

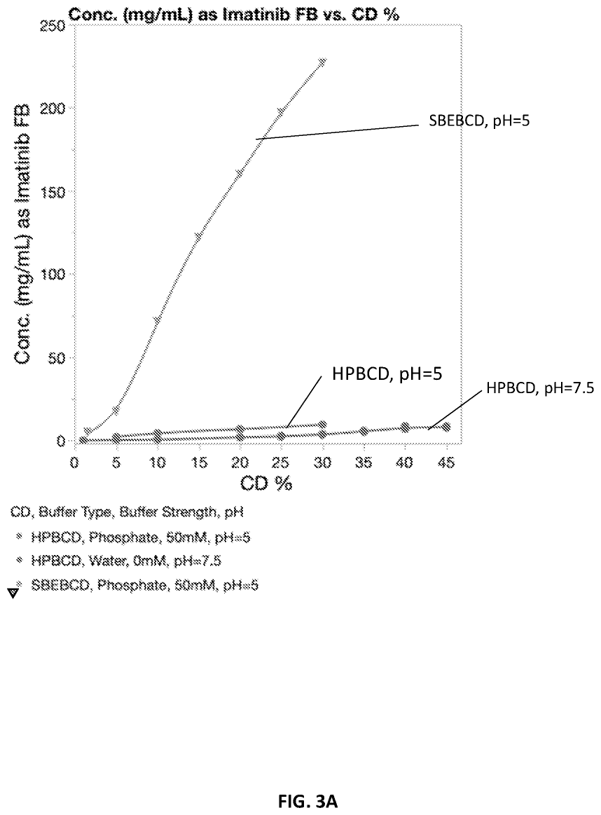

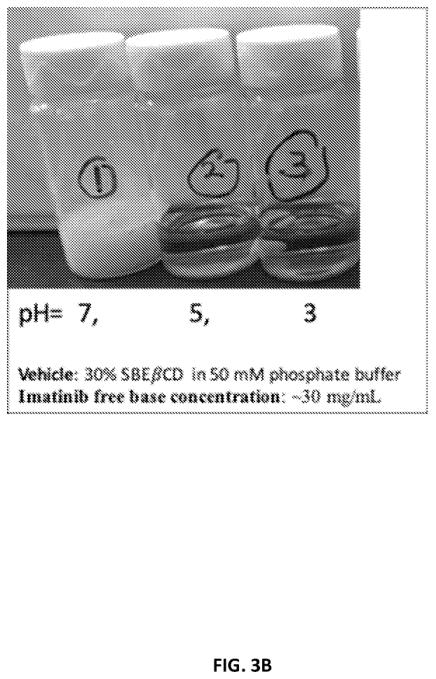

Solubility of imatinib or derivative thereof (e.g., imatinib free base) in an acidic solution according to some embodiments of the present disclosure can be negatively correlated with the pH of the solution, for instance, the higher the pH of the solution is, the lower the possibility that imatinib free base is dissolved in the solution is (e.g., as shown in Examples 4 and 5 and FIGS. 2, 3A, and 3B). Additionally or alternatively, solubility of imatinib or derivative thereof (e.g., imatinib free base) in an acidic solution containing cyclodextrin (e.g., SBE®CD) according to some embodiments of the present disclosure can be positively correlated with the concentration of cyclodextrin in the solution, for instance, the higher the concentration of SBE®CD is, the higher the possibility that imatinib freebase is dissolved in the solution is (e.g., as shown in Examples 5-7). Without wishing to be bound to a certain theory, in some cases, the lung lining fluid acts as a buffer that increases the pH to precipitate imatinib or a derivative thereof delivered in the formulation. Without wishing to be bound to a certain theory, in some cases, the lung lining fluid acts as a diluent to precipitate imatinib or a derivative thereof delivered in the formulation. Without wishing to be bound to a certain theory, in some cases, the lung lining fluid acts as a buffer increasing the pH and as a diluent to precipitate imatinib or a derivative thereof delivered in the formulation. Without wishing to be bound to a certain theory, solid precipitated imatinib can lead to extended release of imatinib to the lung tissue over time, lower systemic absorption, and longer lung residence time.

In some cases, within 10 min, 20 min, 30 min, 1 hour, 2 hours, 3 hours, 5 hours, or 10 hours after inhalational administration of the exemplary composition disclosed herein, concentration of imatinib or derivative thereof in systemic circulation, e.g., concentration of imatinib measured in blood samples collected from large blood vessels or heart, is lower than concentration of imatinib in the lung tissue, e.g., concentration of imatinib measured in samples collected from the lung. For instance, within 10 min, 20 min, 30 min, 1 hour, 2 hours, 3 hours, 5 hours, or 10 hours after inhalational administration, the concentration ratio of imatinib measured in blood sample compared to samples collected from the lungs is less than 1, such as about 0.0001 to about 0.9, about 0.001 to about 0.9, about 0.01 to about 0.9, or about 0.1 to about 0.9. In some embodiments, the concentration ratio is about 0.0001, about 0.0005, about 0.001, about 0.005, about 0.008, about 0.01, about 0.015, about 0.02, about 0.03, about 0.04, about 0.05, bout 0.06, about 0.07, about 0.08, about 0.09, about 0.1, about 0.15, about 0.2, about 0.25, about 0.3, about 0.35, about 0.4, about 0.45, about 0.5, about 0.55, about 0.6, about 0.65, about 0.7, about 0.75, about 0.8, about 0.85, or about 0.9.

In some cases, after inhalational administration of the exemplary imatinib composition disclosed herein, concentration of imatinib in the lung tissues, e.g., concentration of imatinib measured in samples collected from the lung, decreases at a lower speed as compared to the concentration of imatinib in systemic circulation, e.g., concentration of imatinib measured in blood samples collected from large blood vessels or heart.

### pH Buffers

In some aspects of the present disclosure, the compositions provided herein further comprises a pH buffer. In some cases, the pH buffer is an organic acid salt, including but not limited to, of citric acid, lactic acid, ascorbic acid, gluconic acid, carbonic acid, tartaric acid, succinic acid, acetic acid, or phthalic acid, Tris, tromethamine hydrochloride, or a phosphate buffer. In some embodiments, the composition comprises more than one pH buffer.

In some embodiments, the pH buffers are present in the compositions (e.g., pharmaceutical compositions) described herein to provide the aqueous solution a pH of about 3 to about 7. In some embodiments, the pH buffers are present in the compositions (e.g., pharmaceutical compositions) described herein to provide the aqueous suspension a pH of about 3 to about 8. In some cases, the pH of the composition (e.g., pharmaceutical composition or formulation) is about 3, about 4, about 5, about 6, about 7, or about 8 In some embodiments, the pH buffers are present in the compositions described herein to provide the aqueous solution a pH of the aqueous solution is from 3 to 6, from 4 to 6, from 4.5 to 5.5, from 5 to 6, from 4 to 7, from 5 to 7, or from 6 to 7.

In some embodiments, the pH buffer is present in the aqueous composition at about 0.001 mg/mL to about 100 mg/mL, for example between about 0.1 mg/mL to about 100 mg/mL, about 0.5 mg/mL to about 50 mg/mL, about 0.5 mg/mL to about 20 mg/mL, about 0.5 mg/mL to about 10 mg/mL, about 0.5 mg/mL to 5 mg/mL, or about 1 mg/mL to about 5 mg/mL. In some embodiments, the pH buffer is present in the aqueous composition at about 0.1 mM to about 500 mM, for example from about 1 mM to 500 mM, 1 mM to 200 mM, 1 mM to 100 mM, 1 mM to 80 mM, 1 mM to 50 mM, 1 mM to 25 mM, 1 mM to 10 mM, 1 mM to 5 mM, 5 mM to 200 mM, 5 mM to 100 mM, 5 mM to 80 mM, 5 mM to 50 mM, 5 mM to 25 mM, 5 mM to 10 mM, 20 mM to 200 mM, 20 mM to 100 mM, 20 mM to 80 mM, or 20 mM to 50 mM.

### Pharmaceutical Acceptable Excipients

In one aspect, provided herein are formulations for treatment of a pulmonary disease. The formulations can include the compositions provided herein and a pharmaceutically acceptable carrier, excipient, diluent, or any other suitable component for the intended administration routes, such as oral or nasal inhalation. Examples of pharmaceutically acceptable excipients include, but are not limited to, lipids, metal ions, surfactants, amino acids, carbohydrates, buffers, salts, polymers, sweeteners, and the like, and combinations thereof.

Examples of carbohydrates include, but are not limited to, monosaccharides, disaccharides, and polysaccharides. For example, monosaccharides such as dextrose (anhydrous and monohydrate), galactose, mannitol, D-mannose, sorbitol, sorbose, and the like; disaccharides such as lactose, maltose, sucrose, trehalose, and the like; trisaccharides such as raffinose and the like; and other carbohydrates such as starches (hydroxyethylstarch), and maltodextrins.

Non-limiting examples of lipids include phospholipids, glycolipids, ganglioside GM1, sphingomyelin, phosphatidic acid, cardiolipin; lipids bearing polymer chains such as polyethylene glycol, chitin, hyaluronic acid, or polyvinylpyrrolidone; lipids bearing sulfonated mono-, di-, and polysaccharides; fatty acids such as palmitic acid, stearic acid, and oleic acid; cholesterol, cholesterol esters, and cholesterol hemisuccinate.

In some cases, the phospholipid comprises a saturated phospholipid, such as one or more phosphatidylcholines. Exemplary acyl chain lengths are 16:0 and 18:0 (e.g., palmitoyl and stearoyl). The phospholipid content can be determined by the active agent activity, the mode of delivery, and other factors.

Phospholipids from both natural and synthetic sources can be used in varying amounts. When phospholipids are present, the amount is typically sufficient to coat the active agent(s) with at least a single molecular layer of phospholipid. In general, the phospholipid content ranges from about 5 wt % to about 99.9 wt %, such as about 20 wt % to about 80 wt %.

Generally, compatible phospholipids can comprise those that have a gel to liquid crystal phase transition greater than about 40° C., such as greater than about 60° C., or greater than about 80° C. The incorporated phospholipids can be relatively long chain (e.g., C₁₆-C₂₂) saturated lipids. Exemplary phospholipids useful in the present disclosure include, but are not limited to, phosphoglycerides such as dipalmitoylphosphatidylcholine, distearoylphosphatidylcholine, diarachidoylphosphatidylcholine, dibehenoylphosphatidylcholine, diphosphatidyl glycerols, short-chain phosphatidylcholines, hydrogenated phosphatidylcholine, E-100-3 (available from Lipoid KG, Ludwigshafen, Germany), long-chain saturated phosphatidylethanolamines, long-chain saturated phosphatidylserines, long-chain saturated phosphatidylglycerols, long-chain saturated phosphatidylinositols, phosphatidic acid, phosphatidylinositol, and sphingomyelin.

Examples of metal ions include, but are not limited to, divalent cations, including calcium, magnesium, zinc, iron, and the like. For instance, when phospholipids are used, the pharmaceutical composition can also comprise a polyvalent cation, as disclosed in U.S. Pat. Nos. 8,709,484 and 7,871,598, which are incorporated herein by reference in their entireties. The polyvalent cation can be present in an amount effective to increase the melting temperature (Tₘ) of the phospholipid such that the pharmaceutical composition exhibits a Tₘ which is greater than its storage temperature (Tₘ) by at least about 20° C., such as at least about 40° C. The molar ratio of polyvalent cation to phospholipid can be at least about 0.05:1, such as about 0.05:1 to about 2.0:1 or about 0.25:1 to about 1.0:1. An example of the molar ratio of polyvalent cation: phospholipid is about 0.50:1. When the polyvalent cation is calcium, it can be in the form of calcium chloride. Although metal ion, such as calcium, is often included with phospholipid, none is required.

The pharmaceutical composition can include one or more surfactants. For instance, one or more surfactants can be in the liquid phase with one or more being associated with solid droplets or droplets of the composition. By “associated with” it is meant that the pharmaceutical compositions can incorporate, adsorb, absorb, be coated with, or be formed by the surfactant. Surfactants include, but are not limited to, fluorinated and nonfluorinated compounds, such as saturated and unsaturated lipids, nonionic detergents, nonionic block copolymers, ionic surfactants, and combinations thereof. It should be emphasized that, in addition to the aforementioned surfactants, suitable fluorinated surfactants are compatible with the teachings herein and can be used to provide the desired preparations. In some aspects, the surfactant in the pharmaceutical composition described herein comprises Tween, sodium lauryl sulfate (SLS), or dipalmitoylphosphatidylcholine (DPPC). Without wishing to be bound to a certain theory, in some cases, the surfactant also serves as a solubility enhancer in the composition.

Examples of nonionic detergents include, but are not limited to, sorbitan esters including sorbitan trioleate (Span™ 85), sorbitan sesquioleate, sorbitan monooleate, sorbitan monolaurate, polyoxyethylene (20) sorbitan monolaurate, and polyoxyethylene (20) sorbitan monooleate, oleyl polyoxyethylene (2) ether, stearyl polyoxyethylene (2) ether, lauryl polyoxyethylene (4) ether, glycerol esters, and sucrose esters. Other suitable nonionic detergents can be easily identified using McCutcheon's Emulsifiers and Detergents (McPublishing Co., Glen Rock, N.J.), which is incorporated herein by reference in its entirety.

Examples of block copolymers include, but are not limited to, diblock and triblock copolymers of polyoxyethylene and polyoxypropylene, including poloxamer 188 (Pluronic™ F-68), poloxamer 407 (Pluronic™ F-127), and poloxamer 338. Examples of ionic surfactants include, but are not limited to, sodium sulfosuccinate, and fatty acid soaps. Examples of amino acids include, but are not limited to hydrophobic amino acids. Use of amino acids as pharmaceutically acceptable excipients is known in the art as disclosed in U.S. Pat. Nos. 6,123,936, 6,358,530, and 6,921,527, which are incorporated herein by reference in their entireties.

The pharmaceutical composition according to one or more embodiments of the disclosure may, if desired, contain a combination of pharmaceutical agent for treatment of pulmonary diseases (e.g., imatinib or a derivative thereof, e.g., imatinib free base or imatinib salt) and one or more additional active agents. Examples of additional active agents include, but are not limited to, agents that can be delivered through the lungs.

Additional active agents can comprise, for example, hypnotics and sedatives, psychic energizers, tranquilizers, respiratory drugs, anticonvulsants, muscle relaxants, antiparkinson agents (dopamine antagonists), analgesics, anti-inflammatories, antianxiety drugs (anxiolytics), appetite suppressants, antimigraine agents, muscle contractants, additional anti-infectives (antivirals, antifungals, vaccines) antiarthritics, antimalarials, antiemetics, antiepileptics, cytokines, growth factors, anti-cancer agents, antithrombotic agents, antihypertensives, cardiovascular drugs, antiarrhythmics, antioxidants, anti-asthma agents, hormonal agents including contraceptives, sympathomimetics, diuretics, lipid regulating agents, antiandrogenic agents, antiparasitic, anticoagulants, neoplastics, antineoplastics, hypoglycemics, nutritional agents and supplements, growth supplements, antienteritis agents, vaccines, antibodies, diagnostic agents, and contrasting agents. The additional active agent, when administered by inhalation, can act locally or systemically.

The additional active agent can fall into one of a number of structural classes, including but not limited to small molecules, peptides, polypeptides, proteins, polysaccharides, steroids, proteins capable of eliciting physiological effects, nucleotides, oligonucleotides, polynucleotides, fats, electrolytes, and the like.

Examples of additional active agents suitable for use in this disclosure include but are not limited to one or more of calcitonin, amphotericin B, erythropoietin (EPO), Factor VIII, Factor IX, ceredase, cerezyme, cyclosporin, granulocyte colony stimulating factor (GCSF), thrombopoietin (TPO), alpha-1 proteinase inhibitor, elcatonin, granulocyte macrophage colony stimulating factor (GMCSF), growth hormone, human growth hormone (HGH), growth hormone releasing hormone (GHRH), heparin, low molecular weight heparin (LMWH), interferon alpha, interferon beta, interferon gamma, interleukin-1 receptor, interleukin-2, interleukin-1 receptor antagonist, interleukin-3, interleukin-4, interleukin-6, luteinizing hormone releasing hormone (LHRH), factor IX, insulin, pro-insulin, insulin analogues (e.g., mono-acylated insulin as described in U.S. Pat. No. 5,922,675, which is incorporated herein by reference in its entirety), amylin, C-peptide, somatostatin, somatostatin analogs including octreotide, vasopressin, follicle stimulating hormone (FSH), insulin-like growth factor (IGF), insulintropin, macrophage colony stimulating factor (M-CSF), nerve growth factor (NGF), tissue growth factors, keratinocyte growth factor (KGF), glial growth factor (GGF), tumor necrosis factor (TNF), endothelial growth factors, parathyroid hormone (PTH), glucagon-like peptide thymosin alpha 1, IIb/IIa inhibitor, alpha-1 antitrypsin, phosphodiesterase (PDE) compounds, VLA-4 inhibitors, bisphosponates, respiratory syncytial virus antibody, cystic fibrosis transmembrane regulator (CFFR) gene, deoxyribonuclease (DNase), bactericidal/permeability increasing protein (BPI), anti-CMV antibody, 13-cis retinoic acid, oleandomycin, troleandomycin, roxithromycin, clarithromycin, davercin, azithromycin, flurithromycin, dirithromycin, josamycin, spiromycin, midecamycin, leucomycin, miocamycin, rokitamycin, andazithromycin, and swinolide A; fluoroquinolones such as ciprofloxacin, ofloxacin, levofloxacin, trovafloxacin, alatrofloxacin, moxifloxicin, norfloxacin, enoxacin, grepafloxacin, gatifloxacin, lomefloxacin, sparfloxacin, temafloxacin, pefloxacin, amifloxacin, fleroxacin, tosufloxacin, prulifloxacin, irloxacin, pazufloxacin, clinafloxacin, and sitafloxacin, teicoplanin, rampolanin, mideplanin, colistin, daptomycin, gramicidin, colistimethate, polymixins such as polymixin B, capreomycin, bacitracin, penems; penicillins including penicllinase-sensitive agents like penicillin G, penicillin V, penicillinase-resistant agents like methicillin, oxacillin, cloxacillin, dicloxacillin, floxacillin, nafcillin; gram negative microorganism active agents like ampicillin, amoxicillin, and hetacillin, cillin, and galampicillin; antipseudomonal penicillins like carbenicillin, ticarcillin, azlocillin, mezlocillin, and piperacillin; cephalosporins like cefpodoxime, cefprozil, ceftbuten, ceftizoxime, ceftriaxone, cephalothin, cephapirin, cephalexin, cephradrine, cefoxitin, cefamandole, cefazolin, cephaloridine, cefaclor, cefadroxil, cephaloglycin, cefuroxime, ceforanide, cefotaxime, cefatrizine, cephacetrile, cefepime, cefixime, cefonicid, cefoperazone, cefotetan, cefinetazole, ceftazidime, loracarbef, and moxalactam, monobactams like aztreonam; and carbapenems such as imipenem, meropenem, pentamidine isethiouate, lidocaine, metaproterenol sulfate, beclomethasone diprepionate, triamcinolone acetamide, budesonide acetonide, fluticasone, ipratropium bromide, flunisolide, cromolyn sodium, ergotamine tartrate and where applicable, analogues, agonists, antagonists, inhibitors, and pharmaceutically acceptable salt forms of the above. In reference to peptides and proteins, the disclosure is intended to encompass synthetic, native, glycosylated, unglycosylated, pegylated forms, and biologically active fragments, derivatives, and analogs thereof.

Additional active agents for use in the disclosure can further include nucleic acids, as bare nucleic acid molecules, vectors, associated viral droplets, plasmid DNA or RNA or other nucleic acid constructions of a type suitable for transfection or transformation of cells, e.g., suitable for gene therapy including antisense. Further, an active agent can comprise live attenuated or killed viruses suitable for use as vaccines. Other useful drugs include those listed within the Physician's Desk Reference (most recent edition), which is incorporated herein by reference in its entirety.

When a combination of active agents is used, the agents can be provided in combination in a single species of pharmaceutical composition or individually in separate species of pharmaceutical compositions.

The pharmaceutical compositions of one or more embodiments of the present disclosure can lack taste. In this regard, although taste masking agents are optionally included within the composition, the compositions in some embodiments do not include a taste masking agent other than a cyclodextrin and lack taste even without a taste masking agent.

In some embodiments, the pharmaceutical composition provided herein comprises a sweetener to improve the organoleptic properties of the composition. The sweetener can be a natural sweet substance, e.g. certain sugars, or an artificial sweetener. Without wishing to be bound to a certain theory, the presence of the sweetener in the pharmaceutical composition can improve the organoleptic properties of the composition. In some cases, the presence of the sweetener in the pharmaceutical composition can improve the compliance of the subject. presence of the sweetener in the pharmaceutical composition can increase the delivery efficiency of the composition. In some embodiments, the presence of the sweetener in the pharmaceutical composition can enhance the therapeutic effects of the composition.

Non-limiting examples of artificial sweeteners that can be used in the pharmaceutical composition include acesulfame potassium, aspartame, cyclamate, mogrosides, saccharin, stevia, sucralose, neotame, and sugar alcohols (e.g., erythritol, hydrogenated starch hydrolysates, isomalt, lactitol, maltitol, mannitol, sorbitol, and xylitol), such as those used in commercial products, like Sweet n' low powder sweetener, Truvia powder sweetener, Equal (aspartame), Stevia powder sachet, Aspen Naturals liquid stevia, Now Better Stevia liquid sweetener, Sweet N' Low liquid sweetener, Quick Sweet: Neotame liquid sweetener, or Splenda powder sachet, or pharmaceutically acceptable salts thereof. In some embodiments, the pharmaceutical composition comprises saccharin. In some embodiments, the pharmaceutical composition comprises a salt of saccharin. In some embodiments, the pharmaceutical composition comprises saccharin sodium.

Natural sweet substances that can be used in the pharmaceutical composition include, but not limited to, sucrose, agave, brown sugar, confectioner's (powdered) sugar, corn syrup, dextrose, fructose, fruit juice concentrate, glucose, high-fructose corn syrup, honey, invert sugar, lactose, malt sugar, maltose, maple syrup, molasses, nectars, raw sugar, and syrup. Sugars can increase the viscosity of the liquid solution, thus the concentration of any sugar added into the pharmaceutical composition, in some embodiments, is tightly controlled below a certain threshold value.

In some embodiments, pharmaceutically acceptable excipient or carrier comprises lactose, mannitol, sorbitol, erythritol, raffinose, sucrose, xylitol, trehalose, dextrose, cyclodextrins, maltitol, maltose, glucose, hydroxyapatite, or any combinations thereof.

Besides the above mentioned pharmaceutically acceptable excipients, it can be desirable to add other pharmaceutically acceptable excipients to the pharmaceutical composition to improve droplet rigidity, production yield, emitted dose and deposition, shelf-life, and patient acceptance. Such optional pharmaceutically acceptable excipients include coloring agents, taste masking agents, buffers, hygroscopic agents, antioxidants, and chemical stabilizers. In some embodiments, the compositions may include one or more osmolarity adjuster, such as sodium chloride. For instance, sodium chloride may be added to solutions to adjust the osmolarity of the solution.

In one or more embodiments, an aqueous pharmaceutical composition described herein comprises of essentially a pharmaceutical agent for treating pulmonary diseases (e.g., imatinib), water, pH buffer, solubility enhancer, and osmolarity adjuster. In some embodiments, the osmolarity adjuster can provide stability of aerosolized pharmaceutical composition, reduce adverse reaction to inhaled aerosolized pharmaceutical compositions (e.g., coughing), efficiency of aerosolization, or influence the droplet size. In some embodiments, the osmolarity of the aqueous pharmaceutical composition described herein are acceptable for pharmaceutical use (e.g., iso-osmolar, physiologic osmolarity, hypo-osmolar, physiologically hypotonic, hyper-osmolar, physiologically hypertonic). In some embodiments, the osmolarity of the aqueous pharmaceutical composition at about 0.001 mOsm to about 2,000 mOsm, for example between about 0.1 mOsm to about 1,000 mOsm, about 1 mOsm to about 200 mOsm, about 100 mOsm to about 200 mOsm, about 100 mOsm to about 500 mOsm, about 200 mOsm to about 400 mOsm, about 250 mOsm to about 350 mOsm, about 300 mOsm to about 400 mOsm, about 300 mOsm to about 2,000 mOsm, or about 1,000 mOsm to 2,000 mOsm. In some cases, the osmolality adjuster is a salt, such as sodium chloride or sodium carbonate.

### Nebulized Aerosol

### Droplets

The distribution of aerosol droplets of an inhalable formulation can be expressed in terms of either: the mass median aerodynamic diameter (MMAD)—the size at which half of the mass of the aerosol is contained in smaller droplets and half in larger droplets; volumetric mean diameter (VMD); mass median diameter (MMD); the fine droplet fraction (FDF)—the percentage of droplets that are <5 μm in diameter. These measures have been used for comparisons of the in vitro performance of different nebulizers and drug combinations. In general, the higher the fine droplet fraction, the higher the proportion of the emitted dose that is likely to deposit the lung. Generally, inhaled droplets are subject to deposition by one of two mechanisms: impaction, which usually predominates for larger droplets, and sedimentation, which is prevalent for smaller droplets. Impaction can occur when the momentum of an inhaled droplet is large enough that the droplet does not follow the air stream and encounters a physiological surface. In contrast, sedimentation can occur primarily in the deep lung when very small droplets which have traveled with the inhaled air stream encounter physiological surfaces as a result of random diffusion within the air stream.

For pulmonary administration, the upper airways are usually avoided in favor of the middle and lower airways. Pulmonary drug delivery can be accomplished by inhalation of an aerosol through the mouth and throat. Droplets having a mass median aerodynamic diameter (MMAD) of greater than about 5 microns generally do not reach the lung; instead, they tend to impact the back of the throat and are swallowed and possibly orally absorbed. Droplets having diameters of about 1 to about 5 microns are small enough to reach the upper-to-mid-pulmonary region (conducting airways), but are too large to reach the alveoli. Smaller droplets, i.e., about 0.5 to about 2 microns, are capable of reaching the alveolar region. Droplets having diameters smaller than about 0.5 microns can also be deposited in the alveolar region by sedimentation, although very small droplets can be exhaled. Measures of droplet size can be referred to as volumetric mean diameter (VMD), mass median diameter (MMD), or MMAD. These measurements can be made by impaction (MMD and MMAD) or by laser (VMD). For liquid particles, VMD, MMD and MMAD may be the same if environmental conditions are maintained, e.g., standard humidity. However, if humidity is not maintained, MMD and MMAD determinations will be smaller than VMD due to dehydration during impactor measurements. For the purposes of this description, VMD, MMD and MMAD measurements are considered to be under standard conditions such that descriptions of VMD, MMD and MMAD will be comparable.

Aerosol particle size may be expressed in terms of the mass median aerodynamic diameter (MMAD). Large droplets (e.g, MMAD-5 um) can deposit in the upper airway because they are too large to navigate the curvature of the upper airway. Small droplets (e.g., MMAD-2 um) can be poorly deposited in the lower airways and thus become exhaled, providing additional opportunity for upper airway deposition. Hence, intolerability (e.g., cough and bronchospasm) can occur from upper airway deposition from both inhalation impaction of large droplets and settling of small liquid droplets during repeated inhalation and expiration.

Thus, in one embodiment, an optimum droplet size is used (e.g., MMAD=1-5 um) in order to maximize deposition at a mid-lung and to minimize intolerability associated with upper airway deposition. Moreover, generation of a defined droplet size with limited geometric standard deviation (GSD) can optimize deposition and tolerability. Narrow GSD limits the number of droplets outside the desired MMAD size range. In one embodiment, an aerosol containing one or more compounds disclosed herein is provided having a MMAD from about 1 microns to about 5 microns with a GSD of less than or equal to about 2.5 microns. In another embodiment, an aerosol having an MMAD from about 2.8 microns to about 4.3 microns with a GSD less than or equal to 2 microns is provided. In another embodiment, an aerosol having an MMAD from about 2.5 microns to about 4.5 microns with a GSD less than or equal to 1.8 microns is provided.

In some embodiments, the nebulizer used in any of the methods described herein is a liquid nebulizer. In some embodiments, the nebulizer used in any of the methods described herein is a jet nebulizer, an ultrasonic nebulizer, a pulsating membrane nebulizer, a nebulizer comprising a vibrating mesh or plate with multiple apertures, or a nebulizer comprising a vibration generator and an aqueous chamber. In some embodiments, the nebulizer used in any of the methods described herein is a nebulizer comprising a vibrating mesh or plate with multiple apertures. In some embodiments, the liquid nebulizer achieves lung deposition of the imatinib or derivative thereof, or a salt thereof administered to the mammal; provides a Geometric Standard Deviation (GSD) of emitted droplet size distribution of the aqueous solution of about 1.0 um to about 2.5 um; provides a mass median aerodynamic diameter (MMAD) of droplet size of the aqueous solution emitted with the high efficiency liquid nebulizer of about 1 um to about 5 um; a volumetric mean diameter (VMD) of about 1 um to about 5 um; and/or a mass median diameter (MMD) of about 1 um to about 5 um; provides a fine droplet fraction (FDF=% s5 microns) of droplets emitted from the liquid nebulizer of at least about 30%; provides an output rate of at least 0.1 mL/min: and/or provides at least about 25% of the aqueous solution to the mammal.

In some embodiments, the composition (e.g., pharmaceutical composition or formulation) is aerosolized with nebulized droplets having an average mass median aerodynamic diameter of from 1 μm to 5 μm, from 1 μm to 4 μm, from 1 μm to 3 μm, from 1 μm to 2 μm, from 2 μm to 5 μm, from 2 μm to 4 μm, from 2 μm to 3 μm, or from 3 μm to 4 μm.

### Nebulizer

In one embodiment, a nebulizer is selected on the basis of allowing the formation of an aerosol of the composition described herein. In some embodiments, the MMAD of nebulized or aerosolized composition has a predominately MMAD of between about 1 to about 5 microns.

Efficient drug delivery to the lungs through nebulizers is dependent on several factors including inhaler device, formulation, and inhalation maneuver. The pharmaceutical compositions can be administered using an aerosolization device. The aerosolization device can be a nebulizer, a metered dose inhaler, or a liquid dose instillation device. The aerosolization device can comprise the extrusion of the pharmaceutical preparation through micron or submicron-sized holes with subsequent Rayleigh break-up into fine droplets. The pharmaceutical composition can be delivered by a nebulizer as described in WO 99/16420, by a metered dose inhaler as described in WO 99/16422, by a liquid dose instillation apparatus as described in WO 99/16421, which are incorporated herein by reference in their entireties. As such, an inhaler can comprise a canister containing the droplets or droplets and propellant, and wherein the inhaler comprises a metering valve in communication with an interior of the canister. The propellant can be a hydrofluoroalkane.

For instance, the pharmaceutical composition can be in liquid solution, and can be administered with nebulizers, such as that disclosed in WO 99/16420, the disclosure of which is hereby incorporated in its entirety by reference, in order to provide an aerosolized medicament that can be administered to the pulmonary air passages of a patient in need thereof. Nebulizers known in the art can easily be employed for administration of the claimed formulations. Breath-activated or breath-actuated nebulizers, as well as those comprising other types of improvements which have been, or will be, developed are also compatible with the formulations of the present disclosure and are contemplated as being within the scope thereof.

In some cases, the nebulizer is a breath activated or breath-actuated nebulizer. In some cases, the nebulizer is a hand-held inhaler device (e.g., AeroEclipse® Breath Actuated Nebulizer (BAN)). In some cases, the nebulizer has a compressed air source. In some cases, the nebulizer converts liquid medication into an aerosol. In some cases, the nebulizer converts liquid medication into an aerosol by extruding the pharmaceutical preparation through micron or submicron-sized holes. In some cases, the nebulizer converts liquid medication into an aerosol so it can be inhaled into the lungs. In some cases, the nebulizer is a small volume nebulizer. In some cases, the nebulizer is a small volume jet nebulizer. In some cases, aerosolized medication is only produced when inhaled through the device. In some cases, the medication is contained in the cup between breaths or during breaks in treatment. In some cases, the medication is contained in the cup until ready to be inhaled.

Nebulizers can impart energy into a liquid composition to aerosolize the liquid, and to allow delivery to the pulmonary system, e.g., the lungs, of a patient. A nebulizer comprises a liquid delivery system, such as a container having a reservoir that contains a liquid composition. The liquid composition generally comprises an active agent that is either in solution or suspended within a liquid medium.

In one type of nebulizer that can be used in the subject methods and kits, generally referred to as a jet nebulizer, compressed gas is forced through an orifice in the container. The compressed gas forces liquid to be withdrawn through a nozzle, and the withdrawn liquid can mix with the flowing gas to form aerosol droplets. A cloud of droplets can then be administered to the patients respiratory tract. In another type of nebulizer that can be used in the subject methods and kits, generally referred to as a vibrating mesh nebulizer, energy, such as mechanical energy, vibrates a mesh. This vibration of the mesh aerosolizes the liquid composition to create an aerosol cloud that is administered to the patient's lungs. In another type of nebulizer that can be used in the subject methods and kits, the nebulizing comprises extrusion through micron or submicron-sized holes followed by Rayleigh break-up into fine droplets. Alternatively or additionally, the composition may be in a liquid form and may be aerosolized using a nebulizer as described in WO 2004/071368, which is herein incorporated by reference in its entirety, as well as U.S. Published application Nos. 2004/0011358 and 2004/0035413, which are both herein incorporated by reference in their entireties. Other examples of nebulizers include, but are not limited to, the Aeroneb®Go or Aeroneb®Pro nebulizers, available from Aerogen Ltd. of Galway, Ireland; the PARI eFlow and other PARI nebulizers available from PARI Respiratory Equipment, Inc. of Midlothian, Va.; the Lumiscope® Nebulizer 6600 or 6610 available from Lumiscope Company, Inc. of East Brunswick, N.J.; the Omron NE-U22 available from Omron Healthcare, Inc. of Kyoto, Japan; and the Deepro®, Pulmogine™, and AdheResp™ devices available from HCmed Innovations Co., Ltd. of Taipei, Taiwan. Other examples of nebulizers include devices produced by Medspray® (Enschede, The Netherlands) (such as PFSI™ and Pulmospray™) and Pulmotree Medical GmbH (München, Germany). The Medspray® inhaler comprises a spray nozzle in combination of a pump system whereby liquid is dispersed into droplets by pressing a liquid pharmaceutical composition through a nozzle array via mechanical means, and a mouthpiece that mixes the resulting droplets with air. Such devices are described, for example, in de Boer et al. (2008), Pharmaceutical Research 25(5): 1186-1192; Wissink et al.

A nebulizer of the vibrating mesh type, such as one that that forms droplets without the use of compressed gas, such as the Aeroneb® Pro, can provide unexpected improvement in dosing efficiency and consistency. By generating fine droplets by using a vibrating perforated or unperforated membrane, rather than by introducing compressed air, the aerosolized composition can be introduced without substantially affecting the flow characteristics. In addition, the generated droplets when using a nebulizer of this type can be introduced at a low velocity, thereby decreasing the likelihood of the droplets being driven to an undesired region. When using a nebulizer of the extrusion/Rayleigh jet breakup type, the generated droplets can also be introduced at a low velocity, thereby decreasing the likelihood of the droplets being driven to an undesired region.

In some cases, the nebulizer that can be used in the subject methods and kits is of the vibrating mesh type. In some cases, the nebulizer that can be used in the subject methods and kits is of the pressurized jet type. In some cases, the nebulizer that can be used in the subject methods and kits is of the extrusion/Rayleigh breakup type. In some cases, the nebulizer is lightweight (at most 60 g, at most 100 g, at most 200 g, at most 250 g) and nearly silent. In some cases, the nebulizer has a sound level less than 35 A-weighted decibels (dBA) at 1 meter. In some cases, the nebulizer has a medication cup capacity of 6 mL. In some cases, the nebulizer has a residual volume of less than 0.3 mL. In some cases, the nebulizer generates an average flow rate of 0.4 mL/min. In some cases, the nebulizer generates an average flow rate of 0.5 mL/min. In some cases, the nebulizer generates an average flow rate of 0.6 mL/min. In some cases, the nebulizer generates an average flow rate of 0.7 mL/min. In some cases, the nebulizer generates an average flow rate of 0.8 mL/min. In some cases, the nebulizer generates an average flow rate of 0.9 mL/min. In some cases, the nebulizer generates an average flow rate of 1.0 mL/min. In some cases, the nebulizer generates an average flow rate of 1.1 mL/min. In some cases, the nebulizer generates an average flow rate of 1.2 mL/min. In some cases, the nebulizer generates an average droplet size of 3.0 μm MMAD. In some cases, the nebulizer generates an average droplet size between 3.0 μm MMAD and 4.0 μm MMAD. In some cases, the nebulizer generates an average droplet size of 3.0 μm MMAD. In some cases, the nebulizer generates an average droplet size between 3.0 μm MMAD and 5.0 μm MMAD. In some cases, the nebulizer generates an average droplet size of 3.0 μm MMAD. In some cases, the nebulizer generates an average droplet size between 3.0 μm MMAD and 6.0 μm MMAD. In still another type of nebulizer that can be used in the subject methods and kits, ultrasonic waves are generated to directly vibrate and aerosolize the composition. The compositions disclosed herein can also be administered to the lungs of a patient via aerosolization, such as with a metered dose inhaler. The use of such formulations provides for superior dose reproducibility and improved lung deposition as disclosed in WO99/16422, hereby incorporated in its entirety by reference. Metered dose inhalers (MDIs) known in the art can be employed for administration of the claimed compositions. Breath-activated or breath-actuated MDIs and pressurized MDIs (pMDIs), as well as those comprising other types of improvements which have been, or will be, developed are also compatible with the formulations of the present disclosure and, as such, are contemplated as being within the scope thereof. Along with MDIs and nebulizers, it will be appreciated that the formulations of one or more embodiments of the present disclosure can be used in conjunction with liquid dose instillation or LDI techniques as disclosed in, for example, WO 99/16421, which is incorporated herein by reference in its entirety. Liquid dose instillation involves the direct administration of a formulation to the lung. With respect to LDI the formulations are preferably used in conjunction with partial liquid ventilation or total liquid ventilation. Moreover, one or more embodiments of the present disclosure may further comprise introducing a therapeutically beneficial amount of a physiologically acceptable gas (such as nitric oxide or oxygen) into the pharmaceutical microdispersion prior to, during or following administration.

Aqueous formulations may be aerosolized by liquid nebulizers employing either hydraulic or ultrasonic atomization. Propellant-based systems may use suitable pressurized metered-dose inhalers (pMDIs). A desired particle size and distribution may be obtained by choosing an appropriate device.

In some cases, the nebulizer that can be used in the subject methods and kits is a hydraulic atomization device, which atomizes a liquid under increased hydraulic pressure (in some cases, without any need of propellant). In some cases, a hydraulic atomization device comprises an ultrathin membrane with micron-size pores (e.g., of pore size between 1.5 and 15 micrometers). When a hydraulic pressure is applied, liquid jet (e.g., soft mist) formed from the liquid formulation contained in the device can emerge from the pores of the ultrathin membrane. The ultrathin membrane with micron-size pores can be manufactured using lithography. Examples of hydraulic atomization devices that can be used in the subject methods and kits include those described in U.S. Ser. No. 11/045,819, US20080245758, US2018353980, US2021387209, US2021268523, US2017281880, US2011049262, US2012012105, US2003178507, US2018353977, US20220143638, US2021008577, US2022379047, and US2022370734.

In some embodiments, the nebulizer is a jet nebulizer, a vibrating mesh nebulizer, an ultrasonic nebulizer, an electrospray nebulizer, or a hydraulic atomization device.

### Ready-to-Use Formulation

The pharmaceutical compositions provided herein is ready-to-use for treatment of pulmonary diseases for immediate use. In contrast, other drug formulations comprising imatinib are in powder form for liquid suspension for oral use or in concentrated liquid form/stock solution that require dilution prior to usage necessitate additional components and steps. The pharmaceutical compositions described herein are in aqueous liquid form that provides immediate usage for a pulmonary disease related symptom without need of additional steps. The ready-to-use formulation of the pharmaceutical compositions described herein invasive procedures such as intravenous administration.

In some embodiments, the ready-to-use pharmaceutical composition is in a single use dose to treat a pulmonary disease or symptom of a pulmonary disease. In some embodiments, the single use dosage is in a cartridge or container with a volume for the single usage. In some embodiments, the single use dosage is in a cartridge or container with a volume for additional application of the pharmaceutical composition. In some embodiments, the ready-to-use pharmaceutical composition is in a multiple dosage formulation. In some embodiments, the multiple dosage formulation of the ready-to-use pharmaceutical formulation is in a cartridge or container wherein the volume contains the multiple dosages. In some embodiments, the multiple dosage formulations require refilling a cartridge or container of the ready-to-use pharmaceutical composition for nebulization. In some embodiments, a measuring tool and/or transfer tool is included in kits for refilling a cartridge or container for multiple dosage usage.

### Methods of Treatment

### Pulmonary Diseases

The methods, compositions, and kits provided herein can be suitable for treatment of a pulmonary disease. Examples of pulmonary diseases can include, but not limited to, asthma, emphysema, chronic obstructive pulmonary disease (COPD), infections (e.g., pneumonia, tuberculosis, influenza), coccidioidomycosis, corona virus, cytogenetic organizing pneumonia (COP), pulmonary hypertension (such as pulmonary arterial hypertension, pulmonary hypertension due to left heart diseases, pulmonary hypertension due to lung diseases and/or hypoxemia, chronic thromboembolic pulmonary hypertension (CTEPH), and pulmonary hypertension from unclear multifactorial mechanisms), respiratory syncytial virus (RSV), hantavirus pulmonary syndrome (HPS), Mycobacterium avium complex lung disease, Middle Eastern Respiratory Syndrome (MERS), mesothelioma, nontuberculous mycobacteria lung disease (NTM), severe acute respiratory syndrome (SARS), cancer (e.g., lung cancer, e.g., small cell lung cancer, or non-small cell lung cancer, such as adenocarcinoma and squamous cell carcinoma), acute respiratory distress syndrome (ARDS), alpha-1 antitrypsin deficiency (AAT), asbestosis, aspergillosis, bronchiectasis, bronchiolitis, bronchiolitis obliterans, acute bronchitis, bronchopulmonary dysplasia, chronic bronchitis, chronic cough, Coal Worker's pneumoconiosis (Black Lung Disease), cystic fibrosis, e-cigarette or vaping use-associated lung injury (EVALI), eosinophilic granulomatosis with polyangiitis, histoplasmosis, human metapneumovirus (hMPV), hypersensitivity pneumonitis, idiopathic pulmonary fibrosis (IPF), interstitial lung disease (ILD), pulmonary hypertension associated with interstitial lung disease (PH-ILD), sarcoidosis, Legionnaires' disease, pertussis (whooping cough), primary ciliary dyskinesia (PCT), pulmonary arterial hypertension (PAH), silicosis, or pulmonary fibrosis. The pharmaceutical compositions described herein can be used to treat diseases and symptoms relating to a lung disorder, respiratory diseases, and trachea or bronchi disorders. In some cases, a lung disorder may arise from inhalation and/or exposure to tobacco smoke, infections (e.g., bacterial, viral, fungal), radon, asbestos, air pollution, particulates, debris. In some cases, a lung disorder or symptom of the lung disorder may arise from lung damage, sleep apnea, collapsed lung, or pulmonary embolism.

In some cases, the methods, compositions, and kits provided herein are suitable for treatment of pulmonary hypertension. Pulmonary hypertension (PH) is a general term that can be used to describe high blood pressure within the lungs, which can also impact the arteries that transport the blood between lungs and heart. In the case of pulmonary hypertension, the arteries can constrict and force the blood to flow through the vessels. Symptoms of pulmonary hypertension can include shortness of breath, fainting, dizziness, chest pressure, increased pulse. The methods, compositions, and kits provided herein can ameliorate one or more symptoms of pulmonary hypertension (e.g., PAH).

It is accepted that there are five subclassifications (i.e. “groups”) of pulmonary hypertension, each one of which differs slightly from the others: group 1 (PAH), group 2 (pulmonary hypertension due to left heart diseases), group 3 (pulmonary hypertension due to lung diseases and/or hypoxemia), group 4 (chronic thromboembolic pulmonary hypertension (CTEPH)), and group 5 (pulmonary hypertension from unclear multifactorial mechanisms). In some cases, the methods, compositions, and kits provided herein are suitable for treatment of one or more of the five different groups of pulmonary hypertension, e.g., effective in ameliorating one or more symptoms of one or more of the five different groups of pulmonary hypertension. Pulmonary arterial hypertension (PAH) is a chronic disease that can make the artery walls of the lungs thicken and, as a result, can prevent the blood from circulating properly. The methods, compositions, and kits provided herein are suitable for treatment of one or more of the three types of pulmonary arterial hypertension: idiopathic, heritable, or drug and toxin-induced. Pulmonary hypertension due to left heart diseases can be characterized by defects with the left side of the heart and can be caused by numerous heart disorders like left ventricular diastolic dysfunction, coronary artery disease, high blood pressure, heart valve disease, damage to heart muscle, and more. Pulmonary hypertension due to lung diseases and/or hypoxemia can be developed as a secondary disease after suffering from hypoxia, COPD, interstitial lung disease, or any other lung disease which causes blood vessels to tighten and blood oxygen to drop. CTEPH can be caused by old, organized blood clots in the lungs that physically block the flow of the blood through the pulmonary arteries. Pulmonary hypertension in group 5, pulmonary hypertension from unclear multifactorial mechanisms, are pulmonary hypertension that have all other causes that are not defined in the other four groups, such as causes like hematological and metabolic disorders, various blood diseases, and system disorders.

Pharmaceutical compositions disclosed herein can be more effective in subjects that include or lack certain physiological or demographic factors, such as, for example, age at clinical presentation, certain hemodynamic criteria, electrophysiological features, and prior treatments. In some embodiments, a subject treated with a pharmaceutical composition of the disclosure suffers from recurrent pulmonary disease. In some embodiments, a subject treated with a pharmaceutical composition of the disclosure has an ongoing prescription medication for a pulmonary disease. In some embodiments, the oral medication for treating the pulmonary disease is imatinib, or a pharmaceutically acceptable salt thereof.

In some embodiments, a subject treated with a pharmaceutical composition of the disclosure is over 18 years in age. In some embodiments, a subject treated with a pharmaceutical composition of the disclosure is under the age of 18. In some embodiments, a subject treated with a pharmaceutical composition of the disclosure is no more than 85 years in age. In some embodiments, a subject treated with a pharmaceutical composition of the disclosure is from 18 years old to 85 years old.

In some embodiments, a subject treated with a pharmaceutical composition of the disclosure does not exhibit severe renal impairment, wherein a eGFR of the subject is less than 30 mL/min/1.73 m² at the time of treating. In some embodiments, a subject treated with a pharmaceutical composition of the disclosure is not on dialysis at the time of treating. In some embodiments, a subject treated with a pharmaceutical composition of the disclosure does not exhibit abnormal liver function at the time of treating. In some embodiments, the abnormal liver function is hepatic disease or biochemical evidence of significant liver derangement. In some embodiments, a subject treated with a pharmaceutical composition of the disclosure does not exhibit uncorrected hypokalemia at the time of treating. In some embodiments, a subject treated with a pharmaceutical composition of the disclosure does not exhibit a serum potassium less than 3.6 mEq/L at the time of treating.

In some embodiments, a subject treated with a pharmaceutical composition of the disclosure does not exhibit an established pulmonary disease in need of inhalation medication at the time of treating. In some embodiments, a subject treated with a pharmaceutical composition of the disclosure does not have a hypersensitivity to imatinib or any of its active metabolites, or a history thereof. In some embodiments, a subject treated with a pharmaceutical composition of the disclosure is not concomitantly administered a systemic drug that is an inhibitor of CYP 2D6. In some embodiments, the inhibitor of CYP 2D6 is an antidepressant, a neuroleptic, or an antihistamine. In some embodiments, the inhibitor of CYP 2D6 is propranolol or ritonavir. In some embodiments, a subject treated with a pharmaceutical composition of the disclosure is not concomitantly administered a systemic drug that is a CYP 2D6 inducer. In some embodiments, the CYP 2D6 inducer is phenytoin, phenobarbital, or carbamazepine.

In some embodiments, a subject treated with a pharmaceutical composition of the disclosure does not exhibit a congenital lung disease at the time of treating. In some embodiments, a subject treated with a pharmaceutical composition of the disclosure does not exhibit syncope at the time of treating.

In some embodiments, a subject treated with a pharmaceutical composition of the disclosure does not exhibit any serious or life threatening medical condition other than a pulmonary disease symptoms at the time of treating. In some embodiments, a subject treated with a pharmaceutical composition of the disclosure does not exhibit an acute pathogenic infection at the time of treating.

In some embodiments, a subject treated with a pharmaceutical composition of the disclosure has not exhibited a drug or alcohol dependence within 12 months prior to administration of the pharmaceutical composition. In some embodiments, a subject treated with a pharmaceutical composition of the disclosure does not exhibit a body mass index greater than 40 Kg/m² at the time of treating.

The therapy provided herein can comprise or be suitable for inhalation, e.g., oral or nasal inhalation. In some cases, during administration via oral inhalation, the pharmaceutical agent is inhaled by the patient through the mouth and absorbed by the lungs. In some cases, during administration via nasal inhalation, the pharmaceutical agent is inhaled by the patient through the nose and absorbed by the nasal mucous and/or the lungs.

The inhalation route can avoid first-pass hepatic metabolism, hence dosing variability can be eliminated. Unlike the case for oral tablets or pills, the patient's metabolic rates may not matter as the administration is independent of the metabolic paths experienced when a drug is administered via oral route through gastrointestinal tract, e.g., as tablets, pills, solution, or suspension. A fast onset of action, a potential improvement in efficacy, and/or a reduction in dose can be achieved with the fast absorption of drugs from the nasal mucosa and/or lungs.

The fast absorption rate of drugs through the lungs can be achieved because of the large surface area available in the lungs for aerosols small enough to penetrate central and peripheral lung regions. Consequently, the rate and extent of absorption of drugs delivered via inhalation can yield plasma concentrations vs. time profiles that are comparable with the IV route of administration.

In some cases, the therapy provided herein is provided to a subject for more than once on an as-needed basis. For instance, the therapy can be administered to a subject, e.g., the pharmaceutical composition is inhaled by the subject, for at least once per day, e.g., for 1, 2, 3, 4, 5, 6, 8, or 10 times per day. In some cases, the therapy can be administered to a subject for an extended period of time, for instance, for a period of at least 5, 10, 20, 30, 60, 100, or 300 days, at least 1, 2, 3, 4, or 5 years, during which time the subject receives administration of the therapy on a daily basis, or every other day, or every 2, 3, 4, 5, 6, 7, or 10 days. On each day that the therapy is administered to the subject, the subject can receive administration of the therapy, e.g., inhale the pharmaceutical composition provided herein, for at least once, e.g., 1, 2, 3, 4, 5, 6, 8, or 10 times.

### Dosage

The pharmaceutical composition can be administered to the patient on an as-needed basis.

In some embodiments, a pharmaceutical agent of the disclosure suitable for treatment of pulmonary disease (e.g. imatinib) is administered in unit dose form. In some embodiments, the unit dosage is about 20 mg to about 500 mg of the pharmaceutical agent. In some embodiments, the unit dosage is about 20 mg to about 500 mg of the pharmaceutical agent wherein the pharmaceutical agent is imatinib or a pharmaceutically acceptable salt thereof. In some embodiments, the unit dosage is about 30 mg to about 500 mg of the pharmaceutical agent. In some embodiments, the unit dosage is about 40 mg to about 500 mg of the pharmaceutical agent. In some embodiments, the unit dosage is about 50 mg to about 500 mg of the pharmaceutical agent. In some embodiments, the unit dosage is about 100 mg to about 500 mg of the pharmaceutical agent. In some embodiments, the unit dosage is about 120 mg to about 500 mg of the pharmaceutical agent. In some embodiments, the unit dosage is about 150 mg to about 500 mg of the pharmaceutical agent. In some embodiments, the unit dosage is about 200 mg to about 500 mg of the pharmaceutical agent. In some embodiments, the unit dosage is about 250 mg to about 500 mg of the pharmaceutical agent. In some embodiments, the unit dosage is about 300 mg to about 500 mg of the pharmaceutical agent. In some embodiments, the unit dosage is about 350 mg to about 500 mg of the pharmaceutical agent. In some embodiments, the unit dosage is about 400 mg to about 500 mg of the pharmaceutical agent. In some embodiments, the unit dosage is about 450 mg to about 500 mg of the pharmaceutical agent. In some embodiments, the unit dosage is about 30 mg to about 500 mg of the pharmaceutical agent. In some embodiments, the unit dosage is about 20 mg to about 450 mg of the pharmaceutical agent. In some embodiments, the unit dosage is about 20 mg to about 400 mg of the pharmaceutical agent. In some embodiments, the unit dosage is about 20 mg to about 350 mg of the pharmaceutical agent. In some embodiments, the unit dosage is about 20 mg to about 300 mg of the pharmaceutical agent. In some embodiments, the unit dosage is about 20 mg to about 250 mg of the pharmaceutical agent. In some embodiments, the unit dosage is about 20 mg to about 200 mg of the pharmaceutical agent. In some embodiments, the unit dosage is about 20 mg to about 150 mg of the pharmaceutical agent. In some embodiments, the unit dosage is about 20 mg to about 120 mg of the pharmaceutical agent. In some embodiments, the unit dosage is about 20 mg to about 100 mg of the pharmaceutical agent. In some embodiments, the unit dosage is about 20 mg to about 50 mg of the pharmaceutical agent. In some embodiments, the unit dosage is about 20 mg to about 40 mg of the pharmaceutical agent. In some embodiments, the unit dosage is about 20 mg to about 30 mg of the pharmaceutical agent. In some embodiments, the unit dosage is about 30 mg to about 450 mg of the pharmaceutical agent. In some embodiments, the unit dosage is about 40 mg to about 400 mg of the pharmaceutical agent. In some embodiments, the unit dosage is about 50 mg to about 350 mg of the pharmaceutical agent. In some embodiments, the unit dosage is about 100 mg to about 300 mg of the pharmaceutical agent. In some embodiments, the unit dosage is about 150 mg to about 250 mg of the pharmaceutical agent. In some embodiments, the unit dosage is about 100 mg to about 150 mg of the pharmaceutical agent. In some embodiments, the unit dosage is about 20 mg to about 200 mg of the pharmaceutical agent. In some embodiments, the unit dosage is about 200 mg to about 500 mg of the pharmaceutical agent. In some embodiments, the unit dosage is about 30 mg to about 100 mg of the pharmaceutical agent. In some embodiments, the unit dosage is about 30 mg to about 90 mg of the pharmaceutical agent. In some embodiments, the unit dosage is about 30 mg to about 80 mg of the pharmaceutical agent. In some embodiments, the unit dosage is about 40 mg to about 80 mg of the pharmaceutical agent. In some embodiments, the unit dosage is about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, or about 500 mg of the pharmaceutical agent.

In some cases, the unit dose of the pharmaceutical composition described herein in aqueous solution is from 20 mg/mL to 500 mg/mL of imatinib. In some cases, the unit dose is 20 to 500 mg/mL, from 20 to 450 mg/mL, from 20 to 400 mg/mL, from 20 to 350 mg/mL, from 20 to 300 mg/mL, from 20 to 250 mg/mL, from 20 to 200 mg/mL, from 20 to 150 mg/mL, from 20 to 120 mg/mL, from 20 to 100 mg/mL, from 20 mg/mL to 80 mg/mL, from 20 mg/mL to 60 mg/mL, from 20 mg/mL to 40 mg/mL, from 20 mg/mL to 30 mg/mL, from 30 mg/mL to 40 mg/mL, from 40 mg/mL to 60 mg/mL, from 40 mg/mL to 80 mg/mL, from 40 mg/mL to 100 mg/mL, from 40 mg/mL to 120 mg/mL, from 40 mg/mL to 150 mg/mL, from 40 mg/mL to 200 mg/mL, from 40 mg/mL to 250 mg/mL, from 40 mg/mL to 300 mg/mL, from 40 mg/mL to 350 mg/mL, from 40 mg/mL to 400 mg/mL, from 40 mg/mL to 450 mg/mL, from 40 mg/mL to 500 mg/mL, from 60 mg/mL to 80 mg/mL, from 60 mg/mL to 100 mg/mL, from 60 mg/mL to 120 mg/mL, from 60 mg/mL to 150 mg/mL, from 60 mg/mL to 200 mg/mL, from 60 mg/mL to 250 mg/mL, from 60 mg/mL to 300 mg/mL, from 60 mg/mL to 350 mg/mL, from 60 mg/mL to 400 mg/mL, from 60 mg/mL to 450 mg/mL, from 60 mg/mL to 500 mg/mL, from 80 mg/mL to 100 mg/mL, from 80 mg/mL to 120 mg/mL, from 80 mg/mL to 150 mg/mL, from 80 mg/mL to 200 mg/mL, from 80 mg/mL to 250 mg/mL, from 80 mg/mL to 300 mg/mL, from 80 mg/mL to 350 mg/mL, from 80 mg/mL to 400 mg/mL, from 80 mg/mL to 450 mg/mL, from 80 mg/mL to 500 mg/mL, from 100 mg/mL to 120 mg/mL, from 100 mg/mL to 150 mg/mL, from 100 mg/mL to 200 mg/mL, from 100 mg/mL to 250 mg/mL, from 100 mg/mL to 300 mg/mL, from 100 mg/mL to 350 mg/mL, from 100 mg/mL to 400 mg/mL, from 100 mg/mL to 450 mg/mL, from 100 mg/mL to 500 mg/mL, from 120 mg/mL to 150 mg/mL, from 120 mg/mL to 200 mg/mL, from 120 mg/mL to 250 mg/mL, from 120 mg/mL to 300 mg/mL, from 120 mg/mL to 350 mg/mL, from 120 mg/mL to 400 mg/mL, from 120 mg/mL to 450 mg/mL, from 120 mg/mL to 500 mg/mL, from 150 mg/mL to 200 mg/mL, from 150 mg/mL to 250 mg/mL, from 150 mg/mL to 300 mg/mL, from 150 mg/mL to 350 mg/mL, from 150 mg/mL to 400 mg/mL, from 150 mg/mL to 450 mg/mL, from 150 mg/mL to 500 mg/mL, from 200 mg/mL to 250 mg/mL, from 200 mg/mL to 300 mg/mL, from 200 mg/mL to 350 mg/mL, from 200 mg/mL to 400 mg/mL, from 200 mg/mL to 450 mg/mL, from 200 mg/mL to 500 mg/mL, from 250 mg/mL to 300 mg/mL, from 250 mg/mL to 350 mg/mL, from 250 mg/mL to 400 mg/mL, from 250 mg/mL to 450 mg/mL, from 250 mg/mL to 500 mg/mL, from 300 mg/mL to 350 mg/mL, from 300 mg/mL to 400 mg/mL, from 300 mg/mL to 450 mg/mL, from 300 mg/mL to 500 mg/mL from 350 mg/mL to 400 mg/mL, from 350 mg/mL to 450 mg/mL, from 350 mg/mL to 500 mg/mL, from 400 mg/mL to 450 mg/mL, from 400 mg/mL to 500 mg/mL, mg/mL, or from 450 mg/mL to 500 mg/mL of imatinib.

The pharmaceutical composition of one or more embodiments of the present disclosure can have improved emitted dose efficiency. The emitted dose (ED) of the aerosolized liquid droplets of the present disclosure can be greater than about 30%, such as greater than about 40%, greater than about 50%, greater than about 60%, or greater than about 70%. The dose of the pharmaceutical agent for treatment of pulmonary diseases (e.g., imatinib free base, imatinib salt, imatinib mesylate, imatinib derivative) can be administered during a single inhalation or can be administered during several inhalations. The fluctuations of the pharmaceutical agent can be reduced by administering the pharmaceutical composition more often or can be increased by administering the pharmaceutical composition less often. Therefore, the pharmaceutical composition provided herein can be administered from about four times daily to about once a month, such as about once daily to about once every two weeks, about once every two days to about once a week, and about once per week.

In some cases, the pharmaceutical agent for treatment of pulmonary diseases is delivered over two or more inhalations. In some cases, time between the two or more inhalations is from about 0.1 to 10 minutes. The pharmaceutical agent for treatment of pulmonary diseases is administered in the described dose in less than 60 minutes, less than 50 minutes, less than 40 minutes, less than 30 minutes, less than 20 minutes, less than 15 minutes, less than 10 minutes, less than 7 minutes, less than 5 minutes, in less than 3 minutes, in less than 2 minutes, or in less than 1 minute. In some cases, delivery of the required dose of pharmaceutical agent for treating pulmonary diseases (e.g., imatinib) is completed with 1, 2, 3, 4, 5, or 6 inhalations. In some cases, each inhalation is performed for about 0.5, 1, 1.2, 1.5, 1.8, 2, 2.2, 2.5, 2.8, 3, 3.2, 3.5, 3.8, 4, 4.2, 4.5, 4.8, or 5 minutes. In some cases, each inhalation is performed for longer than 5 minutes. In some cases, each inhalation is performed for up to 4.5 minutes. In some cases, each inhalation comprises at least 60 inhalation breaths, 50 inhalation breaths, 40 inhalation breaths, 30 inhalation breaths, 20 inhalation breaths, 10 inhalation breaths, 8 inhalation breaths, 6 inhalation breaths, 4 inhalation breaths, 3 inhalation breaths, 2 inhalation breaths or 1 inhalation breath. In some cases, each inhalation comprises no more than 100 inhalation breaths, 90 inhalation breaths, 80 inhalation breaths, 70 inhalation breaths, 60 inhalation breaths, 50 inhalation breaths, 40 inhalation breaths, 30 inhalation breaths, or 20 inhalation breaths. In some cases, inhalation of the pharmaceutical agent is performed with deep lung breath that lasts for longer than 1 second, 2 seconds, 3 seconds, or 4 seconds. In some cases, inhalation of the pharmaceutical agent for treatment of pulmonary diseases is performed with deep lung breath that lasts for about 1 second, 2 seconds, 3 seconds, or 4 seconds.

In some embodiments, during inhalational delivery of the pharmaceutical agent for treatment of pulmonary diseases, the subject takes, or is instructed to take, a break between two inhalations. In such embodiments, the break between two inhalations lasts for about 0.1 to 10 minutes, such as, 0.2 to 5, 1 to 5, 1.5 to 5, 2 to 5, 3 to 5, 4 to 5, 1 to 1.5, 1 to 2, 1 to 2.5, 1 to 3, 1 to 3.5, 1 to 4, 1.5 to 2, 1.5 to 2.5, or 1.5 to 3 minutes. In some cases, the subject takes, or is instructed to take, a break for about 1 minute between two inhalations. In some cases, the inhalation pattern for delivery of a single dose goes as follows: a first inhalation for about 4 to 4.5 minutes, a break for about 1 minute, and a second inhalation for about 4 to 4.5 minutes; a first inhalation for about 4 to 4.5 minutes, a break for about 30 seconds, and a second inhalation for about 4 to 4.5 minutes; a first inhalation for about 4 to 4.5 minutes, a first break for about 1 minute, and a second inhalation for about 4 to 4.5 minutes; a second break for about 1 minutes, and a third inhalation for about 4 to 4.5 minutes; or a first inhalation for about 4 to 4.5 minutes, a first break for about 30 seconds, and a second inhalation for about 4 to 4.5 minutes; a second break for about 30 seconds, and a third inhalation for about 4 to 4.5 minutes.

In one version, the pharmaceutical composition described herein can be administered daily. In this case, the daily dosage of the imatinib ranges from about 0.1 mg to about 600 mg, such as about 0.5 mg to about 500 mg, about 1 mg to about 400 mg, about 2 mg to about 300 mg, and about 3 mg to about 200 mg.

In some cases, the therapy provided herein is provided to a subject for more than once on an as-needed basis. For instance, the present disclosure can involve a follow-up inhalation if symptoms of the pulmonary disease have not subsided and occurs after an initial inhalation. In some instances, if symptoms of the pulmonary disease have not subsided within 30 minutes of the initial inhalation, the follow-up dosage is higher or the same as the initial dosage.

In another version, the pharmaceutical composition is administered prophylactically to a subject who is likely to develop a pulmonary disease. For example, a patient who has a history of pulmonary diseases can be prophylactically treated with a pharmaceutical composition comprising imatinib to reduce the likelihood of developing a pulmonary disease.

The pharmaceutical composition can be administered to a patient in any regimen which is effective to prevent a pulmonary disease. Illustrative prophylactic regimes include administering a pharmaceutical agent for treating pulmonary diseases as described herein 1 to 21 times per week.

The amount of the imatinib that is delivered to the subject (e.g., approximately the amount of the imatinib free base exiting a mouthpiece when being inhaled by the subject) for the treatment of a pulmonary disease can be from about 20 mg to about 500 mg, such as 20 mg to 30 mg, 20 mg to 40 mg, 20 mg to 50 mg, 20 mg to 60 mg, 20 mg to 70 mg, 20 mg to 80 mg, 20 mg to 90 mg, 20 mg to 100 mg, 20 mg to 110 mg, 20 mg to 120 mg, 20 mg to 130 mg, 20 mg to 140 mg, 20 mg to 150 mg, 20 mg to 160 mg, 20 mg to 170 mg, 20 mg to 180 mg, 20 mg to 200 mg, 20 mg to 250 mg, 20 mg to 300 mg, 20 mg to 350 mg, 20 mg to 400 mg, 20 mg to 500 mg, 50 mg to 60 mg, 50 mg to 70 mg, 50 mg to 80 mg, 50 mg to 90 mg, 50 mg to 100 mg, 50 mg to 120 mg, 50 mg to 150 mg, 50 mg to 200 mg, 50 mg to 250 mg, 50 mg to 300 mg, 50 mg to 350 mg, 50 mg to 400 mg, 50 mg to 450 mg, 50 mg to 500 mg, 100 mg to 120 mg, 100 mg to 150 mg, 100 mg to 200 mg, 100 mg to 250 mg, 100 mg to 300 mg, 100 mg to 350 mg, 100 mg to 400 mg, 100 mg to 450 mg, 100 mg to 500 mg, 150 mg to 200 mg, 150 mg to 250 mg, 150 mg to 300 mg, 150 mg to 350 mg, 150 mg to 400 mg, 150 mg to 450 mg, 150 mg to 500 mg, 200 mg to 250 mg, 200 mg to 300 mg, 200 mg to 350 mg, 200 mg to 400 mg, 200 mg to 450 mg, 200 mg to 500 mg, 40 mg to 150 mg, 50 mg to 150 mg, 60 mg to 150 mg, 70 mg to 150 mg, 80 mg to 150 mg, 90 mg to 150 mg, 100 mg to 150 mg, 110 mg to 150 mg, 120 mg to 150 mg, 130 mg to 150 mg, 140 mg to 150 mg, 30 mg to 140 mg, 40 mg to 130 mg, 50 mg to 120 mg, 60 mg to 110 mg, 70 mg to 110 mg, or 80 mg to 100 mg.

In one version, the amount of the imatinib that is delivered to the subject (e.g., approximately the amount of the imatinib exiting the aerosolization device when being inhaled by the subject) for the treatment of pulmonary diseases, is at least about 20 mg, at least about 30 mg, at least about 40 mg, at least about 50 mg, at least about 60 mg, at least about 70 mg, at least about 80 mg, at least about 90 mg, at least about 100 mg, at least about 110 mg, at least about 120 mg, at least about 130 mg, at least about 140 mg, at least about 150 mg, at least about 160 mg, at least about 170 mg, at least about 180 mg, at least about 190 mg, at least about 200 mg, at least about 225 mg, at least about 250 mg, at least about 275 mg, at least about 300 mg, at least about 325 mg, at least about 350 mg, at least about 375 mg, at least about 400 mg, at least about 425 mg, at least about 450 mg, at least about 470 mg, or at least about 500 mg.

In one version, the amount of the imatinib that is delivered to the subject (e.g., approximately the amount of the imatinib exiting a mouthpiece when being inhaled by the subject) for the treatment of pulmonary diseases is at most about 20 mg, at most about 30 mg, at most about 40 mg, at most about 50 mg, at most about 60 mg, at most about 70 mg, at most about 80 mg, at most about 90 mg, at most about 100 mg, at most about 110 mg, at most about 120 mg, at most about 130 mg, at most about 140 mg, at most about 150 mg, at most about 160 mg, at most about 170 mg, at most about 180 mg, at most about 190 mg, at most about 200 mg, at most about 225 mg, at most about 250 mg, at most about 275 mg, at most about 300 mg, at most about 325 mg, at most about 350 mg, at most about 375 mg, at most about 400 mg, at most about 425 mg, at most about 450 mg, at most about 475 mg, or at most about 500 mg.

In some cases, the amount of the imatinib that is delivered to the subject (e.g., approximately the amount of the imatinib exiting a mouthpiece when being inhaled by the subject) for the treatment of pulmonary diseases is about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 225 mg, about 250 mg, about 275 mg, about 300 mg, about 325 mg, about 350 mg, about 375 mg, about 400 mg, about 425 mg, about 450 mg, about 475 mg, or about 500 mg.

A pharmaceutical composition described herein can be administered in a single dose or in multiple doses to a subject.

In some cases, the subject in need thereof is administered with a single dose of the pharmaceutical composition disclosed herein. For instance, the subject is administered with a single dose of the pharmaceutical composition disclosed herein on an as needed basis.

In some cases, the subject in need thereof is administered with multiple doses of the pharmaceutical composition disclosed herein. For instance, the subject can be administered with a multiple-dose treatment regimen, e.g., administered with multiple doses of the pharmaceutical composition disclosed herein within a period of time, such as within one, two, three, four, five, six, or seven days. In some cases, the multiple-dose regimen comprises administration of the pharmaceutical composition provided herein at least once a day, such as once, twice, three times, or four times every day, for a period of time, e.g., at least 3 consecutive days, such as 3, 4, 5, 7, 10, 12, 14, 20, 21, 25, 28, or 35 consecutive days. In some cases, the multiple-dose treatment regimen comprises single-dose administration for every other day, every three, four, five, six, seven, or more days. In some cases, the multiple-dose treatment regimen comprises administration of 2, 3, 4, or 5 doses for every other day, every three, four, five, six, seven, or more days.

A method of administering multiple doses of a pharmaceutical composition described herein comprises providing two or more compositions, over a period of time, to a subject. In some cases, multiple doses of a pharmaceutical composition described herein can be administered to a subject over a defined time course. The methods according to this aspect of the disclosure comprise sequentially administering to a subject multiple doses of a pharmaceutical composition described herein. As used herein, “sequentially administering” can refer to administration regimen where each dose of the pharmaceutical composition described herein is administered to the subject at a different point in time, e.g., on different days separated by a predetermined interval (e.g., hours, days, weeks or months). In some cases, the methods comprise sequentially administering to the subject a single initial dose of a pharmaceutical composition described herein, followed by one or more secondary doses of the composition, and optionally followed by one or more tertiary doses of the composition.

The terms “initial dose,” “secondary doses,” and “tertiary doses,” can refer to the temporal sequence of administration of a pharmaceutical composition described herein. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses can all contain the same amount of a pharmaceutical composition described herein, and in some cases, may differ from one another in terms of frequency of administration. In certain cases, the amount of a pharmaceutical composition described herein contained in the initial, secondary and/or tertiary doses varies from one another (e.g., adjusted up or down as appropriate) during the course of treatment. In certain cases, one or more (e.g., 2, 3, 4, or 5) doses are administered at the beginning of the treatment regimen as “loading doses” followed by subsequent doses that are administered on a less frequent basis (e.g., “maintenance doses”).

In certain cases, each secondary and/or tertiary dose is administered after the immediately preceding dose. The term “the immediately preceding dose,” as used herein, can mean, in a sequence of multiple administrations, the dose of the pharmaceutical composition described herein which is administered to a subject prior to the administration of the very next dose in the sequence with no intervening doses. In certain embodiments, each secondary and/or tertiary dose is administered every day, every 2 days, 3 days, 4 days, 5 days, 6 days, or 7 days after the immediately preceding dose. In certain cases, each secondary and/or tertiary dose is administered every 0.5 weeks, 1 week, 2 weeks, 3 weeks, or 4 weeks after the immediately preceding dose.

The methods can comprise administering to a subject any number of secondary and/or tertiary doses of a pharmaceutical composition described herein. For example, in certain cases, only a single secondary dose is administered to the subject. In other cases, two or more (e.g., 2, 3, 4, 5, 6, 7, 8, or more) secondary doses are administered to the subject. Likewise, in certain cases, only a single tertiary dose is administered to the subject. In other cases, two or more (e.g., 2, 3, 4, 5, 6, 7, 8, or more) tertiary doses are administered to the subject.

In certain cases, the frequency at which the secondary and/or tertiary doses are administered to a subject can vary over the course of the treatment regimen. The frequency of administration can also be adjusted during the course of treatment.

In some cases, multiple doses are provided to produce a level of imatinib in the lungs of the subject. In some cases, multiple doses are provided to produce or maintain a level of imatinib in the subject for a period of time, for instance, for at least 20, 30, 40, 50, 60, 70, 80, 90, 100, 120, 150 days, or at least 1, 2, 3, 4, 5, 6, 7, 8, 9, 10, 12, 15, 18, 21, or 24 months, or at least 1, 2, 3, 4, or 5 years.

In some cases, in a multiple-dose regimen, the method of administering the pharmaceutical composition provided herein includes administering to a subject in need thereof the pharmaceutical composition for multiple times (multiple doses), e.g., at least 3, 4, 5, 6, 7, 8, 9, 10, 12, 15, 20, 30, 40, 50, 60, 100, 150, 200, or 500 times, with an interval of from 1 day to 56 days, such as about 49 days, 42 days, 35 days, 28 days, 21 days, 14 days, or 7 days. In some embodiments, in a multiple-dose regimen, the method provided herein comprises administering to a subject in need thereof the pharmaceutical composition described herein for at least 3 times, with an interval of about 1, 2, 3, 4, 5, 6, or 7 days. In some embodiments, in a subject that receives administration of multiple doses of the pharmaceutical composition provided herein (e.g., at least 3, 4, 5, 6, 7, 8, or 9 doses), a level of imatinib in plasma of the subject is maintained at a level with variation of less than 50%, 40%, 30%, 20%, or 10% for a period of longer than 1, 2, 3, 4, 5, 6, 7, 8, 9, 10, 11, 12, 13, 14, 15, 16, 18, or 20 weeks after the last dose. In some embodiments, in a subject that receives administration of multiple doses of the pharmaceutical composition (e.g., at least 3, 4, 5, 6, 7, 8, or 9 doses) provided herein, a level of imatinib in plasma of the subject is maintained at a first level for a period of longer than 1, 2, 3, 4, 5, 6, 7, 8, 9, 10, 11, 12, 13, 14, 15, 16, 18, 19, or 20 weeks after the second, third, fourth, fifth, sixth, seventh, eight, or the last dose, wherein the first level is higher than a level of imatinib measured shortly after the first dose (e.g., measured about 12, 24, 36, or 48 hours after the first dose).

### PK/PD

The pharmaceutical composition provided herein, when administered to a subject, can lead to advantageous therapeutic effects. The pharmaceutical composition provided herein can lead to higher retention of imatinib in the lungs of the subject, which, without wishing to be bound by a certain theory, can result in more exposure of the pulmonary artery to imatinib.

Cₘₐₓ can be calculated as the maximum measured plasma concentration of imatinib or derivative thereof. In some cases, the Cₘₐₓ is calculated as the peak measured plasma concentration that imatinib or derivative thereof achieves after the drug has been administrated. In some cases, Cₘₐₓ is calculated from lung concentration of imatinib or derivative thereof. In some cases, the Cₘₐₓ is measured in a human PK/PD study in which human subjects are administered with imatinib or derivative thereof according to the method disclosed herein, e.g., receive a single dose of the pharmaceutical composition provided herein or a multiple-dose regimen of the pharmaceutical composition provided herein.

In some cases, the Cₘₐₓ after administration of a single administration of the pharmaceutical composition provided herein is from about 50 ng/mL to about 1900 ng/mL, such as from about 50-70, 50-80, 50-90, 50-100, 50-110, 50-120, 50-130, 50-140, 50-150, 50-160, 50-170, 50-180, 50-190, 50-200, 50-250, 50-300, 50-350, 50-400, 50-450, 50-500, 50-550, 50-600, 50-650, 50-700, 50-800, 50-900, 50-1000, 50-1500, 50-1900, 70-80, 70-90, 70-100, 70-110, 70-120, 70-130, 70-140, 70-150, 70-160, 70-170, 70-180, 70-190, 70-200, 70-250, 70-300, 70-350, 70-400, 70-450, 70-500, 70-550, 70-600, 70-650, 70-700, 70-800, 70-900, 70-1000, 70-1500, 70-1900, 80-90, 80-100, 80-110, 80-120, 80-130, 80-140, 80-150, 80-160, 80-170, 80-180, 80-190, 80-200, 80-250, 80-300, 80-350, 80-400, 80-450, 80-500, 80-550, 80-600, 80-650, 80-700, 80-800, 80-900, 80-1000, 80-1500, 80-1900, 90-100, 90-110, 90-120, 90-130, 90-140, 90-150, 90-160, 90-170, 90-180, 90-190, 90-200, 90-250, 90-300, 90-350, 90-400, 90-450, 90-500, 90-550, 90-600, 90-650, 90-700, 90-800, 90-900, 90-1000, 90-1500, 90-1900, 100-110, 100-120, 100-130, 100-140, 100-150, 100-160, 100-170, 100-180, 100-190, 100-200, 100-250, 100-300, 100-350, 100-400, 100-450, 100-500, 100-550, 100-600, 100-650, 100-700, 100-800, 100-900, 100-1000, 100-1500, 100-1900, 110-120, 110-130, 110-140, 110-150, 110-160, 110-170, 110-180, 110-190, 110-200, 110-250, 110-300, 110-350, 110-400, 110-450, 110-500, 110-550, 110-600, 110-650, 110-700, 110-800, 110-900, 110-1000, 110-1500, 110-1900, 120-130, 120-140, 120-150, 120-160, 120-170, 120-180, 120-190, 120-200, 120-250, 120-300, 120-350, 120-400, 120-450, 120-500, 120-550, 120-600, 120-650, 120-700, 120-800, 120-900, 120-1000, 120-1500, 120-1900, 130-140, 130-150, 130-160, 130-170, 130-180, 130-190, 130-200, 130-250, 130-300, 130-350, 130-400, 130-450, 130-500, 130-550, 130-600, 130-650, 130-700, 130-800, 130-900, 130-1000, 130-1500, 130-1900, 140-150, 140-160, 140-170, 140-180, 140-190, 140-200, 140-250, 140-300, 140-350, 140-400, 140-450, 140-500, 140-550, 140-600, 140-650, 140-700, 140-800, 140-900, 140-1000, 140-1500, 140-1900, 150-160, 150-170, 150-180, 150-190, 150-200, 150-250, 150-300, 150-350, 150-400, 150-450, 150-500, 150-550, 150-600, 150-650, 150-700, 150-800, 150-900, 150-1000, 150-1500, 150-1900, 160-170, 160-180, 160-190, 160-200, 160-250, 160-300, 160-350, 160-400, 160-450, 160-500, 160-550, 160-600, 160-650, 160-700, 160-800, 160-900, 160-1000, 160-1500, 160-1900, 170-180, 170-190, 170-200, 170-250, 170-300, 170-350, 170-400, 170-450, 170-500, 170-550, 170-600, 170-650, 170-700, 170-800, 170-900, 170-1000, 170-1500, 170-1900, 180-190, 180-200, 180-250, 180-300, 180-350, 180-400, 180-450, 180-500, 180-550, 180-600, 180-650, 180-700, 180-800, 180-900, 180-1000, 180-1500, 180-1900, 190-200, 190-250, 190-300, 190-350, 190-400, 190-450, 190-500, 190-550, 190-600, 190-650, 190-700, 190-800, 190-900, 190-1000, 190-1500, 190-1900, 200-250, 200-300, 200-350, 200-400, 200-450, 200-500, 200-550, 200-600, 200-650, 200-700, 200-800, 200-900, 200-1000, 200-1500, 200-1900, 250-300, 250-350, 250-400, 250-450, 250-500, 250-550, 250-600, 250-650, 250-700, 250-800, 250-900, 250-1000, 250-1500, 250-1900, 300-350, 300-400, 300-450, 300-500, 300-550, 300-600, 300-650, 300-700, 300-800, 300-900, 300-1000, 300-1500, 300-1900, 350-400, 350-450, 350-500, 350-550, 350-600, 350-650, 350-700,350-800, 350-900, 350-1000, 350-1500, 350-1900, 400-450, 400-500, 400-550, 400-600, 400-650, 400-700, 400-800, 400-900, 400-1000, 400-1500, 400-1900, 450-500, 450-550, 450-600, 450-650, 450-700, 450-800, 450-900, 450-1000, 450-1500, 450-1900, 500-550, 500-600, 500-650, 500-700, 500-800, 500-900, 500-1000, 500-1500, 500-1900, 550-600, 550-650, 550-700, 550-800, 550-900, 550-1000, 550-1500, 550-1900, 600-650, 600-700, 600-800, 600-900, 600-1000, 600-1500, 600-1900, 650-700, 650-800, 650-900, 650-1000, 650-1500, 650-1900, 700-800, 700-900, 700-1000, 700-1500, 700-1900, 800-900, 800-1000, 800-1500, 800-1900, 900-1000, 900-1500, 900-1900, 1000-1500, 1000-1900, or 1500-1900 ng/mL. In some cases, the Cₘₐₓ after administration of a single administration of the pharmaceutical composition provided herein is from about 100 ng/mL to about 1500 ng/mL, such as 100-120, 100-150, 100-200, 100-250, 100-300, 100-350, 100-400, 100-450, 100-500, 100-550, 100-600, 100-650, 100-700, 100-800, 100-900, 100-1000, 100-1200, 100-1300, 100-1400, 100-1500, 150-200, 150-250, 150-300, 150-350, 150-400, 150-450, 150-500, 150-550, 150-600, 150-650, 150-700, 150-800, 150-900, 150-1000, 150-1100, 150-1200, 150-1300, 150-1400, 150-1500, 200-250, 200-300, 200-350, 200-400, 200-450, 200-500, 200-550, 200-600, 200-650, 200-700, 200-800, 200-900, 200-1000, 200-1500, 200-1900, 250-300, 250-350, 250-400, 250-450, 250-500, 250-550, 250-600, 250-650, 250-700, 250-800, 250-900, 250-1000, 250-1200, 250-1400, 250-1500, 300-350, 300-400, 300-450, 300-500, 300-550, 300-600, 300-650, 300-700, 300-800, 300-900, 300-1000, 300-1200, 300-1400, 300-1500, 350-400, 350-450, 350-500, 350-550, 350-600, 350-650, 350-700, 350-800, 350-900, 350-1000, 350-1200, 350-1400, 350-1500, 400-450, 400-500, 400-550, 400-600, 400-650, 400-700, 400-800, 400-900, 400-1000, 400-1200, 400-1300, 400-1500, 450-500, 450-550, 450-600, 450-650, 450-700, 450-800, 450-900, 450-1000, 450-1200, 450-1400, 450-1500, 500-550, 500-600, 500-650, 500-700, 500-800, 500-900, 500-1000, 500-1200, 500-1400, 500-1500550-600, 550-650, 550-700, 550-800, 550-900, 550-1000, 550-1200, 550-1300, 550-1500, 600-650, 600-700, 600-800, 600-900, 600-1000, 600-1200, 600-1400, 600-1500, 650-700, 650-800, 650-900, 650-1000, 650-1200, 650-1400, 650-1500, 700-800, 700-900, 700-1000, 700-1100, 700-1200, 700-1300, 700-1400, 700-1500, 800-900, 800-1000, 800-1100, 800-1200, 800-1300, 800-1400, 800-1500, 900-1000, 900-1100, 900-1200, 900-1300, 900-1400, 900-1500, 1000-1500, 1000-1100, 1000-1200, 1000-1300, 1000-1400, 1100-1200, 1100-1300, 1100-1400, 1100-1500, 1200-1300, 1200-1400, 1200-1500, 1300-1400, 1300-1500, or 1400-1500 ng/mL.

In some cases, the Cₘₐₓ after administration of a multiple-dose regimen of the pharmaceutical composition provided herein (e.g., administration of multiple doses of the composition disclosed herein within a period of time, such as within one, two, three, four, five, six, or seven consecutive days) is from about 50 ng/mL to about 2500 ng/mL, such as from about 50-70, 50-80, 50-90, 50-100, 50-110, 50-120, 50-130, 50-140, 50-150, 50-160, 50-170, 50-180, 50-190, 50-200, 50-250, 50-300, 50-350, 50-400, 50-450, 50-500, 50-550, 50-600, 50-650, 50-700, 50-800, 50-900, 50-1000, 50-1500, 50-2000, 50-2500, 70-80, 70-90, 70-100, 70-110, 70-120, 70-130, 70-140, 70-150, 70-160, 70-170, 70-180, 70-190, 70-200, 70-250, 70-300, 70-350, 70-400, 70-450, 70-500, 70-550, 70-600, 70-650, 70-700, 70-800, 70-900, 70-1000, 70-1500, 70-2000, 70-2500, 80-90, 80-100, 80-110, 80-120, 80-130, 80-140, 80-150, 80-160, 80-170, 80-180, 80-190, 80-200, 80-250, 80-300, 80-350, 80-400, 80-450, 80-500, 80-550, 80-600, 80-650, 80-700, 80-800, 80-900, 80-1000, 80-1500, 80-2000, 80-2500, 90-100, 90-110, 90-120, 90-130, 90-140, 90-150, 90-160, 90-170, 90-180, 90-190, 90-200, 90-250, 90-300, 90-350, 90-400, 90-450, 90-500, 90-550, 90-600, 90-650, 90-700, 90-800, 90-900, 90-1000, 90-1500, 90-2000, 90-2500, 100-110, 100-120, 100-130, 100-140, 100-150, 100-160, 100-170, 100-180, 100-190, 100-200, 100-250, 100-300, 100-350, 100-400, 100-450, 100-500, 100-550, 100-600, 100-650, 100-700, 100-800, 100-900, 100-1000, 100-1500, 100-2000, 100-2500, 110-120, 110-130, 110-140, 110-150, 110-160, 110-170, 110-180, 110-190, 110-200, 110-250, 110-300, 110-350, 110-400, 110-450, 110-500, 110-550, 110-600, 110-650, 110-700, 110-800, 110-900, 110-1000, 110-1500, 110-2000, 110-2500, 120-130, 120-140, 120-150, 120-160, 120-170, 120-180, 120-190, 120-200, 120-250, 120-300, 120-350, 120-400, 120-450, 120-500, 120-550, 120-600, 120-650, 120-700, 120-800, 120-900, 120-1000, 120-1500, 120-2000, 120-2500, 130-140, 130-150, 130-160, 130-170, 130-180, 130-190, 130-200, 130-250, 130-300, 130-350, 130-400, 130-450, 130-500, 130-550, 130-600, 130-650, 130-700, 130-800, 130-900, 130-1000, 130-1500, 130-2000, 130-2500, 140-150, 140-160, 140-170, 140-180, 140-190, 140-200, 140-250, 140-300, 140-350, 140-400, 140-450, 140-500, 140-550, 140-600, 140-650, 140-700, 140-800, 140-900, 140-1000, 140-1500, 140-2000, 140-2500, 150-160, 150-170, 150-180, 150-190, 150-200, 150-250, 150-300, 150-350, 150-400, 150-450, 150-500, 150-550, 150-600, 150-650, 150-700, 150-800, 150-900, 150-1000, 150-1500, 150-2000, 150-2500, 160-170, 160-180, 160-190, 160-200, 160-250, 160-300, 160-350, 160-400, 160-450, 160-500, 160-550, 160-600, 160-650, 160-700, 160-800, 160-900, 160-1000, 160-1500, 160-2000, 160-2500, 170-180, 170-190, 170-200, 170-250, 170-300, 170-350, 170-400, 170-450, 170-500, 170-550, 170-600, 170-650, 170-700, 170-800, 170-900, 170-1000, 170-1500, 170-2000, 170-2500, 180-190, 180-200, 180-250, 180-300, 180-350, 180-400, 180-450, 180-500, 180-550, 180-600, 180-650, 180-700, 180-800, 180-900, 180-1000, 180-1500, 180-2000, 180-2500, 190-200, 190-250, 190-300, 190-350, 190-400, 190-450, 190-500, 190-550, 190-600, 190-650, 190-700, 190-800, 190-900, 190-1000, 190-1500, 190-2000, 190-2500, 200-250, 200-300, 200-350, 200-400, 200-450, 200-500, 200-550, 200-600, 200-650, 200-700, 200-800, 200-900, 200-1000, 200-1500, 200-2000, 200-2500, 250-300, 250-350, 250-400, 250-450, 250-500, 250-550, 250-600, 250-650, 250-700, 250-800, 250-900, 250-1000, 250-1500, 250-2000, 250-2500, 300-350, 300-400, 300-450, 300-500, 300-550, 300-600, 300-650, 300-700, 300-800, 300-900, 300-1000, 300-1500, 300-2000, 300-2500, 350-400, 350-450, 350-500, 350-550, 350-600, 350-650, 350-700, 350-800, 350-900, 350-1000, 350-1500, 350-2000, 350-2500, 400-450, 400-500, 400-550, 400-600, 400-650, 400-700, 400-800, 400-900, 400-1000, 400-1500, 400-2000, 400-2500, 450-500, 450-550, 450-600, 450-650, 450-700, 450-800, 450-900, 450-1000, 450-1500, 450-2000, 450-2500, 500-550, 500-600, 500-650, 500-700, 500-800, 500-900, 500-1000, 500-1500, 500-2000, 550-600, 550-650, 550-700, 550-800, 550-900, 550-1000, 550-1500, 550-2000, 550-2500, 600-650, 600-700, 600-800, 600-900, 600-1000, 600-1500, 600-2000, 600-2500, 650-700, 650-800, 650-900, 650-1000, 650-1500, 650-2000, 700-800, 700-900, 700-1000, 700-1500, 700-2000, 700-2500, 800-900, 800-1000, 800-1500, 800-2000, 800-2500, 900-1000, 900-1500, 900-2000, 900-2500, 1000-1500, 1000-2000, 1000-2500, 1500-2000, 1500-2500, or 2000-2500 ng/mL. In some cases, the Cₘₐₓ after administration of a multiple-dose regimen of the pharmaceutical composition provided herein is from about 100 ng/mL to about 1800 ng/mL, such as 100-1800, 110-1800, 120-1800, 130-1800, 140-1800, 150-2800, 200-1800, 300-1800, 400-1800, 500-1800, 600-1800, 700-1800, 800-1800, 900-1800, 1000-1800, 1200-1800, 1400-1800, or 1500-1800 ng/mL.

In some cases, the AUC₀₋₂₄ after administration of a single dose of the pharmaceutical composition provided herein is from about 1 hr*μg/mL to about 24 hr*μg/mL, such as from 1-2, 1-3, 1-4, 1-5, 1-6, 1-7, 1-8, 1-9, 1-10, 1-15, 1-20, 1-24, 2-3, 2-4, 2-5, 2-6, 2-7, 2-8, 2-9, 2-10, 2-15, 2-20, 2-24, 3-4, 3-5, 3-6, 3-7, 3-8, 3-9, 3-10, 3-15, 3-20, 3-24, 4-5, 4-6, 4-7, 4-8, 4-9, 4-10, 4-15, 4-20, 4-24, 5-6, 5-7, 5-8, 5-9, 5-10, 5-15, 5-20, 5-24, 6-7, 6-8, 6-9, 6-10, 6-15, 6-20, 6-24, 7-8, 7-9, 7-10, 7-15, 7-20, 7-24, 8-9, 8-10, 8-15, 8-20, 8-24, 9-10, 9-15, 9-20, 9-24, 10-15, 20-20, 10-24, 15-20, 15-24, or 20-24 hr*μg/mL. In some cases, the AUC₀₋₂₄ after administration of a single dose of the pharmaceutical composition provided herein is from about 1 to about 17 hr*μg/mL.

In some cases, the AUC₀₋₂₄ after administration of a multiple-dose regimen of the pharmaceutical composition provided herein is from about 1 hr*μg/mL to about 40 hr*μg/mL, such as from 1-2, 1-3, 1-4, 1-5, 1-6, 1-7, 1-8, 1-9, 1-10, 1-15, 1-20, 1-25, 1-30, 1-35, 1-40, 2-3, 2-4, 2-5, 2-6, 2-7, 2-8, 2-9, 2-10, 2-15, 2-20, 2-25, 2-30, 2-35, 2-40, 3-4, 3-5, 3-6, 3-7, 3-8, 3-9, 3-10, 3-15, 3-20, 3-25, 3-30, 3-35, 3-40, 4-5, 4-6, 4-7, 4-8, 4-9, 4-10, 4-15, 4-20, 4-25, 4-30, 4-35, 4-40, 5-6, 5-7, 5-8, 5-9, 5-10, 5-15, 5-20, 5-25, 5-30, 5-35, 5-40, 6-7, 6-8, 6-9, 6-10, 6-15, 6-20, 6-25, 6-30, 6-35, 6-40, 7-8, 7-9, 7-10, 7-15, 7-20, 7-25, 7-30, 7-35, 7-40, 8-9, 8-10, 8-15, 8-20, 8-25, 8-30, 8-35, 8-40, 9-10, 9-15, 9-20, 9-25, 9-30, 9-35, 9-40, 10-15, 20-20, 10-25, 10-30, 10-35, 10-40, 15-20, 15-25, 15-30, 15-35, 15-40, 20-25, 20-30, 20-35, 20-40, 25-30, 25-35, 25-40, 30-35, 30-40 or 35-40 hr*g/mL. In some cases, the AUC₀₋₂₄ after administration of a multiple-dose regimen of the pharmaceutical composition provided herein is from about 1 to about 24 hr*μg/mL.

In some cases, the AUC_{0-∞} after administration of a single dose of the pharmaceutical composition provided herein is from about 1 hr*g/mL to about 38 hr*g/mL, such as from 1-2, 1-3, 1-4, 1-5, 1-6, 1-7, 1-8, 1-9, 1-10, 1-15, 1-20, 1-25, 1-30, 1-35, 1-38, 2-3, 2-4, 2-5, 2-6, 2-7, 2-8, 2-9, 2-10, 2-15, 2-20, 2-25, 2-30, 2-35, 2-38, 3-4, 3-5, 3-6, 3-7, 3-8, 3-9, 3-10, 3-15, 3-20, 3-25, 3-30, 3-35, 3-38, 4-5, 4-6, 4-7, 4-8, 4-9, 4-10, 4-15, 4-20, 4-25, 4-30, 4-35, 4-38, 5-6, 5-7, 5-8, 5-9, 5-10, 5-15, 5-20, 5-25, 5-30, 5-35, 5-38, 6-7, 6-8, 6-9, 6-10, 6-15, 6-20, 6-25, 6-30, 6-35, 6-38, 7-8, 7-9, 7-10, 7-15, 7-20, 7-25, 7-30, 7-35, 7-38, 8-9, 8-10, 8-15, 8-20, 8-25, 8-30, 8-35, 8-38, 9-10, 9-15, 9-20, 9-25, 9-30, 9-35, 9-38, 10-15, 20-20, 10-25, 10-30, 10-35, 10-38, 15-20, 15-25, 15-30, 15-35, 15-38, 20-25, 20-30, 20-35, 20-38, 25-30, 25-35, 25-38, 30-35, 30-38, or 35-38 hr*μg/mL. In some cases, the AUC_{0-∞} after administration of a single dose of the pharmaceutical composition provided herein is from about 1 to about 23 hr*μg/mL.

In some cases, the AUC_{0-∞} after administration of a multiple-dose regimen of the pharmaceutical composition provided herein is from about 1 hr*μg/mL to about 80 hr*μg/mL, such as from 1-2, 1-3, 1-4, 1-5, 1-6, 1-7, 1-8, 1-9, 1-10, 1-15, 1-20, 1-25, 1-30, 1-35, 1-40, 1-45, 1-50, 1-55, 1-60, 1-65, 1-70, 1-75, 1-80, 2-3, 2-4, 2-5, 2-6, 2-7, 2-8, 2-9, 2-10, 2-15, 2-20, 2-25, 2-30, 2-35, 2-40, 2-45, 2-50, 2-55, 2-60, 2-65, 2-70, 2-75, 2-80, 3-4, 3-5, 3-6, 3-7, 3-8, 3-9, 3-10, 3-15, 3-20, 3-25, 3-30, 3-35, 3-40, 3-45, 3-50, 3-55, 3-60, 3-65, 3-70, 3-75, 3-80, 4-5, 4-6, 4-7, 4-8, 4-9, 4-10, 4-15, 4-20, 4-25, 4-30, 4-35, 4-40, 4-45, 4-50, 4-55, 4-60, 4-65, 4-70, 4-75, 4-80, 5-6, 5-7, 5-8, 5-9, 5-10, 5-15, 5-20, 5-25, 5-30, 5-35, 5-40, 5-45, 5-50, 5-55, 5-60, 5-65, 5-70, 5-75, 5-80, 6-7, 6-8, 6-9, 6-10, 6-15, 6-20, 6-25, 6-30, 6-35, 6-40, 6-45, 6-50, 6-55, 6-60, 6-65, 6-70, 6-75, 6-80, 7-8, 7-9, 7-10, 7-15, 7-20, 7-25, 7-30, 7-35, 7-40, 7-45, 7-50, 7-55, 7-60, 7-65, 7-70, 7-75, 7-80, 8-9, 8-10, 8-15, 8-20, 8-25, 8-30, 8-35, 8-40, 8-45, 8-50, 8-55, 8-60, 8-65, 8-70, 8-75, 8-80, 9-10, 9-15, 9-20, 9-25, 9-30, 9-35, 9-40, 9-45, 9-50, 9-55, 9-60, 9-65, 9-70, 9-75, 9-80, 10-15, 20-20, 10-25, 10-30, 10-35, 10-40, 10-45, 10-50, 10-55, 10-60, 10-65, 10-70, 10-75, 10-80, 15-20, 15-25, 15-30, 15-35, 15-40, 15-45, 15-50, 15-55, 15-60, 15-65, 15-70, 15-75, 15-80, 20-25, 20-30, 20-35, 20-40, 20-45, 20-50, 20-55, 20-60, 20-65, 20-70, 20-75, 20-80, 25-30, 25-35, 25-40, 25-45, 25-50, 25-55, 25-60, 25-65, 25-70, 25-75, 25-80, 30-35, 30-40, 30-45, 30-50, 30-55, 30-60, 30-65, 30-70, 30-75, 30-80, 35-40, 35-45, 35-50, 35-55, 35-60, 35-65, 35-70, 35-75, 35-80, 40-45, 40-50, 40-55, 40-60, 40-65, 40-70, 40-75, 40-80, 55-60, 55-65, 55-70, 55-75, 55-80, 60-65, 60-70, 60-75, 60-80, 65-70, 65-75, 65-80, 70-75, 70-80 or 75-80 hr*μg/mL. In some cases, the AUC_{0-∞} after administration of a multiple-dose regimen of the pharmaceutical composition provided herein is from about 1 to about 37 hr*μg/mL.

When examined in an animal study in which mammalian animal subjects receive a single dose of the pharmaceutical composition provided herein, or a diluted version of the pharmaceutical composition provided herein, via inhalational administration or oral administration, the pharmaceutical composition can result in a ratio of maximum measured lung concentration of the imatinib or derivative thereof post the inhalational administration (C_{max,inhalation,lung}) to maximum plasma concentration of the imatinib or derivative thereof the inhalational administration (C_{max,inhalation,plasma}) ranging from about 20 to about 1000, such as from about 20 to about 50, from about 20 to about 100, from about 20 to about 200, from about 20 to about 300, from about 20 to about 400, from about 20 to about 500, from about 20 to about 600, from about 20 to about 700, from about 20 to about 800, from about 20 to about 900, from about 50 to about 100, from about 50 to about 150, from about 50 to about 200, from about 50 to about 300, from about 50 to about 500, from about 50 to about 750, from about 50 to about 900, from about 100 to about 150, from about 100 to about 200, from about 100 to about 300, from about 100 to about 500, from about 100 to about 750, from about 100 to about 900, from about 150 to about 200, from about 150 to about 300, from about 150 to about 500, from about 150 to about 750, from about 150 to about 900, from about 200 to about 250, from about 200 to about 300, from about 200 to about 400, from about 200 to about 500, from about 200 to about 600, from about 200 to about 700, from about 200 to about 800, from about 200 to about 900, from about 300 to about 400, from about 300 to about 500, from about 300 to about 600, from about 300 to about 700, from about 300 to about 800, from about 300 to about 900, from about 400 to about 500, from about 400 to about 600, from about 400 to about 700, from about 400 to about 800, from about 400 to about 900, from about 500 to about 600, from about 500 to about 700, from about 500 to about 800, from about 500 to about 900, from about 600 to about 700, from about 600 to about 800, from about 600 to about 900, from about 700 to about 800, from about 700 to about 900, or from about 800 to about 900, such as about 20, about 30, about 40, about 50, about 75, about 100, about 120, about 150, about 200, about 250, about 300, about 350, about 400, about 450, about 500, about 550, about 600, about 700, about 800, about 900, or about 1000. When examined in the animal study, the pharmaceutical composition can result in a ratio of maximum measured lung concentration of the imatinib or derivative thereof post the inhalational administration (C_{max,inhalation,lung}) to maximum measured lung concentration of the imatinib or derivative thereof post the oral administration (C_{max,po,lung}) ranging from about 20 to about 1000, such as from about 20 to about 50, from about 20 to about 100, from about 20 to about 200, from about 20 to about 300, from about 20 to about 400, from about 20 to about 500, from about 20 to about 600, from about 20 to about 700, from about 20 to about 800, from about 20 to about 900, from about 50 to about 100, from about 50 to about 150, from about 50 to about 200, from about 50 to about 300, from about 50 to about 500, from about 50 to about 750, from about 50 to about 900, from about 100 to about 150, from about 100 to about 200, from about 100 to about 300, from about 100 to about 500, from about 100 to about 750, from about 100 to about 900, from about 150 to about 200, from about 150 to about 300, from about 150 to about 500, from about 150 to about 750, from about 150 to about 900, from about 200 to about 250, from about 200 to about 300, from about 200 to about 400, from about 200 to about 500, from about 200 to about 600, from about 200 to about 700, from about 200 to about 800, from about 200 to about 900, from about 300 to about 400, from about 300 to about 500, from about 300 to about 600, from about 300 to about 700, from about 300 to about 800, from about 300 to about 900, from about 400 to about 500, from about 400 to about 600, from about 400 to about 700, from about 400 to about 800, from about 400 to about 900, from about 500 to about 600, from about 500 to about 700, from about 500 to about 800, from about 500 to about 900, from about 600 to about 700, from about 600 to about 800, from about 600 to about 900, from about 700 to about 800, from about 700 to about 900, or from about 800 to about 900, such as about 20, about 30, about 40, about 50, about 75, about 100, about 120, about 150, about 200, about 250, about 300, about 350, about 400, about 450, about 500, about 550, about 600, about 700, about 800, about 900, or about 1000.

When examined in the animal study, the pharmaceutical composition can result in a ratio of area under lung concentration of the imatinib or derivative thereof-time curve from 0 to 24 hours post the inhalational administration (AUC_{inhalation,lung,0-24}) to area under plasma concentration of the imatinib or derivative thereof-time curve from 0 to 24 hours post the inhalational administration (AUC_{inhalation,plasma,0-24}) ranging from about 10 to about 1000, such as from about 10 to about 50, from about 10 to about 100, from about 10 to about 200, from about 10 to about 300, from about 10 to about 400, from about 10 to about 500, from about 10 to about 600, from about 10 to about 700, from about 10 to about 800, from about 10 to about 900, from about 20 to about 50, from about 20 to about 100, from about 20 to about 200, from about 20 to about 300, from about 20 to about 400, from about 20 to about 500, from about 20 to about 600, from about 20 to about 700, from about 20 to about 800, from about 20 to about 900, from about 50 to about 100, from about 50 to about 150, from about 50 to about 200, from about 50 to about 300, from about 50 to about 500, from about 50 to about 750, from about 50 to about 900, from about 100 to about 150, from about 100 to about 200, from about 100 to about 300, from about 100 to about 500, from about 100 to about 750, from about 100 to about 900, from about 150 to about 200, from about 150 to about 300, from about 150 to about 500, from about 150 to about 750, from about 150 to about 900, from about 200 to about 250, from about 200 to about 300, from about 200 to about 400, from about 200 to about 500, from about 200 to about 600, from about 200 to about 700, from about 200 to about 800, from about 200 to about 900, from about 300 to about 400, from about 300 to about 500, from about 300 to about 600, from about 300 to about 700, from about 300 to about 800, from about 300 to about 900, from about 400 to about 500, from about 400 to about 600, from about 400 to about 700, from about 400 to about 800, from about 400 to about 900, from about 500 to about 600, from about 500 to about 700, from about 500 to about 800, from about 500 to about 900, from about 600 to about 700, from about 600 to about 800, from about 600 to about 900, from about 700 to about 800, from about 700 to about 900, or from about 800 to about 900, such as about 20, about 30, about 40, about 50, about 75, about 100, about 120, about 150, about 200, about 250, about 300, about 350, about 400, about 450, about 500, about 550, about 600, about 700, about 800, about 900, or about 1000. When examined in the animal study, the pharmaceutical composition can result in a ratio of area under lung concentration of the imatinib or derivative thereof-time curve from 0 to 24 hours post the inhalational administration (AUC_{inhalation,lung,0-24}) to area under lung concentration of the imatinib or derivative thereof-time curve from 0 to 24 hours post the oral administration (AUC_{po,lung,0-24}) ranging from about 10 to about 1000, such as from about 10 to about 50, from about 10 to about 100, from about 10 to about 200, from about 10 to about 300, from about 10 to about 400, from about 10 to about 500, from about 10 to about 600, from about 10 to about 700, from about 10 to about 800, from about 10 to about 900, from about 20 to about 50, from about 20 to about 100, from about 20 to about 200, from about 20 to about 300, from about 20 to about 400, from about 20 to about 500, from about 20 to about 600, from about 20 to about 700, from about 20 to about 800, from about 20 to about 900, from about 50 to about 100, from about 50 to about 150, from about 50 to about 200, from about 50 to about 300, from about 50 to about 500, from about 50 to about 750, from about 50 to about 900, from about 100 to about 150, from about 100 to about 200, from about 100 to about 300, from about 100 to about 500, from about 100 to about 750, from about 100 to about 900, from about 150 to about 200, from about 150 to about 300, from about 150 to about 500, from about 150 to about 750, from about 150 to about 900, from about 200 to about 250, from about 200 to about 300, from about 200 to about 400, from about 200 to about 500, from about 200 to about 600, from about 200 to about 700, from about 200 to about 800, from about 200 to about 900, from about 300 to about 400, from about 300 to about 500, from about 300 to about 600, from about 300 to about 700, from about 300 to about 800, from about 300 to about 900, from about 400 to about 500, from about 400 to about 600, from about 400 to about 700, from about 400 to about 800, from about 400 to about 900, from about 500 to about 600, from about 500 to about 700, from about 500 to about 800, from about 500 to about 900, from about 600 to about 700, from about 600 to about 800, from about 600 to about 900, from about 700 to about 800, from about 700 to about 900, or from about 800 to about 900, such as about 20, about 30, about 40, about 50, about 75, about 100, about 120, about 150, about 200, about 250, about 300, about 350, about 400, about 450, about 500, about 550, about 600, about 700, about 800, about 900, or about 1000.

When examined in the animal study, the pharmaceutical composition can result in a therapeutic advantage (Rd) of inhalational administration compared to oral administration ranging from about 2 to about 200, such as from about 2 to about 5, from about 2 to about 10, from about 2 to about 20, from about 2 to about 30, from about 2 to about 40, from about 2 to about 50, from about 2 to about 60, from about 2 to about 80, from about 2 to about 100, from about 2 to about 120, from about 2 to about 140, from about 2 to about 160, from about 2 to about 180, from about 5 to about 10, from about 5 to about 20, from about 5 to about 30, from about 5 to about 40, from about 5 to about 50, from about 5 to about 60, from about 5 to about 80, from about 5 to about 100, from about 5 to about 120, from about 5 to about 140, from about 5 to about 160, from about 5 to about 180, from about 10 to about 20, from about 10 to about 30, from about 10 to about 40, from about 10 to about 50, from about 10 to about 60, from about 10 to about 80, from about 10 to about 100, from about 10 to about 120, from about 10 to about 140, from about 10 to about 160, from about 10 to about 180, from about 20 to about 30, from about 20 to about 40, from about 20 to about 50, from about 20 to about 60, from about 20 to about 80, from about 20 to about 100, from about 20 to about 120, from about 20 to about 140, from about 20 to about 160, from about 20 to about 180, from about 30 to about 50, from about 30 to about 60, from about 30 to about 80, from about 30 to about 100, from about 30 to about 120, from about 30 to about 140, from about 30 to about 160, from about 30 to about 180, from about 50 to about 60, from about 50 to about 80, from about 50 to about 100, from about 50 to about 120, from about 50 to about 140, from about 50 to about 160, from about 50 to about 180, from about 60 to about 80, from about 60 to about 100, from about 60 to about 120, from about 60 to about 140, from about 60 to about 160, from about 60 to about 180, from about 80 to about 100, from about 80 to about 120, from about 80 to about 140, from about 80 to about 160, from about 80 to about 180, from about 100 to about 120, from about 120 to about 140, from about 120 to about 160, from about 120 to about 180, from about 140 to about 160, from about 140 to about 180, or from about 160 to about 180, such as about 2, about 5, about 10, about 12, about 15, about 20, about 25, about 30, about 40, about 50, about 60, about 70, about 80, about 90, about 100, about 120, about 140, about 160, about 180, or about 200. Rd can be calculated according to the following equation:

$$Rd = \frac{( {AUC}_{inhalation , lung , 0 - 24} / {AUC}_{inhalation , plasma , 0 - 24} )}{( {AUC}_{po , lung , 0 - 24} / {AUC}_{po , plasma , 0 - 24} )} .$$

In some cases, the diluted version of the pharmaceutical composition used in the animal study is diluted by at least about 2 times with water as compared to the pharmaceutical composition provided herein, for instance, diluted by from about 2 to about 20 times, e.g., diluted by about 2, 3, 4, 5, 6, 7, 8, 9, or 10 times. In some cases, the diluted version of the pharmaceutical composition used in the animal study is diluted from the pharmaceutical composition provided herein immediately before delivery to the animals, because the imatinib or derivative thereof contained therein can precipitate as result of the dilution, thus resulting in suspension of imatinib or derivative thereof.

In general, when discussing the anatomy and physiology of the lungs, the respiratory system can be divided into two main regions: the conducting zone (Central Lung) and the respiratory zone (Deep Lung or Peripheral Lung). As used herein, the term “conducting zone” refers to the area of the lungs containing the upper respiratory tract (nasal cavity, pharynx, larynx) and the lower respiratory tract (trachea, bronchi, bronchioles). The primary function of the conducting zone is to transport air to and from the respiratory zone. As used herein, the term “respiratory zone” refers to the area of the lungs containing the respiratory bronchioles, alveolar ducts, and alveoli. This is where the actual gas exchange occurs, with oxygen being absorbed into the bloodstream and carbon dioxide being released from the bloodstream into the alveoli to be exhaled.

In some cases, the method provided herein results in a peripheral lung dose of imatinib or a derivative thereof ranging from about 10 mg to 40 mg, such as from about 15 mg to 35 mg, from about 20 mg to about 30 mg, in the subject administered with the pharmaceutical composition provided herein. In some cases, the method results in a peripheral lung dose of imatinib or a derivative thereof about 12 mg, about 16 mg, about 20 mg, about 24 mg, about 28 mg, about 32 mg, or about 36 mg. In some cases, the method provided herein results in a peripheral lung dose of imatinib or a derivative thereof ranging from about 10 mg to 100 mg, such as from about 15 mg to 80 mg, from about 30 mg to about 60 mg, in the subject administered with the pharmaceutical composition provided herein. In some cases, the method results in a peripheral lung dose of imatinib or a derivative thereof about 15 mg, about 20 mg, about 30 mg, about 40 mg, about 45 mg, about 50 mg, or about 60 mg. In some cases, the peripheral lung dose measures the amount of the imatinib or derivative thereof delivered to tissues of interest within the respiratory zone of the lungs, which includes the respiratory bronchioles, alveolar ducts, and alveoli, from where the imatinib or derivative thereof can reach tissues of interest such as the pulmonary arterial tissues or arterioles.

The relative deposition and dispersion of the formulation after inhalation can be estimated by scintigraphy. Scintigraphy can be conducted when imatinib or derivative thereof in pharmaceutical composition provided herein is labeled by a radioactive isotope, administered to subjects via inhalation, and then detected by a scintillation detector after administration. Scintigraphy can be conducted according to routine procedures available in the art, such as the procedures described in Davis et al. Eur J Nucl Med 1992; 19(11):971-86.

Tissue distribution of a radiolabeled drug derivative can also be measured by quantitative whole-body autoradiography (QWBA), whereby whole body sections of a study animal administered previously dosed with the radiolabeled drug are assayed for radioactivity by application of a radiosensitive imaging plate to the sections. QWBA can be conducted according to routine procedures available in the art, such as the procedures described in Whitby, B. (2006). Quantitative Whole-Body Autoradiography (QWBA). In G. Lappin & S. Temple (Eds.), Radiotracers in Drug Development (1st ed., pp. 26). Taylor & Francis.

Tissue distribution of a radiolabeled drug derivative can also be measured in tissue samples via micro-autoradiography (MARG), whereby fresh tissue samples are snap-frozen, cryosectioned, and exposed to radiosensitive substance applied to a flat surface such as a microscope slide. MARG can be conducted according to routine procedures available in the art, such as the procedures described in Solon E G, Schweitzer A, Stoeckli M, Prideaux B. Autoradiography, MALDI-MS, and SIMS-MS imaging in pharmaceutical discovery and development, AAPS J., 2010, vol. 12 (pg. 11-26). In one example, a cohort of study animals (e.g. Sprague Dawley or Han Wistar rats) are each administered a dose of a pharmaceutical composition of the disclosure containing 14C-labeled imatinib via inhalation and sacrificed at varying time points post administration (e.g. 1, 2, 4, and 6 hours post-administration). Lung vasculature and pulmonary arteriole samples can then be analyzed by MARG to evaluate the change spatial distribution of radiolabeled drug in lung tissue over time.

In some cases, the method provided herein results in a central lung dose of imatinib or a derivative thereof ranging from about 10 mg to 100 mg, such as from about 15 mg to 80 mg, from about 30 mg to about 60 mg, in the subject administered with the pharmaceutical composition provided herein. In some cases, the method results in a central lung dose of imatinib or a derivative thereof about 15 mg, about 20 mg, about 30 mg, about 40 mg, about 45 mg, about 50 mg, or about 60 mg. The central lung dose measures the amount of the imatinib or derivative thereof delivered to tissues of interest within the conducting zone of the lungs, which includes the upper respiratory tract (nasal cavity, pharynx, larynx) and the lower respiratory tract (trachea, bronchi, bronchioles).

### Kits and Systems

In one aspect, also provided herein are kits for treatment of pulmonary diseases via inhalation. The kits can include one or more pharmaceutical agents, for instance, a salt of imatinib, or some additional active agent(s) as described herein. In some cases, the kits include container for the pharmaceutical agents or compositions. In some cases, unit doses of the pharmaceutical agents as discussed above are provided in the kits. In some cases, the kits also include containers/receptacles for containing the pharmaceutical agents.

The pharmaceutical composition according to one or more embodiments of the disclosure may, if desired, contain a combination of pharmaceutical agent for treatment of pulmonary diseases (e.g., imatinib free base, imatinib salt) and one or more additional active agents. The pharmaceutical compositions can be aerosolized prior to administration or can be presented to a user in the form of an aerosol.

In some cases, all the starting materials are sterilized by established technologies that meet the standards for medical use. Typically, manufacturing equipment is sterilized before use. Some or all of other additional pharmaceutically acceptable carrier or excipient, solubilizer, or other additional ingredients of the pharmaceutical composition (e.g., cyclodextrin, e.g., SBEβCD or HPβCD; e.g., acids, e.g., acetic acid, hydrochloric acid, nitric acid, or citric acid; e.g., saccharin, e.g., saccharin sodium, e.g., lipid or fatty acid, e.g., co-solvent) can added into a suitable container.

In some cases, the kits include separate containers/receptacles for containing the pharmaceutical composition as described herein. In some other cases, the kits include a single container for containing the pharmaceutical composition. The kits can further include instructions for methods of using the kit. The instructions can be presented in the form of a data sheet, a manual, in a piece of paper, printed on one or more containers or devices of the kit. Alternatively, the instructions can be provided in electronic form, for instance, available in a disc or online with a weblink available from the kit. The instructions for use of the kit can comprise instructions for use of the pharmaceutical composition and the aerosolization device (e.g., a nebulizer) to treat any applicable indication, e.g., pulmonary disease. The instructions for use of the kit can comprise instructions for use of the pharmaceutical composition and the aerosolization device (e.g., a nebulizer) to treat a pulmonary disease. In some cases, the kits include a nose clip. A nose clip can be used to hinder passage of air through a nose of a subject during inhalation and increase the proportion of a total inhaled volume that is the aerosol issued by the nebulizer.

Unit doses of the pharmaceutical compositions can be placed in a container. The container can be inserted into an aerosolization device. The container can be of a suitable shape, size, and material to contain the pharmaceutical composition and to provide the pharmaceutical composition in a usable condition. For example, the container can comprise a wall which comprises a material that does not adversely react with the pharmaceutical composition. In addition, the wall can comprise a material that allows the capsule to be opened to allow the pharmaceutical composition to be aerosolized.

In some cases, the pharmaceutical composition in the container is stable in reduced temperatures (e.g., 2-8° C.) for an extended period of time and may prolong the stability of the pharmaceutical composition.

### Terminology

As used herein, the singular forms “a,” “an,” and “the” can include plural referents unless the context clearly dictates otherwise. Thus, for example, reference to “pharmaceutical agent for treatment of pulmonary diseases” can include not only a single active agent but also a combination or mixture of two or more different active agents.

Reference herein to “one embodiment,” “one version,” or “one aspect” can include one or more such embodiments, versions or aspects, unless otherwise clear from the context.

As used herein, the term “pharmaceutically acceptable solvate” can refer to a solvate that retains one or more of the biological activities and/or properties of the pharmaceutical agent and that is not biologically or otherwise undesirable. Examples of pharmaceutically acceptable solvates include, but are not limited to, pharmaceutical agents for treatment of pulmonary diseases in combination with water, isopropanol, ethanol, methanol, DMSO, ethyl acetate, acetic acid, ethanolamine, or combinations thereof.

As used herein, the term “salt” is equivalent to the term “pharmaceutically acceptable salt,” and can refer to those salts that retain one or more of the biological activities and properties of the free acids and bases and that are not biologically or otherwise undesirable. Illustrative examples of pharmaceutically acceptable salts include, but are not limited to, sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, di nitrobenzoates, hydroxybenzoates, methoxybenzoates, phthalates, sulfonates, xylenesulfonates, phenylacetates, phenyipropionates, phenylbutyrates, citrates, lactates, γ-hydroxybutyrates, glycolates, tartrates, methanesulfonates, propanesulfonates, naphthalene-1-sulfonates, naphthalene-2-sulfonates, and mandelates.

The term “about” in relation to a reference numerical value can include a range of values plus or minus 10% from that value. For example, the amount “about 10” includes amounts from 9 to 11, including the reference numbers of 9, 10, and 11. The term “about” in relation to a reference numerical value can also include a range of values plus or minus 10%, 9%, 8%, 7%, 6%, 5%, 4%, 3%, 2%, or 1% from that value.

As used herein, the terms “treating” and “treatment” can refer to reduction in severity and/or frequency of symptoms, elimination of symptoms and/or underlying cause, reduction in likelihood of the occurrence of symptoms and/or underlying cause, and/or remediation of damage. Thus, “treating” a patient with an active agent as provided herein can include prevention of a particular condition, disease, or disorder in a susceptible individual as well as treatment of a clinically symptomatic individual.

As used herein, “nominal amount” can refer to the amount contained within the unit dose receptacle(s) that are administered.

As used herein, “effective amount” can refer to an amount covering both therapeutically effective amounts and prophylactically effective amounts.

As used herein, a “therapeutically effective amount” of an active agent can refer to an amount that is effective to achieve a desired therapeutic result. A therapeutically effective amount of a given active agent can vary with respect to factors such as the type and severity of the disorder or disease being treated and the age, gender, and weight of the patient. In some cases, “inhalation” (e.g., “oral inhalation” or “nasal inhalation”) refers to inhalation delivery of a therapeutically effective amount of a pharmaceutical agent contained in one unit dose receptacle, which, in some instance, can require one or more breaths, like 1, 2, 3, 4, 5, 6, 7, 8, 9, or more breaths. For example, if the effective amount is 90 mg, and each unit dose receptacle contains 30 mg, the delivery of the effective amount can require 3 inhalations.

Unless otherwise specified, the term “therapeutically effective amount” can include a “prophylactically effective amount,” e.g., an amount of active agent that is effective to prevent the onset or recurrence of a particular condition, disease, or disorder in a susceptible individual.

As used herein, the phrase “minimum effective amount” can mean the minimum amount of a pharmaceutical agent necessary to achieve an effective amount.

As used herein, “mass median diameter” or “MMD” can refer to the median diameter of a plurality of droplets, typically in a polydisperse droplet population, e.g., consisting of a range of droplet sizes.

As used herein, “particle” can refer to “droplet” of aerosolized pharmaceutical composition.

As used herein, “geometric diameter” can refer to the diameter of a single droplet, as determined by microscopy, unless the context indicates otherwise.

As used herein, “mass median aerodynamic diameter” or “MMAD” can refer to the median aerodynamic size of a plurality of droplets or droplets, typically in a polydisperse population. The “aerodynamic diameter” can be the diameter of a unit density sphere having the same settling velocity, generally in air, as a powder and is therefore a useful way to characterize an aerosolized powder or other dispersed droplet or droplet formulation in terms of its settling behavior. The aerodynamic diameter encompasses droplet or droplet shape, density, and physical size of the droplet or droplet. As used herein, MMAD refers to the median of the aerodynamic droplet or droplet size distribution of aerosolized droplets determined by cascade impaction, unless the context indicates otherwise.

By a “pharmaceutically acceptable” component is meant a component that is not biologically or otherwise undesirable, e.g., the component can be incorporated into a pharmaceutical composition of the disclosure and administered to a patient as described herein without causing any significant undesirable biological effects or interacting in a deleterious manner with any of the other components of the formulation in which it is contained. When the term “pharmaceutically acceptable” is used to refer to an excipient, it can imply that the component has met the required standards of toxicological and manufacturing testing or that it is included on the Inactive Ingredient Guide prepared by the U.S. Food and Drug Administration.

As used herein, “active nebulizer” or “nebulizer” refers to an inhalation device that does not rely solely on a patient's inspiratory effort to disperse and aerosolize a pharmaceutical composition contained within the device in a reservoir or in a unit dose form and does include inhaler devices that comprise a means for providing energy to disperse and aerosolize the drug composition, Such as pressurized gas and vibrating or rotating elements.

As used herein, “room temperature” can refer to a temperature that is from 18° C. to 25° C.

### CERTAIN EMBODIMENTS

Embodiment 1. A composition, comprising an aqueous solution or suspension that comprises:
(1) imatinib or a derivative thereof,
(2) a solubility enhancer, and
(3) a pH buffer,
wherein said aqueous solution or suspension:
(a) has a concentration of said imatinib or derivative thereof of from 20 to 500 mg/mL;
(b) has a viscosity of at most 10 centipoise; and
(c) has a pH of 3 to 8.

Embodiment 2. The composition of Embodiment 1, wherein the solubility enhancer is selected from the group consisting of: cyclodextrins, lipids, co-solvents, organic acids, and surfactants.

Embodiment 3. The composition of Embodiment 1, wherein the solubility enhancer comprises a cyclodextrin.

Embodiment 4. The composition of Embodiment 3, wherein said aqueous solution or suspension has said cyclodextrin at a concentration of from about 1% (w/v) to about 80% (w/v).

Embodiment 5. The composition of Embodiment 1, wherein the solubility enhancer comprises a lipid or a fatty acid.

Embodiment 6. The composition of Embodiment 5, wherein said lipid or fatty acid is selected from the group consisting of: polyethoxylated castor oil, phospholipids, glycolipids, ganglioside GM1, sphingomyelin, phosphatidic acid, cardiolipin; lipids bearing polymer chains such as polyethylene glycol (PEG), chitin, hyaluronic acid, and polyvinylpyrrolidone; lipids bearing sulfonated monosaccharides, lipid-bearing sulfonated disaccharides, lipid bearing sulfonated polysaccharides; fatty acids such as palmitic acid, stearic acid, and oleic acid; cholesterol, cholesterol esters, and cholesterol hemisuccinate.

Embodiment 7. The composition of Embodiment 1, wherein said solubility enhancer comprises a co-solvent.

Embodiment 8. The composition of Embodiment 7, wherein said co-solvent comprises glycerol or ethanol.

Embodiment 9. The composition of Embodiment 1, wherein said solubility enhancer comprises an organic acid.

Embodiment 10. The composition of Embodiment 9, wherein said organic acid is selected from the group consisting of: acetic acid, acid modified starch, aconitic acid, adipic acid, hexanedioic acid, L-ascorbic acid, benzoic acid, caprylic acid, octanoic acid, cholic acid, citric acid, desoxycholic acid, erythorbic acid (D-isoascorbic acid), formic acid, L-glutamic acid, L-glutamic acid hydrochloride, glycocholic acid, hydrochloric acid, iron naphthenate, iron tallate, D(−)-lactic acid, lactic acid, L(+)-lactic acid, linoleic acid, malic acid, L-malic acid, niacin (nicotinic acid), oleic acid, pectin, pectinic acid, phosphoric acid, L(+)-potassium acid tartrate, propionic acid, acid hydrolyzed proteins, sodium acid pyrophosphate, acidic sodium aluminum phosphate, sorbic acid, stearic acid, succinic acid, sulfamic acid, sulfuric acid, tannic acid, L(+)-tartaric acid, taurocholic acid, and thiodipropionic acid.

Embodiment 11. The composition of Embodiment 1, wherein said solubility enhancer comprises a surfactant.

Embodiment 12. The composition of Embodiment 11, wherein said surfactant comprises Tween, sodium lauryl sulfate (SLS), or dipalmitoylphosphatidylcholine (DPPC).

Embodiment 13. A composition, comprising an aqueous solution or suspension that comprises imatinib or a derivative thereof and cyclodextrin, wherein said aqueous solution or suspension has said cyclodextrin at a concentration of from about 1% (w/v) to about 80% (w/v).

Embodiment 14. The composition of any one of Embodiments 3, 4 or 13, wherein said cyclodextrin is selected from the group consisting of: α-cyclodextrin, β-cyclodextrin, 7-cyclodextrin, hydroxypropyl-β-cyclodextrin, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin dimaltosyl-β-cyclodextrin, methyl-β-cyclodextrin, 6A-amino-6A-deoxy-N-(3-hydroxypropyl)-β-cyclodextrin, succinyl-α-cyclodextrin, succinyl-β-cyclodextrin, succinyl-γ-cyclodextrin, sulfobutylether-α-cyclodextrin, sulfobutylether-β-cyclodextrin, sulfobutylether-γ-cyclodextrin, carboxymethyl-α-cyclodextrin, carboxymethyl-β-cyclodextrin, carboxymethyl-γ-cyclodextrin, 2-carboxyethyl-α-cyclodextrin, 2-carboxyethyl-β-cyclodextrin, 2-carboxyethyl-γ-cyclodextrin, phosphate-α-cyclodextrin, phosphate-β-cyclodextrin, phosphate-γ-cyclodextrin, sulfoalkylether-β-cyclodextrins, and sulfoalkylether-γ-cyclodextrins.

Embodiment 15. The composition of any one of Embodiments 3, 4 or 13, wherein said cyclodextrin comprises succinyl-α-cyclodextrin, succinyl-β-cyclodextrin, succinyl-γ-cyclodextrin, sulfobutylether-α-cyclodextrin, sulfobutylether-β-cyclodextrin, sulfobutylether-γ-cyclodextrin, carboxymethyl-α-cyclodextrin, carboxymethyl-β-cyclodextrin, carboxymethyl-γ-cyclodextrin, 2-carboxyethyl-α-cyclodextrin, 2-carboxyethyl-β-cyclodextrin, 2-carboxyethyl-γ-cyclodextrin, phosphate-α-cyclodextrin, phosphate-β-cyclodextrin, or phosphate-γ-cyclodextrin.

Embodiment 16. The composition of any one of Embodiments 3, 4 or 13, wherein said cyclodextrin comprises an anionic cyclodextrin.

Embodiment 17. A composition, comprising an aqueous solution or suspension that comprises imatinib or a derivative thereof and cyclodextrin, wherein said cyclodextrin comprises an anionic cyclodextrin.

Embodiment 18. A composition, comprising an aqueous solution or suspension that comprises imatinib or a derivative thereof, cyclodextrin, a pH buffer, and a surfactant.

Embodiment 19. The composition of any one of 3, 4 or 13-18, wherein said aqueous solution or suspension comprises a salt of said cyclodextrin.

Embodiment 20. The composition of Embodiment 19, wherein said salt of said cyclodextrin is a salt selected from the group consisting of: sodium salt, calcium salt, magnesium salt, iron salt, chromium salt, copper salt, zinc salt, lysine salt, arginine salt, and histidine salt.

Embodiment 21. The composition of any one of Embodiments 3, 4 or 13-18, wherein said cyclodextrin comprises sulfobutylether-β-cyclodextrin.

Embodiment 22. The composition of any one of Embodiments 3, 4 or 13-18, wherein said aqueous solution or suspension comprises sulfobutylether-β-cyclodextrin sodium.

Embodiment 23. The composition of any one of Embodiments 3, 4 or 13-18, wherein said cyclodextrin comprises hydroxypropyl-β-cyclodextrin.

Embodiment 24. The composition of any one of Embodiments 13-17, 21, or 23, wherein said aqueous solution or suspension further comprises a pH buffer.

Embodiment 25. The composition of any one of Embodiments 1-12 or 24, wherein said pH buffer comprises an organic acid salt of citric acid, lactic acid, ascorbic acid, gluconic acid, carbonic acid, tartaric acid, succinic acid, acetic acid, or phthalic acid, Tris, tromethamine hydrochloride, or a phosphate buffer.

Embodiment 26. The composition of any one of Embodiments 1-12 or 24, wherein said pH buffer comprises a phosphate buffer.

Embodiment 27. The composition of any one of Embodiments 1-26, wherein said aqueous solution or suspension further comprises an artificial sweetener.

Embodiment 28. The composition of Embodiment 27, wherein said artificial sweetener is selected from the group consisting of: acesulfame potassium, aspartame, cyclamate, mogrosides, saccharin, stevia, sucralose, neotame, and sugar alcohols, and combinations thereof.

Embodiment 29. The composition of any one of Embodiments 13-17 or 21-28, wherein said aqueous solution or suspension further comprises a surfactant.

Embodiment 30. The composition of any one of Embodiments 18-29, wherein said surfactant comprises Tween, sodium lauryl sulfate (SLS), or dipalmitoylphosphatidylcholine (DPPC).

Embodiment 31. The composition of any one of Embodiments 13-30, wherein said aqueous solution or suspension has a viscosity of at most 10 centipoise.

Embodiment 32. The composition of any one of Embodiments 13-30, wherein said aqueous solution or suspension has a viscosity of at most 9.5 centipoise, at most 9.0 centipoise, at most 8.5 centipoise, at most 8.0 centipoise, at most 7.6 centipoise, at most 7.4 centipoise, at most 7.2 centipoise, at most 7.0 centipoise, at most 6.8 centipoise, at most 6.6 centipoise, at most 6.4 centipoise, at most 6.2 centipoise, at most 6.0 centipoise, at most 5.8 centipoise, at most 5.6 centipoise, at most 5.4 centipoise, at most 5.2 centipoise, at most 5.0 centipoise, at most 4.8 centipoise, at most 4.6 centipoise, at most 4.4 centipoise, at most 4.2 centipoise, at most 4.0 centipoise, at most 3.8 centipoise, at most 3.6 centipoise, at most 3.4 centipoise, at most 3.2 centipoise, at most 3.0 centipoise, at most 2.8 centipoise, at most 2.6 centipoise, at most 2.4 centipoise, at most 2.2 centipoise, at most 2.0 centipoise, at most 1.8 centipoise, at most 1.6 centipoise, at most 1.4 centipoise, at most 1.2 centipoise, at most 1.0 centipoise, at most 0.8 centipoise, at most 0.6 centipoise, at most 0.4 centipoise, or at most 0.2 centipoise.

Embodiment 33. The composition of any one of Embodiments 13-30, wherein said aqueous solution or suspension has a viscosity of about 0.1 centipoise, 0.2 centipoise, 0.3 centipoise, 0.4 centipoise, 0.5 centipoise, 0.6 centipoise, 0.7 centipoise, 0.8 centipoise, 0.9 centipoise, 1.0 centipoise, 1.1 centipoise, 1.2 centipoise, 1.3 centipoise, 1.4 centipoise, 1.5 centipoise, 1.6 centipoise, 1.7 centipoise, 1.8 centipoise, 1.9 centipoise, 2.0 centipoise, 2.1 centipoise, 2.2 centipoise, 2.3 centipoise, 2.4 centipoise, 2.5 centipoise, 2.6 centipoise, 2.8 centipoise, 3.0 centipoise, 3.2 centipoise, 3.5 centipoise, 3.8 centipoise, 4.0 centipoise, 4.2 centipoise, 4.5 centipoise, 4.8 centipoise, 5.0 centipoise, 5.5 centipoise, 6.0 centipoise, 6.5 centipoise, 7.0 centipoise, 7.5 centipoise, 8.0 centipoise, or 8.5 centipoise.

Embodiment 34. The composition of any one of Embodiments 13-33, wherein said aqueous solution or suspension has from 20 to 500 mg/mL of said imatinib or derivative thereof.

Embodiment 35. The composition of any one of Embodiments 1-12 or 34, wherein said aqueous solution or suspension has from 20 mg/mL to 400 mg/mL, from 20 mg/mL to 300 mg/mL, from 20 mg/mL to 200 mg/mL, from 100 mg/mL to 500 mg/mL, from 200 mg/mL to 500 mg/mL, from 300 mg/mL to 500 mg/mL, from 400 mg/mL to 500 mg/mL, from 100 mg/mL to 400 mg/mL, from 100 mg/mL to 300 mg/mL, from 100 mg/mL to 200 mg/mL, from 200 mg/mL to 400 mg/mL, from 200 mg/mL to 300 mg/mL, from 20 to 100, from 20 mg/mL to 80 mg/mL, from 20 mg/mL to 60 mg/mL, from 20 mg/mL to 40 mg/mL, from 20 mg/mL to 30 mg/mL, from 30 mg/mL to 40 mg/mL, from 40 mg/mL to 60 mg/mL, from 40 mg/mL to 80 mg/mL, from 40 mg/mL to 100 mg/mL, from 40 mg/mL to 120 mg/mL, from 40 mg/mL to 150 mg/mL, from 60 mg/mL to 80 mg/mL, from 60 mg/mL to 100 mg/mL, from 60 mg/mL to 120 mg/mL, or from 60 mg/mL to 150 mg/mL of said imatinib or derivative thereof.

Embodiment 36. The composition of any one of Embodiments 1-12 or 34, wherein said aqueous solution or suspension has about 50 mg/mL, about 60 mg/mL, about 70 mg/mL, about 80 mg/mL, about 90 mg/mL, about 100 mg/mL, about 110 mg/mL, about 120 mg/mL, about 130 mg/mL, about 140 mg/mL, or about 150 mg/mL of said imatinib or derivative thereof.

Embodiment 37. The composition of any one of Embodiments 1-12 or 34, wherein said aqueous solution or suspension has about 80 mg/mL of said imatinib or derivative thereof.

Embodiment 38. The composition of any one of Embodiments 13-37, wherein said aqueous solution or suspension has a pH of 3 to 8.

Embodiment 39. The composition of any one of Embodiments 1-12 or 38, wherein said pH of said aqueous solution or suspension is from 3 to 6, from 4 to 6, from 4.5 to 5.5, from 5 to 6, from 4 to 7, from 5 to 7, or from 6 to 7.

Embodiment 40. The composition of any one of Embodiments 1-12 or 38, wherein said pH of said aqueous solution or suspension is about 4.5, about 4.7, about 4.8, about 4.9, about 5.0, about 5.1, about 5.2, about 5.4, about 5.5, or about 5.6.

Embodiment 41. The composition of any one of Embodiments 1-12 or 38, wherein said pH of said aqueous solution or suspension is from 7 to 8.

Embodiment 42. The composition of any one of Embodiments 1-12 or 38, wherein said pH of said aqueous solution or suspension is about 7.0, about 7.2, about 7.4, about 7.6, about 7.8, or about 8.0.

Embodiment 43. The composition of any one of Embodiments 3, 4 or 13-42, wherein said aqueous solution or suspension has said cyclodextrin at a concentration of from about 2% (w/v) to about 70% (w/v), from about 2% (w/v) to about 60% (w/v), from about 2% (w/v) to about 50% (w/v), from about 2% (w/v) to about 40% (w/v), from about 2% (w/v) to about 30% (w/v), from about 2% (w/v) to about 20% (w/v), from about 2% (w/v) to about 15% (w/v), from about 2% (w/v) to about 10% (w/v), from about 2% (w/v) to about 8% (w/v), from about 2% (w/v) to about 5% (w/v), from about 5% (w/v) to about 80% (w/v), from about 5% (w/v) to about 70% (w/v), from about 5% (w/v) to about 60% (w/v), from about 5% (w/v) to about 50% (w/v), from about 5% (w/v) to about 40% (w/v), from about 5% (w/v) to about 30% (w/v), from about 5% (w/v) to about 20% (w/v), from about 5% (w/v) to about 15% (w/v), from about 5% (w/v) to about 12% (w/v), from about 5% (w/v) to about 10% (w/v), from about 10% (w/v) to about 60% (w/v), from about 10% (w/v) to about 50% (w/v), from about 10% (w/v) to about 40% (w/v), from about 10% (w/v) to about 30% (w/v), from about 20% (w/v) to about 30% (w/v), from about 10% (w/v) to about 25% (w/v), from about 19% (w/v) to about 25% (w/v), from about 19.5% (w/v) to about 25% (w/v), from about 20% (w/v) to about 25% (w/v), from about 20.5% (w/v) to about 25% (w/v), from about 21% (w/v) to about 25% (w/v), from about 21.5% (w/v) to about 25% (w/v), from about 22% (w/v) to about 25% (w/v), from about 22.5% (w/v) to about 25% (w/v), from about 23% (w/v) to about 25% (w/v), from about 10% (w/v) to about 20% (w/v), or from about 10% (w/v) to about 15% (w/v).

Embodiment 44. The composition of any one of Embodiments 3, 4 or 13-42, wherein said aqueous solution or suspension has said cyclodextrin at a concentration of from 5% (w/v) to 40% (w/v).

Embodiment 45. The composition of any one of Embodiments 3, 4 or 13-42, wherein said aqueous solution or suspension has said cyclodextrin at a concentration of from 10% (w/v) to 20% (w/v).

Embodiment 46. The composition of any one of Embodiments 3, 4 or 13-42, wherein said aqueous solution or suspension has said cyclodextrin at a concentration of from 25% (w/v) to 40% (w/v).

Embodiment 47. The composition of any one of Embodiments 3, 4 or 13-42, wherein said aqueous solution or suspension has said cyclodextrin at a concentration of about 10% (w/v), about 12% (w/v), about 14% (w/v), about 15% (w/v), about 16% (w/v), about 18% (w/v), or about 20% (w/v).

Embodiment 48. The composition of any one of Embodiments 3, 4 or 13-42, wherein said aqueous solution or suspension has said cyclodextrin at a concentration of about 22% (w/v), about 24% (w/v), about 26% (w/v), about 28% (w/v), about 30% (w/v), about 32% (w/v), about 34% (w/v), about 36% (w/v), about 38% (w/v), or about 40% (w/v).

Embodiment 49. The composition of any one of Embodiments 1-48, wherein said composition comprises said aqueous solution.

Embodiment 50. The composition of Embodiment 49, wherein solubility of the imatinib or derivative thereof in the aqueous solution is negatively correlated with the pH of the aqueous solution.

Embodiment 51. The composition of Embodiment 49 or 50, wherein solubility of the imatinib or derivative thereof in the aqueous solution is positively correlated with concentration of the cyclodextrin in the aqueous solution.

Embodiment 52. The composition of any one of Embodiments 1-48, wherein said composition comprises said aqueous suspension.

Embodiment 53. The composition of any one of Embodiments 1-52, wherein said composition comprises less than 1 mg/mL, less than 0.5 mg/mL, less than 0.1 mg/mL, less than 0.05 mg/mL, less than 0.01 mg/mL, less than 0.005 mg/mL, less than 0.001 mg/mL, or less than 0.0001 mg/mL imatinib mesylate.

Embodiment 54. The composition of any one of Embodiments 1-52, wherein said composition does not comprise imatinib mesylate.

Embodiment 55. The composition of any one of Embodiments 1-54, wherein said imatinib or derivative thereof comprises imatinib free base.

Embodiment 56. The composition of any one of Embodiments 1-54, wherein said imatinib or derivative thereof is imatinib free base.

Embodiment 57. The composition of any one of Embodiments 1-54, wherein said composition comprises a salt of said imatinib or derivative thereof selected from the group consisting of: acetate salt, formate salt, citrate salt, phosphate salt, maleate salt, fumarate salt, tartrate salt, malonate salt, lactic salt, and succinate salt.

Embodiment 58. A pharmaceutical composition, comprising the composition of any one of Embodiments 1 to 57.

Embodiment 59. The pharmaceutical composition of Embodiment 58, wherein said pharmaceutical composition is formulated for inhalational administration.

Embodiment 60. The pharmaceutical composition of Embodiment 58 or 59, wherein said aqueous solution further comprises a pharmaceutically acceptable excipient.

Embodiment 61. The pharmaceutical composition of Embodiment 60, wherein said pharmaceutically acceptable excipient comprises a surfactant.

Embodiment 62. The pharmaceutical composition of Embodiment 61, wherein said surfactant comprises Tween, sodium lauryl sulfate (SLS), or dipalmitoylphosphatidylcholine (DPPC).

Embodiment 63. The pharmaceutical composition of Embodiment 60, wherein said pharmaceutically acceptable excipient comprises a lipid.

Embodiment 64. The pharmaceutical composition of Embodiment 63, wherein said lipid comprises a polymeric lipid, a sulfonated poly saccharide, or a fatty acid.

Embodiment 65. The pharmaceutical composition of Embodiment 63, wherein said lipid comprises a polymeric lipid, a sulfonated poly saccharide, or a fatty acid.

Embodiment 66. The pharmaceutical composition of any one of Embodiments 61-65, wherein said pharmaceutical composition is organoleptically tolerated when inhaled by a human subject.

Embodiment 67. The pharmaceutical composition of any one of Embodiments 61-66, wherein said pharmaceutical composition does not induce cough reflex when inhaled by a human subject.

Embodiment 68. The pharmaceutical composition of any one of Embodiments 61-67, wherein said pharmaceutical composition is not or minimally irritative to mouth or throat when inhaled by a human subject.

Embodiment 69. A pharmaceutical composition, comprising an aqueous solution that comprises cyclodextrin and a therapeutically effective amount of imatinib or a derivative thereof, wherein said aqueous solution is formulated for inhalational administration.

Embodiment 70. The pharmaceutical composition of Embodiment 69, wherein said aqueous solution has a viscosity of at most 10 centipoise.

Embodiment 71. The pharmaceutical composition of Embodiment 69, wherein said aqueous solution has a viscosity of at most 2.5 centipoise.

Embodiment 72. The pharmaceutical composition of any one of Embodiments 69-71, wherein said aqueous solution has from 20 to 500 mg/mL of said imatinib or derivative thereof.

Embodiment 73. The pharmaceutical composition of Embodiment 72, wherein said aqueous solution has from 20 mg/mL to 400 mg/mL, from 20 mg/mL to 300 mg/mL, from 20 mg/mL to 200 mg/mL, from 100 mg/mL to 500 mg/mL, from 200 mg/mL to 500 mg/mL, from 300 mg/mL to 500 mg/mL, from 400 mg/mL to 500 mg/mL, from 100 mg/mL to 400 mg/mL, from 100 mg/mL to 300 mg/mL, from 100 mg/mL to 200 mg/mL, from 200 mg/mL to 400 mg/mL, from 200 mg/mL to 300 mg/mL, from 20 to 100, from 20 mg/mL to 80 mg/mL, from 20 mg/mL to 60 mg/mL, from 20 mg/mL to 40 mg/mL, from 20 mg/mL to 30 mg/mL, from 30 mg/mL to 40 mg/mL, from 40 mg/mL to 60 mg/mL, from 40 mg/mL to 80 mg/mL, from 40 mg/mL to 100 mg/mL, from 40 mg/mL to 120 mg/mL, from 40 mg/mL to 150 mg/mL, from 60 mg/mL to 80 mg/mL, from 60 mg/mL to 100 mg/mL, from 60 mg/mL to 120 mg/mL, or from 60 mg/mL to 150 mg/mL of said imatinib or derivative thereof.

Embodiment 74. The pharmaceutical composition of Embodiment 72, wherein said aqueous solution has about 50 mg/mL, about 60 mg/mL, about 70 mg/mL, about 80 mg/mL, about 90 mg/mL, about 100 mg/mL, about 110 mg/mL, about 120 mg/mL, about 130 mg/mL, about 140 mg/mL, or about 150 mg/mL of said imatinib or derivative thereof.

Embodiment 75. The pharmaceutical composition of Embodiment 72, wherein said aqueous solution has about 80 mg/mL of said imatinib or derivative thereof.

Embodiment 76. The pharmaceutical composition of any one of Embodiments 69-75, wherein said aqueous solution has a pH of 3 to 8.

Embodiment 77. The pharmaceutical composition of Embodiment 76, wherein said pH of said aqueous solution is from 3 to 6, from 4 to 6, from 4.5 to 5.5, from 5 to 6, from 4 to 7, from 5 to 7, or from 6 to 7.

Embodiment 78. The pharmaceutical composition of Embodiment 76, wherein said pH of said aqueous solution is about 4.5, about 4.7, about 4.8, about 4.9, about 5.0, about 5.1, about 5.2, about 5.4, about 5.5, or about 5.6.

Embodiment 79. The pharmaceutical composition of Embodiment 76, wherein said pH of said aqueous solution or suspension is from 7 to 8.

Embodiment 80. The pharmaceutical composition of Embodiment 76, wherein said pH of said aqueous solution is about 7.0, about 7.2, about 7.4, about 7.6, about 7.8, or about 8.0.

Embodiment 81. The pharmaceutical composition of any one of Embodiments 69-80, wherein said aqueous solution further comprises a pH buffer.

Embodiment 82. The pharmaceutical composition of Embodiment 81, wherein said pH buffer comprises an organic acid salt of citric acid, lactic acid, ascorbic acid, gluconic acid, carbonic acid, tartaric acid, succinic acid, acetic acid, or phthalic acid, Tris, tromethamine hydrochloride, or a phosphate buffer.

Embodiment 83. The pharmaceutical composition of any one of Embodiments 69-82, wherein said aqueous solution or suspension further comprises an artificial sweetener.

Embodiment 84. The pharmaceutical composition of Embodiment 83, wherein said artificial sweetener is selected from the group consisting of: acesulfame potassium, aspartame, cyclamate, mogrosides, saccharin, stevia, sucralose, neotame, and sugar alcohols, and combinations thereof.

Embodiment 85. The pharmaceutical composition of any one of Embodiments 69-84, wherein said cyclodextrin is selected from the group consisting of: α-cyclodextrin, β-cyclodextrin, γ-cyclodextrin, hydroxypropyl-β-cyclodextrin, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin dimaltosyl-β-cyclodextrin, methyl-β-cyclodextrin, 6A-amino-6A-deoxy-N-(3-hydroxypropyl)-β-cyclodextrin, succinyl-α-cyclodextrin, succinyl-β-cyclodextrin, succinyl-γ-cyclodextrin, sulfobutylether-α-cyclodextrin, sulfobutylether-β-cyclodextrin, sulfobutylether-γ-cyclodextrin, carboxymethyl-α-cyclodextrin, carboxymethyl-β-cyclodextrin, carboxymethyl-γ-cyclodextrin, 2-carboxyethyl-α-cyclodextrin, 2-carboxyethyl-β-cyclodextrin, 2-carboxyethyl-γ-cyclodextrin, phosphate-α-cyclodextrin, phosphate-β-cyclodextrin, phosphate-γ-cyclodextrin, sulfoalkylether-β-cyclodextrins, and sulfoalkylether-γ-cyclodextrins.

Embodiment 86. The pharmaceutical composition of any one of Embodiments 69-84, wherein said cyclodextrin comprises succinyl-α-cyclodextrin, succinyl-β-cyclodextrin, succinyl-γ-cyclodextrin, sulfobutylether-α-cyclodextrin, sulfobutylether-β-cyclodextrin, sulfobutylether-γ-cyclodextrin, carboxymethyl-α-cyclodextrin, carboxymethyl-β-cyclodextrin, carboxymethyl-γ-cyclodextrin, 2-carboxyethyl-α-cyclodextrin, 2-carboxyethyl-β-cyclodextrin, 2-carboxyethyl-γ-cyclodextrin, phosphate-α-cyclodextrin, phosphate-β-cyclodextrin, or phosphate-γ-cyclodextrin.

Embodiment 87. The pharmaceutical composition of any one of Embodiments 69-84, wherein said cyclodextrin comprises an anionic cyclodextrin.

Embodiment 88. The pharmaceutical composition of any one of Embodiments 69-84, wherein said cyclodextrin comprises sulfobutylether-β-cyclodextrin.

Embodiment 89. The pharmaceutical composition of any one of Embodiments 69-84, wherein said cyclodextrin comprises hydroxypropyl-β-cyclodextrin.

Embodiment 90. The pharmaceutical composition of any one of Embodiments 69-84, wherein said aqueous solution comprises a salt of said cyclodextrin.

Embodiment 91. The pharmaceutical composition of Embodiment 90, wherein said salt of said cyclodextrin is a salt selected from the group consisting of: sodium salt, calcium salt, magnesium salt, iron salt, chromium salt, copper salt, zinc salt, lysine salt, arginine salt, and histidine salt.

Embodiment 92. The pharmaceutical composition of any one of Embodiments 69-82, wherein said aqueous solution comprises sulfobutylether-β-cyclodextrin sodium.

Embodiment 93. The pharmaceutical composition of any one of Embodiments 69-92, wherein said aqueous solution has said cyclodextrin at a concentration of from about 1% (w/v) to about 80% (w/v), from about 2% (w/v) to about 70% (w/v), from about 2% (w/v) to about 60% (w/v), from about 2% (w/v) to about 50% (w/v), from about 2% (w/v) to about 40% (w/v), from about 2% (w/v) to about 30% (w/v), from about 2% (w/v) to about 20% (w/v), from about 2% (w/v) to about 15% (w/v), from about 2% (w/v) to about 10% (w/v), from about 2% (w/v) to about 8% (w/v), from about 2% (w/v) to about 5% (w/v), from about 5% (w/v) to about 80% (w/v), from about 5% (w/v) to about 70% (w/v), from about 5% (w/v) to about 60% (w/v), from about 5% (w/v) to about 50% (w/v), from about 5% (w/v) to about 40% (w/v), from about 5% (w/v) to about 30% (w/v), from about 5% (w/v) to about 20% (w/v), from about 5% (w/v) to about 15% (w/v), from about 5% (w/v) to about 12% (w/v), from about 5% (w/v) to about 10% (w/v), from about 10% (w/v) to about 60% (w/v), from about 10% (w/v) to about 50% (w/v), from about 10% (w/v) to about 40% (w/v), from about 10% (w/v) to about 30% (w/v), from about 20% (w/v) to about 30% (w/v), from about 10% (w/v) to about 25% (w/v), from about 19% (w/v) to about 25% (w/v), from about 19.5% (w/v) to about 25% (w/v), from about 20% (w/v) to about 25% (w/v), from about 20.5% (w/v) to about 25% (w/v), from about 21% (w/v) to about 25% (w/v), from about 21.5% (w/v) to about 25% (w/v), from about 22% (w/v) to about 25% (w/v), from about 22.5% (w/v) to about 25% (w/v), from about 23% (w/v) to about 25% (w/v), from about 10% (w/v) to about 20% (w/v), or from about 10% (w/v) to about 15% (w/v).

Embodiment 94. The pharmaceutical composition of any one of Embodiments 69-93, wherein said aqueous solution has said cyclodextrin at a concentration of from 5% (w/v) to 40% (w/v).

Embodiment 95. The pharmaceutical composition of any one of Embodiments 69-93, wherein said aqueous solution has said cyclodextrin at a concentration of from 10% (w/v) to 20% (w/v).

Embodiment 96. The pharmaceutical composition of any one of Embodiments 69-93, wherein said aqueous solution has said cyclodextrin at a concentration of from 25% (w/v) to 40% (w/v).

Embodiment 97. The pharmaceutical composition of any one of Embodiments 69-93, wherein said aqueous solution has said cyclodextrin at a concentration of about 10% (w/v), about 12% (w/v), about 14% (w/v), about 15% (w/v), about 16% (w/v), about 18% (w/v), or about 20% (w/v).

Embodiment 98. The pharmaceutical composition of any one of Embodiments 69-93, wherein said aqueous solution has said cyclodextrin at a concentration of about 22% (w/v), about 24% (w/v), about 26% (w/v), about 28% (w/v), about 30% (w/v), about 32% (w/v), about 34% (w/v), about 36% (w/v), about 38% (w/v), or about 40% (w/v).

Embodiment 99. The pharmaceutical composition of any one of Embodiments 69-98, wherein said pharmaceutical composition is organoleptically tolerated when inhaled by a human subject.

Embodiment 100. The pharmaceutical composition of any one of Embodiments 69-99, wherein said pharmaceutical composition does not induce cough reflex when inhaled by a human subject.

Embodiment 101. The pharmaceutical composition of any one of Embodiments 69-100, wherein said pharmaceutical composition is not or minimally irritative to mouth or throat when inhaled by a human subject.

Embodiment 102. The pharmaceutical composition of any one of Embodiments 69-101, wherein said pharmaceutical composition comprises less than 1 mg/mL, less than 0.5 mg/mL, less than 0.1 mg/mL, less than 0.005 mg/mL, less than 0.001 mg/mL, or less than 0.0001 mg/mL imatinib mesylate.

Embodiment 103. The pharmaceutical composition of any one of Embodiments 69-101, wherein said pharmaceutical composition does not comprise imatinib mesylate.

Embodiment 104. The pharmaceutical composition of any one of Embodiments 69-103, wherein said imatinib or derivative thereof comprises imatinib free base.

Embodiment 105. The pharmaceutical composition of any one of Embodiments 69-103, wherein said imatinib or derivative thereof is imatinib free base.

Embodiment 106. The pharmaceutical composition of any one of Embodiments 69-103, wherein said pharmaceutical composition comprises a salt of said imatinib or derivative thereof selected from the group consisting of: acetate salt, formate salt, citrate salt, phosphate salt, maleate salt, fumarate salt, tartrate salt, malonate salt, lactic salt, and succinate salt.

Embodiment 107. The pharmaceutical composition of any one of Embodiments 69-106, wherein said aqueous solution further comprises a pharmaceutically acceptable excipient.

Embodiment 108. The pharmaceutical composition of Embodiment 107, wherein said pharmaceutically acceptable excipient comprises a surfactant.

Embodiment 109. The pharmaceutical composition of Embodiment 108, wherein said surfactant comprises Tween, sodium lauryl sulfate (SLS), or dipalmitoylphosphatidylcholine (DPPC).

Embodiment 110. The pharmaceutical composition of Embodiment 107, wherein said pharmaceutically acceptable excipient comprises a lipid.

Embodiment 111. The pharmaceutical composition of Embodiment 110, wherein said lipid comprises a polymeric lipid, a sulfonated poly saccharide, or a fatty acid.

Embodiment 112. The pharmaceutical composition of Embodiment 110, wherein said lipid comprises a polymeric lipid, a sulfonated poly saccharide, or a fatty acid.

Embodiment 113. The pharmaceutical composition of Embodiments 69-112, wherein solubility of the imatinib or derivative thereof in the aqueous solution is negatively correlated with the pH of the aqueous solution.

Embodiment 114. The pharmaceutical composition of Embodiments 69-113, wherein solubility of the imatinib or derivative thereof in the aqueous solution is positively correlated with concentration of the cyclodextrin in the aqueous solution.

Embodiment 115. An aerosol composition, comprising nebulized droplets of the pharmaceutical composition of any one of Embodiments 58-114, or nebulized droplets of the composition of any one of Embodiments 1-57.

Embodiment 116. The aerosol composition of Embodiment 115, wherein said nebulized droplets have an average mass median aerodynamic diameter of from 1 μm to 5 μm, from 1 μm to 4 μm, from 1 μm to 3 μm, from 1 μm to 2 μm, from 2 μm to 5 μm, from 2 μm to 4 μm, from 2 μm to 3 μm, or from 3 μm to 4 μm.

Embodiment 117. A unit dose of the pharmaceutical composition of any one of Embodiments 58-114, or the composition of any one of Embodiments 1-57, or the aerosol composition of Embodiment 115 or 116, comprising from about 10 mg to about 500 mg of imatinib or a derivative thereof.

Embodiment 118. The unit dose of Embodiment 117, comprising from 20 mg to 180 mg, from 20 mg to 150 mg, from 20 mg to 120 mg, from 20 mg to 100 mg, from 20 mg to 80 mg, from 20 mg to 60 mg, from 20 mg to 40 mg, from 40 mg to 120 mg, from 60 mg to 100 mg, or from 60 mg to 80 mg of said imatinib or derivative thereof.

Embodiment 119. A method of treating a subject having a pulmonary disease, comprising administering to said subject in need thereof via inhalation said pharmaceutical composition of any one of Embodiments 58-114.

Embodiment 120. The method of Embodiment 119, comprising administering to said subject from about 10 mg to about 500 mg of said imatinib or derivative thereof via inhalation.

Embodiment 121. The method of Embodiment 119, comprising administering to said subject from 20 mg to 180 mg, from 20 mg to 150 mg, from 20 mg to 120 mg, from 20 mg to 100 mg, from 20 mg to 80 mg, from 20 mg to 60 mg, from 20 mg to 40 mg, from 40 mg to 120 mg, from 60 mg to 100 mg, or from 60 mg to 80 mg of said imatinib or derivative thereof.

Embodiment 122. The method of any one of Embodiments 119-121, wherein said pulmonary disease comprises lung fibrosis, lung cancer, or pulmonary hypertension.

Embodiment 123. The method of any one of Embodiments 119-121, wherein said pulmonary disease comprises pulmonary arterial hypertension.

Embodiment 124. The method of any one of Embodiments 119-123, comprising administering to said subject said pharmaceutical composition at least once per day.

Embodiment 125. The method of any one of Embodiments 119-123, comprising administering to said subject said pharmaceutical composition 2, 3, 4, or 5 times per day.

Embodiment 126. The method of any one of Embodiments 119-125, comprising administering to said subject said pharmaceutical composition for a period of at least 5, 10, 20, 30, 60, 100, or 300 days, at least 1, 2, 3, 4, or 5 years.

Embodiment 127. The method of any one of Embodiments 119-126, wherein said administering is performed using a nebulizer.

Embodiment 128. The method of Embodiment 127, wherein said nebulizer is a jet nebulizer, a vibrating mesh nebulizer, an ultrasonic nebulizer, an electrospray nebulizer, or a hydraulic atomization device.

Embodiment 129. The method of any one of Embodiments 119-128, wherein administration of a single unit dose of said pharmaceutical composition takes place within 30 minutes.

Embodiment 130. The method of any one of Embodiments 119-128, wherein administration of a single unit dosage of said pharmaceutical composition takes place within 15 minutes, 10 minutes, or 5 minutes.

Embodiment 131. The method of any one of Embodiments 119-130, wherein said administration of said pharmaceutical composition does not induce cough reflex of said subject.

Embodiment 132. The method of any one of Embodiments 119-131, wherein said pharmaceutical composition is not or minimally irritative to mouth or throat of said subject.

Embodiment 133. A kit, comprising: said pharmaceutical composition any one of Embodiments 58-114 or said unit dose of Embodiment 117 or 118, and instructions for use of said pharmaceutical composition for treatment of a pulmonary disease.

Embodiment 134. A kit, comprising:
(a) said pharmaceutical composition of any one of Embodiments 58-114;
(b) a receptacle containing said pharmaceutical composition; and
(c) instructions for administering said pharmaceutical composition to a subject in need thereof via a nebulizer.

Embodiment 135. A system comprising: said pharmaceutical composition of any one of Embodiments 58-114 and a nebulizer.

Embodiment 136. The system of Embodiment 135, wherein said nebulizer is a jet nebulizer, a vibrating mesh nebulizer, an ultrasonic nebulizer, an electrospray nebulizer, or a hydraulic atomization device.

Embodiment 137. A method of manufacturing a pharmaceutical composition that comprises imatinib or a derivative thereof, comprising:
a) providing an aqueous solution comprising a solubility enhancer;
b) dissolving said imatinib or derivative thereof, or a pharmaceutically acceptable salt thereof in said aqueous solution comprising said solubility enhancer, thereby producing an aqueous solution containing imatinib or derivative thereof; and
c) adjusting volume, pH, osmolality, or viscosity of said aqueous solution containing imatinib or derivative thereof, thereby producing said pharmaceutical composition that comprises imatinib or derivative thereof.

Embodiment 138. The method of Embodiment 137, wherein said imatinib or derivative thereof, or pharmaceutically acceptable salt thereof comprises imatinib free base.

Embodiment 139. The method of Embodiment 137, wherein said imatinib or derivative thereof, or pharmaceutically acceptable salt thereof is imatinib free base.

Embodiment 140. The method of Embodiment 137, wherein said imatinib or derivative thereof, or pharmaceutically acceptable salt thereof comprises salt of imatinib selected from the group consisting of: acetate salt, formate salt, citrate salt, phosphate salt, maleate salt, fumarate salt, tartrate salt, malonate salt, lactic salt, and succinate salt.

Embodiment 141. The method of any one of Embodiments 137-140, wherein said imatinib or derivative thereof, or pharmaceutically acceptable salt thereof comprises less than 0.2%, less than 0.1%, less than 0.05%, less than 0.02%, less than 0.01%, or less than 0.001% imatinib mesylate.

Embodiment 142. The method of any one of Embodiments 137-141, wherein said pharmaceutical composition comprises less than 1 mg/mL, less than 0.5 mg/mL, less than 0.1 mg/mL, less than 0.005 mg/mL, less than 0.001 mg/mL, or less than 0.0001 mg/mL imatinib mesylate.

Embodiment 143. The method of any one of Embodiments 137-140, wherein said imatinib or derivative thereof, or pharmaceutically acceptable salt thereof does not comprise imatinib mesylate.

Embodiment 144. The method of any one of Embodiments 137-143, wherein said pharmaceutical composition does not comprise imatinib mesylate.

Embodiment 145. The method of any one of Embodiments 137-144, wherein said solubility enhancer is selected from the group consisting of: cyclodextrins, lipids, co-solvents, and organic acids.

Embodiment 146. The method of Embodiment 145, wherein the solubility enhancer comprises a cyclodextrin.

Embodiment 147. The method of Embodiment 146, wherein the cyclodextrin is selected from the group consisting of: α-cyclodextrin, β-cyclodextrin, γ-cyclodextrin, hydroxypropyl-β-cyclodextrin, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin dimaltosyl-β-cyclodextrin, 6A-amino-6A-deoxy-N-(3-hydroxypropyl)-β-cyclodextrin.

Embodiment 148. The method of Embodiment 146, wherein said cyclodextrin comprises succinyl-α-cyclodextrin, succinyl-β-cyclodextrin, succinyl-γ-cyclodextrin, sulfobutylether-α-cyclodextrin, sulfobutylether-β-cyclodextrin, sulfobutylether-γ-cyclodextrin, carboxymethyl-α-cyclodextrin, carboxymethyl-β-cyclodextrin, carboxymethyl-γ-cyclodextrin, 2-carboxyethyl-α-cyclodextrin, 2-carboxyethyl-β-cyclodextrin, 2-carboxyethyl-γ-cyclodextrin, phosphate-α-cyclodextrin, phosphate-β-cyclodextrin, phosphate-γ-cyclodextrin, sulfobutylether-γ-cyclodextrin, or sulfobutylether-γ-cyclodextrin.

Embodiment 149. The method of Embodiment 146, wherein said cyclodextrin comprises an anionic cyclodextrin.

Embodiment 150. The method of Embodiment 146, wherein said cyclodextrin comprises hydroxypropyl-β-cyclodextrin.

Embodiment 151. The method of Embodiment 146, wherein said aqueous solution comprises a salt of said cyclodextrin.

Embodiment 152. The method of Embodiment 151, wherein said salt of said cyclodextrin is a salt selected from the group consisting of: sodium salt, calcium salt, magnesium salt, iron salt, chromium salt, copper salt, zinc salt, lysine salt, arginine salt, and histidine salt.

Embodiment 153. The method of Embodiment 146, wherein said aqueous solution comprises sulfobutylether-β-cyclodextrin sodium.

Embodiment 154. The method of Embodiment 146, wherein said cyclodextrin comprises hydroxypropyl-β-cyclodextrin.

Embodiment 155. The method of any one of Embodiments 146-154, wherein said pharmaceutical composition comprises said cyclodextrin at a concentration of from about 1% (w/v) to about 80% (w/v), from about 2% (w/v) to about 70% (w/v), from about 2% (w/v) to about 60% (w/v), from about 2% (w/v) to about 50% (w/v), from about 2% (w/v) to about 40% (w/v), from about 2% (w/v) to about 30% (w/v), from about 2% (w/v) to about 20% (w/v), from about 2% (w/v) to about 15% (w/v), from about 2% (w/v) to about 10% (w/v), from about 2% (w/v) to about 8% (w/v), from about 2% (w/v) to about 5% (w/v), from about 5% (w/v) to about 80% (w/v), from about 5% (w/v) to about 70% (w/v), from about 5% (w/v) to about 60% (w/v), from about 5% (w/v) to about 50% (w/v), from about 5% (w/v) to about 40% (w/v), from about 5% (w/v) to about 30% (w/v), from about 5% (w/v) to about 20% (w/v), from about 5% (w/v) to about 15% (w/v), from about 5% (w/v) to about 12% (w/v), from about 5% (w/v) to about 10% (w/v), from about 10% (w/v) to about 60% (w/v), from about 10% (w/v) to about 50% (w/v), from about 10% (w/v) to about 40% (w/v), from about 10% (w/v) to about 30% (w/v), from about 20% (w/v) to about 30% (w/v), from about 10% (w/v) to about 25% (w/v), from about 19% (w/v) to about 25% (w/v), from about 19.5% (w/v) to about 25% (w/v), from about 20% (w/v) to about 25% (w/v), from about 20.5% (w/v) to about 25% (w/v), from about 21% (w/v) to about 25% (w/v), from about 21.5% (w/v) to about 25% (w/v), from about 22% (w/v) to about 25% (w/v), from about 22.5% (w/v) to about 25% (w/v), from about 23% (w/v) to about 25% (w/v), from about 10% (w/v) to about 20% (w/v), or from about 10% (w/v) to about 15% (w/v).

Embodiment 156. The method of any one of Embodiments 146-154, wherein said pharmaceutical composition comprises said cyclodextrin at a concentration of from 5% (w/v) to 40% (w/v).

Embodiment 157. The method of any one of Embodiments 146-154, wherein said pharmaceutical composition comprises said cyclodextrin at a concentration of from 10% (w/v) to 20% (w/v).

Embodiment 158. The method of any one of Embodiments 146-154, wherein said pharmaceutical composition comprises said cyclodextrin at a concentration of from 25% (w/v) to 40% (w/v).

Embodiment 159. The method of any one of Embodiments 146-154, wherein said pharmaceutical composition comprises said cyclodextrin at a concentration of about 10% (w/v), about 12% (w/v), about 14% (w/v), about 15% (w/v), about 16% (w/v), about 18% (w/v), or about 20% (w/v).

Embodiment 160. The method of any one of Embodiments 146-154, wherein said pharmaceutical composition comprises said cyclodextrin at a concentration of about 22% (w/v), about 24% (w/v), about 26% (w/v), about 28% (w/v), about 30% (w/v), about 32% (w/v), about 34% (w/v), about 36% (w/v), about 38% (w/v), or about 40% (w/v).

Embodiment 161. The method of Embodiment 145, wherein said solubility enhancer comprises a lipid or a fatty acid.

Embodiment 162. The method of Embodiment 161, wherein said lipid or fatty acid is selected from the group consisting of: polyethoxylated castor oil, phospholipids, glycolipids, ganglioside GM1, sphingomyelin, phosphatidic acid, cardiolipin; lipids bearing polymer chains such as polyethylene glycol (PEG), chitin, hyaluronic acid, and polyvinylpyrrolidone; lipids bearing sulfonated monosaccharides, lipid-bearing sulfonated disaccharides, lipid bearing sulfonated polysaccharides; fatty acids such as palmitic acid, stearic acid, and oleic acid; cholesterol, cholesterol esters, and cholesterol hemisuccinate.

Embodiment 163. The method of Embodiment 145, wherein the solubility enhancer comprises a co-solvent.

Embodiment 164. The method of Embodiment 163, wherein the co-solvent comprises glycerol or ethanol.

Embodiment 165. The method of Embodiment 145, wherein the solubility enhancer comprises an organic acid.

Embodiment 166. The method of Embodiment 165, wherein said organic acid is selected from the group consisting of: acetic acid, acid modified starch, aconitic acid, adipic acid, hexanedioic acid, L-ascorbic acid, benzoic acid, caprylic acid, octanoic acid, cholic acid, citric acid, desoxycholic acid, erythorbic acid (D-isoascorbic acid), formic acid, L-glutamic acid, L-glutamic acid hydrochloride, glycocholic acid, hydrochloric acid, iron naphthenate, iron tallate, D(−)-lactic acid, lactic acid, L(+)-lactic acid, linoleic acid, malic acid, L-malic acid, niacin (nicotinic acid), oleic acid, pectin, pectinic acid, phosphoric acid, L(+)-potassium acid tartrate, propionic acid, acid hydrolyzed proteins, sodium acid pyrophosphate, acidic sodium aluminum phosphate, sorbic acid, stearic acid, succinic acid, sulfamic acid, sulfuric Acid, tannic acid, L(+)-tartaric acid, taurocholic acid, and thiodipropionic acid.

Embodiment 167. The method of any one of Embodiments 137-166, wherein said pharmaceutical composition comprises from 20 to 500 mg/mL of said imatinib or derivative thereof.

Embodiment 168. The method of any one of Embodiments 137-166, wherein said pharmaceutical composition comprises from 20 mg/mL to 400 mg/mL, from 20 mg/mL to 300 mg/mL, from 20 mg/mL to 200 mg/mL, from 100 mg/mL to 500 mg/mL, from 200 mg/mL to 500 mg/mL, from 300 mg/mL to 500 mg/mL, from 400 mg/mL to 500 mg/mL, from 100 mg/mL to 400 mg/mL, from 100 mg/mL to 300 mg/mL, from 100 mg/mL to 200 mg/mL, from 200 mg/mL to 400 mg/mL, from 200 mg/mL to 300 mg/mL, from 20 to 100, from 20 mg/mL to 80 mg/mL, from 20 mg/mL to 60 mg/mL, from 20 mg/mL to 40 mg/mL, from 20 mg/mL to 30 mg/mL, from 30 mg/mL to 40 mg/mL, from 40 mg/mL to 60 mg/mL, from 40 mg/mL to 80 mg/mL, from 40 mg/mL to 100 mg/mL, from 40 mg/mL to 120 mg/mL, from 40 mg/mL to 150 mg/mL, from 60 mg/mL to 80 mg/mL, from 60 mg/mL to 100 mg/mL, from 60 mg/mL to 120 mg/mL, or from 60 mg/mL to 150 mg/mL of said imatinib or derivative thereof.

Embodiment 169. The method of any one of Embodiments 137-166, wherein said pharmaceutical composition comprises about 50 mg/mL, about 60 mg/mL, about 70 mg/mL, about 80 mg/mL, about 90 mg/mL, about 100 mg/mL, about 110 mg/mL, about 120 mg/mL, about 130 mg/mL, about 140 mg/mL, or about 150 mg/mL of said imatinib or derivative thereof.

Embodiment 170. The method of any one of Embodiments 137-166, wherein said pharmaceutical composition comprises about 80 mg/mL of said imatinib or derivative thereof.

Embodiment 171. The method of any one of Embodiments 137-170, wherein said aqueous solution comprising said solubility enhancer further comprises a pH buffer.

Embodiment 172. The method of Embodiment 171, wherein said pH buffer comprises an organic acid salt of citric acid, lactic acid, ascorbic acid, gluconic acid, carbonic acid, tartaric acid, succinic acid, acetic acid, or phthalic acid, Tris, tromethamine hydrochloride, or a phosphate buffer.

Embodiment 173. The method of Embodiment 171, wherein said pH buffer comprises a phosphate buffer.

Embodiment 174. The method of any one of Embodiments 137-173, wherein said pharmaceutical composition has a pH of 3 to 8.

Embodiment 175. The method of any one of Embodiments 137-173, wherein said pharmaceutical composition has a pH of from 3 to 6, from 4 to 6, from 4.5 to 5.5, from 5 to 6, from 4 to 7, from 5 to 7, or from 6 to 7.

Embodiment 176. The method of any one of Embodiments 137-173, wherein said pharmaceutical composition has a pH of about 4.7, about 4.8, about 4.9, about 5.0, about 5.1, about 5.2, about 5.4, about 5.5, or about 5.6.

Embodiment 177. The method of any one of Embodiments 137-173, wherein said pharmaceutical composition has a pH of from 7 to 8.

Embodiment 178. The method of any one of Embodiments 137-173, wherein said pharmaceutical composition has a pH of about 7.0, about 7.2, about 7.4, about 7.6, about 7.8, or about 8.0.

Embodiment 179. The method of any one of Embodiments 137-178, wherein said aqueous solution or suspension further comprises an artificial sweetener.

Embodiment 180. The method of Embodiment 179, wherein said artificial sweetener is selected from the group consisting of: acesulfame potassium, aspartame, cyclamate, mogrosides, saccharin, stevia, sucralose, neotame, and sugar alcohols, and combinations thereof.

Embodiment 181. The method of any one of Embodiments 137-180, wherein said pharmaceutical composition has a viscosity of at most 10 centipoise.

Embodiment 182. The method of any one of Embodiments 137-180, wherein said pharmaceutical composition has a viscosity of at most 2.5 centipoise.

Embodiment 183. The method of any one of Embodiments 137-182, wherein said pharmaceutical composition further comprises a pharmaceutically acceptable excipient.

Embodiment 184. The method of Embodiment 183, wherein said pharmaceutically acceptable excipient comprises a surfactant.

Embodiment 185. The method of Embodiment 184, wherein said surfactant comprises Tween, sodium lauryl sulfate (SLS), or dipalmitoylphosphatidylcholine (DPPC).

Embodiment 186. The method of Embodiment 183, wherein said pharmaceutically acceptable excipient comprises a lipid.

Embodiment 187. The method of Embodiment 186, wherein said lipid comprises a polymeric lipid, a sulfonated poly saccharide, or a fatty acid.

Embodiment 188. The method of Embodiment 186, wherein said lipid comprises a polymeric lipid, a sulfonated poly saccharide, or a fatty acid.

Embodiment 189. The method of any one of Embodiments 137-188, wherein solubility of the imatinib or derivative thereof in the aqueous solution is negatively correlated with the pH of the aqueous solution.

Embodiment 190. The method of any one of Embodiments 146-189, wherein solubility of the imatinib or derivative thereof in the aqueous solution is positively correlated with concentration of the cyclodextrin in the aqueous solution.
[1]A method of treating a subject in need thereof, comprising administering to said subject a pharmaceutical composition,
   wherein said administering comprises delivering said pharmaceutical composition via inhalation,
   wherein said pharmaceutical composition comprises an aqueous solution or suspension that comprises imatinib or a derivative thereof and cyclodextrin,
   wherein said pharmaceutical composition provides a pharmacokinetic profile when measured in a human clinical trial in which human subjects receive said administering of a single dose of said pharmaceutical composition, and
   wherein said pharmacokinetic profile is characterized by:
   (a) a maximum measured plasma concentration of said imatinib or derivative thereof (Cₘₐₓ) ranging from about 50 ng/mL to about 1900 ng/mL;
   (b) an area under the plasma concentration of said imatinib or derivative thereof-time curve from 0 to 24 hour (AUC₀₋₂₄) ranging from about 1 μg*h/mL to about 24 μg*h/mL;
   (c) an area under the plasma concentration of said imatinib or derivative thereof-time curve from 0 to infinity (AUC_{0-∞}) ranging from about 1 μg*h/mL to about 38 μg*h/mL; or
   (d) any combination of (a)-(c).
[2] The method of paragraph [1], wherein said Cₘₐₓ is from about 50 ng/mL to about 1900 ng/mL.
[3] The method of paragraph [1], wherein said Cₘₐₓ is from about 100 ng/mL to about 1500 ng/mL.
[4] The method of any one of paragraphs [1]-[3], wherein said AUC₀₋₂4 is from about 1 μg*h/mL to about 24 μg*h/mL.
[5] The method of any one of paragraphs [1]-[3], wherein said AUC₀₋₂₄ is from about 1 μg*h/mL to about 17 μg*h/mL.
[6] The method of any one of paragraphs [1]-[5], wherein said AUC_{0-∞} is from about 1 μg*h/mL to about 38 μg*h/mL.
[7] The method of any one of paragraphs [1]-[5], wherein said AUC_{0-∞} is from about 1 μg*h/mL to about 23 μg*h/mL.
[8]A method of treating a subject in need thereof, comprising administering to said subject a pharmaceutical composition,
   wherein said administering comprises delivering said pharmaceutical composition via inhalation,
   wherein said pharmaceutical composition comprises an aqueous solution or suspension that comprises imatinib or a derivative thereof and cyclodextrin,
   wherein said pharmaceutical composition provides a pharmacokinetic profile when measured in a human clinical trial in which human subjects receive said administering of a multiple-dose regimen of said pharmaceutical composition, wherein said multiple-dose regimen comprises at least one dose of said pharmaceutical composition every day for at least 3 consecutive days, and
   wherein said pharmacokinetic profile is characterized by:
   (1) a maximum measured plasma concentration of said imatinib or derivative thereof (Cₘₐₓ) ranging from about 50 ng/mL to about 1900 ng/mL;
   (2) an area under the plasma concentration of said imatinib or derivative thereof-time curve from 0 to 24 hour (AUC₀₋₂₄) ranging from about 1 μg*h/mL to about 24 μg*h/mL; (3) an area under the plasma concentration of said imatinib or derivative thereof-time curve from 0 to infinity (AUC_{0-∞}) ranging from about 1 μg*h/mL to about 38 μg*h/mL; or
   (4) any combination of (1)-(3).
[9] The method of paragraph [8], wherein said Cₘₐₓ is from about 50 ng/mL to about 2500 ng/mL.
[10] The method of paragraph [8], wherein said Cₘₐₓ is from about 100 ng/mL to about 1800 ng/mL.
[11] The method of any one of paragraphs [8]-[10], wherein said AUC₀₋₂₄ is from about 1 μg*h/mL to about 40 μg*h/mL.
[12] The method of any one of paragraphs [8]-[10], wherein said AUC₀₋₂₄ is from about 1 μg*h/mL to about 24 μg*h/mL.
[13] The method of any one of paragraphs [8]-[12], wherein said AUC_{0-∞} is from about 1 μg*h/mL to about 80 μg*h/mL.
[14] The method of any one of paragraphs [8]-[12], wherein said AUC_{0-∞} is from about 1 μg*h/mL to about 37 μg*h/mL.
[15] The method of any one of paragraphs [8]-[14], wherein said multiple-dose regimen comprises two doses of said pharmaceutical composition every day for at least 3 consecutive days, optionally for 3, 4, 5, 7, 10, 12, 14, 20, 21, 25, 28, or 35 consecutive days.
[16] The method of any one of paragraphs [8]-[14], wherein said multiple-dose regimen comprises three doses of said pharmaceutical composition every day for at least 3 consecutive days, optionally for 3, 4, 5, 7, 10, 12, 14, 20, 21, 25, 28, or 35 consecutive days.
[17]A method of treating a subject in need thereof, comprising administering to said subject in need thereof a pharmaceutical composition,
   wherein said administering comprises delivering said pharmaceutical composition via inhalation,
   wherein said pharmaceutical composition comprises an aqueous solution or suspension that comprises imatinib or a derivative thereof and cyclodextrin,
   wherein said pharmaceutical composition provides a pharmacokinetic profile when measured in an animal study in which mammalian animal subjects receive a single dose of said pharmaceutical composition or a diluted version thereof via inhalational administration or oral administration, and
   wherein said pharmacokinetic profile is characterized by:
   (i) a ratio of maximum measured lung concentration of said imatinib or derivative thereof post said inhalational administration (C_{max,inhalation,lung}) to maximum plasma concentration of said imatinib or derivative thereof said inhalational administration (C_{max,inhalation,plasma}) ranging from about 20 to about 1000; or
   (ii) a ratio of maximum measured lung concentration of said imatinib or derivative thereof post said inhalational administration (C_{max,inhalation,lung}) to maximum measured lung concentration of said imatinib or derivative thereof post said oral administration (C_{max,po,lung}) ranging from about 20 to about 1000; or
   (iii) a ratio of area under lung concentration of said imatinib or derivative thereof-time curve from 0 to 24 hours post said inhalational administration (AUC_{inhalation,lung,0-24}) to area under plasma concentration of said imatinib or derivative thereof-time curve from 0 to 24 hours post said inhalational administration (AUC_{inhalation,plasma,0-24}) ranging from about 10 to about 1000; or
   (iv) a ratio of area under lung concentration of said imatinib or derivative thereof-time curve from 0 to 24 hours post said inhalational administration (AUC_{inhalation,lung,0-24}) to area under lung concentration of said imatinib or derivative thereof-time curve from 0 to 24 hours post said oral administration (AUC_{po,lung,0-24}) ranging from about 10 to about 1000; or
   (v) a therapeutic advantage (Rd) of inhalational administration compared to oral administration ranging from about 2 to about 200, wherein Rd is calculated according to the following equation:

$$Rd = \frac{( {AUC}_{inhalation , lung , 0 - 24} / {AUC}_{inhalation , plasma , 0 - 24} )}{( {AUC}_{po , lung , 0 - 24} / {AUC}_{po , plasma , 0 - 24} )} ,$$

wherein AUC_{po,plasma,0-24} is area under plasma concentration of said imatinib or derivative thereof-time curve from 0 to 24 hours post said oral administration; or
(vi) any combination of (i)-(v).
[18] The method of paragraph [17], wherein in said animal study, said mammalian animal subjects receive a single dose of a diluted version of said pharmaceutical composition via inhalational administration or oral administration.
[19] The method of paragraph [18], wherein said diluted version of said pharmaceutical composition is diluted by at least 2 times with water as compared to said pharmaceutical composition.
[20] The method of paragraph [18], wherein said diluted version of said pharmaceutical composition is diluted by 2 to 20 times with water as compared to said pharmaceutical composition.
[21] The method of paragraph [18], wherein said diluted version of said pharmaceutical composition is diluted by about 2, 3, 4, 5, 6, 7, 8, 9, or 10 times with water as compared to said pharmaceutical composition.
[22] The method of paragraph [18], wherein said diluted version of said pharmaceutical composition is diluted by about 5 times with water as compared to said pharmaceutical composition.
[23] The method of any one of paragraphs [17]-[22], wherein said mammalian subjects are rats.
[24] The method of any one of paragraphs [17]-[22], wherein said mammalian subjects are dogs or pigs.
[25] The method of any one of paragraphs [17]-[24], wherein said mammalian subjects receive administration of 0.1 mg/kg to 10 mg/kg of said imatinib or derivative thereof for said animal study, optionally 0.3 mg/kg, 1 mg/kg, or 3 mg/kg of said imatinib or derivative thereof.
[26] The method of any one of paragraphs [1]-[25], wherein said pharmaceutical composition comprises from about 10 mg to about 250 mg of said imatinib or derivative thereof.
[27] The method of any one of paragraphs [1]-[25], wherein said pharmaceutical composition comprises about 20 mg of said imatinib or derivative thereof.
[28] The method of any one of paragraphs [1]-[25], wherein said pharmaceutical composition comprises about 60 mg of said imatinib or derivative thereof.
[29] The method of any one of paragraphs [1]-[25], wherein said pharmaceutical composition comprises about 80 mg of said imatinib or derivative thereof.
[30] The method of any one of paragraphs [1]-[25], wherein said pharmaceutical composition comprises about 100 mg of said imatinib or derivative thereof.
[31] The method of any one of paragraphs [1]-[25], wherein said pharmaceutical composition comprises about 120 mg of said imatinib or derivative thereof.
[32] The method of any one of paragraphs [1]-[25], wherein said pharmaceutical composition comprises about 160 mg of said imatinib or derivative thereof.
[33] The method of any one of paragraphs [1]-[25], wherein said pharmaceutical composition comprises about 200 mg of said imatinib or derivative thereof.
[34] The method of any one of paragraphs [1]-[25], wherein said pharmaceutical composition comprises about 240 mg of said imatinib or derivative thereof.
[35] The method of any one of paragraphs [1]-[34], wherein the method results in a central lung dose of said imatinib or derivative thereof ranging from about 10 mg to 100 mg, wherein said central lung dose measures the amount of said imatinib or derivative thereof delivered to the conducting zone of the subject's lungs.
[36] The method of paragraph [35], wherein said central lung dose is from about 15 mg to 80 mg.
[37] The method of paragraph [35], wherein said central lung dose is from about 30 mg to 60 mg.
[38] The method of paragraph [35], wherein said central lung dose is about 15 mg.
[39] The method of paragraph [35], wherein said central lung dose is about 20 mg.
[40] The method of paragraph [35], wherein said central lung dose is about 30 mg.
[41] The method of paragraph [35], wherein said central lung dose is about 45 mg.
[42] The method of paragraph [35], wherein said central lung dose is about 60 mg.
[43] The method of any one of paragraphs [1]-[42], wherein the method results in a peripheral lung dose of said imatinib or derivative thereof ranging from about 15 mg to 120 mg, wherein said peripheral lung dose measures the amount of said imatinib or derivative thereof delivered to respiratory zone of the subject's lungs.
[44] The method of paragraph [43], wherein said peripheral lung dose is from about 20 mg to 100 mg.
[45] The method of paragraph [43], wherein said peripheral lung dose is from about 25 mg to 90 mg.
[46] The method of paragraph [43], wherein said peripheral lung dose is about 20 mg.
[47] The method of paragraph [43], wherein said peripheral lung dose is about 30 mg.
[48] The method of paragraph [43], wherein said peripheral lung dose is about 45 mg.
[49] The method of paragraph [43], wherein said peripheral lung dose is about 50 mg.
[50] The method of paragraph [43], wherein said peripheral lung dose is about 65 mg.
[51] The method of paragraph [43], wherein said peripheral lung dose is about 80 mg.
[52] The method of paragraph [43], wherein said peripheral lung dose is about 90 mg.
[53] The method of paragraph [43], wherein said peripheral lung dose is from about 15 mg to 35 mg.
[54] The method of paragraph [43], wherein said peripheral lung dose is from about 20 mg to 30 mg.
[55] The method of paragraph [43], wherein said peripheral lung dose is about 12 mg.
[56] The method of paragraph [43], wherein said peripheral lung dose is about 16 mg.
[57] The method of paragraph [43], wherein said peripheral lung dose is about 20 mg.
[58] The method of paragraph [43], wherein said peripheral lung dose is about 24 mg.
[59] The method of paragraph [43], wherein said peripheral lung dose is about 28 mg.
[60] The method of paragraph [43], wherein said peripheral lung dose is about 32 mg.
[61] The method of paragraph [43], wherein said peripheral lung dose is about 36 mg.
[62] The method of any one of paragraphs [1]-[61], wherein said subject suffers from a pulmonary disease.
[63] The method of paragraph [62], wherein said pulmonary disease comprises lung fibrosis, lung cancer, or pulmonary hypertension.
[64] The method of paragraph [62], wherein said pulmonary disease comprises pulmonary arterial hypertension.
[65] The method of any one of paragraphs [1]-[64], comprising administering to said subject a dose of said pharmaceutical composition at least once per day.
[66] The method of any one of paragraphs [1]-[64], comprising administering to said subject a dose of said pharmaceutical composition 2, 3, 4, or 5 times per day.
[67] The method of any one of paragraphs [1]-[66], comprising administering to said subject at least one dose of said pharmaceutical composition daily for a period of at least 5, 10, 20, 30, 60, 100, or 300 days, at least 1, 2, 3, 4, or 5 years.
[68] The method of any one of paragraphs [1]-[67], wherein said administering is performed using a nebulizer.
[69] The method of paragraph [68], wherein said nebulizer is a jet nebulizer, a vibrating mesh nebulizer, an ultrasonic nebulizer, an electrospray nebulizer, or a hydraulic atomization device.
[70] The method of paragraph [68], wherein said nebulizer is a vibrating mesh nebulizer.
[71] The method of paragraph [68], wherein said nebulizer is a hydraulic atomization device.
[72] The method of paragraph [68], wherein said nebulizer is an electrospray nebulizer.
[73] The method of any one of paragraphs [1]-[72], wherein said administering of a single dose of said pharmaceutical composition takes place within 30 minutes.
[74] The method of any one of paragraphs [1]-[72], wherein said administering of a single dose of said pharmaceutical composition takes place within 15 minutes, 10 minutes, or 5 minutes.
[75] The method of any one of paragraphs [1]-[72], wherein said administering of a single dose of said pharmaceutical composition takes place for about 2 minutes, 4 minutes, 6 minutes, or 8 minutes.
[76] The method of any one of paragraphs [1]-[75], wherein said aqueous solution or suspension has said cyclodextrin at a concentration of from about 1% (w/v) to about 80% (w/v).
[77] The method of any one of paragraphs [1]-[76], wherein said cyclodextrin is selected from the group consisting of: α-cyclodextrin, β-cyclodextrin, γ-cyclodextrin, hydroxypropyl-β-cyclodextrin, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin dimaltosyl-β-cyclodextrin, methyl-β-cyclodextrin, 6A-amino-6A-deoxy-N-(3-hydroxypropyl)-β-cyclodextrin, succinyl-α-cyclodextrin, succinyl-β-cyclodextrin, succinyl-γ-cyclodextrin, sulfobutylether-α-cyclodextrin, sulfobutylether-β-cyclodextrin, sulfobutylether-γ-cyclodextrin, carboxymethyl-α-cyclodextrin, carboxymethyl-β-cyclodextrin, carboxymethyl-γ-cyclodextrin, 2-carboxyethyl-α-cyclodextrin, 2-carboxyethyl-β-cyclodextrin, 2-carboxyethyl-γ-cyclodextrin, phosphate-α-cyclodextrin, phosphate-β-cyclodextrin, phosphate-γ-cyclodextrin, sulfoalkylether-β-cyclodextrins, and sulfoalkylether-γ-cyclodextrins.
[78] The method of any one of paragraphs [1]-[76], wherein said cyclodextrin comprises succinyl-α-cyclodextrin, succinyl-β-cyclodextrin, succinyl-γ-cyclodextrin, sulfobutylether-α-cyclodextrin, sulfobutylether-β-cyclodextrin, sulfobutylether-γ-cyclodextrin, carboxymethyl-α-cyclodextrin, carboxymethyl-β-cyclodextrin, carboxymethyl-γ-cyclodextrin, 2-carboxyethyl-α-cyclodextrin, 2-carboxyethyl-β-cyclodextrin, 2-carboxyethyl-γ-cyclodextrin, phosphate-α-cyclodextrin, phosphate-β-cyclodextrin, or phosphate-γ-cyclodextrin.
[79] The method of any one of paragraphs [1]-[76], wherein said cyclodextrin comprises an anionic cyclodextrin.
[80] The method of any one of paragraphs [1]-[79], wherein said aqueous solution or suspension comprises a salt of said cyclodextrin.
[81] The composition of paragraph [80], wherein said salt of said cyclodextrin is a salt selected from the group consisting of: sodium salt, calcium salt, magnesium salt, iron salt, chromium salt, copper salt, zinc salt, lysine salt, arginine salt, and histidine salt.
[82] The method of any one of paragraphs [1]-[80], wherein said cyclodextrin comprises sulfobutylether-β-cyclodextrin.
[83] The method of any one of paragraphs [1]-[80], wherein said aqueous solution or suspension comprises sulfobutylether-β-cyclodextrin sodium.
[84] The method of any one of paragraphs [1]-[80], wherein said cyclodextrin comprises hydroxypropyl-β-cyclodextrin.
[85] The method of any one of paragraphs [1]-[84], wherein said aqueous solution or suspension further comprises a pH buffer.
[86] The method of paragraph [85], wherein said pH buffer comprises an organic acid salt of citric acid, lactic acid, ascorbic acid, gluconic acid, carbonic acid, tartaric acid, succinic acid, acetic acid, or phthalic acid, Tris, tromethamine hydrochloride, or a phosphate buffer.
[87] The method of paragraph [85], wherein said pH buffer comprises a phosphate buffer.
[88] The method of any one of paragraphs [1]-[87], wherein said aqueous solution or suspension further comprises an artificial sweetener.
[89] The method of paragraph [88], wherein said artificial sweetener is selected from the group consisting of: acesulfame potassium, aspartame, cyclamate, mogrosides, saccharin, stevia, sucralose, neotame, and sugar alcohols, and combinations thereof.
[90] The method of any one of paragraphs [1]-[89], wherein said aqueous solution or suspension has a viscosity of at most 10 centipoise.
[91] The method of any one of paragraphs [1]-[89], wherein said aqueous solution or suspension has a viscosity of at most 9.5 centipoise, at most 9.0 centipoise, at most 8.5 centipoise, at most 8.0 centipoise, at most 7.6 centipoise, at most 7.4 centipoise, at most 7.2 centipoise, at most 7.0 centipoise, at most 6.8 centipoise, at most 6.6 centipoise, at most 6.4 centipoise, at most 6.2 centipoise, at most 6.0 centipoise, at most 5.8 centipoise, at most 5.6 centipoise, at most 5.4 centipoise, at most 5.2 centipoise, at most 5.0 centipoise, at most 4.8 centipoise, at most 4.6 centipoise, at most 4.4 centipoise, at most 4.2 centipoise, at most 4.0 centipoise, at most 3.8 centipoise, at most 3.6 centipoise, at most 3.4 centipoise, at most 3.2 centipoise, at most 3.0 centipoise, at most 2.8 centipoise, at most 2.6 centipoise, at most 2.4 centipoise, at most 2.2 centipoise, at most 2.0 centipoise, at most 1.8 centipoise, at most 1.6 centipoise, at most 1.4 centipoise, at most 1.2 centipoise, at most 1.0 centipoise, at most 0.8 centipoise, at most 0.6 centipoise, at most 0.4 centipoise, or at most 0.2 centipoise.
[92] The method of any one of paragraphs [1]-[89], wherein said aqueous solution or suspension has a viscosity of about 0.1 centipoise, 0.2 centipoise, 0.3 centipoise, 0.4 centipoise, 0.5 centipoise, 0.6 centipoise, 0.7 centipoise, 0.8 centipoise, 0.9 centipoise, 1.0 centipoise, 1.1 centipoise, 1.2 centipoise, 1.3 centipoise, 1.4 centipoise, 1.5 centipoise, 1.6 centipoise, 1.7 centipoise, 1.8 centipoise, 1.9 centipoise, 2.0 centipoise, 2.1 centipoise, 2.2 centipoise, 2.3 centipoise, 2.4 centipoise, 2.5 centipoise, 2.6 centipoise, 2.8 centipoise, 3.0 centipoise, 3.2 centipoise, 3.5 centipoise, 3.8 centipoise, 4.0 centipoise, 4.2 centipoise, 4.5 centipoise, 4.8 centipoise, 5.0 centipoise, 5.5 centipoise, 6.0 centipoise, 6.5 centipoise, 7.0 centipoise, 7.5 centipoise, 8.0 centipoise, or 8.5 centipoise.
[93] The method of any one of paragraphs [1]-[92], wherein said aqueous solution or suspension has from 1 to 250 mg/mL of said imatinib or derivative thereof.
[94] The method of any one of paragraphs [1]-[93], wherein said aqueous solution or suspension has from 60 mg/mL to 120 mg/mL of said imatinib or derivative thereof.
[95] The method of any one of paragraphs [1]-[93], wherein said aqueous solution or suspension has about 60 mg/mL of said imatinib or derivative thereof.
[96] The method of any one of paragraphs [1]-[93], wherein said aqueous solution or suspension has about 120 mg/mL of said imatinib or derivative thereof
[97] The method of any one of paragraphs [1]-[96], wherein said aqueous solution or suspension has a pH of about 3 to about 8.
[98] The method of paragraph [97], wherein said pH of said aqueous solution or suspension is from about 4 to about 8.
[99] The method of paragraph [97], wherein said pH of said aqueous solution or suspension is from about 4 to about 6.
[100] The method of paragraph [97], wherein said pH of said aqueous solution or suspension is about 5.
[101] The method of any one of paragraphs [1]-[100], wherein said aqueous solution or suspension has said cyclodextrin at a concentration of from about 2% (w/v) to about 70% (w/v), from about 2% (w/v) to about 60% (w/v), from about 2% (w/v) to about 50% (w/v), from about 2% (w/v) to about 40% (w/v), from about 2% (w/v) to about 30% (w/v), from about 2% (w/v) to about 20% (w/v), from about 2% (w/v) to about 15% (w/v), from about 2% (w/v) to about 10% (w/v), from about 2% (w/v) to about 8% (w/v), from about 2% (w/v) to about 5% (w/v), from about 5% (w/v) to about 80% (w/v), from about 5% (w/v) to about 70% (w/v), from about 5% (w/v) to about 60% (w/v), from about 5% (w/v) to about 50% (w/v), from about 5% (w/v) to about 40% (w/v), from about 5% (w/v) to about 30% (w/v), from about 5% (w/v) to about 20% (w/v), from about 5% (w/v) to about 15% (w/v), from about 5% (w/v) to about 12% (w/v), from about 5% (w/v) to about 10% (w/v), from about 10% (w/v) to about 60% (w/v), from about 10% (w/v) to about 50% (w/v), from about 10% (w/v) to about 40% (w/v), from about 10% (w/v) to about 30% (w/v), from about 20% (w/v) to about 30% (w/v), from about 10% (w/v) to about 25% (w/v), from about 19% (w/v) to about 25% (w/v), from about 19.5% (w/v) to about 25% (w/v), from about 20% (w/v) to about 25% (w/v), from about 20.5% (w/v) to about 25% (w/v), from about 21% (w/v) to about 25% (w/v), from about 21.5% (w/v) to about 25% (w/v), from about 22% (w/v) to about 25% (w/v), from about 22.5% (w/v) to about 25% (w/v), from about 23% (w/v) to about 25% (w/v), from about 10% (w/v) to about 20% (w/v), or from about 10% (w/v) to about 15% (w/v).
[102] The method of any one of paragraphs [1]-[100], wherein said aqueous solution or suspension has said cyclodextrin at a concentration of from 5% (w/v) to 40% (w/v).
[103] The method of any one of paragraphs [1]-[100], wherein said aqueous solution or suspension has said cyclodextrin at a concentration of from 10% (w/v) to 20% (w/v).
[104] The method of any one of paragraphs [1]-[100], wherein said aqueous solution or suspension has said cyclodextrin at a concentration of from 25% (w/v) to 40% (w/v).
[105] The method of any one of paragraphs [1]-[100], wherein said aqueous solution or suspension has said cyclodextrin at a concentration of about 10% (w/v), about 12% (w/v), about 14% (w/v), about 15% (w/v), about 16% (w/v), about 18% (w/v), or about 20% (w/v).
[106] The method of any one of paragraphs [1]-[100], wherein said aqueous solution or suspension has said cyclodextrin at a concentration of about 22% (w/v), about 24% (w/v), about 26% (w/v), about 28% (w/v), about 30% (w/v), about 32% (w/v), about 34% (w/v), about 36% (w/v), about 38% (w/v), or about 40% (w/v).
[107] The method of any one of paragraphs [1]-[106], wherein said composition comprises said aqueous solution.
[108] The composition of paragraph [107], wherein solubility of the imatinib or derivative thereof in the aqueous solution is negatively correlated with the pH of the aqueous solution.
[109] The composition of paragraph [107] or [108], wherein solubility of the imatinib or derivative thereof in the aqueous solution is positively correlated with concentration of the cyclodextrin in the aqueous solution.
[110] The method of any one of paragraphs [1]-[106], wherein said pharmaceutical composition comprises said aqueous suspension.
[111] The method of any one of paragraphs [1]-[110], wherein said pharmaceutical composition comprises less than 1 mg/mL, less than 0.5 mg/mL, less than 0.1 mg/mL, less than 0.05 mg/mL, less than 0.01 mg/mL, less than 0.005 mg/mL, less than 0.001 mg/mL, or less than 0.0001 mg/mL imatinib mesylate.
[112] The method of any one of paragraphs [1]-[110], wherein said pharmaceutical composition does not comprise imatinib mesylate.
[113] The method of any one of paragraphs [1]-[112], wherein said imatinib or derivative thereof comprises imatinib free base.
[114] The method of any one of paragraphs [1]-[112], wherein said imatinib or derivative thereof is imatinib free base.
[115] The method of any one of paragraphs [1]-[112], wherein said pharmaceutical composition comprises a salt of said imatinib or derivative thereof selected from the group consisting of: acetate salt, formate salt, citrate salt, phosphate salt, maleate salt, fumarate salt, tartrate salt, malonate salt, lactic salt, and succinate salt.
[116] The method of any one of paragraphs [1]-[115], wherein said pharmaceutical composition further comprises a pharmaceutically acceptable excipient.
[117] The method of paragraph [116], wherein said pharmaceutically acceptable excipient comprises a surfactant.
[118] The method of paragraph [117], wherein said surfactant comprises Tween, sodium lauryl sulfate (SLS), or dipalmitoylphosphatidylcholine (DPPC).
[119] The method of paragraph [116], wherein said pharmaceutically acceptable excipient comprises a lipid.
[120] The method of paragraph [119], wherein said lipid comprises a polymeric lipid, a sulfonated poly saccharide, or a fatty acid.
[121] The method of paragraph [119], wherein said lipid comprises a polymeric lipid, a sulfonated poly saccharide, or a fatty acid.
[122] The method of any one of paragraphs [1]-[121], wherein said pharmaceutical composition is organoleptically tolerated when inhaled by a human subject.
[123] The method of any one of paragraphs [1]-[122], wherein said pharmaceutical composition does not induce cough reflex when inhaled by a human subject.
[124] The method of any one of paragraphs [1]-[123], wherein said pharmaceutical composition is not or minimally irritative to mouth or throat when inhaled by a human subject.

### EXAMPLES

The following examples are provided to further illustrate some embodiments of the present disclosure, but are not intended to limit the scope of the disclosure; it will be understood by their exemplary nature that other procedures, methodologies, or techniques known to those skilled in the art can alternatively be used.

### Example 1: Organoleptic Testing of Formulations in Solution

This example illustrates organoleptic properties of certain exemplary formulations of imatinib in liquid solution.

In one experiment, exemplary formulations of imatinib according to some embodiments of the present disclosure were prepared as shown in Table 1 and tested by volunteer subjects at a minimum volume for their organoleptic properties.

To prepare the exemplary formulations, imatinib mesylate or imatinib freebase was dissolved directly in sterile water or sterile aqueous solution that contains the designated excipient (e.g., propylene glycol, sodium saccharin, sodium chloride, lactose monohydrate, phosphate, dextrose anhydrous, or hydroxypropyl-β-cyclodextrin). The pH of the resultant solutions was measured, and when needed, water was added to make up the final solution. Each of the solutions were filtered through a 0.22 μm filter and the osmolality of the final solution was measured and determined.

Four individuals were instructed to conduct deep lung inhalation of a minimal volume (<1.5 mL) of one or more exemplary formulations at an interval of 1 to 2 minutes. Each individual was asked to describe the taste of the inhaled solution and record their cough reflex, if any, and their sensational feeling of the solution in throat and mouth both during and after inhalation. Table 2 is a summary of the observations made by the individuals. It was found that among the tested formulations, most of the formulations that were made of imatinib mesylate induced cough reflex of the tested individuals, and were reported to be irritative. In some cases, the testers could not complete the inhalation because of the strong cough reflex or irritation. In contrast, exemplary formulation 13 that was made of imatinib freebase did not induce cough reflex or induced very little cough or irritational sensation in the throat or mouth.

In another experiment, an individual was instructed to conduct deep lung inhalation of each one of the formulations listed in Table TA using the same method as described above. Table 2A summarizes the observations made by this individual.

### Example 2: Exemplary Imatinib Formulations

Table 3 is list of exemplary imatinib formulations with various solubility enhancers according to certain embodiments of the present disclosure.

TABLE 1
Exemplary imatinib formulations for organoleptic test

| | Formula- | | | | | | | | | | | | | |
|---|---|---|---|---|---|---|---|---|---|---|---|---|---|---|
| Component | tion 0 (F0) | F1 | F2 | F3 | F4 | F5 | F6 | F7 | F8 | F9 | F10 | F11 | F12 | F13 |
| Imatinib mesylate in | 5 | 40 | 40 | 40 | 40 | 40 | 40 | 40 | 20 | 40 | 40 | 40 | 40 | — |
| water (mg/mL) | | | | | | | | | | | | | | |
| imatinib freebase in | — | — | — | — | — | — | — | — | — | — | — | — | — | 7 |
| water (mg/mL) | | | | | | | | | | | | | | |
| Propylene glycol (mM) | — | — | 100 | — | 100 | 200 | 250 | 300 | 100 | — | — | — | — | — |
| Sodium Saccharin (mM) | — | — | — | 0.75 | 0.75 | 0.75 | 0.75 | 0.75 | 0.375 | — | — | 0.75 | — | — |
| Sodium Chloride (mM) | — | — | — | — | — | — | — | — | 77 | — | — | — | — | — |
| Lactose Monohydrate | — | — | — | — | — | — | — | — | — | 50 | 150 | 150 | 75 | — |
| (mg/mL) | | | | | | | | | | | | | | |
| Phosphate (mM) | — | — | — | — | — | — | — | — | — | 75 | — | — | — | — |
| Dextrose Anhydrous | — | — | — | — | — | — | — | — | — | — | — | — | 25 | — |
| (mg/mL) | | | | | | | | | | | | | | |
| Hydroxypropyl-b- | — | — | — | — | — | — | — | — | — | — | — | — | — | 40 |
| cyclodextrin (% w/v) | | | | | | | | | | | | | | |
| pH | 5.05 | 5.05 | 5.07 | 5.07 | 5.0 | 5 | 5 | 5 | 5 | 5 | 5 | 5 | 5 | 5 |
| Osmolality | 11 | 67.8 | 167.8 | 69.3 | 269 | 269 | 319 | 302 | 322.6 | 337 | 316 | 318 | 329 | 303 |

TABLE 1A
Exemplary imatinib formulations for organoleptic test

| Component | F14 | F15 | F16 | F17 |
|---|---|---|---|---|
| Imatinib mesylate in | 40 | 40 | — | — |
| water (mg/mL) | | | | |
| imatinib freebase in | — | | 80 | 80 |
| water (mg/mL) | | | | |
| Propylene glycol (mM) | 300 | 100 | — | — |
| Sodium Saccharin (mM) | 0.75 | 0.75 | — | 0.75 |
| Sodium Chloride (mM) | — | 150 | — | — |
| Lactose Monohydrate | — | — | — | — |
| (mg/mL) | | | | |
| Phosphate (mM) | — | — | 50 | 50 |
| Dextrose Anhydrous | — | — | — | — |
| (mg/mL) | | | | |
| Hydroxypropyl-b- | 10 | — | — | — |
| cyclodextrin (% w/v) | | | | |
| sulfobutylether-b- | — | — | 20 | 20 |
| cyclodextrin (% w/v) | | | | |
| pH | — | — | — | — |
| Osmolality | — | — | — | — |

TABLE 2
Summary of organoleptic testing of exemplary formulations

| Formulation | | F0 | F1 | F4 | F4 | F6 | F7 |
|---|---|---|---|---|---|---|---|
| Tester | | Tester 1 | T1 | T1 | T3 | T1 | T1 |
| | | (T1) | | | | | |
| Inhalation | Taste | No | No | No | No | No | No |
| | Cough reflex | Yes | Strong urge | Strong urge to | Yes | Strong urge | No |
| | | | to cough | cough after two | | to cough | |
| | | | immediately | inhalations | | after about | |
| | | | during first | | | a minute of | |
| | | | deep | | | inhalation | |
| | | | inhalation | | | (four or | |
| | | | | | | five deep | |
| | | | | | | breaths) | |
| | Cough? | Yes - 35 s into | Yes, | Yes, | Intense and | Yes, | No |
| | | inhalation, not | persistent, | persistent, | sustained | persistent, | |
| | | severe | strong | strong | | strong | |
| | Sensation in | No | No | No | no irritation | No | No |
| | the throat | | | | no burning | | |
| | (Irritation, | | | | no numbness | | |
| | Tingling, | | | | Tingling? | | |
| | Numbness, | | | | | | |
| | Burning) | | | | | | |
| | Mouthfeel | No | No | No | n/a | No | No |
| | (smooth? | | | | | | |
| | Astringent?) | | | | | | |
| | Other? | | | | | | |
| Post- | Taste | none | No | No | none | No | No |
| inhalation | Cough reflex | Slight | Persisted for | Persisted for | | Persisted for | No |
| (for any of the | | | about 30 | about 5 to | | about 5 | |
| observations | | | minutes | 10 minutes, | | minutes, | |
| above) | | | | ameliorated | | stopped upon | |
| | | | | by inhaling | | inhaling | |
| | | | | saline | | saline | |
| | Cough? | None | Persisted for | Persisted for | cough | Persisted for | No |
| | | | about 30 | about 5 to | continued | about 5 | |
| | | | minutes | 10 minutes, | for ~5 min | minutes, | |
| | | | | ameliorated | | stopped upon | |
| | | | | by inhaling | | inhaling | |
| | | | | saline | | saline | |
| | Sensation in the | | Persisted for | Persisted for | none | Persisted for | No |
| | throat | | about 30 | about 5 to | | about 5 | |
| | (Irritation, | | minutes | 10 minutes, | | minutes, | |
| | Tingling, | | | ameliorated | | stopped upon | |
| | Numbness, or | | | by inhaling | | inhaling | |
| | Burning) | | | saline | | saline | |
| | Mouthfeel | | No | No | none | No | No |
| | (Astringent?) | | | | | | |
| | Other feeling? | | | | | | |
| How long | | NA | About 30 | Persisted for | ~10 min | Persisted for | NA |
| does it take | | | minutes | about 5 to 10 | | about 5 | |
| for the throat | | | | minutes, | | minutes, | |
| to feel | | | | ameliorated | | stopped upon | |
| normal | | | | by inhaling | | inhaling | |
| again? | | | | saline | | saline | |

| Formulation | | F8 | F8 | F9 | F12 | F13 | F13 |
|---|---|---|---|---|---|---|---|
| Tester | | T1 | T2 | T4 | T1 | T1 | T4 |
| Inhalation | Taste | No | Smell of vapor | No | Somewhat | No | No |
| | | | like “new | | Bitter | | |
| | | | plastic” | | | | |
| | Cough reflex | Strong urge | Yes, From lungs, | Yes after | After 1 | No | Slightly |
| | | to cough | not from throat. | 5-10 | minute, | | tickling in |
| | | after about a | | breaths | induced a | | throat |
| | | minute and | | | persistent | | after 7 |
| | | a half of | | | cough and | | breaths, |
| | | inhalation | | | strong mucous | | coughed |
| | | (More than | | | reaction | | and was |
| | | six breaths) | | | | | fine |
| | Cough? | Yes, | Yes | Yes, | Yes, | No | 1 or 2 |
| | | persistent, | | lasting 5- | persistent, | | |
| | | strong | | 10 secs | strong | | |
| | Sensation in | No | Can feel vapors | No | No | No | No |
| | the throat | | going down but, | | | | |
| | (Irritation, | | no throat | | | | |
| | Tingling, | | irritation during. | | | | |
| | Numbness, | | | | | | |
| | Burning) | | | | | | |
| | Mouthfeel | No | Mostly vapory | No | No | No | No |
| | (smooth? | | and slightly | | | | |
| | Astringent?) | | sweet after | | | | |
| | Other? | | Uncontrollable | | | | |
| | | | cough reflex | | | | |
| | | | after 5 deep | | | | |
| | | | inhalations (~30 | | | | |
| | | | sec); Came on | | | | |
| | | | almost | | | | |
| | | | instanteously, did | | | | |
| | | | not feel it | | | | |
| | | | building, but | | | | |
| | | | once set off, it | | | | |
| | | | could not be | | | | |
| | | | suppressed or | | | | |
| | | | controlled. | | | | |
| Post- | Taste | No | Little sweetness | No | No | No | No |
| inhalation | Cough reflex | Persisted for | Continued deep | yes | Persisted, | No | No |
| (for any of the | | about 5 | lung cough for 5 | | stopped upon | reaction | |
| observations | | minutes, | minutes, tapering | | inhaling saline | after 2 | |
| above) | | stopped upon | down. Still feel | | | mins | |
| | | inhaling | urge to cough up | | | | |
| | | saline | to 20 min after. | | | | |
| | Cough? | Persisted for | Yes, diminishing | mild | Persisted, | No | No |
| | | about 5 | after 5 min, but | | stopped upon | reaction | |
| | | minutes, | still residual up | | inhaling saline | after 2 | |
| | | stopped upon | to 20 min post | | | mins | |
| | | inhaling | completion. | | | | |
| | | saline | | | | | |
| | Sensation in the | Persisted for | None | No | Persisted, | No | No |
| | throat | about 5 | | | stopped upon | reaction | |
| | (Irritation, | minutes, | | | inhaling saline | after 2 | |
| | Tingling, | stopped upon | | | | mins | |
| | Numbness, or | inhaling | | | | | |
| | Burning) | saline | | | | | |
| | Mouthfeel | No | None | No | No | No | No |
| | (Astringent?) | | | | | | |
| | Other feeling? | | | | | | |
| How long | | Persisted for | NA | NA | NA | NA | NA |
| does it take | | about 5 | | | | | |
| for the throat | | minutes, | | | | | |
| to feel | | stopped upon | | | | | |
| normal | | inhaling | | | | | |
| again? | | saline | | | | | |

TABLE 2A
Summary of organoleptic testing

| Formulation | F14 | F15 | F16 | F17 |
|---|---|---|---|---|
| Cough? | Strong cough | Strong couph | No cough | No cough |
| | during | after inhalation | | |
| | inhalation | | | |
| Tolerable for | Not tolerable | Well tolerated | Well tolerated | Well tolerated |
| inhalation? | | | | |

TABLE 3
Exemplary imatinib formulations in aqueous solution

| | | | | | | | | PEG | PEG | | | | | |
|---|---|---|---|---|---|---|---|---|---|---|---|---|---|---|
| | Imatinib | α-CD | β-CD | γ-CD | HPβCD | MβCD | SBEβCD | 300 | 400 | PG | Ethanol | | | |
| | freebase | (% | (% | (% | (% | (% | (% | (% | (% | (% | (% | Saccharin | | pH |
| Formulations | (mg/mL) | w/v) | w/v) | w/v) | w/v) | w/v) | w/v) | v/v) | v/v) | v/v) | v/v) | (mM) | pH | buffer |
| 14 | 20 | 5 | | | | | | | | | | | 5 | phosphate |
| 15 | 20 | 10 | | | | | | | | | | | 5 | phosphate |
| 16 | 20 | 20 | | | | | | | | | | | 5 | phosphate |
| 17 | 20 | 30 | | | | | | | | | | | 5 | phosphate |
| 18 | 20 | | 5 | | | | | | | | | | 5 | phosphate |
| 19 | 20 | | 10 | | | | | | | | | | 5 | phosphate |
| 20 | 20 | | 20 | | | | | | | | | | 5 | phosphate |
| 21 | 20 | | 30 | | | | | | | | | | 5 | phosphate |
| 22 | 20 | | | 5 | | | | | | | | | 5 | phosphate |
| 23 | 20 | | | 10 | | | | | | | | | 5 | phosphate |
| 24 | 20 | | | 20 | | | | | | | | | 5 | phosphate |
| 25 | 20 | | | 30 | | | | | | | | | 5 | phosphate |
| 26 | 20 | | | | 5 | | | | | | | | 5 | phosphate |
| 27 | 20 | | | | 10 | | | | | | | | 5 | phosphate |
| 28 | 20 | | | | 20 | | | | | | | | 5 | phosphate |
| 29 | 20 | | | | 30 | | | | | | | | 5 | phosphate |
| 30 | 20 | | | | | 5 | | | | | | | 5 | phosphate |
| 31 | 20 | | | | | 10 | | | | | | | 5 | phosphate |
| 32 | 20 | | | | | 20 | | | | | | | 5 | phosphate |
| 33 | 20 | | | | | 30 | | | | | | | 5 | phosphate |
| 34 | 20 | | | | | | 5 | | | | | | 5 | phosphate |
| 35 | 20 | | | | | | 10 | | | | | | 5 | phosphate |
| 36 | 20 | | | | | | 20 | | | | | | 5 | phosphate |
| 37 | 20 | | | | | | 30 | | | | | | 5 | phosphate |
| 38 | 20 | | | | | | | 10 | | | | | 5 | phosphate |
| 39 | 20 | | | | | | | 20 | | | | | 5 | phosphate |
| 40 | 20 | | | | | | | 100 | | | | | 5 | phosphate |
| 41 | 20 | | | | | | | | 10 | | | | 5 | phosphate |
| 42 | 20 | | | | | | | | 20 | | | | 5 | phosphate |
| 43 | 20 | | | | | | | | 100 | | | | 5 | phosphate |
| 44 | 20 | | | | | | | | | 10 | | | 5 | phosphate |
| 45 | 20 | | | | | | | | | 20 | | | 5 | phosphate |
| 46 | 20 | | | | | | | | | 100 | | | 5 | phosphate |
| 47 | 20 | | | | | | | | | | 5 | | 5 | phosphate |
| 48 | 20 | | | | | | | | | | 10 | | 5 | phosphate |
| 49 | 20 | | | | | | | | | | 20 | | 5 | phosphate |
| 50 | 20 | | | | | | | | | | 100 | | 5 | phosphate |
| 51 | 30 | 5 | | | | | | | | | | | 5 | phosphate |
| 52 | 30 | 10 | | | | | | | | | | | 5 | phosphate |
| 53 | 30 | 20 | | | | | | | | | | | 5 | phosphate |
| 54 | 30 | 30 | | | | | | | | | | | 5 | phosphate |
| 55 | 30 | | 5 | | | | | | | | | | 5 | phosphate |
| 56 | 30 | | 10 | | | | | | | | | | 5 | phosphate |
| 57 | 30 | | 20 | | | | | | | | | | 5 | phosphate |
| 58 | 30 | | 30 | | | | | | | | | | 5 | phosphate |
| 59 | 30 | | | 5 | | | | | | | | | 5 | phosphate |
| 60 | 30 | | | 10 | | | | | | | | | 5 | phosphate |
| 61 | 30 | | | 20 | | | | | | | | | 5 | phosphate |
| 62 | 30 | | | 30 | | | | | | | | | 5 | phosphate |
| 63 | 30 | | | | 5 | | | | | | | | 5 | phosphate |
| 64 | 30 | | | | 10 | | | | | | | | 5 | phosphate |
| 65 | 30 | | | | 20 | | | | | | | | 5 | phosphate |
| 66 | 30 | | | | 30 | | | | | | | | 5 | phosphate |
| 67 | 30 | | | | | 5 | | | | | | | 5 | phosphate |
| 68 | 30 | | | | | 10 | | | | | | | 5 | phosphate |
| 69 | 30 | | | | | 20 | | | | | | | 5 | phosphate |
| 70 | 30 | | | | | 30 | | | | | | | 5 | phosphate |
| 71 | 30 | | | | | | 5 | | | | | | 5 | phosphate |
| 72 | 30 | | | | | | 10 | | | | | | 5 | phosphate |
| 73 | 30 | | | | | | 20 | | | | | | 5 | phosphate |
| 74 | 30 | | | | | | 30 | | | | | | 5 | phosphate |
| 75 | 30 | | | | | | | 10 | | | | | 5 | phosphate |
| 76 | 30 | | | | | | | 20 | | | | | 5 | phosphate |
| 77 | 30 | | | | | | | 100 | | | | | 5 | phosphate |
| 78 | 30 | | | | | | | | 10 | | | | 5 | phosphate |
| 79 | 30 | | | | | | | | 20 | | | | 5 | phosphate |
| 80 | 30 | | | | | | | | 100 | | | | 5 | phosphate |
| 81 | 30 | | | | | | | | | 10 | | | 5 | phosphate |
| 82 | 30 | | | | | | | | | 20 | | | 5 | phosphate |
| 83 | 30 | | | | | | | | | 100 | | | 5 | phosphate |
| 84 | 30 | | | | | | | | | | 5 | | 5 | phosphate |
| 85 | 30 | | | | | | | | | | 10 | | 5 | phosphate |
| 86 | 30 | | | | | | | | | | 20 | | 5 | phosphate |
| 87 | 30 | | | | | | | | | | 100 | | 5 | phosphate |
| 88 | 40 | 5 | | | | | | | | | | | 5 | phosphate |
| 89 | 40 | 10 | | | | | | | | | | | 5 | phosphate |
| 90 | 40 | 20 | | | | | | | | | | | 5 | phosphate |
| 91 | 40 | 30 | | | | | | | | | | | 5 | phosphate |
| 92 | 40 | | 5 | | | | | | | | | | 5 | phosphate |
| 93 | 40 | | 10 | | | | | | | | | | 5 | phosphate |
| 94 | 40 | | 20 | | | | | | | | | | 5 | phosphate |
| 95 | 40 | | 30 | | | | | | | | | | 5 | phosphate |
| 96 | 40 | | | 5 | | | | | | | | | 5 | phosphate |
| 97 | 40 | | | 10 | | | | | | | | | 5 | phosphate |
| 98 | 40 | | | 20 | | | | | | | | | 5 | phosphate |
| 99 | 40 | | | 30 | | | | | | | | | 5 | phosphate |
| 100 | 40 | | | | 5 | | | | | | | | 5 | phosphate |
| 101 | 40 | | | | 10 | | | | | | | | 5 | phosphate |
| 102 | 40 | | | | 20 | | | | | | | | 5 | phosphate |
| 103 | 40 | | | | 30 | | | | | | | | 5 | phosphate |
| 104 | 40 | | | | | 5 | | | | | | | 5 | phosphate |
| 105 | 40 | | | | | 10 | | | | | | | 5 | phosphate |
| 106 | 40 | | | | | 20 | | | | | | | 5 | phosphate |
| 107 | 40 | | | | | 30 | | | | | | | 5 | phosphate |
| 108 | 40 | | | | | | 5 | | | | | | 5 | phosphate |
| 109 | 40 | | | | | | 10 | | | | | | 5 | phosphate |
| 110 | 40 | | | | | | 20 | | | | | | 5 | phosphate |
| 111 | 40 | | | | | | 30 | | | | | | 5 | phosphate |
| 112 | 40 | | | | | | | 10 | | | | | 5 | phosphate |
| 113 | 40 | | | | | | | 20 | | | | | 5 | phosphate |
| 114 | 40 | | | | | | | 100 | | | | | 5 | phosphate |
| 115 | 40 | | | | | | | | 10 | | | | 5 | phosphate |
| 116 | 40 | | | | | | | | 20 | | | | 5 | phosphate |
| 117 | 40 | | | | | | | | 100 | | | | 5 | phosphate |
| 118 | 40 | | | | | | | | | 10 | | | 5 | phosphate |
| 119 | 40 | | | | | | | | | 20 | | | 5 | phosphate |
| 120 | 40 | | | | | | | | | 100 | | | 5 | phosphate |
| 121 | 40 | | | | | | | | | | 5 | | 5 | phosphate |
| 122 | 40 | | | | | | | | | | 10 | | 5 | phosphate |
| 123 | 40 | | | | | | | | | | 20 | | 5 | phosphate |
| 124 | 40 | | | | | | | | | | 100 | | 5 | phosphate |
| 125 | 50 | | | | | | 10 | | | | | | 5 | phosphate |
| 126 | 50 | | | | | | 20 | | | | | | 5 | phosphate |
| 127 | 50 | | | | | | 30 | | | | | | 5 | phosphate |
| 128 | 50 | | | | | | 40 | | | | | | 5 | phosphate |
| 129 | 50 | | | | | | 50 | | | | | | 5 | phosphate |
| 130 | 100 | | | | | | 20 | | | | | | 5 | phosphate |
| 131 | 100 | | | | | | 30 | | | | | | 5 | phosphate |
| 132 | 100 | | | | | | 40 | | | | | | 5 | phosphate |
| 133 | 100 | | | | | | 50 | | | | | | 5 | phosphate |
| 134 | 150 | | | | | | 20 | | | | | | 5 | phosphate |
| 135 | 150 | | | | | | 30 | | | | | | 5 | phosphate |
| 136 | 150 | | | | | | 40 | | | | | | 5 | phosphate |
| 137 | 150 | | | | | | 50 | | | | | | 5 | phosphate |
| 138 | 200 | | | | | | 30 | | | | | | 5 | phosphate |
| 139 | 200 | | | | | | 40 | | | | | | 5 | phosphate |
| 140 | 200 | | | | | | 50 | | | | | | 5 | phosphate |
| 141 | 250 | | | | | | 30 | | | | | | 5 | phosphate |
| 142 | 250 | | | | | | 40 | | | | | | 5 | phosphate |
| 143 | 250 | | | | | | 50 | | | | | | 5 | phosphate |
| 144 | 300 | | | | | | 30 | | | | | | 5 | phosphate |
| 145 | 300 | | | | | | 40 | | | | | | 5 | phosphate |
| 146 | 300 | | | | | | 50 | | | | | | 5 | phosphate |
| 147 | 350 | | | | | | 40 | | | | | | 5 | phosphate |
| 148 | 400 | | | | | | 50 | | | | | | 5 | phosphate |
| 149 | 400 | | | | | | 40 | | | | | | 5 | phosphate |
| 150 | 450 | | | | | | 50 | | | | | | 5 | phosphate |
| 151 | 500 | | | | | | 50 | | | | | | 5 | phosphate |
| 152 | 20 | 5 | | | | | | | | | | | 6 | phosphate |
| 153 | 20 | 10 | | | | | | | | | | | 6 | phosphate |
| 154 | 20 | 20 | | | | | | | | | | | 6 | phosphate |
| 155 | 20 | 30 | | | | | | | | | | | 6 | phosphate |
| 156 | 20 | | 5 | | | | | | | | | | 6 | phosphate |
| 157 | 20 | | 10 | | | | | | | | | | 6 | phosphate |
| 158 | 20 | | 20 | | | | | | | | | | 6 | phosphate |
| 159 | 20 | | 30 | | | | | | | | | | 6 | phosphate |
| 160 | 20 | | | 5 | | | | | | | | | 6 | phosphate |
| 161 | 20 | | | 10 | | | | | | | | | 6 | phosphate |
| 162 | 20 | | | 20 | | | | | | | | | 6 | phosphate |
| 163 | 20 | | | 30 | | | | | | | | | 6 | phosphate |
| 164 | 20 | | | | 5 | | | | | | | | 6 | phosphate |
| 165 | 20 | | | | 10 | | | | | | | | 6 | phosphate |
| 166 | 20 | | | | 20 | | | | | | | | 6 | phosphate |
| 167 | 20 | | | | 30 | | | | | | | | 6 | phosphate |
| 168 | 20 | | | | | 5 | | | | | | | 6 | phosphate |
| 169 | 20 | | | | | 10 | | | | | | | 6 | phosphate |
| 170 | 20 | | | | | 20 | | | | | | | 6 | phosphate |
| 171 | 20 | | | | | 30 | | | | | | | 6 | phosphate |
| 172 | 20 | | | | | | 5 | | | | | | 6 | phosphate |
| 173 | 20 | | | | | | 10 | | | | | | 6 | phosphate |
| 174 | 20 | | | | | | 20 | | | | | | 6 | phosphate |
| 175 | 20 | | | | | | 30 | | | | | | 6 | phosphate |
| 176 | 20 | | | | | | | 10 | | | | | 6 | phosphate |
| 177 | 20 | | | | | | | 20 | | | | | 6 | phosphate |
| 178 | 20 | | | | | | | 100 | | | | | 6 | phosphate |
| 179 | 20 | | | | | | | | 10 | | | | 6 | phosphate |
| 180 | 20 | | | | | | | | 20 | | | | 6 | phosphate |
| 181 | 20 | | | | | | | | 100 | | | | 6 | phosphate |
| 182 | 20 | | | | | | | | | 10 | | | 6 | phosphate |
| 183 | 20 | | | | | | | | | 20 | | | 6 | phosphate |
| 184 | 20 | | | | | | | | | 100 | | | 6 | phosphate |
| 185 | 20 | | | | | | | | | | 5 | | 6 | phosphate |
| 186 | 20 | | | | | | | | | | 10 | | 6 | phosphate |
| 187 | 20 | | | | | | | | | | 20 | | 6 | phosphate |
| 188 | 20 | | | | | | | | | | 100 | | 6 | phosphate |
| 189 | 30 | 5 | | | | | | | | | | | 6 | phosphate |
| 190 | 30 | 10 | | | | | | | | | | | 6 | phosphate |
| 191 | 30 | 20 | | | | | | | | | | | 6 | phosphate |
| 192 | 30 | 30 | | | | | | | | | | | 6 | phosphate |
| 193 | 30 | | 5 | | | | | | | | | | 6 | phosphate |
| 194 | 30 | | 10 | | | | | | | | | | 6 | phosphate |
| 195 | 30 | | 20 | | | | | | | | | | 6 | phosphate |
| 196 | 30 | | 30 | | | | | | | | | | 6 | phosphate |
| 197 | 30 | | | 5 | | | | | | | | | 6 | phosphate |
| 198 | 30 | | | 10 | | | | | | | | | 6 | phosphate |
| 199 | 30 | | | 20 | | | | | | | | | 6 | phosphate |
| 200 | 30 | | | 30 | | | | | | | | | 6 | phosphate |
| 201 | 30 | | | | 5 | | | | | | | | 6 | phosphate |
| 202 | 30 | | | | 10 | | | | | | | | 6 | phosphate |
| 203 | 30 | | | | 20 | | | | | | | | 6 | phosphate |
| 204 | 30 | | | | 30 | | | | | | | | 6 | phosphate |
| 205 | 30 | | | | | 5 | | | | | | | 6 | phosphate |
| 206 | 30 | | | | | 10 | | | | | | | 6 | phosphate |
| 207 | 30 | | | | | 20 | | | | | | | 6 | phosphate |
| 208 | 30 | | | | | 30 | | | | | | | 6 | phosphate |
| 209 | 30 | | | | | | 5 | | | | | | 6 | phosphate |
| 210 | 30 | | | | | | 10 | | | | | | 6 | phosphate |
| 211 | 30 | | | | | | 20 | | | | | | 6 | phosphate |
| 212 | 30 | | | | | | 30 | | | | | | 6 | phosphate |
| 213 | 30 | | | | | | | 10 | | | | | 6 | phosphate |
| 214 | 30 | | | | | | | 20 | | | | | 6 | phosphate |
| 215 | 30 | | | | | | | 100 | | | | | 6 | phosphate |
| 216 | 30 | | | | | | | | 10 | | | | 6 | phosphate |
| 217 | 30 | | | | | | | | 20 | | | | 6 | phosphate |
| 218 | 30 | | | | | | | | 100 | | | | 6 | phosphate |
| 219 | 30 | | | | | | | | | 10 | | | 6 | phosphate |
| 220 | 30 | | | | | | | | | 20 | | | 6 | phosphate |
| 221 | 30 | | | | | | | | | 100 | | | 6 | phosphate |
| 222 | 30 | | | | | | | | | | 5 | | 6 | phosphate |
| 223 | 30 | | | | | | | | | | 10 | | 6 | phosphate |
| 224 | 30 | | | | | | | | | | 20 | | 6 | phosphate |
| 225 | 30 | | | | | | | | | | 100 | | 6 | phosphate |
| 226 | 40 | 5 | | | | | | | | | | | 6 | phosphate |
| 227 | 40 | 10 | | | | | | | | | | | 6 | phosphate |
| 228 | 40 | 20 | | | | | | | | | | | 6 | phosphate |
| 229 | 40 | 30 | | | | | | | | | | | 6 | phosphate |
| 230 | 40 | | 5 | | | | | | | | | | 6 | phosphate |
| 231 | 40 | | 10 | | | | | | | | | | 6 | phosphate |
| 232 | 40 | | 20 | | | | | | | | | | 6 | phosphate |
| 233 | 40 | | 30 | | | | | | | | | | 6 | phosphate |
| 234 | 40 | | | 5 | | | | | | | | | 6 | phosphate |
| 235 | 40 | | | 10 | | | | | | | | | 6 | phosphate |
| 236 | 40 | | | 20 | | | | | | | | | 6 | phosphate |
| 237 | 40 | | | 30 | | | | | | | | | 6 | phosphate |
| 238 | 40 | | | | 5 | | | | | | | | 6 | phosphate |
| 239 | 40 | | | | 10 | | | | | | | | 6 | phosphate |
| 240 | 40 | | | | 20 | | | | | | | | 6 | phosphate |
| 241 | 40 | | | | 30 | | | | | | | | 6 | phosphate |
| 242 | 40 | | | | | 5 | | | | | | | 6 | phosphate |
| 243 | 40 | | | | | 10 | | | | | | | 6 | phosphate |
| 244 | 40 | | | | | 20 | | | | | | | 6 | phosphate |
| 245 | 40 | | | | | 30 | | | | | | | 6 | phosphate |
| 246 | 40 | | | | | | 5 | | | | | | 6 | phosphate |
| 247 | 40 | | | | | | 10 | | | | | | 6 | phosphate |
| 248 | 40 | | | | | | 20 | | | | | | 6 | phosphate |
| 249 | 40 | | | | | | 30 | | | | | | 6 | phosphate |
| 250 | 40 | | | | | | | 10 | | | | | 6 | phosphate |
| 251 | 40 | | | | | | | 20 | | | | | 6 | phosphate |
| 252 | 40 | | | | | | | 100 | | | | | 6 | phosphate |
| 253 | 40 | | | | | | | | 10 | | | | 6 | phosphate |
| 254 | 40 | | | | | | | | 20 | | | | 6 | phosphate |
| 255 | 40 | | | | | | | | 100 | | | | 6 | phosphate |
| 256 | 40 | | | | | | | | | 10 | | | 6 | phosphate |
| 257 | 40 | | | | | | | | | 20 | | | 6 | phosphate |
| 258 | 40 | | | | | | | | | 100 | | | 6 | phosphate |
| 259 | 40 | | | | | | | | | | 5 | | 6 | phosphate |
| 260 | 40 | | | | | | | | | | 10 | | 6 | phosphate |
| 261 | 40 | | | | | | | | | | 20 | | 6 | phosphate |
| 262 | 40 | | | | | | | | | | 100 | | 6 | phosphate |
| 263 | 50 | | | | | | 10 | | | | | | 6 | phosphate |
| 264 | 50 | | | | | | 20 | | | | | | 6 | phosphate |
| 265 | 50 | | | | | | 30 | | | | | | 6 | phosphate |
| 266 | 50 | | | | | | 40 | | | | | | 6 | phosphate |
| 267 | 50 | | | | | | 50 | | | | | | 6 | phosphate |
| 268 | 100 | | | | | | 20 | | | | | | 6 | phosphate |
| 269 | 100 | | | | | | 30 | | | | | | 6 | phosphate |
| 270 | 100 | | | | | | 40 | | | | | | 6 | phosphate |
| 271 | 100 | | | | | | 50 | | | | | | 6 | phosphate |
| 272 | 150 | | | | | | 20 | | | | | | 6 | phosphate |
| 273 | 150 | | | | | | 30 | | | | | | 6 | phosphate |
| 274 | 150 | | | | | | 40 | | | | | | 6 | phosphate |
| 275 | 150 | | | | | | 50 | | | | | | 6 | phosphate |
| 276 | 200 | | | | | | 30 | | | | | | 6 | phosphate |
| 277 | 200 | | | | | | 40 | | | | | | 6 | phosphate |
| 278 | 200 | | | | | | 50 | | | | | | 6 | phosphate |
| 279 | 250 | | | | | | 30 | | | | | | 6 | phosphate |
| 280 | 250 | | | | | | 40 | | | | | | 6 | phosphate |
| 281 | 250 | | | | | | 50 | | | | | | 6 | phosphate |
| 282 | 300 | | | | | | 30 | | | | | | 6 | phosphate |
| 283 | 300 | | | | | | 40 | | | | | | 6 | phosphate |
| 284 | 300 | | | | | | 50 | | | | | | 6 | phosphate |
| 285 | 350 | | | | | | 40 | | | | | | 6 | phosphate |
| 286 | 400 | | | | | | 50 | | | | | | 6 | phosphate |
| 287 | 400 | | | | | | 40 | | | | | | 6 | phosphate |
| 288 | 450 | | | | | | 50 | | | | | | 6 | phosphate |
| 289 | 500 | | | | | | 50 | | | | | | 6 | phosphate |
| 290 | 20 | 5 | | | | | | | | | | | 5 | citrate |
| 291 | 20 | 10 | | | | | | | | | | | 5 | citrate |
| 292 | 20 | 20 | | | | | | | | | | | 5 | citrate |
| 293 | 20 | 30 | | | | | | | | | | | 5 | citrate |
| 294 | 20 | | 5 | | | | | | | | | | 5 | citrate |
| 295 | 20 | | 10 | | | | | | | | | | 5 | citrate |
| 296 | 20 | | 20 | | | | | | | | | | 5 | citrate |
| 297 | 20 | | 30 | | | | | | | | | | 5 | citrate |
| 298 | 20 | | | 5 | | | | | | | | | 5 | citrate |
| 299 | 20 | | | 10 | | | | | | | | | 5 | citrate |
| 300 | 20 | | | 20 | | | | | | | | | 5 | citrate |
| 301 | 20 | | | 30 | | | | | | | | | 5 | citrate |
| 302 | 20 | | | | 5 | | | | | | | | 5 | citrate |
| 303 | 20 | | | | 10 | | | | | | | | 5 | citrate |
| 304 | 20 | | | | 20 | | | | | | | | 5 | citrate |
| 305 | 20 | | | | 30 | | | | | | | | 5 | citrate |
| 306 | 20 | | | | | 5 | | | | | | | 5 | citrate |
| 307 | 20 | | | | | 10 | | | | | | | 5 | citrate |
| 308 | 20 | | | | | 20 | | | | | | | 5 | citrate |
| 309 | 20 | | | | | 30 | | | | | | | 5 | citrate |
| 310 | 20 | | | | | | 5 | | | | | | 5 | citrate |
| 311 | 20 | | | | | | 10 | | | | | | 5 | citrate |
| 312 | 20 | | | | | | 20 | | | | | | 5 | citrate |
| 313 | 20 | | | | | | 30 | | | | | | 5 | citrate |
| 314 | 20 | | | | | | | 10 | | | | | 5 | citrate |
| 315 | 20 | | | | | | | 20 | | | | | 5 | citrate |
| 316 | 20 | | | | | | | 100 | | | | | 5 | citrate |
| 317 | 20 | | | | | | | | 10 | | | | 5 | citrate |
| 318 | 20 | | | | | | | | 20 | | | | 5 | citrate |
| 319 | 20 | | | | | | | | 100 | | | | 5 | citrate |
| 320 | 20 | | | | | | | | | 10 | | | 5 | citrate |
| 321 | 20 | | | | | | | | | 20 | | | 5 | citrate |
| 322 | 20 | | | | | | | | | 100 | | | 5 | citrate |
| 323 | 20 | | | | | | | | | | 5 | | 5 | citrate |
| 324 | 20 | | | | | | | | | | 10 | | 5 | citrate |
| 325 | 20 | | | | | | | | | | 20 | | 5 | citrate |
| 326 | 20 | | | | | | | | | | 100 | | 5 | citrate |
| 327 | 30 | 5 | | | | | | | | | | | 5 | citrate |
| 328 | 30 | 10 | | | | | | | | | | | 5 | citrate |
| 329 | 30 | 20 | | | | | | | | | | | 5 | citrate |
| 330 | 30 | 30 | | | | | | | | | | | 5 | citrate |
| 331 | 30 | | 5 | | | | | | | | | | 5 | citrate |
| 332 | 30 | | 10 | | | | | | | | | | 5 | citrate |
| 333 | 30 | | 20 | | | | | | | | | | 5 | citrate |
| 334 | 30 | | 30 | | | | | | | | | | 5 | citrate |
| 335 | 30 | | | 5 | | | | | | | | | 5 | citrate |
| 336 | 30 | | | 10 | | | | | | | | | 5 | citrate |
| 337 | 30 | | | 20 | | | | | | | | | 5 | citrate |
| 338 | 30 | | | 30 | | | | | | | | | 5 | citrate |
| 339 | 30 | | | | 5 | | | | | | | | 5 | citrate |
| 340 | 30 | | | | 10 | | | | | | | | 5 | citrate |
| 341 | 30 | | | | 20 | | | | | | | | 5 | citrate |
| 342 | 30 | | | | 30 | | | | | | | | 5 | citrate |
| 343 | 30 | | | | | 5 | | | | | | | 5 | citrate |
| 344 | 30 | | | | | 10 | | | | | | | 5 | citrate |
| 345 | 30 | | | | | 20 | | | | | | | 5 | citrate |
| 346 | 30 | | | | | 30 | | | | | | | 5 | citrate |
| 347 | 30 | | | | | | 5 | | | | | | 5 | citrate |
| 348 | 30 | | | | | | 10 | | | | | | 5 | citrate |
| 349 | 30 | | | | | | 20 | | | | | | 5 | citrate |
| 350 | 30 | | | | | | 30 | | | | | | 5 | citrate |
| 351 | 30 | | | | | | | 10 | | | | | 5 | citrate |
| 352 | 30 | | | | | | | 20 | | | | | 5 | citrate |
| 353 | 30 | | | | | | | 100 | | | | | 5 | citrate |
| 354 | 30 | | | | | | | | 10 | | | | 5 | citrate |
| 355 | 30 | | | | | | | | 20 | | | | 5 | citrate |
| 356 | 30 | | | | | | | | 100 | | | | 5 | citrate |
| 357 | 30 | | | | | | | | | 10 | | | 5 | citrate |
| 358 | 30 | | | | | | | | | 20 | | | 5 | citrate |
| 359 | 30 | | | | | | | | | 100 | | | 5 | citrate |
| 360 | 30 | | | | | | | | | | | | 5 | citrate |
| 361 | 30 | | | | | | | | | | | | 5 | citrate |
| 362 | 30 | | | | | | | | | | 5 | | 5 | citrate |
| 363 | 30 | | | | | | | | | | 10 | | 5 | citrate |
| 364 | 30 | | | | | | | | | | 20 | | 5 | citrate |
| 365 | 30 | | | | | | | | | | 100 | | 5 | citrate |
| 366 | 40 | 5 | | | | | | | | | | | 5 | citrate |
| 367 | 40 | 10 | | | | | | | | | | | 5 | citrate |
| 368 | 40 | 20 | | | | | | | | | | | 5 | citrate |
| 369 | 40 | 30 | | | | | | | | | | | 5 | citrate |
| 370 | 40 | | 5 | | | | | | | | | | 5 | citrate |
| 371 | 40 | | 10 | | | | | | | | | | 5 | citrate |
| 372 | 40 | | 20 | | | | | | | | | | 5 | citrate |
| 373 | 40 | | 30 | | | | | | | | | | 5 | citrate |
| 374 | 40 | | | 5 | | | | | | | | | 5 | citrate |
| 375 | 40 | | | 10 | | | | | | | | | 5 | citrate |
| 376 | 40 | | | 20 | | | | | | | | | 5 | citrate |
| 377 | 40 | | | 30 | | | | | | | | | 5 | citrate |
| 378 | 40 | | | | 5 | | | | | | | | 5 | citrate |
| 379 | 40 | | | | 10 | | | | | | | | 5 | citrate |
| 380 | 40 | | | | 20 | | | | | | | | 5 | citrate |
| 381 | 40 | | | | 30 | | | | | | | | 5 | citrate |
| 382 | 40 | | | | | 5 | | | | | | | 5 | citrate |
| 383 | 40 | | | | | 10 | | | | | | | 5 | citrate |
| 384 | 40 | | | | | 20 | | | | | | | 5 | citrate |
| 385 | 40 | | | | | 30 | | | | | | | 5 | citrate |
| 386 | 40 | | | | | | 5 | | | | | | 5 | citrate |
| 387 | 40 | | | | | | 10 | | | | | | 5 | citrate |
| 388 | 40 | | | | | | 20 | | | | | | 5 | citrate |
| 389 | 40 | | | | | | 30 | | | | | | 5 | citrate |
| 390 | 40 | | | | | | | 10 | | | | | 5 | citrate |
| 391 | 40 | | | | | | | 20 | | | | | 5 | citrate |
| 392 | 40 | | | | | | | 100 | | | | | 5 | citrate |
| 393 | 40 | | | | | | | | 10 | | | | 5 | citrate |
| 394 | 40 | | | | | | | | 20 | | | | 5 | citrate |
| 395 | 40 | | | | | | | | 100 | | | | 5 | citrate |
| 396 | 40 | | | | | | | | | 10 | | | 5 | citrate |
| 397 | 40 | | | | | | | | | 20 | | | 5 | citrate |
| 398 | 40 | | | | | | | | | 100 | | | 5 | citrate |
| 399 | 40 | | | | | | | | | | 5 | | 5 | citrate |
| 400 | 40 | | | | | | | | | | 10 | | 5 | citrate |
| 401 | 40 | | | | | | | | | | 20 | | 5 | citrate |
| 402 | 40 | | | | | | | | | | 100 | | 5 | citrate |
| 403 | 50 | | | | | | 10 | | | | | | 5 | citrate |
| 404 | 50 | | | | | | 20 | | | | | | 5 | citrate |
| 405 | 50 | | | | | | 30 | | | | | | 5 | citrate |
| 406 | 50 | | | | | | 40 | | | | | | 5 | citrate |
| 407 | 50 | | | | | | 50 | | | | | | 5 | citrate |
| 408 | 100 | | | | | | 20 | | | | | | 5 | citrate |
| 409 | 100 | | | | | | 30 | | | | | | 5 | citrate |
| 410 | 100 | | | | | | 40 | | | | | | 5 | citrate |
| 411 | 100 | | | | | | 50 | | | | | | 5 | citrate |
| 412 | 150 | | | | | | 20 | | | | | | 5 | citrate |
| 413 | 150 | | | | | | 30 | | | | | | 5 | citrate |
| 414 | 150 | | | | | | 40 | | | | | | 5 | citrate |
| 415 | 150 | | | | | | 50 | | | | | | 5 | citrate |
| 416 | 200 | | | | | | 30 | | | | | | 5 | citrate |
| 417 | 200 | | | | | | 40 | | | | | | 5 | citrate |
| 418 | 200 | | | | | | 50 | | | | | | 5 | citrate |
| 419 | 250 | | | | | | 30 | | | | | | 5 | citrate |
| 420 | 250 | | | | | | 40 | | | | | | 5 | citrate |
| 421 | 250 | | | | | | 50 | | | | | | 5 | citrate |
| 422 | 300 | | | | | | 30 | | | | | | 5 | citrate |
| 423 | 300 | | | | | | 40 | | | | | | 5 | citrate |
| 424 | 300 | | | | | | 50 | | | | | | 5 | citrate |
| 425 | 350 | | | | | | 40 | | | | | | 5 | citrate |
| 426 | 400 | | | | | | 50 | | | | | | 5 | citrate |
| 427 | 400 | | | | | | 40 | | | | | | 5 | citrate |
| 428 | 450 | | | | | | 50 | | | | | | 5 | citrate |
| 429 | 500 | | | | | | 50 | | | | | | 5 | citrate |
| 430 | 20 | 5 | | | | | | | | | | | 6 | citrate |
| 431 | 20 | 10 | | | | | | | | | | | 6 | citrate |
| 432 | 20 | 20 | | | | | | | | | | | 6 | citrate |
| 433 | 20 | 30 | | | | | | | | | | | 6 | citrate |
| 434 | 20 | | 5 | | | | | | | | | | 6 | citrate |
| 435 | 20 | | 10 | | | | | | | | | | 6 | citrate |
| 436 | 20 | | 20 | | | | | | | | | | 6 | citrate |
| 437 | 20 | | 30 | | | | | | | | | | 6 | citrate |
| 438 | 20 | | | 5 | | | | | | | | | 6 | citrate |
| 439 | 20 | | | 10 | | | | | | | | | 6 | citrate |
| 440 | 20 | | | 20 | | | | | | | | | 6 | citrate |
| 441 | 20 | | | 30 | | | | | | | | | 6 | citrate |
| 442 | 20 | | | | 5 | | | | | | | | 6 | citrate |
| 443 | 20 | | | | 10 | | | | | | | | 6 | citrate |
| 444 | 20 | | | | 20 | | | | | | | | 6 | citrate |
| 445 | 20 | | | | 30 | | | | | | | | 6 | citrate |
| 446 | 20 | | | | | 5 | | | | | | | 6 | citrate |
| 447 | 20 | | | | | 10 | | | | | | | 6 | citrate |
| 448 | 20 | | | | | 20 | | | | | | | 6 | citrate |
| 449 | 20 | | | | | 30 | | | | | | | 6 | citrate |
| 450 | 20 | | | | | | 5 | | | | | | 6 | citrate |
| 451 | 20 | | | | | | 10 | | | | | | 6 | citrate |
| 452 | 20 | | | | | | 20 | | | | | | 6 | citrate |
| 453 | 20 | | | | | | 30 | | | | | | 6 | citrate |
| 454 | 20 | | | | | | | 10 | | | | | 6 | citrate |
| 455 | 20 | | | | | | | 20 | | | | | 6 | citrate |
| 456 | 20 | | | | | | | 100 | | | | | 6 | citrate |
| 457 | 20 | | | | | | | | 10 | | | | 6 | citrate |
| 458 | 20 | | | | | | | | 20 | | | | 6 | citrate |
| 459 | 20 | | | | | | | | 100 | | | | 6 | citrate |
| 460 | 20 | | | | | | | | | 10 | | | 6 | citrate |
| 461 | 20 | | | | | | | | | 20 | | | 6 | citrate |
| 462 | 20 | | | | | | | | | 100 | | | 6 | citrate |
| 463 | 20 | | | | | | | | | | 5 | | 6 | citrate |
| 464 | 20 | | | | | | | | | | 10 | | 6 | citrate |
| 465 | 20 | | | | | | | | | | 20 | | 6 | citrate |
| 466 | 20 | | | | | | | | | | 100 | | 6 | citrate |
| 467 | 30 | 5 | | | | | | | | | | | 6 | citrate |
| 468 | 30 | 10 | | | | | | | | | | | 6 | citrate |
| 469 | 30 | 20 | | | | | | | | | | | 6 | citrate |
| 470 | 30 | 30 | | | | | | | | | | | 6 | citrate |
| 471 | 30 | | 5 | | | | | | | | | | 6 | citrate |
| 472 | 30 | | 10 | | | | | | | | | | 6 | citrate |
| 473 | 30 | | 20 | | | | | | | | | | 6 | citrate |
| 474 | 30 | | 30 | | | | | | | | | | 6 | citrate |
| 475 | 30 | | | 5 | | | | | | | | | 6 | citrate |
| 476 | 30 | | | 10 | | | | | | | | | 6 | citrate |
| 477 | 30 | | | 20 | | | | | | | | | 6 | citrate |
| 478 | 30 | | | 30 | | | | | | | | | 6 | citrate |
| 479 | 30 | | | | 5 | | | | | | | | 6 | citrate |
| 480 | 30 | | | | 10 | | | | | | | | 6 | citrate |
| 481 | 30 | | | | 20 | | | | | | | | 6 | citrate |
| 482 | 30 | | | | 30 | | | | | | | | 6 | citrate |
| 483 | 30 | | | | | 5 | | | | | | | 6 | citrate |
| 484 | 30 | | | | | 10 | | | | | | | 6 | citrate |
| 485 | 30 | | | | | 20 | | | | | | | 6 | citrate |
| 486 | 30 | | | | | 30 | | | | | | | 6 | citrate |
| 487 | 30 | | | | | | 5 | | | | | | 6 | citrate |
| 488 | 30 | | | | | | 10 | | | | | | 6 | citrate |
| 489 | 30 | | | | | | 20 | | | | | | 6 | citrate |
| 490 | 30 | | | | | | 30 | | | | | | 6 | citrate |
| 491 | 30 | | | | | | | 10 | | | | | 6 | citrate |
| 492 | 30 | | | | | | | 20 | | | | | 6 | citrate |
| 493 | 30 | | | | | | | 100 | | | | | 6 | citrate |
| 494 | 30 | | | | | | | | 10 | | | | 6 | citrate |
| 495 | 30 | | | | | | | | 20 | | | | 6 | citrate |
| 496 | 30 | | | | | | | | 100 | | | | 6 | citrate |
| 497 | 30 | | | | | | | | | 10 | | | 6 | citrate |
| 498 | 30 | | | | | | | | | 20 | | | 6 | citrate |
| 499 | 30 | | | | | | | | | 100 | | | 6 | citrate |
| 500 | 30 | | | | | | | | | | | | 6 | citrate |
| 501 | 30 | | | | | | | | | | | | 6 | citrate |
| 502 | 30 | | | | | | | | | | 5 | | 6 | citrate |
| 503 | 30 | | | | | | | | | | 10 | | 6 | citrate |
| 504 | 30 | | | | | | | | | | 20 | | 6 | citrate |
| 505 | 30 | | | | | | | | | | 100 | | 6 | citrate |
| 506 | 40 | 5 | | | | | | | | | | | 6 | citrate |
| 507 | 40 | 10 | | | | | | | | | | | 6 | citrate |
| 508 | 40 | 20 | | | | | | | | | | | 6 | citrate |
| 509 | 40 | 30 | | | | | | | | | | | 6 | citrate |
| 510 | 40 | | 5 | | | | | | | | | | 6 | citrate |
| 511 | 40 | | 10 | | | | | | | | | | 6 | citrate |
| 512 | 40 | | 20 | | | | | | | | | | 6 | citrate |
| 513 | 40 | | 30 | | | | | | | | | | 6 | citrate |
| 514 | 40 | | | 5 | | | | | | | | | 6 | citrate |
| 515 | 40 | | | 10 | | | | | | | | | 6 | citrate |
| 516 | 40 | | | 20 | | | | | | | | | 6 | citrate |
| 517 | 40 | | | 30 | | | | | | | | | 6 | citrate |
| 518 | 40 | | | | 5 | | | | | | | | 6 | citrate |
| 519 | 40 | | | | 10 | | | | | | | | 6 | citrate |
| 520 | 40 | | | | 20 | | | | | | | | 6 | citrate |
| 521 | 40 | | | | 30 | | | | | | | | 6 | citrate |
| 522 | 40 | | | | | 5 | | | | | | | 6 | citrate |
| 523 | 40 | | | | | 10 | | | | | | | 6 | citrate |
| 524 | 40 | | | | | 20 | | | | | | | 6 | citrate |
| 525 | 40 | | | | | 30 | | | | | | | 6 | citrate |
| 526 | 40 | | | | | | 5 | | | | | | 6 | citrate |
| 527 | 40 | | | | | | 10 | | | | | | 6 | citrate |
| 528 | 40 | | | | | | 20 | | | | | | 6 | citrate |
| 529 | 40 | | | | | | 30 | | | | | | 6 | citrate |
| 530 | 40 | | | | | | | 10 | | | | | 6 | citrate |
| 531 | 40 | | | | | | | 20 | | | | | 6 | citrate |
| 532 | 40 | | | | | | | 100 | | | | | 6 | citrate |
| 533 | 40 | | | | | | | | 10 | | | | 6 | citrate |
| 534 | 40 | | | | | | | | 20 | | | | 6 | citrate |
| 535 | 40 | | | | | | | | 100 | | | | 6 | citrate |
| 536 | 40 | | | | | | | | | 10 | | | 6 | citrate |
| 537 | 40 | | | | | | | | | 20 | | | 6 | citrate |
| 538 | 40 | | | | | | | | | 100 | | | 6 | citrate |
| 539 | 40 | | | | | | | | | | 5 | | 6 | citrate |
| 540 | 40 | | | | | | | | | | 10 | | 6 | citrate |
| 541 | 40 | | | | | | | | | | 20 | | 6 | citrate |
| 542 | 40 | | | | | | | | | | 100 | | 6 | citrate |
| 543 | 50 | | | | | | 10 | | | | | | 6 | citrate |
| 544 | 50 | | | | | | 20 | | | | | | 6 | citrate |
| 545 | 50 | | | | | | 30 | | | | | | 6 | citrate |
| 546 | 50 | | | | | | 40 | | | | | | 6 | citrate |
| 547 | 50 | | | | | | 50 | | | | | | 6 | citrate |
| 548 | 100 | | | | | | 20 | | | | | | 6 | citrate |
| 549 | 100 | | | | | | 30 | | | | | | 6 | citrate |
| 550 | 100 | | | | | | 40 | | | | | | 6 | citrate |
| 551 | 100 | | | | | | 50 | | | | | | 6 | citrate |
| 552 | 150 | | | | | | 20 | | | | | | 6 | citrate |
| 553 | 150 | | | | | | 30 | | | | | | 6 | citrate |
| 554 | 150 | | | | | | 40 | | | | | | 6 | citrate |
| 555 | 150 | | | | | | 50 | | | | | | 6 | citrate |
| 556 | 200 | | | | | | 30 | | | | | | 6 | citrate |
| 557 | 200 | | | | | | 40 | | | | | | 6 | citrate |
| 558 | 200 | | | | | | 50 | | | | | | 6 | citrate |
| 559 | 250 | | | | | | 30 | | | | | | 6 | citrate |
| 560 | 250 | | | | | | 40 | | | | | | 6 | citrate |
| 561 | 250 | | | | | | 50 | | | | | | 6 | citrate |
| 562 | 300 | | | | | | 30 | | | | | | 6 | citrate |
| 563 | 300 | | | | | | 40 | | | | | | 6 | citrate |
| 564 | 300 | | | | | | 50 | | | | | | 6 | citrate |
| 565 | 350 | | | | | | 40 | | | | | | 6 | citrate |
| 566 | 400 | | | | | | 50 | | | | | | 6 | citrate |
| 567 | 400 | | | | | | 40 | | | | | | 6 | citrate |
| 568 | 450 | | | | | | 50 | | | | | | 6 | citrate |
| 569 | 500 | | | | | | 50 | | | | | | 6 | citrate |
| 570 | 20 | 5 | | | | | | | | | | | 5 | Tartrate |
| 571 | 20 | 10 | | | | | | | | | | | 5 | Tartrate |
| 572 | 20 | 20 | | | | | | | | | | | 5 | Tartrate |
| 573 | 20 | 30 | | | | | | | | | | | 5 | Tartrate |
| 574 | 20 | | 5 | | | | | | | | | | 5 | Tartrate |
| 575 | 20 | | 10 | | | | | | | | | | 5 | Tartrate |
| 576 | 20 | | 20 | | | | | | | | | | 5 | Tartrate |
| 577 | 20 | | 30 | | | | | | | | | | 5 | Tartrate |
| 578 | 20 | | | 5 | | | | | | | | | 5 | Tartrate |
| 579 | 20 | | | 10 | | | | | | | | | 5 | Tartrate |
| 580 | 20 | | | 20 | | | | | | | | | 5 | Tartrate |
| 581 | 20 | | | 30 | | | | | | | | | 5 | Tartrate |
| 582 | 20 | | | | 5 | | | | | | | | 5 | Tartrate |
| 583 | 20 | | | | 10 | | | | | | | | 5 | Tartrate |
| 584 | 20 | | | | 20 | | | | | | | | 5 | Tartrate |
| 585 | 20 | | | | 30 | | | | | | | | 5 | Tartrate |
| 586 | 20 | | | | | 5 | | | | | | | 5 | Tartrate |
| 587 | 20 | | | | | 10 | | | | | | | 5 | Tartrate |
| 588 | 20 | | | | | 20 | | | | | | | 5 | Tartrate |
| 589 | 20 | | | | | 30 | | | | | | | 5 | Tartrate |
| 590 | 20 | | | | | | 5 | | | | | | 5 | Tartrate |
| 591 | 20 | | | | | | 10 | | | | | | 5 | Tartrate |
| 592 | 20 | | | | | | 20 | | | | | | 5 | Tartrate |
| 593 | 20 | | | | | | 30 | | | | | | 5 | Tartrate |
| 594 | 20 | | | | | | | 10 | | | | | 5 | Tartrate |
| 595 | 20 | | | | | | | 20 | | | | | 5 | Tartrate |
| 596 | 20 | | | | | | | 100 | | | | | 5 | Tartrate |
| 597 | 20 | | | | | | | | 10 | | | | 5 | Tartrate |
| 598 | 20 | | | | | | | | 20 | | | | 5 | Tartrate |
| 599 | 20 | | | | | | | | 100 | | | | 5 | Tartrate |
| 600 | 20 | | | | | | | | | 10 | | | 5 | Tartrate |
| 601 | 20 | | | | | | | | | 20 | | | 5 | Tartrate |
| 602 | 20 | | | | | | | | | 100 | | | 5 | Tartrate |
| 603 | 20 | | | | | | | | | | 5 | | 5 | Tartrate |
| 604 | 20 | | | | | | | | | | 10 | | 5 | Tartrate |
| 605 | 20 | | | | | | | | | | 20 | | 5 | Tartrate |
| 606 | 20 | | | | | | | | | | 100 | | 5 | Tartrate |
| 607 | 30 | 5 | | | | | | | | | | | 5 | Tartrate |
| 608 | 30 | 10 | | | | | | | | | | | 5 | Tartrate |
| 609 | 30 | 20 | | | | | | | | | | | 5 | Tartrate |
| 610 | 30 | 30 | | | | | | | | | | | 5 | Tartrate |
| 611 | 30 | | 5 | | | | | | | | | | 5 | Tartrate |
| 612 | 30 | | 10 | | | | | | | | | | 5 | Tartrate |
| 613 | 30 | | 20 | | | | | | | | | | 5 | Tartrate |
| 614 | 30 | | 30 | | | | | | | | | | 5 | Tartrate |
| 615 | 30 | | | 5 | | | | | | | | | 5 | Tartrate |
| 616 | 30 | | | 10 | | | | | | | | | 5 | Tartrate |
| 617 | 30 | | | 20 | | | | | | | | | 5 | Tartrate |
| 618 | 30 | | | 30 | | | | | | | | | 5 | Tartrate |
| 619 | 30 | | | | 5 | | | | | | | | 5 | Tartrate |
| 620 | 30 | | | | 10 | | | | | | | | 5 | Tartrate |
| 621 | 30 | | | | 20 | | | | | | | | 5 | Tartrate |
| 622 | 30 | | | | 30 | | | | | | | | 5 | Tartrate |
| 623 | 30 | | | | | 5 | | | | | | | 5 | Tartrate |
| 624 | 30 | | | | | 10 | | | | | | | 5 | Tartrate |
| 625 | 30 | | | | | 20 | | | | | | | 5 | Tartrate |
| 626 | 30 | | | | | 30 | | | | | | | 5 | Tartrate |
| 627 | 30 | | | | | | 5 | | | | | | 5 | Tartrate |
| 628 | 30 | | | | | | 10 | | | | | | 5 | Tartrate |
| 629 | 30 | | | | | | 20 | | | | | | 5 | Tartrate |
| 630 | 30 | | | | | | 30 | | | | | | 5 | Tartrate |
| 631 | 30 | | | | | | | 10 | | | | | 5 | Tartrate |
| 632 | 30 | | | | | | | 20 | | | | | 5 | Tartrate |
| 633 | 30 | | | | | | | 100 | | | | | 5 | Tartrate |
| 634 | 30 | | | | | | | | 10 | | | | 5 | Tartrate |
| 635 | 30 | | | | | | | | 20 | | | | 5 | Tartrate |
| 636 | 30 | | | | | | | | 100 | | | | 5 | Tartrate |
| 637 | 30 | | | | | | | | | 10 | | | 5 | Tartrate |
| 638 | 30 | | | | | | | | | 20 | | | 5 | Tartrate |
| 639 | 30 | | | | | | | | | 100 | | | 5 | Tartrate |
| 640 | 30 | | | | | | | | | | | | 5 | Tartrate |
| 641 | 30 | | | | | | | | | | | | 5 | Tartrate |
| 642 | 30 | | | | | | | | | | 5 | | 5 | Tartrate |
| 643 | 30 | | | | | | | | | | 10 | | 5 | Tartrate |
| 644 | 30 | | | | | | | | | | 20 | | 5 | Tartrate |
| 645 | 30 | | | | | | | | | | 100 | | 5 | Tartrate |
| 646 | 40 | 5 | | | | | | | | | | | 5 | Tartrate |
| 647 | 40 | 10 | | | | | | | | | | | 5 | Tartrate |
| 648 | 40 | 20 | | | | | | | | | | | 5 | Tartrate |
| 649 | 40 | 30 | | | | | | | | | | | 5 | Tartrate |
| 650 | 40 | | 5 | | | | | | | | | | 5 | Tartrate |
| 651 | 40 | | 10 | | | | | | | | | | 5 | Tartrate |
| 652 | 40 | | 20 | | | | | | | | | | 5 | Tartrate |
| 653 | 40 | | 30 | | | | | | | | | | 5 | Tartrate |
| 654 | 40 | | | 5 | | | | | | | | | 5 | Tartrate |
| 655 | 40 | | | 10 | | | | | | | | | 5 | Tartrate |
| 656 | 40 | | | 20 | | | | | | | | | 5 | Tartrate |
| 657 | 40 | | | 30 | | | | | | | | | 5 | Tartrate |
| 658 | 40 | | | | 5 | | | | | | | | 5 | Tartrate |
| 659 | 40 | | | | 10 | | | | | | | | 5 | Tartrate |
| 660 | 40 | | | | 20 | | | | | | | | 5 | Tartrate |
| 661 | 40 | | | | 30 | | | | | | | | 5 | Tartrate |
| 662 | 40 | | | | | 5 | | | | | | | 5 | Tartrate |
| 663 | 40 | | | | | 10 | | | | | | | 5 | Tartrate |
| 664 | 40 | | | | | 20 | | | | | | | 5 | Tartrate |
| 665 | 40 | | | | | 30 | | | | | | | 5 | Tartrate |
| 666 | 40 | | | | | | 5 | | | | | | 5 | Tartrate |
| 667 | 40 | | | | | | 10 | | | | | | 5 | Tartrate |
| 668 | 40 | | | | | | 20 | | | | | | 5 | Tartrate |
| 669 | 40 | | | | | | 30 | | | | | | 5 | Tartrate |
| 670 | 40 | | | | | | | 10 | | | | | 5 | Tartrate |
| 671 | 40 | | | | | | | 20 | | | | | 5 | Tartrate |
| 672 | 40 | | | | | | | 100 | | | | | 5 | Tartrate |
| 673 | 40 | | | | | | | | 10 | | | | 5 | Tartrate |
| 674 | 40 | | | | | | | | 20 | | | | 5 | Tartrate |
| 675 | 40 | | | | | | | | 100 | | | | 5 | Tartrate |
| 676 | 40 | | | | | | | | | 10 | | | 5 | Tartrate |
| 677 | 40 | | | | | | | | | 20 | | | 5 | Tartrate |
| 678 | 40 | | | | | | | | | 100 | | | 5 | Tartrate |
| 679 | 40 | | | | | | | | | | 5 | | 5 | Tartrate |
| 680 | 40 | | | | | | | | | | 10 | | 5 | Tartrate |
| 681 | 40 | | | | | | | | | | 20 | | 5 | Tartrate |
| 682 | 40 | | | | | | | | | | 100 | | 5 | Tartrate |
| 683 | 50 | | | | | | 10 | | | | | | 5 | Tartrate |
| 684 | 50 | | | | | | 20 | | | | | | 5 | Tartrate |
| 685 | 50 | | | | | | 30 | | | | | | 5 | Tartrate |
| 686 | 50 | | | | | | 40 | | | | | | 5 | Tartrate |
| 687 | 50 | | | | | | 50 | | | | | | 5 | Tartrate |
| 688 | 100 | | | | | | 20 | | | | | | 5 | Tartrate |
| 689 | 100 | | | | | | 30 | | | | | | 5 | Tartrate |
| 690 | 100 | | | | | | 40 | | | | | | 5 | Tartrate |
| 691 | 100 | | | | | | 50 | | | | | | 5 | Tartrate |
| 692 | 150 | | | | | | 20 | | | | | | 5 | Tartrate |
| 693 | 150 | | | | | | 30 | | | | | | 5 | Tartrate |
| 694 | 150 | | | | | | 40 | | | | | | 5 | Tartrate |
| 695 | 150 | | | | | | 50 | | | | | | 5 | Tartrate |
| 696 | 200 | | | | | | 30 | | | | | | 5 | Tartrate |
| 697 | 200 | | | | | | 40 | | | | | | 5 | Tartrate |
| 698 | 200 | | | | | | 50 | | | | | | 5 | Tartrate |
| 699 | 250 | | | | | | 30 | | | | | | 5 | Tartrate |
| 700 | 250 | | | | | | 40 | | | | | | 5 | Tartrate |
| 701 | 250 | | | | | | 50 | | | | | | 5 | Tartrate |
| 702 | 300 | | | | | | 30 | | | | | | 5 | Tartrate |
| 703 | 300 | | | | | | 40 | | | | | | 5 | Tartrate |
| 704 | 300 | | | | | | 50 | | | | | | 5 | Tartrate |
| 705 | 350 | | | | | | 40 | | | | | | 5 | Tartrate |
| 706 | 400 | | | | | | 50 | | | | | | 5 | Tartrate |
| 707 | 400 | | | | | | 40 | | | | | | 5 | Tartrate |
| 708 | 450 | | | | | | 50 | | | | | | 5 | Tartrate |
| 709 | 500 | | | | | | 50 | | | | | | 5 | Tartrate |
| 710 | 20 | 5 | | | | | | | | | | | 6 | Tartrate |
| 711 | 20 | 10 | | | | | | | | | | | 6 | Tartrate |
| 712 | 20 | 20 | | | | | | | | | | | 6 | Tartrate |
| 713 | 20 | 30 | | | | | | | | | | | 6 | Tartrate |
| 714 | 20 | | 5 | | | | | | | | | | 6 | Tartrate |
| 715 | 20 | | 10 | | | | | | | | | | 6 | Tartrate |
| 716 | 20 | | 20 | | | | | | | | | | 6 | Tartrate |
| 717 | 20 | | 30 | | | | | | | | | | 6 | Tartrate |
| 718 | 20 | | | 5 | | | | | | | | | 6 | Tartrate |
| 719 | 20 | | | 10 | | | | | | | | | 6 | Tartrate |
| 720 | 20 | | | 20 | | | | | | | | | 6 | Tartrate |
| 721 | 20 | | | 30 | | | | | | | | | 6 | Tartrate |
| 722 | 20 | | | | 5 | | | | | | | | 6 | Tartrate |
| 723 | 20 | | | | 10 | | | | | | | | 6 | Tartrate |
| 724 | 20 | | | | 20 | | | | | | | | 6 | Tartrate |
| 725 | 20 | | | | 30 | | | | | | | | 6 | Tartrate |
| 726 | 20 | | | | | 5 | | | | | | | 6 | Tartrate |
| 727 | 20 | | | | | 10 | | | | | | | 6 | Tartrate |
| 728 | 20 | | | | | 20 | | | | | | | 6 | Tartrate |
| 729 | 20 | | | | | 30 | | | | | | | 6 | Tartrate |
| 730 | 20 | | | | | | 5 | | | | | | 6 | Tartrate |
| 731 | 20 | | | | | | 10 | | | | | | 6 | Tartrate |
| 732 | 20 | | | | | | 20 | | | | | | 6 | Tartrate |
| 733 | 20 | | | | | | 30 | | | | | | 6 | Tartrate |
| 734 | 20 | | | | | | | 10 | | | | | 6 | Tartrate |
| 735 | 20 | | | | | | | 20 | | | | | 6 | Tartrate |
| 736 | 20 | | | | | | | 100 | | | | | 6 | Tartrate |
| 737 | 20 | | | | | | | | 10 | | | | 6 | Tartrate |
| 738 | 20 | | | | | | | | 20 | | | | 6 | Tartrate |
| 739 | 20 | | | | | | | | 100 | | | | 6 | Tartrate |
| 740 | 20 | | | | | | | | | 10 | | | 6 | Tartrate |
| 741 | 20 | | | | | | | | | 20 | | | 6 | Tartrate |
| 742 | 20 | | | | | | | | | 100 | | | 6 | Tartrate |
| 743 | 20 | | | | | | | | | | 5 | | 6 | Tartrate |
| 744 | 20 | | | | | | | | | | 10 | | 6 | Tartrate |
| 745 | 20 | | | | | | | | | | 20 | | 6 | Tartrate |
| 746 | 20 | | | | | | | | | | 100 | | 6 | Tartrate |
| 747 | 30 | 5 | | | | | | | | | | | 6 | Tartrate |
| 748 | 30 | 10 | | | | | | | | | | | 6 | Tartrate |
| 749 | 30 | 20 | | | | | | | | | | | 6 | Tartrate |
| 750 | 30 | 30 | | | | | | | | | | | 6 | Tartrate |
| 751 | 30 | | 5 | | | | | | | | | | 6 | Tartrate |
| 752 | 30 | | 10 | | | | | | | | | | 6 | Tartrate |
| 753 | 30 | | 20 | | | | | | | | | | 6 | Tartrate |
| 754 | 30 | | 30 | | | | | | | | | | 6 | Tartrate |
| 755 | 30 | | | 5 | | | | | | | | | 6 | Tartrate |
| 756 | 30 | | | 10 | | | | | | | | | 6 | Tartrate |
| 757 | 30 | | | 20 | | | | | | | | | 6 | Tartrate |
| 758 | 30 | | | 30 | | | | | | | | | 6 | Tartrate |
| 759 | 30 | | | | 5 | | | | | | | | 6 | Tartrate |
| 760 | 30 | | | | 10 | | | | | | | | 6 | Tartrate |
| 761 | 30 | | | | 20 | | | | | | | | 6 | Tartrate |
| 762 | 30 | | | | 30 | | | | | | | | 6 | Tartrate |
| 763 | 30 | | | | | 5 | | | | | | | 6 | Tartrate |
| 764 | 30 | | | | | 10 | | | | | | | 6 | Tartrate |
| 765 | 30 | | | | | 20 | | | | | | | 6 | Tartrate |
| 766 | 30 | | | | | 30 | | | | | | | 6 | Tartrate |
| 767 | 30 | | | | | | 5 | | | | | | 6 | Tartrate |
| 768 | 30 | | | | | | 10 | | | | | | 6 | Tartrate |
| 769 | 30 | | | | | | 20 | | | | | | 6 | Tartrate |
| 770 | 30 | | | | | | 30 | | | | | | 6 | Tartrate |
| 771 | 30 | | | | | | | 10 | | | | | 6 | Tartrate |
| 772 | 30 | | | | | | | 20 | | | | | 6 | Tartrate |
| 773 | 30 | | | | | | | 100 | | | | | 6 | Tartrate |
| 774 | 30 | | | | | | | | 10 | | | | 6 | Tartrate |
| 775 | 30 | | | | | | | | 20 | | | | 6 | Tartrate |
| 776 | 30 | | | | | | | | 100 | | | | 6 | Tartrate |
| 777 | 30 | | | | | | | | | 10 | | | 6 | Tartrate |
| 778 | 30 | | | | | | | | | 20 | | | 6 | Tartrate |
| 779 | 30 | | | | | | | | | 100 | | | 6 | Tartrate |
| 780 | 30 | | | | | | | | | | | | 6 | Tartrate |
| 781 | 30 | | | | | | | | | | | | 6 | Tartrate |
| 782 | 30 | | | | | | | | | | 5 | | 6 | Tartrate |
| 783 | 30 | | | | | | | | | | 10 | | 6 | Tartrate |
| 784 | 30 | | | | | | | | | | 20 | | 6 | Tartrate |
| 785 | 30 | | | | | | | | | | 100 | | 6 | Tartrate |
| 786 | 40 | 5 | | | | | | | | | | | 6 | Tartrate |
| 787 | 40 | 10 | | | | | | | | | | | 6 | Tartrate |
| 788 | 40 | 20 | | | | | | | | | | | 6 | Tartrate |
| 789 | 40 | 30 | | | | | | | | | | | 6 | Tartrate |
| 790 | 40 | | 5 | | | | | | | | | | 6 | Tartrate |
| 791 | 40 | | 10 | | | | | | | | | | 6 | Tartrate |
| 792 | 40 | | 20 | | | | | | | | | | 6 | Tartrate |
| 793 | 40 | | 30 | | | | | | | | | | 6 | Tartrate |
| 794 | 40 | | | 5 | | | | | | | | | 6 | Tartrate |
| 795 | 40 | | | 10 | | | | | | | | | 6 | Tartrate |
| 796 | 40 | | | 20 | | | | | | | | | 6 | Tartrate |
| 797 | 40 | | | 30 | | | | | | | | | 6 | Tartrate |
| 798 | 40 | | | | 5 | | | | | | | | 6 | Tartrate |
| 799 | 40 | | | | 10 | | | | | | | | 6 | Tartrate |
| 800 | 40 | | | | 20 | | | | | | | | 6 | Tartrate |
| 801 | 40 | | | | 30 | | | | | | | | 6 | Tartrate |
| 802 | 40 | | | | | 5 | | | | | | | 6 | Tartrate |
| 803 | 40 | | | | | 10 | | | | | | | 6 | Tartrate |
| 804 | 40 | | | | | 20 | | | | | | | 6 | Tartrate |
| 805 | 40 | | | | | 30 | | | | | | | 6 | Tartrate |
| 806 | 40 | | | | | | 5 | | | | | | 6 | Tartrate |
| 807 | 40 | | | | | | 10 | | | | | | 6 | Tartrate |
| 808 | 40 | | | | | | 20 | | | | | | 6 | Tartrate |
| 809 | 40 | | | | | | 30 | | | | | | 6 | Tartrate |
| 810 | 40 | | | | | | | 10 | | | | | 6 | Tartrate |
| 811 | 40 | | | | | | | 20 | | | | | 6 | Tartrate |
| 812 | 40 | | | | | | | 100 | | | | | 6 | Tartrate |
| 813 | 40 | | | | | | | | 10 | | | | 6 | Tartrate |
| 814 | 40 | | | | | | | | 20 | | | | 6 | Tartrate |
| 815 | 40 | | | | | | | | 100 | | | | 6 | Tartrate |
| 816 | 40 | | | | | | | | | 10 | | | 6 | Tartrate |
| 817 | 40 | | | | | | | | | 20 | | | 6 | Tartrate |
| 818 | 40 | | | | | | | | | 100 | | | 6 | Tartrate |
| 819 | 40 | | | | | | | | | | 5 | | 6 | Tartrate |
| 820 | 40 | | | | | | | | | | 10 | | 6 | Tartrate |
| 821 | 40 | | | | | | | | | | 20 | | 6 | Tartrate |
| 822 | 40 | | | | | | | | | | 100 | | 6 | Tartrate |
| 823 | 50 | | | | | | 10 | | | | | | 6 | Tartrate |
| 824 | 50 | | | | | | 20 | | | | | | 6 | Tartrate |
| 825 | 50 | | | | | | 30 | | | | | | 6 | Tartrate |
| 826 | 50 | | | | | | 40 | | | | | | 6 | Tartrate |
| 827 | 50 | | | | | | 50 | | | | | | 6 | Tartrate |
| 828 | 100 | | | | | | 20 | | | | | | 6 | Tartrate |
| 829 | 100 | | | | | | 30 | | | | | | 6 | Tartrate |
| 830 | 100 | | | | | | 40 | | | | | | 6 | Tartrate |
| 831 | 100 | | | | | | 50 | | | | | | 6 | Tartrate |
| 832 | 150 | | | | | | 20 | | | | | | 6 | Tartrate |
| 833 | 150 | | | | | | 30 | | | | | | 6 | Tartrate |
| 834 | 150 | | | | | | 40 | | | | | | 6 | Tartrate |
| 835 | 150 | | | | | | 50 | | | | | | 6 | Tartrate |
| 836 | 200 | | | | | | 30 | | | | | | 6 | Tartrate |
| 837 | 200 | | | | | | 40 | | | | | | 6 | Tartrate |
| 838 | 200 | | | | | | 50 | | | | | | 6 | Tartrate |
| 839 | 250 | | | | | | 30 | | | | | | 6 | Tartrate |
| 840 | 250 | | | | | | 40 | | | | | | 6 | Tartrate |
| 841 | 250 | | | | | | 50 | | | | | | 6 | Tartrate |
| 842 | 300 | | | | | | 30 | | | | | | 6 | Tartrate |
| 843 | 300 | | | | | | 40 | | | | | | 6 | Tartrate |
| 844 | 300 | | | | | | 50 | | | | | | 6 | Tartrate |
| 845 | 350 | | | | | | 40 | | | | | | 6 | Tartrate |
| 846 | 400 | | | | | | 50 | | | | | | 6 | Tartrate |
| 847 | 400 | | | | | | 40 | | | | | | 6 | Tartrate |
| 848 | 450 | | | | | | 50 | | | | | | 6 | Tartrate |
| 849 | 500 | | | | | | 50 | | | | | | 6 | Tartrate |
| 850 | 20 | 5 | | | | | | | | | | | 5 | Lactate |
| 851 | 20 | 10 | | | | | | | | | | | 5 | Lactate |
| 852 | 20 | 20 | | | | | | | | | | | 5 | Lactate |
| 853 | 20 | 30 | | | | | | | | | | | 5 | Lactate |
| 854 | 20 | | 5 | | | | | | | | | | 5 | Lactate |
| 855 | 20 | | 10 | | | | | | | | | | 5 | Lactate |
| 856 | 20 | | 20 | | | | | | | | | | 5 | Lactate |
| 857 | 20 | | 30 | | | | | | | | | | 5 | Lactate |
| 858 | 20 | | | 5 | | | | | | | | | 5 | Lactate |
| 859 | 20 | | | 10 | | | | | | | | | 5 | Lactate |
| 860 | 20 | | | 20 | | | | | | | | | 5 | Lactate |
| 861 | 20 | | | 30 | | | | | | | | | 5 | Lactate |
| 862 | 20 | | | | 5 | | | | | | | | 5 | Lactate |
| 863 | 20 | | | | 10 | | | | | | | | 5 | Lactate |
| 864 | 20 | | | | 20 | | | | | | | | 5 | Lactate |
| 865 | 20 | | | | 30 | | | | | | | | 5 | Lactate |
| 866 | 20 | | | | | 5 | | | | | | | 5 | Lactate |
| 867 | 20 | | | | | 10 | | | | | | | 5 | Lactate |
| 868 | 20 | | | | | 20 | | | | | | | 5 | Lactate |
| 869 | 20 | | | | | 30 | | | | | | | 5 | Lactate |
| 870 | 20 | | | | | | 5 | | | | | | 5 | Lactate |
| 871 | 20 | | | | | | 10 | | | | | | 5 | Lactate |
| 872 | 20 | | | | | | 20 | | | | | | 5 | Lactate |
| 873 | 20 | | | | | | 30 | | | | | | 5 | Lactate |
| 874 | 20 | | | | | | | 10 | | | | | 5 | Lactate |
| 875 | 20 | | | | | | | 20 | | | | | 5 | Lactate |
| 876 | 20 | | | | | | | 100 | | | | | 5 | Lactate |
| 877 | 20 | | | | | | | | 10 | | | | 5 | Lactate |
| 878 | 20 | | | | | | | | 20 | | | | 5 | Lactate |
| 879 | 20 | | | | | | | | 100 | | | | 5 | Lactate |
| 880 | 20 | | | | | | | | | 10 | | | 5 | Lactate |
| 881 | 20 | | | | | | | | | 20 | | | 5 | Lactate |
| 882 | 20 | | | | | | | | | 100 | | | 5 | Lactate |
| 883 | 20 | | | | | | | | | | 5 | | 5 | Lactate |
| 884 | 20 | | | | | | | | | | 10 | | 5 | Lactate |
| 885 | 20 | | | | | | | | | | 20 | | 5 | Lactate |
| 886 | 20 | | | | | | | | | | 100 | | 5 | Lactate |
| 887 | 30 | 5 | | | | | | | | | | | 5 | Lactate |
| 888 | 30 | 10 | | | | | | | | | | | 5 | Lactate |
| 889 | 30 | 20 | | | | | | | | | | | 5 | Lactate |
| 890 | 30 | 30 | | | | | | | | | | | 5 | Lactate |
| 891 | 30 | | 5 | | | | | | | | | | 5 | Lactate |
| 892 | 30 | | 10 | | | | | | | | | | 5 | Lactate |
| 893 | 30 | | 20 | | | | | | | | | | 5 | Lactate |
| 894 | 30 | | 30 | | | | | | | | | | 5 | Lactate |
| 895 | 30 | | | 5 | | | | | | | | | 5 | Lactate |
| 896 | 30 | | | 10 | | | | | | | | | 5 | Lactate |
| 897 | 30 | | | 20 | | | | | | | | | 5 | Lactate |
| 898 | 30 | | | 30 | | | | | | | | | 5 | Lactate |
| 899 | 30 | | | | 5 | | | | | | | | 5 | Lactate |
| 900 | 30 | | | | 10 | | | | | | | | 5 | Lactate |
| 901 | 30 | | | | 20 | | | | | | | | 5 | Lactate |
| 902 | 30 | | | | 30 | | | | | | | | 5 | Lactate |
| 903 | 30 | | | | | 5 | | | | | | | 5 | Lactate |
| 904 | 30 | | | | | 10 | | | | | | | 5 | Lactate |
| 905 | 30 | | | | | 20 | | | | | | | 5 | Lactate |
| 906 | 30 | | | | | 30 | | | | | | | 5 | Lactate |
| 907 | 30 | | | | | | 5 | | | | | | 5 | Lactate |
| 908 | 30 | | | | | | 10 | | | | | | 5 | Lactate |
| 909 | 30 | | | | | | 20 | | | | | | 5 | Lactate |
| 910 | 30 | | | | | | 30 | | | | | | 5 | Lactate |
| 911 | 30 | | | | | | | 10 | | | | | 5 | Lactate |
| 912 | 30 | | | | | | | 20 | | | | | 5 | Lactate |
| 913 | 30 | | | | | | | 100 | | | | | 5 | Lactate |
| 914 | 30 | | | | | | | | 10 | | | | 5 | Lactate |
| 915 | 30 | | | | | | | | 20 | | | | 5 | Lactate |
| 916 | 30 | | | | | | | | 100 | | | | 5 | Lactate |
| 917 | 30 | | | | | | | | | 10 | | | 5 | Lactate |
| 918 | 30 | | | | | | | | | 20 | | | 5 | Lactate |
| 919 | 30 | | | | | | | | | 100 | | | 5 | Lactate |
| 920 | 30 | | | | | | | | | | | | 5 | Lactate |
| 921 | 30 | | | | | | | | | | | | 5 | Lactate |
| 922 | 30 | | | | | | | | | | 5 | | 5 | Lactate |
| 923 | 30 | | | | | | | | | | 10 | | 5 | Lactate |
| 924 | 30 | | | | | | | | | | 20 | | 5 | Lactate |
| 925 | 30 | | | | | | | | | | 100 | | 5 | Lactate |
| 926 | 40 | 5 | | | | | | | | | | | 5 | Lactate |
| 927 | 40 | 10 | | | | | | | | | | | 5 | Lactate |
| 928 | 40 | 20 | | | | | | | | | | | 5 | Lactate |
| 929 | 40 | 30 | | | | | | | | | | | 5 | Lactate |
| 930 | 40 | | 5 | | | | | | | | | | 5 | Lactate |
| 931 | 40 | | 10 | | | | | | | | | | 5 | Lactate |
| 932 | 40 | | 20 | | | | | | | | | | 5 | Lactate |
| 933 | 40 | | 30 | | | | | | | | | | 5 | Lactate |
| 934 | 40 | | | 5 | | | | | | | | | 5 | Lactate |
| 935 | 40 | | | 10 | | | | | | | | | 5 | Lactate |
| 936 | 40 | | | 20 | | | | | | | | | 5 | Lactate |
| 937 | 40 | | | 30 | | | | | | | | | 5 | Lactate |
| 938 | 40 | | | | 5 | | | | | | | | 5 | Lactate |
| 939 | 40 | | | | 10 | | | | | | | | 5 | Lactate |
| 940 | 40 | | | | 20 | | | | | | | | 5 | Lactate |
| 941 | 40 | | | | 30 | | | | | | | | 5 | Lactate |
| 942 | 40 | | | | | 5 | | | | | | | 5 | Lactate |
| 943 | 40 | | | | | 10 | | | | | | | 5 | Lactate |
| 944 | 40 | | | | | 20 | | | | | | | 5 | Lactate |
| 945 | 40 | | | | | 30 | | | | | | | 5 | Lactate |
| 946 | 40 | | | | | | 5 | | | | | | 5 | Lactate |
| 947 | 40 | | | | | | 10 | | | | | | 5 | Lactate |
| 948 | 40 | | | | | | 20 | | | | | | 5 | Lactate |
| 949 | 40 | | | | | | 30 | | | | | | 5 | Lactate |
| 950 | 40 | | | | | | | 10 | | | | | 5 | Lactate |
| 951 | 40 | | | | | | | 20 | | | | | 5 | Lactate |
| 952 | 40 | | | | | | | 100 | | | | | 5 | Lactate |
| 953 | 40 | | | | | | | | 10 | | | | 5 | Lactate |
| 954 | 40 | | | | | | | | 20 | | | | 5 | Lactate |
| 955 | 40 | | | | | | | | 100 | | | | 5 | Lactate |
| 956 | 40 | | | | | | | | | 10 | | | 5 | Lactate |
| 957 | 40 | | | | | | | | | 20 | | | 5 | Lactate |
| 958 | 40 | | | | | | | | | 100 | | | 5 | Lactate |
| 959 | 40 | | | | | | | | | | 5 | | 5 | Lactate |
| 960 | 40 | | | | | | | | | | 10 | | 5 | Lactate |
| 961 | 40 | | | | | | | | | | 20 | | 5 | Lactate |
| 962 | 40 | | | | | | | | | | 100 | | 5 | Lactate |
| 963 | 50 | | | | | | 10 | | | | | | 5 | Lactate |
| 964 | 50 | | | | | | 20 | | | | | | 5 | Lactate |
| 965 | 50 | | | | | | 30 | | | | | | 5 | Lactate |
| 966 | 50 | | | | | | 40 | | | | | | 5 | Lactate |
| 967 | 50 | | | | | | 50 | | | | | | 5 | Lactate |
| 968 | 100 | | | | | | 20 | | | | | | 5 | Lactate |
| 969 | 100 | | | | | | 30 | | | | | | 5 | Lactate |
| 970 | 100 | | | | | | 40 | | | | | | 5 | Lactate |
| 971 | 100 | | | | | | 50 | | | | | | 5 | Lactate |
| 972 | 150 | | | | | | 20 | | | | | | 5 | Lactate |
| 973 | 150 | | | | | | 30 | | | | | | 5 | Lactate |
| 974 | 150 | | | | | | 40 | | | | | | 5 | Lactate |
| 975 | 150 | | | | | | 50 | | | | | | 5 | Lactate |
| 976 | 200 | | | | | | 30 | | | | | | 5 | Lactate |
| 977 | 200 | | | | | | 40 | | | | | | 5 | Lactate |
| 978 | 200 | | | | | | 50 | | | | | | 5 | Lactate |
| 979 | 250 | | | | | | 30 | | | | | | 5 | Lactate |
| 980 | 250 | | | | | | 40 | | | | | | 5 | Lactate |
| 981 | 250 | | | | | | 50 | | | | | | 5 | Lactate |
| 982 | 300 | | | | | | 30 | | | | | | 5 | Lactate |
| 983 | 300 | | | | | | 40 | | | | | | 5 | Lactate |
| 984 | 300 | | | | | | 50 | | | | | | 5 | Lactate |
| 985 | 350 | | | | | | 40 | | | | | | 5 | Lactate |
| 986 | 400 | | | | | | 50 | | | | | | 5 | Lactate |
| 987 | 400 | | | | | | 40 | | | | | | 5 | Lactate |
| 988 | 450 | | | | | | 50 | | | | | | 5 | Lactate |
| 989 | 500 | | | | | | 50 | | | | | | 5 | Lactate |
| 990 | 20 | 5 | | | | | | | | | | | 6 | Tartrate |
| 991 | 20 | 10 | | | | | | | | | | | 6 | Tartrate |
| 992 | 20 | 20 | | | | | | | | | | | 6 | Tartrate |
| 993 | 20 | 30 | | | | | | | | | | | 6 | Tartrate |
| 994 | 20 | | 5 | | | | | | | | | | 6 | Tartrate |
| 995 | 20 | | 10 | | | | | | | | | | 6 | Tartrate |
| 996 | 20 | | 20 | | | | | | | | | | 6 | Tartrate |
| 997 | 20 | | 30 | | | | | | | | | | 6 | Tartrate |
| 998 | 20 | | | 5 | | | | | | | | | 6 | Tartrate |
| 999 | 20 | | | 10 | | | | | | | | | 6 | Tartrate |
| 1000 | 20 | | | 20 | | | | | | | | | 6 | Tartrate |
| 1001 | 20 | | | 30 | | | | | | | | | 6 | Tartrate |
| 1002 | 20 | | | | 5 | | | | | | | | 6 | Tartrate |
| 1003 | 20 | | | | 10 | | | | | | | | 6 | Tartrate |
| 1004 | 20 | | | | 20 | | | | | | | | 6 | Tartrate |
| 1005 | 20 | | | | 30 | | | | | | | | 6 | Tartrate |
| 1006 | 20 | | | | | 5 | | | | | | | 6 | Tartrate |
| 1007 | 20 | | | | | 10 | | | | | | | 6 | Tartrate |
| 1008 | 20 | | | | | 20 | | | | | | | 6 | Tartrate |
| 1009 | 20 | | | | | 30 | | | | | | | 6 | Tartrate |
| 1010 | 20 | | | | | | 5 | | | | | | 6 | Tartrate |
| 1011 | 20 | | | | | | 10 | | | | | | 6 | Tartrate |
| 1012 | 20 | | | | | | 20 | | | | | | 6 | Tartrate |
| 1013 | 20 | | | | | | 30 | | | | | | 6 | Tartrate |
| 1014 | 20 | | | | | | | 10 | | | | | 6 | Tartrate |
| 1015 | 20 | | | | | | | 20 | | | | | 6 | Tartrate |
| 1016 | 20 | | | | | | | 100 | | | | | 6 | Tartrate |
| 1017 | 20 | | | | | | | | 10 | | | | 6 | Tartrate |
| 1018 | 20 | | | | | | | | 20 | | | | 6 | Tartrate |
| 1019 | 20 | | | | | | | | 100 | | | | 6 | Tartrate |
| 1020 | 20 | | | | | | | | | 10 | | | 6 | Tartrate |
| 1021 | 20 | | | | | | | | | 20 | | | 6 | Tartrate |
| 1022 | 20 | | | | | | | | | 100 | | | 6 | Tartrate |
| 1023 | 20 | | | | | | | | | | 5 | | 6 | Tartrate |
| 1024 | 20 | | | | | | | | | | 10 | | 6 | Tartrate |
| 1025 | 20 | | | | | | | | | | 20 | | 6 | Tartrate |
| 1026 | 20 | | | | | | | | | | 100 | | 6 | Tartrate |
| 1027 | 30 | 5 | | | | | | | | | | | 6 | Tartrate |
| 1028 | 30 | 10 | | | | | | | | | | | 6 | Tartrate |
| 1029 | 30 | 20 | | | | | | | | | | | 6 | Tartrate |
| 1030 | 30 | 30 | | | | | | | | | | | 6 | Tartrate |
| 1031 | 30 | | 5 | | | | | | | | | | 6 | Tartrate |
| 1032 | 30 | | 10 | | | | | | | | | | 6 | Tartrate |
| 1033 | 30 | | 20 | | | | | | | | | | 6 | Tartrate |
| 1034 | 30 | | 30 | | | | | | | | | | 6 | Tartrate |
| 1035 | 30 | | | 5 | | | | | | | | | 6 | Tartrate |
| 1036 | 30 | | | 10 | | | | | | | | | 6 | Tartrate |
| 1037 | 30 | | | 20 | | | | | | | | | 6 | Tartrate |
| 1038 | 30 | | | 30 | | | | | | | | | 6 | Tartrate |
| 1039 | 30 | | | | 5 | | | | | | | | 6 | Tartrate |
| 1040 | 30 | | | | 10 | | | | | | | | 6 | Tartrate |
| 1041 | 30 | | | | 20 | | | | | | | | 6 | Tartrate |
| 1042 | 30 | | | | 30 | | | | | | | | 6 | Tartrate |
| 1043 | 30 | | | | | 5 | | | | | | | 6 | Tartrate |
| 1044 | 30 | | | | | 10 | | | | | | | 6 | Tartrate |
| 1045 | 30 | | | | | 20 | | | | | | | 6 | Tartrate |
| 1046 | 30 | | | | | 30 | | | | | | | 6 | Tartrate |
| 1047 | 30 | | | | | | 5 | | | | | | 6 | Tartrate |
| 1048 | 30 | | | | | | 10 | | | | | | 6 | Tartrate |
| 1049 | 30 | | | | | | 20 | | | | | | 6 | Tartrate |
| 1050 | 30 | | | | | | 30 | | | | | | 6 | Tartrate |
| 1051 | 30 | | | | | | | 10 | | | | | 6 | Tartrate |
| 1052 | 30 | | | | | | | 20 | | | | | 6 | Tartrate |
| 1053 | 30 | | | | | | | 100 | | | | | 6 | Tartrate |
| 1054 | 30 | | | | | | | | 10 | | | | 6 | Tartrate |
| 1055 | 30 | | | | | | | | 20 | | | | 6 | Tartrate |
| 1056 | 30 | | | | | | | | 100 | | | | 6 | Tartrate |
| 1057 | 30 | | | | | | | | | 10 | | | 6 | Tartrate |
| 1058 | 30 | | | | | | | | | 20 | | | 6 | Tartrate |
| 1059 | 30 | | | | | | | | | 100 | | | 6 | Tartrate |
| 1060 | 30 | | | | | | | | | | | | 6 | Tartrate |
| 1061 | 30 | | | | | | | | | | | | 6 | Tartrate |
| 1062 | 30 | | | | | | | | | | 5 | | 6 | Tartrate |
| 1063 | 30 | | | | | | | | | | 10 | | 6 | Tartrate |
| 1064 | 30 | | | | | | | | | | 20 | | 6 | Tartrate |
| 1065 | 30 | | | | | | | | | | 100 | | 6 | Tartrate |
| 1066 | 40 | 5 | | | | | | | | | | | 6 | Tartrate |
| 1067 | 40 | 10 | | | | | | | | | | | 6 | Tartrate |
| 1068 | 40 | 20 | | | | | | | | | | | 6 | Tartrate |
| 1069 | 40 | 30 | | | | | | | | | | | 6 | Tartrate |
| 1070 | 40 | | 5 | | | | | | | | | | 6 | Tartrate |
| 1071 | 40 | | 10 | | | | | | | | | | 6 | Tartrate |
| 1072 | 40 | | 20 | | | | | | | | | | 6 | Tartrate |
| 1073 | 40 | | 30 | | | | | | | | | | 6 | Tartrate |
| 1074 | 40 | | | 5 | | | | | | | | | 6 | Tartrate |
| 1075 | 40 | | | 10 | | | | | | | | | 6 | Tartrate |
| 1076 | 40 | | | 20 | | | | | | | | | 6 | Tartrate |
| 1077 | 40 | | | 30 | | | | | | | | | 6 | Tartrate |
| 1078 | 40 | | | | 5 | | | | | | | | 6 | Tartrate |
| 1079 | 40 | | | | 10 | | | | | | | | 6 | Tartrate |
| 1080 | 40 | | | | 20 | | | | | | | | 6 | Tartrate |
| 1081 | 40 | | | | 30 | | | | | | | | 6 | Tartrate |
| 1082 | 40 | | | | | 5 | | | | | | | 6 | Tartrate |
| 1083 | 40 | | | | | 10 | | | | | | | 6 | Tartrate |
| 1084 | 40 | | | | | 20 | | | | | | | 6 | Tartrate |
| 1085 | 40 | | | | | 30 | | | | | | | 6 | Tartrate |
| 1086 | 40 | | | | | | 5 | | | | | | 6 | Tartrate |
| 1087 | 40 | | | | | | 10 | | | | | | 6 | Tartrate |
| 1088 | 40 | | | | | | 20 | | | | | | 6 | Tartrate |
| 1089 | 40 | | | | | | 30 | | | | | | 6 | Tartrate |
| 1090 | 40 | | | | | | | 10 | | | | | 6 | Tartrate |
| 1091 | 40 | | | | | | | 20 | | | | | 6 | Tartrate |
| 1092 | 40 | | | | | | | 100 | | | | | 6 | Tartrate |
| 1093 | 40 | | | | | | | | 10 | | | | 6 | Tartrate |
| 1094 | 40 | | | | | | | | 20 | | | | 6 | Tartrate |
| 1095 | 40 | | | | | | | | 100 | | | | 6 | Tartrate |
| 1096 | 40 | | | | | | | | | 10 | | | 6 | Tartrate |
| 1097 | 40 | | | | | | | | | 20 | | | 6 | Tartrate |
| 1098 | 40 | | | | | | | | | 100 | | | 6 | Tartrate |
| 1099 | 40 | | | | | | | | | | 5 | | 6 | Tartrate |
| 1100 | 40 | | | | | | | | | | 10 | | 6 | Tartrate |
| 1101 | 40 | | | | | | | | | | 20 | | 6 | Tartrate |
| 1102 | 40 | | | | | | | | | | 100 | | 6 | Tartrate |
| 1103 | 50 | | | | | | 10 | | | | | | 6 | Tartrate |
| 1104 | 50 | | | | | | 20 | | | | | | 6 | Tartrate |
| 1105 | 50 | | | | | | 30 | | | | | | 6 | Tartrate |
| 1106 | 50 | | | | | | 40 | | | | | | 6 | Tartrate |
| 1107 | 50 | | | | | | 50 | | | | | | 6 | Tartrate |
| 1108 | 100 | | | | | | 20 | | | | | | 6 | Tartrate |
| 1109 | 100 | | | | | | 30 | | | | | | 6 | Tartrate |
| 1110 | 100 | | | | | | 40 | | | | | | 6 | Tartrate |
| 1111 | 100 | | | | | | 50 | | | | | | 6 | Tartrate |
| 1112 | 150 | | | | | | 20 | | | | | | 6 | Tartrate |
| 1113 | 150 | | | | | | 30 | | | | | | 6 | Tartrate |
| 1114 | 150 | | | | | | 40 | | | | | | 6 | Tartrate |
| 1115 | 150 | | | | | | 50 | | | | | | 6 | Tartrate |
| 1116 | 200 | | | | | | 30 | | | | | | 6 | Tartrate |
| 1117 | 200 | | | | | | 40 | | | | | | 6 | Tartrate |
| 1118 | 200 | | | | | | 50 | | | | | | 6 | Tartrate |
| 1119 | 250 | | | | | | 30 | | | | | | 6 | Tartrate |
| 1120 | 250 | | | | | | 40 | | | | | | 6 | Tartrate |
| 1121 | 250 | | | | | | 50 | | | | | | 6 | Tartrate |
| 1122 | 300 | | | | | | 30 | | | | | | 6 | Tartrate |
| 1123 | 300 | | | | | | 40 | | | | | | 6 | Tartrate |
| 1124 | 300 | | | | | | 50 | | | | | | 6 | Tartrate |
| 1125 | 350 | | | | | | 40 | | | | | | 6 | Tartrate |
| 1126 | 400 | | | | | | 50 | | | | | | 6 | Tartrate |
| 1127 | 400 | | | | | | 40 | | | | | | 6 | Tartrate |
| 1128 | 450 | | | | | | 50 | | | | | | 6 | Tartrate |
| 1129 | 500 | | | | | | 50 | | | | | | 6 | Tartrate |
| 1130 | 20 | | | | | | 5 | | | | | 0.75 | 5 | phosphate |
| 1131 | 20 | | | | | | 10 | | | | | 0.75 | 5 | phosphate |
| 1132 | 20 | | | | | | 20 | | | | | 0.75 | 5 | phosphate |
| 1133 | 20 | | | | | | 30 | | | | | 0.75 | 5 | phosphate |
| 1134 | 30 | | | | | | 5 | | | | | 0.75 | 5 | phosphate |
| 1135 | 30 | | | | | | 10 | | | | | 0.75 | 5 | phosphate |
| 1136 | 30 | | | | | | 20 | | | | | 0.75 | 5 | phosphate |
| 1137 | 30 | | | | | | 30 | | | | | 0.75 | 5 | phosphate |
| 1138 | 40 | | | | | | 5 | | | | | 0.75 | 5 | phosphate |
| 1139 | 40 | | | | | | 10 | | | | | 0.75 | 5 | phosphate |
| 1140 | 40 | | | | | | 20 | | | | | 0.75 | 5 | phosphate |
| 1141 | 40 | | | | | | 30 | | | | | 0.75 | 5 | phosphate |
| 1142 | 50 | | | | | | 10 | | | | | 0.75 | 5 | phosphate |
| 1143 | 50 | | | | | | 20 | | | | | 0.75 | 5 | phosphate |
| 1144 | 50 | | | | | | 30 | | | | | 0.75 | 5 | phosphate |
| 1145 | 50 | | | | | | 40 | | | | | 0.75 | 5 | phosphate |
| 1146 | 50 | | | | | | 50 | | | | | 0.75 | 5 | phosphate |
| 1147 | 100 | | | | | | 20 | | | | | 0.75 | 5 | phosphate |
| 1148 | 100 | | | | | | 30 | | | | | 0.75 | 5 | phosphate |
| 1149 | 100 | | | | | | 40 | | | | | 0.75 | 5 | phosphate |
| 1150 | 100 | | | | | | 50 | | | | | 0.75 | 5 | phosphate |
| 1151 | 150 | | | | | | 20 | | | | | 0.75 | 5 | phosphate |
| 1152 | 150 | | | | | | 30 | | | | | 0.75 | 5 | phosphate |
| 1153 | 150 | | | | | | 40 | | | | | 0.75 | 5 | phosphate |
| 1154 | 150 | | | | | | 50 | | | | | 0.75 | 5 | phosphate |
| 1155 | 200 | | | | | | 30 | | | | | 0.75 | 5 | phosphate |
| 1156 | 200 | | | | | | 40 | | | | | 0.75 | 5 | phosphate |
| 1157 | 200 | | | | | | 50 | | | | | 0.75 | 5 | phosphate |
| 1158 | 250 | | | | | | 30 | | | | | 0.75 | 5 | phosphate |
| 1159 | 250 | | | | | | 40 | | | | | 0.75 | 5 | phosphate |
| 1160 | 250 | | | | | | 50 | | | | | 0.75 | 5 | phosphate |
| 1161 | 300 | | | | | | 30 | | | | | 0.75 | 5 | phosphate |
| 1162 | 300 | | | | | | 40 | | | | | 0.75 | 5 | phosphate |
| 1163 | 300 | | | | | | 50 | | | | | 0.75 | 5 | phosphate |
| 1164 | 350 | | | | | | 40 | | | | | 0.75 | 5 | phosphate |
| 1165 | 400 | | | | | | 50 | | | | | 0.75 | 5 | phosphate |
| 1166 | 400 | | | | | | 40 | | | | | 0.75 | 5 | phosphate |
| 1167 | 450 | | | | | | 50 | | | | | 0.75 | 5 | phosphate |
| 1168 | 20 | | | | | | 5 | | | | | 0.75 | 6 | phosphate |
| 1169 | 20 | | | | | | 10 | | | | | 0.75 | 6 | phosphate |
| 1170 | 20 | | | | | | 20 | | | | | 0.75 | 6 | phosphate |
| 1171 | 20 | | | | | | 30 | | | | | 0.75 | 6 | phosphate |
| 1172 | 30 | | | | | | 5 | | | | | 0.75 | 6 | phosphate |
| 1173 | 30 | | | | | | 10 | | | | | 0.75 | 6 | phosphate |
| 1174 | 30 | | | | | | 20 | | | | | 0.75 | 6 | phosphate |
| 1175 | 30 | | | | | | 30 | | | | | 0.75 | 6 | phosphate |
| 1176 | 40 | | | | | | 5 | | | | | 0.75 | 6 | phosphate |
| 1177 | 40 | | | | | | 10 | | | | | 0.75 | 6 | phosphate |
| 1178 | 40 | | | | | | 20 | | | | | 0.75 | 6 | phosphate |
| 1179 | 40 | | | | | | 30 | | | | | 0.75 | 6 | phosphate |
| 1180 | 50 | | | | | | 10 | | | | | 0.75 | 6 | phosphate |
| 1181 | 50 | | | | | | 20 | | | | | 0.75 | 6 | phosphate |
| 1182 | 50 | | | | | | 30 | | | | | 0.75 | 6 | phosphate |
| 1183 | 50 | | | | | | 40 | | | | | 0.75 | 6 | phosphate |
| 1184 | 50 | | | | | | 50 | | | | | 0.75 | 6 | phosphate |
| 1185 | 100 | | | | | | 20 | | | | | 0.75 | 6 | phosphate |
| 1186 | 100 | | | | | | 30 | | | | | 0.75 | 6 | phosphate |
| 1187 | 100 | | | | | | 40 | | | | | 0.75 | 6 | phosphate |
| 1188 | 100 | | | | | | 50 | | | | | 0.75 | 6 | phosphate |
| 1189 | 150 | | | | | | 20 | | | | | 0.75 | 6 | phosphate |
| 1190 | 150 | | | | | | 30 | | | | | 0.75 | 6 | phosphate |
| 1191 | 150 | | | | | | 40 | | | | | 0.75 | 6 | phosphate |
| 1192 | 150 | | | | | | 50 | | | | | 0.75 | 6 | phosphate |
| 1193 | 200 | | | | | | 30 | | | | | 0.75 | 6 | phosphate |
| 1194 | 200 | | | | | | 40 | | | | | 0.75 | 6 | phosphate |
| 1195 | 200 | | | | | | 50 | | | | | 0.75 | 6 | phosphate |
| 1196 | 250 | | | | | | 30 | | | | | 0.75 | 6 | phosphate |
| 1197 | 250 | | | | | | 40 | | | | | 0.75 | 6 | phosphate |
| 1198 | 250 | | | | | | 50 | | | | | 0.75 | 6 | phosphate |
| 1199 | 300 | | | | | | 30 | | | | | 0.75 | 6 | phosphate |
| 1200 | 300 | | | | | | 40 | | | | | 0.75 | 6 | phosphate |
| 1201 | 300 | | | | | | 50 | | | | | 0.75 | 6 | phosphate |
| 1202 | 350 | | | | | | 40 | | | | | 0.75 | 6 | phosphate |
| 1203 | 400 | | | | | | 50 | | | | | 0.75 | 6 | phosphate |
| 1204 | 400 | | | | | | 40 | | | | | 0.75 | 6 | phosphate |
| 1205 | 450 | | | | | | 50 | | | | | 0.75 | 6 | phosphate |
| 1206 | 500 | | | | | | 50 | | | | | 0.75 | 6 | phosphate |
| 1207 | 20 | | | | | | 5 | | | | | 0.75 | 5 | citrate |
| 1208 | 20 | | | | | | 10 | | | | | 0.75 | 5 | citrate |
| 1209 | 20 | | | | | | 20 | | | | | 0.75 | 5 | citrate |
| 1210 | 20 | | | | | | 30 | | | | | 0.75 | 5 | citrate |
| 1211 | 30 | | | | | | 5 | | | | | 0.75 | 5 | citrate |
| 1212 | 30 | | | | | | 10 | | | | | 0.75 | 5 | citrate |
| 1213 | 30 | | | | | | 20 | | | | | 0.75 | 5 | citrate |
| 1214 | 30 | | | | | | 30 | | | | | 0.75 | 5 | citrate |
| 1215 | 40 | | | | | | 5 | | | | | 0.75 | 5 | citrate |
| 1216 | 40 | | | | | | 10 | | | | | 0.75 | 5 | citrate |
| 1217 | 40 | | | | | | 20 | | | | | 0.75 | 5 | citrate |
| 1218 | 40 | | | | | | 30 | | | | | 0.75 | 5 | citrate |
| 1219 | 50 | | | | | | 10 | | | | | 0.75 | 5 | citrate |
| 1220 | 50 | | | | | | 20 | | | | | 0.75 | 5 | citrate |
| 1221 | 50 | | | | | | 30 | | | | | 0.75 | 5 | citrate |
| 1222 | 50 | | | | | | 40 | | | | | 0.75 | 5 | citrate |
| 1223 | 50 | | | | | | 50 | | | | | 0.75 | 5 | citrate |
| 1224 | 100 | | | | | | 20 | | | | | 0.75 | 5 | citrate |
| 1225 | 100 | | | | | | 30 | | | | | 0.75 | 5 | citrate |
| 1226 | 100 | | | | | | 40 | | | | | 0.75 | 5 | citrate |
| 1227 | 100 | | | | | | 50 | | | | | 0.75 | 5 | citrate |
| 1228 | 150 | | | | | | 20 | | | | | 0.75 | 5 | citrate |
| 1229 | 150 | | | | | | 30 | | | | | 0.75 | 5 | citrate |
| 1230 | 150 | | | | | | 40 | | | | | 0.75 | 5 | citrate |
| 1231 | 150 | | | | | | 50 | | | | | 0.75 | 5 | citrate |
| 1232 | 200 | | | | | | 30 | | | | | 0.75 | 5 | citrate |
| 1233 | 200 | | | | | | 40 | | | | | 0.75 | 5 | citrate |
| 1234 | 200 | | | | | | 50 | | | | | 0.75 | 5 | citrate |
| 1235 | 250 | | | | | | 30 | | | | | 0.75 | 5 | citrate |
| 1236 | 250 | | | | | | 40 | | | | | 0.75 | 5 | citrate |
| 1237 | 250 | | | | | | 50 | | | | | 0.75 | 5 | citrate |
| 1238 | 300 | | | | | | 30 | | | | | 0.75 | 5 | citrate |
| 1239 | 300 | | | | | | 40 | | | | | 0.75 | 5 | citrate |
| 1240 | 300 | | | | | | 50 | | | | | 0.75 | 5 | citrate |
| 1241 | 350 | | | | | | 40 | | | | | 0.75 | 5 | citrate |
| 1242 | 400 | | | | | | 50 | | | | | 0.75 | 5 | citrate |
| 1243 | 400 | | | | | | 40 | | | | | 0.75 | 5 | citrate |
| 1244 | 450 | | | | | | 50 | | | | | 0.75 | 5 | citrate |
| 1245 | 20 | | | | | | 5 | | | | | 0.75 | 6 | citrate |
| 1246 | 20 | | | | | | 10 | | | | | 0.75 | 6 | citrate |
| 1247 | 20 | | | | | | 20 | | | | | 0.75 | 6 | citrate |
| 1248 | 20 | | | | | | 30 | | | | | 0.75 | 6 | citrate |
| 1249 | 30 | | | | | | 5 | | | | | 0.75 | 6 | citrate |
| 1250 | 30 | | | | | | 10 | | | | | 0.75 | 6 | citrate |
| 1251 | 30 | | | | | | 20 | | | | | 0.75 | 6 | citrate |
| 1252 | 30 | | | | | | 30 | | | | | 0.75 | 6 | citrate |
| 1253 | 40 | | | | | | 5 | | | | | 0.75 | 6 | citrate |
| 1254 | 40 | | | | | | 10 | | | | | 0.75 | 6 | citrate |
| 1255 | 40 | | | | | | 20 | | | | | 0.75 | 6 | citrate |
| 1256 | 40 | | | | | | 30 | | | | | 0.75 | 6 | citrate |
| 1257 | 50 | | | | | | 10 | | | | | 0.75 | 6 | citrate |
| 1258 | 50 | | | | | | 20 | | | | | 0.75 | 6 | citrate |
| 1259 | 50 | | | | | | 30 | | | | | 0.75 | 6 | citrate |
| 1260 | 50 | | | | | | 40 | | | | | 0.75 | 6 | citrate |
| 1261 | 50 | | | | | | 50 | | | | | 0.75 | 6 | citrate |
| 1262 | 100 | | | | | | 20 | | | | | 0.75 | 6 | citrate |
| 1263 | 100 | | | | | | 30 | | | | | 0.75 | 6 | citrate |
| 1264 | 100 | | | | | | 40 | | | | | 0.75 | 6 | citrate |
| 1265 | 100 | | | | | | 50 | | | | | 0.75 | 6 | citrate |
| 1266 | 150 | | | | | | 20 | | | | | 0.75 | 6 | citrate |
| 1267 | 150 | | | | | | 30 | | | | | 0.75 | 6 | citrate |
| 1268 | 150 | | | | | | 40 | | | | | 0.75 | 6 | citrate |
| 1269 | 150 | | | | | | 50 | | | | | 0.75 | 6 | citrate |
| 1270 | 200 | | | | | | 30 | | | | | 0.75 | 6 | citrate |
| 1271 | 200 | | | | | | 40 | | | | | 0.75 | 6 | citrate |
| 1272 | 200 | | | | | | 50 | | | | | 0.75 | 6 | citrate |
| 1273 | 250 | | | | | | 30 | | | | | 0.75 | 6 | citrate |
| 1274 | 250 | | | | | | 40 | | | | | 0.75 | 6 | citrate |
| 1275 | 250 | | | | | | 50 | | | | | 0.75 | 6 | citrate |
| 1276 | 300 | | | | | | 30 | | | | | 0.75 | 6 | citrate |
| 1277 | 300 | | | | | | 40 | | | | | 0.75 | 6 | citrate |
| 1278 | 300 | | | | | | 50 | | | | | 0.75 | 6 | citrate |
| 1279 | 350 | | | | | | 40 | | | | | 0.75 | 6 | citrate |
| 1280 | 400 | | | | | | 50 | | | | | 0.75 | 6 | citrate |
| 1281 | 400 | | | | | | 40 | | | | | 0.75 | 6 | citrate |
| 1282 | 450 | | | | | | 50 | | | | | 0.75 | 6 | citrate |
| 1283 | 500 | | | | | | 50 | | | | | 0.75 | 6 | citrate |
| 1284 | 20 | | | | | | 5 | | | | | 0.75 | 5 | tartrate |
| 1285 | 20 | | | | | | 10 | | | | | 0.75 | 5 | tartrate |
| 1286 | 20 | | | | | | 20 | | | | | 0.75 | 5 | tartrate |
| 1287 | 20 | | | | | | 30 | | | | | 0.75 | 5 | tartrate |
| 1288 | 30 | | | | | | 5 | | | | | 0.75 | 5 | tartrate |
| 1289 | 30 | | | | | | 10 | | | | | 0.75 | 5 | tartrate |
| 1290 | 30 | | | | | | 20 | | | | | 0.75 | 5 | tartrate |
| 1291 | 30 | | | | | | 30 | | | | | 0.75 | 5 | tartrate |
| 1292 | 40 | | | | | | 5 | | | | | 0.75 | 5 | tartrate |
| 1293 | 40 | | | | | | 10 | | | | | 0.75 | 5 | tartrate |
| 1294 | 40 | | | | | | 20 | | | | | 0.75 | 5 | tartrate |
| 1295 | 40 | | | | | | 30 | | | | | 0.75 | 5 | tartrate |
| 1296 | 50 | | | | | | 10 | | | | | 0.75 | 5 | tartrate |
| 1297 | 50 | | | | | | 20 | | | | | 0.75 | 5 | tartrate |
| 1298 | 50 | | | | | | 30 | | | | | 0.75 | 5 | tartrate |
| 1299 | 50 | | | | | | 40 | | | | | 0.75 | 5 | tartrate |
| 1300 | 50 | | | | | | 50 | | | | | 0.75 | 5 | tartrate |
| 1301 | 100 | | | | | | 20 | | | | | 0.75 | 5 | tartrate |
| 1302 | 100 | | | | | | 30 | | | | | 0.75 | 5 | tartrate |
| 1303 | 100 | | | | | | 40 | | | | | 0.75 | 5 | tartrate |
| 1304 | 100 | | | | | | 50 | | | | | 0.75 | 5 | tartrate |
| 1305 | 150 | | | | | | 20 | | | | | 0.75 | 5 | tartrate |
| 1306 | 150 | | | | | | 30 | | | | | 0.75 | 5 | tartrate |
| 1307 | 150 | | | | | | 40 | | | | | 0.75 | 5 | tartrate |
| 1308 | 150 | | | | | | 50 | | | | | 0.75 | 5 | tartrate |
| 1309 | 200 | | | | | | 30 | | | | | 0.75 | 5 | tartrate |
| 1310 | 200 | | | | | | 40 | | | | | 0.75 | 5 | tartrate |
| 1311 | 200 | | | | | | 50 | | | | | 0.75 | 5 | tartrate |
| 1312 | 250 | | | | | | 30 | | | | | 0.75 | 5 | tartrate |
| 1313 | 250 | | | | | | 40 | | | | | 0.75 | 5 | tartrate |
| 1314 | 250 | | | | | | 50 | | | | | 0.75 | 5 | tartrate |
| 1315 | 300 | | | | | | 30 | | | | | 0.75 | 5 | tartrate |
| 1316 | 300 | | | | | | 40 | | | | | 0.75 | 5 | tartrate |
| 1317 | 300 | | | | | | 50 | | | | | 0.75 | 5 | tartrate |
| 1318 | 350 | | | | | | 40 | | | | | 0.75 | 5 | tartrate |
| 1319 | 400 | | | | | | 50 | | | | | 0.75 | 5 | tartrate |
| 1320 | 400 | | | | | | 40 | | | | | 0.75 | 5 | tartrate |
| 1321 | 450 | | | | | | 50 | | | | | 0.75 | 5 | tartrate |
| 1322 | 20 | | | | | | 5 | | | | | 0.75 | 6 | tartrate |
| 1323 | 20 | | | | | | 10 | | | | | 0.75 | 6 | tartrate |
| 1324 | 20 | | | | | | 20 | | | | | 0.75 | 6 | tartrate |
| 1325 | 20 | | | | | | 30 | | | | | 0.75 | 6 | tartrate |
| 1326 | 30 | | | | | | 5 | | | | | 0.75 | 6 | tartrate |
| 1327 | 30 | | | | | | 10 | | | | | 0.75 | 6 | tartrate |
| 1328 | 30 | | | | | | 20 | | | | | 0.75 | 6 | tartrate |
| 1329 | 30 | | | | | | 30 | | | | | 0.75 | 6 | tartrate |
| 1330 | 40 | | | | | | 5 | | | | | 0.75 | 6 | tartrate |
| 1331 | 40 | | | | | | 10 | | | | | 0.75 | 6 | tartrate |
| 1332 | 40 | | | | | | 20 | | | | | 0.75 | 6 | tartrate |
| 1333 | 40 | | | | | | 30 | | | | | 0.75 | 6 | tartrate |
| 1334 | 50 | | | | | | 10 | | | | | 0.75 | 6 | tartrate |
| 1335 | 50 | | | | | | 20 | | | | | 0.75 | 6 | tartrate |
| 1336 | 50 | | | | | | 30 | | | | | 0.75 | 6 | tartrate |
| 1337 | 50 | | | | | | 40 | | | | | 0.75 | 6 | tartrate |
| 1338 | 50 | | | | | | 50 | | | | | 0.75 | 6 | tartrate |
| 1339 | 100 | | | | | | 20 | | | | | 0.75 | 6 | tartrate |
| 1340 | 100 | | | | | | 30 | | | | | 0.75 | 6 | tartrate |
| 1341 | 100 | | | | | | 40 | | | | | 0.75 | 6 | tartrate |
| 1342 | 100 | | | | | | 50 | | | | | 0.75 | 6 | tartrate |
| 1343 | 150 | | | | | | 20 | | | | | 0.75 | 6 | tartrate |
| 1344 | 150 | | | | | | 30 | | | | | 0.75 | 6 | tartrate |
| 1345 | 150 | | | | | | 40 | | | | | 0.75 | 6 | tartrate |
| 1346 | 150 | | | | | | 50 | | | | | 0.75 | 6 | tartrate |
| 1347 | 200 | | | | | | 30 | | | | | 0.75 | 6 | tartrate |
| 1348 | 200 | | | | | | 40 | | | | | 0.75 | 6 | tartrate |
| 1349 | 200 | | | | | | 50 | | | | | 0.75 | 6 | tartrate |
| 1350 | 250 | | | | | | 30 | | | | | 0.75 | 6 | tartrate |
| 1351 | 250 | | | | | | 40 | | | | | 0.75 | 6 | tartrate |
| 1352 | 250 | | | | | | 50 | | | | | 0.75 | 6 | tartrate |
| 1353 | 300 | | | | | | 30 | | | | | 0.75 | 6 | tartrate |
| 1354 | 300 | | | | | | 40 | | | | | 0.75 | 6 | tartrate |
| 1355 | 300 | | | | | | 50 | | | | | 0.75 | 6 | tartrate |
| 1356 | 350 | | | | | | 40 | | | | | 0.75 | 6 | tartrate |
| 1357 | 400 | | | | | | 50 | | | | | 0.75 | 6 | tartrate |
| 1358 | 400 | | | | | | 40 | | | | | 0.75 | 6 | tartrate |
| 1359 | 450 | | | | | | 50 | | | | | 0.75 | 6 | tartrate |
| 1360 | 500 | | | | | | 50 | | | | | 0.75 | 6 | tartrate |
| 1361 | 20 | | | | | | 5 | | | | | 0.75 | 5 | lactate |
| 1362 | 20 | | | | | | 10 | | | | | 0.75 | 5 | lactate |
| 1363 | 20 | | | | | | 20 | | | | | 0.75 | 5 | lactate |
| 1364 | 20 | | | | | | 30 | | | | | 0.75 | 5 | lactate |
| 1365 | 30 | | | | | | 5 | | | | | 0.75 | 5 | lactate |
| 1366 | 30 | | | | | | 10 | | | | | 0.75 | 5 | lactate |
| 1367 | 30 | | | | | | 20 | | | | | 0.75 | 5 | lactate |
| 1368 | 30 | | | | | | 30 | | | | | 0.75 | 5 | lactate |
| 1369 | 40 | | | | | | 5 | | | | | 0.75 | 5 | lactate |
| 1370 | 40 | | | | | | 10 | | | | | 0.75 | 5 | lactate |
| 1371 | 40 | | | | | | 20 | | | | | 0.75 | 5 | lactate |
| 1372 | 40 | | | | | | 30 | | | | | 0.75 | 5 | lactate |
| 1373 | 50 | | | | | | 10 | | | | | 0.75 | 5 | lactate |
| 1374 | 50 | | | | | | 20 | | | | | 0.75 | 5 | lactate |
| 1375 | 50 | | | | | | 30 | | | | | 0.75 | 5 | lactate |
| 1376 | 50 | | | | | | 40 | | | | | 0.75 | 5 | lactate |
| 1377 | 50 | | | | | | 50 | | | | | 0.75 | 5 | lactate |
| 1378 | 100 | | | | | | 20 | | | | | 0.75 | 5 | lactate |
| 1379 | 100 | | | | | | 30 | | | | | 0.75 | 5 | lactate |
| 1380 | 100 | | | | | | 40 | | | | | 0.75 | 5 | lactate |
| 1381 | 100 | | | | | | 50 | | | | | 0.75 | 5 | lactate |
| 1382 | 150 | | | | | | 20 | | | | | 0.75 | 5 | lactate |
| 1383 | 150 | | | | | | 30 | | | | | 0.75 | 5 | lactate |
| 1384 | 150 | | | | | | 40 | | | | | 0.75 | 5 | lactate |
| 1385 | 150 | | | | | | 50 | | | | | 0.75 | 5 | lactate |
| 1386 | 200 | | | | | | 30 | | | | | 0.75 | 5 | lactate |
| 1387 | 200 | | | | | | 40 | | | | | 0.75 | 5 | lactate |
| 1388 | 200 | | | | | | 50 | | | | | 0.75 | 5 | lactate |
| 1389 | 250 | | | | | | 30 | | | | | 0.75 | 5 | lactate |
| 1390 | 250 | | | | | | 40 | | | | | 0.75 | 5 | lactate |
| 1391 | 250 | | | | | | 50 | | | | | 0.75 | 5 | lactate |
| 1392 | 300 | | | | | | 30 | | | | | 0.75 | 5 | lactate |
| 1393 | 300 | | | | | | 40 | | | | | 0.75 | 5 | lactate |
| 1394 | 300 | | | | | | 50 | | | | | 0.75 | 5 | lactate |
| 1395 | 350 | | | | | | 40 | | | | | 0.75 | 5 | lactate |
| 1396 | 400 | | | | | | 50 | | | | | 0.75 | 5 | lactate |
| 1397 | 400 | | | | | | 40 | | | | | 0.75 | 5 | lactate |
| 1398 | 450 | | | | | | 50 | | | | | 0.75 | 5 | lactate |
| 1399 | 20 | | | | | | 5 | | | | | 0.75 | 6 | lactate |
| 1400 | 20 | | | | | | 10 | | | | | 0.75 | 6 | lactate |
| 1401 | 20 | | | | | | 20 | | | | | 0.75 | 6 | lactate |
| 1402 | 20 | | | | | | 30 | | | | | 0.75 | 6 | lactate |
| 1403 | 30 | | | | | | 5 | | | | | 0.75 | 6 | lactate |
| 1404 | 30 | | | | | | 10 | | | | | 0.75 | 6 | lactate |
| 1405 | 30 | | | | | | 20 | | | | | 0.75 | 6 | lactate |
| 1406 | 30 | | | | | | 30 | | | | | 0.75 | 6 | lactate |
| 1407 | 40 | | | | | | 5 | | | | | 0.75 | 6 | lactate |
| 1408 | 40 | | | | | | 10 | | | | | 0.75 | 6 | lactate |
| 1409 | 40 | | | | | | 20 | | | | | 0.75 | 6 | lactate |
| 1410 | 40 | | | | | | 30 | | | | | 0.75 | 6 | lactate |
| 1411 | 50 | | | | | | 10 | | | | | 0.75 | 6 | lactate |
| 1412 | 50 | | | | | | 20 | | | | | 0.75 | 6 | lactate |
| 1413 | 50 | | | | | | 30 | | | | | 0.75 | 6 | lactate |
| 1414 | 50 | | | | | | 40 | | | | | 0.75 | 6 | lactate |
| 1415 | 50 | | | | | | 50 | | | | | 0.75 | 6 | lactate |
| 1416 | 100 | | | | | | 20 | | | | | 0.75 | 6 | lactate |
| 1417 | 100 | | | | | | 30 | | | | | 0.75 | 6 | lactate |
| 1418 | 100 | | | | | | 40 | | | | | 0.75 | 6 | lactate |
| 1419 | 100 | | | | | | 50 | | | | | 0.75 | 6 | lactate |
| 1420 | 150 | | | | | | 20 | | | | | 0.75 | 6 | lactate |
| 1421 | 150 | | | | | | 30 | | | | | 0.75 | 6 | lactate |
| 1422 | 150 | | | | | | 40 | | | | | 0.75 | 6 | lactate |
| 1423 | 150 | | | | | | 50 | | | | | 0.75 | 6 | lactate |
| 1424 | 200 | | | | | | 30 | | | | | 0.75 | 6 | lactate |
| 1425 | 200 | | | | | | 40 | | | | | 0.75 | 6 | lactate |
| 1426 | 200 | | | | | | 50 | | | | | 0.75 | 6 | lactate |
| 1427 | 250 | | | | | | 30 | | | | | 0.75 | 6 | lactate |
| 1428 | 250 | | | | | | 40 | | | | | 0.75 | 6 | lactate |
| 1429 | 250 | | | | | | 50 | | | | | 0.75 | 6 | lactate |
| 1430 | 300 | | | | | | 30 | | | | | 0.75 | 6 | lactate |
| 1431 | 300 | | | | | | 40 | | | | | 0.75 | 6 | lactate |
| 1432 | 300 | | | | | | 50 | | | | | 0.75 | 6 | lactate |
| 1433 | 350 | | | | | | 40 | | | | | 0.75 | 6 | lactate |
| 1434 | 400 | | | | | | 50 | | | | | 0.75 | 6 | lactate |
| 1435 | 400 | | | | | | 40 | | | | | 0.75 | 6 | lactate |
| 1436 | 450 | | | | | | 50 | | | | | 0.75 | 6 | lactate |
| 1437 | 500 | | | | | | 50 | | | | | 0.75 | 6 | lactate |

### Example 3: Solubility Studies with Hydroxypropyl β Cyclodextrin (HPβCD)

This example illustrates solubility properties of certain formulations of imatinib free base in liquid solution.

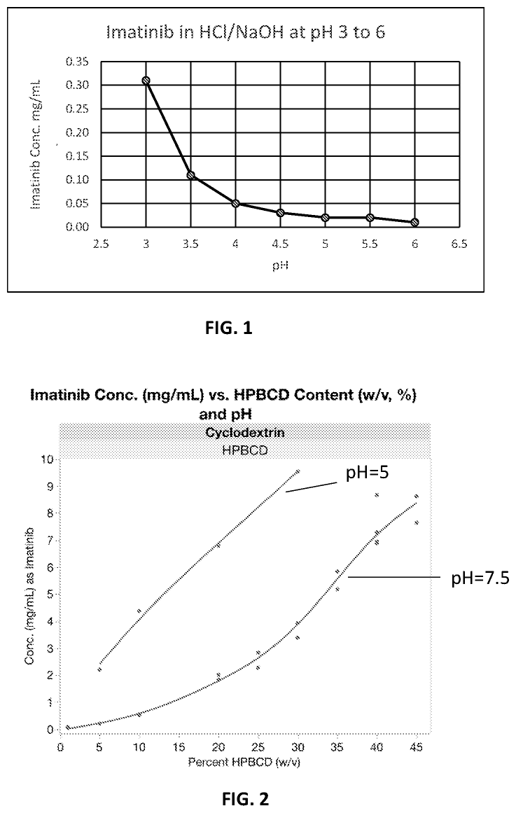

Imatinib free base was poorly soluble in water at physiologically relevant pH range of 4-8 (FIG. 1). FIG. 1 displays the maximum concentration of imatinib free base (mg/mL) as a function of pH. As seen in FIG. 1, a lower pH improved the solubility of imatinib free base. At pH of 3, the concentration of imatinib free base was around 0.30 mg/mL. At pH of 6, the concentration of imatinib free base was <0.01 mg/mL.

### Example 4: Solubility Studies with HPβCD and pH

This example illustrates solubility properties of certain formulations of imatinib free base in liquid solution.

FIG. 2 displays the maximum concentration of imatinib free base as a function of percent HPβCD at pH of 5 and 7.5. An improved solubility of imatinib free base was shown at a pH of 7.5. At 45% HPβCD (w/v), the amount of imatinib free base dissolved was 8.7 mg/mL. The addition of HPβCD improved solubility, for instance, the maximum concentration of imatinib free base was increased from <0.01 mg/mL at 0% HPβCD to 8.7 mg/mL to 45% HPβCD, respectively. At a pH of 5 and about 5% HPβCD (w/v), the concentration of imatinib free base dissolved was around 2 mg/mL. At a pH of 5 and between 25-30% HPβCD (w/v), the concentration of imatinib free base dissolved was 9.5 mg/mL. Decreasing the pH improved the solubility of imatinib free base for all concentrations tested. The decrease of pH from 7.5 to 5.0 provided 2.5 to 5.8 mg/mL concentration increase with HPβCD. The maximum imatinib free base concentration observed was 9.5 mg/mL.

### Example 5: Solubility Studies with HPβCD or Sulfobutylether β Cyclodextrin (SBEβCD) and pH

This example illustrates solubility properties of certain exemplary formulations of imatinib free base in liquid solution.

FIG. 3A displays the maximum concentration of imatinib free base as a function of percent cyclodextrin (HPβCD or sulfobutylether β cyclodextrin (SBEβCD)) at different pH levels. The relationship between solubility of imatinib free base and the concentration of HPβCD (w/v) in the solution was tested at a pH of 5 and 7.5. The relationship between solubility of imatinib free base and the concentration of SBEβCD (w/v) in the solution was tested at a pH of 5. At a pH of 5 and about 5% HPβCD (w/v), the maximum concentration of imatinib free base dissolved was around 2 mg/mL. At a pH of 5 and between 25-30% HPβCD (w/v), the concentration of imatinib free base dissolved was 9.5 mg/mL. At a pH of 5 and 10% SBEβCD (w/v), the concentration of imatinib free base dissolved was about 70 mg/mL. The results showed about 3500× solubility increase compared to imatinib free base in pH 5 water, which was 0.02 mg/mL. The data showed about 16× solubility increase compared to 10% HPβCD (w/v) at pH 5, which was 4.4 mg/mL. The molar ratio of (imatinib/SBEβCD) in the solution was 3.1. At a pH of 5 and 20% SBEβCD (w/v), the concentration of imatinib free base dissolved was about 160 mg/mL. The results showed at least about 8000× solubility increase compared to imatinib free base in pH 5 water. The data showed about 25× solubility increase compared to 20% HPβCD (w/v) at pH 5, which was 6.8 mg/mL. The molar ratio of (imatinib/SBEβCD) in the solution was 3.5.

Further, at a pH of 5 and 25% SBEβCD (w/v), the concentration of imatinib free base dissolved was about 200 mg/mL. At a pH of 5 and 30% SBEβCD (w/v), the concentration of imatinib free base dissolved was about 225 mg/mL.

FIG. 3B shows pictures of exemplary imatinib solutions and suspension under different pH conditions. About 30 mg/mL imatinib free base was mixed with three different vehicles (30% SBEβCD in 50 mM phosphate buffer adjusted at three different pH levels: 7, 5, and 3). As shown by the pictures, at pH=7, white suspension was obtained, while clear solutions were obtained under pH of 5 and pH of 3. This data suggests that solubility of imatinib free base in aqueous solution containing SBEβCD can be pH dependent, e.g., the low the pH of the SBEβCD aqueous solution is, the higher the solubility of imatinib free base in the solution.

### Example 6: Solubility Studies with SBEβCD and Dilution with Water

This example illustrates the effect of dilution with water on the solubility of imatinib free base in cyclodextrin-based formulation.

In one experiment, solubility of imatinib free base in an exemplary imatinib free base formulation upon dilution in water was examined. Briefly, the exemplary imatinib free base solution (50 mg/mL imatinib free base, 10% SBEβCD, pH 5.1) was diluted 40 times in water for injection (WFI). Upon dilution, imatinib precipitated. The pH of the final suspension after dilution was measured as 5.4. Since imatinib is still predominately protonated at pH of 5.4, the observed precipitation of imatinib may not be a pH-dependent effect.

In another experiment, another exemplary imatinib solution (100 mg/mL imatinib free base, 15% w/v SBEβCD, 50 mM phosphate buffer, pH 5.0) was diluted 20 times in water. Upon dilution, imatinib also precipitated. The resulting suspension was used in the intratracheal (IT) arm of the rat pharmacokinetic study shown in Example 9 below.

### Example 7: Solubility Studies with SBEβCD and Dilution

This example examines the effect of dilution while keeping pH constant on the solubility of imatinib free base in cyclodextrin-based formulation.

In one experiment, solubility of imatinib free base in exemplary imatinib free base formulations upon dilution while pH is kept constant is tested. Briefly, each of various exemplary imatinib free base solutions (100 mL in volume, saturated imatinib free base, different SBEβCD concentrations, pH 5) is diluted with phosphate buffer (50 mM, pH 5) to arrive at a formulation with a final SBEβCD concentration of 1.5% (w/v). The solubility of imatinib free base in the SBEβCD solution is predicted to decrease upon dilution despite the pH being constant, based on the measurement as shown in Example 5 and FIG. 3A. Table 4 summarizes the predicted precipitation of imatinib free base in each solution upon dilution to yield a final SBEβCD concentration of 1.5% (w/v).

TABLE 4
Predicted precipitation of imatinib free base upon dilution

| | | Dissolved | | Concentration | Dissolved | | |
|---|---|---|---|---|---|---|---|
| | | Imatinib | | Imatinib | Imatinib | | |
| | | Free Base | | Free Base | Free Base | Precipitated | |
| Imatinib | Percent | Before | | Concentration | Upon | Imatinib | Percent |
| Freebase | SBEβCD | Dilution | Dilution | Upon Dilution | Dilution | Free Base | Precipitation |
| (mg/mL) | (w/v) | (mg) | Factor | (mg/mL) | (mg) | (mg) | (%)* |
| 5 | 1.5 | 500 | n/a | 5 | 500 | 0 | 0 |
| 17.5 | 5 | 1750 | 3.3 | | 1650 | 100 | 5.7 |
| 71.5 | 10 | 7150 | 6.7 | | 3350 | 3800 | 53 |
| 122 | 15 | 12200 | 10.0 | | 5000 | 7200 | 59 |
| 160 | 20 | 16000 | 13.3 | | 6650 | 9350 | 58 |
| 197 | 25 | 19700 | 16.7 | | 8350 | 11350 | 58 |
| 227 | 30 | 22700 | 20.0 | | 10000 | 12700 | 56 |

*Percent Precipitation = 100* (total imatinib − dissolved imatinib)/total imatinib

### Example 8: Taste Test of Sulfobutylether β Cyclodextrin (SBEβCD)-Based Formulation

This example illustrates organoleptic properties of certain exemplary formulations of imatinib free base in liquid solution.

Tester 5 (T5) tested inhaling two formulations:
a. 40 mg/mL imatinib, 10% w/v SBEBCD, 50 mM phosphate buffer (pH 5)
b. 80 mg/mL imatinib, 20% w/v SBEBCD, 50 mM phosphate buffer (pH 5).

T5 did not cough after inhalation of each of the two formulations for about 2 minutes (continuous inhalations). T5 did not experience any cough, any desire to cough, or throat irritation.

T5 stated the imatinib free base formulation with SBEβCD resulted in no coughing (adverse reaction). The same tester T5 also tasted imatinib mesylate formulations and coughed severely when inhaling the imatinib mesylate formulations.

### Example 9: Test of Exemplary Formulation in Rats

This example illustrates and compares pharmacokinetics of an exemplary imatinib free base/cyclodextrin formulation (“imatinib free base”) and imatinib mesylate formulation (“imatinib mesylate”) in rats following intravenously (IV) and intratracheal (IT) administration, respectively.

It was found that a concentration of imatinib free base compatible with IT rat dosing was 5 mg/mL. It was observed, however, exemplary imatinib free base formulation (100 mg/mL imatinib free base, 15% w/v sulfobutylether β cyclodextrin (SBEβCD), 50 mM phosphate buffer, pH 5.0) precipitated upon dilution, thus the 100 mg/mL imatinib freebase formulation was diluted 20-fold in water immediately prior to dosing and administered IT as a 5 mg/mL suspension. 6 mg/mL imatinib mesylate (equivalent to 5 mg/mL imatinib free base) solution formulation was used as a comparator in the IV arm of the study. Both groups received a single dose equivalent to 1 mg/kg dose imatinib free base. Each group had four animals euthanized at 6 time points post administration for sample collections 1-3, 5, 10, 20, 30, or 60 minutes. Lung tissue and blood were collected for analysis.

All animals survived to their scheduled necropsy. No abnormal clinical observations were noted throughout the study. No test article related changes in body weight were observed during the study. In general, animal necropsy results were grossly unremarkable.

### Materials and Methods

TABLE 5
Test Article Formulation R1: Imatinib mesylate

| | |
|---|---|
| Identity: | Imatinib mesylate |
| Description: | 6 mg/mL imatinib mesylate (containing 5 mg/mL imatinib free |
| | base) solution, pH 5.0 |
| Preparation: | Not applicable. Test article used as received. |
| Supplier/Manufacturer: | Nitto Avecia Pharma Services |
| Lot: | 4389-166-2 |
| Storage Conditions: | Room temperature (ambient) |

TABLE 6
Test Article Formulation R2: Imatinib free base/cyclodextrin

| | |
|---|---|
| Identity: | Imatinib free base |
| Description: | 5 mg/mL imatinib free base suspension, 0.75% w/v |
| | sulfobutylether β cyclodextrin, 2.5 mM phosphate buffer, pH 5.0 |
| Preparation: | Test article was prepared as a suspension immediately prior to |
| | administration by diluting a 100 mg/mL imatinib free base, 15% |
| | w/v sulfobutylether-b-cyclodextrin, 50 mM phosphate buffer, |
| | pH 5.0 solution 20x in water. |
| Supplier/Manufacturer: | Nitto Avecia Pharma Services |
| Lot: | 4389-166-1 |
| Storage Conditions: | Room temperature (ambient) |

Test System: 72 male Sprague Dawley rats (Charles River Laboratories) were used for the study, 8 weeks old, body weight range at study start was 245.8-317 g.

Experimental Design and Execution: In brief, after the animals were transferred to study, all animals were randomly assigned to treatment groups per the study design below. Animals were treated either via intravenous (IV) administration of formulation R1, or via intratracheal (IT) administration for formulation R2 of the test article. Each group had four animals euthanized at 6 time points post administration for sample collection as outlined in Table 7.

TABLE 7
Experimental Design

| | | | | Number | |
|---|---|---|---|---|---|
| | Dose | | | of | Time |
| Group | (mg/kg) | Treatment | Route | Animals | points |
| 1 | 1 | Formulation R1: | IV | 24 | 1-3 min, 5 min, |
| | | Imatinib mesylate | | | 10 min, 20 min, |
| 2 | 1 | Formulation R2: | IT | 24 | 30 min, 60 min |
| | | Imatinib free base | | | |

### Pharmacokinetics

Plasma and lung concentration vs. time data was analyzed using JMP version 15.2.0. Averages for each time point are reported for groups/time points that have a minimum of 2 animals. The following PK parameters were estimated using two compartment IV bolus dose model fit using the non-linear model fit platform in JMP version 15.2.0: maximum observed concentration (Cₘₐₓ), time at which Cₘₐₓ is observed (Tₘₐₓ) and area under the plasma concentration vs. time curve from zero through the last measured concentration (AUC₆₀ min). AUC_{60 min} was calculated from the modeled curves using the trapezoidal integration method. Results are shown in Table 8 below.

TABLE 8
PK parameter results from lung and plasma concentration vs time data

| | Route | | | | | |
|---|---|---|---|---|---|---|
| | of | Sample | | | Tₘₐₓ | |
| Treatment | Administration | Location | Cmax | | (min) | AUC_{60 min} |
| Formulation R1: | IV | Lung | 4920 | ng/g | 5 | 153231 |
| Imatinib mesylate | | Plasma | 173 | ng/mL | 5 | 16913 |
| Formulation R2: | IT | Lung | 17925 | ng/g | 2 | 239806 |
| Imatinib free base | | Plasma | 808 | ng/mL | 2 | 6890 |

| | |
|---|---|
| Therapeutic | 4 |
| Advantage (Rd) | |

The Therapeutic Advantage (Rd) in Table 8 was calculated using the following equation:

$$Rd = \frac{{( {AUC}_{lung} / {AUC}_{plasma} )}_{IT}}{{( {AUC}_{lung} / {AUC}_{plasma} )}_{IV}}$$

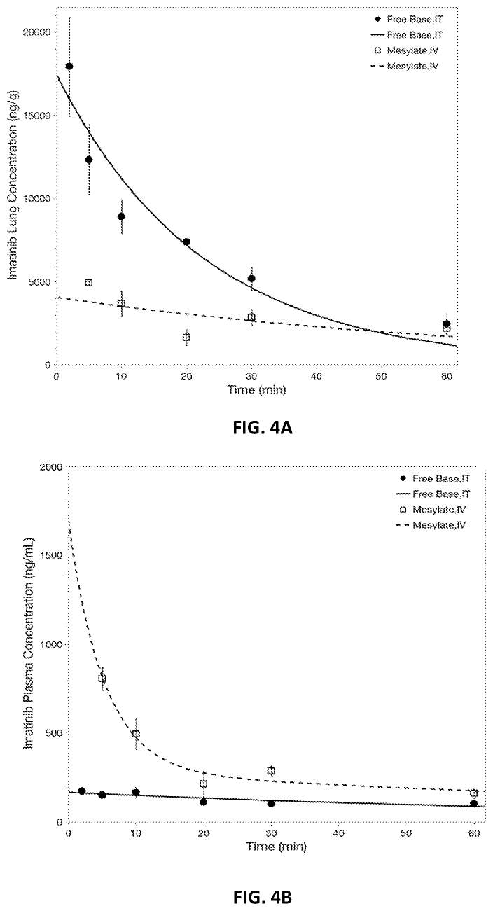

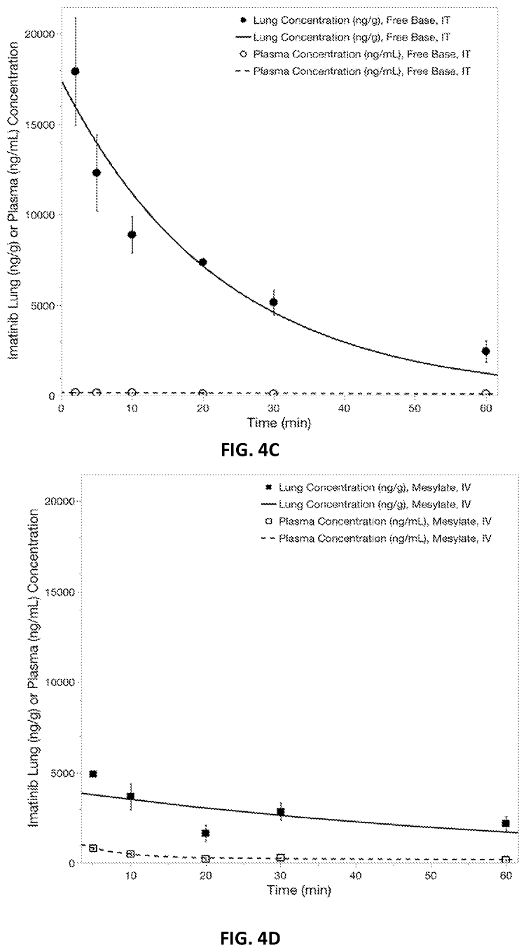

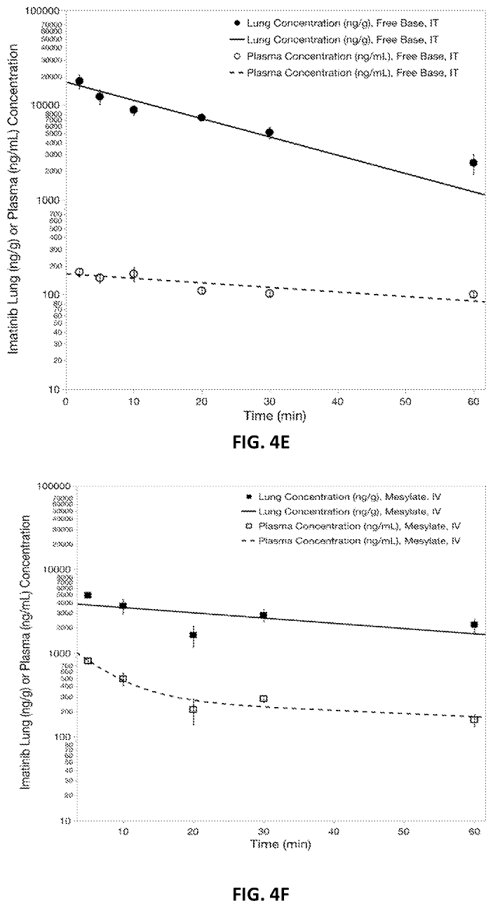

The IV data suggest that based on the high Log P (lipophilicity) of imatinib, the observed volume of distribution of imatinib (calculated as a ratio of amount of imatinib in the body versus plasma concentration of imatinib), and the cardiac output to the lungs, the sample collection period presented the distribution phase of the material preferentially accumulating in the lungs. This observation indicates direct administration of imatinib to the lungs can provide improved safety compared to systemic delivery via oral or IV routes. Since imatinib preferentially accumulates in the lung tissue direct delivery to the lungs via inhalation can minimize systemic exposure. Notably, direct delivery of imatinib to the lungs via IT administration of the imatinib free base/cyclodextrin formulation presents a 4 fold therapeutic advantage compared to IV administration of imatinib mesylate formulation (Rd=4). This therapeutic advantage can also be seen in FIGS. 4A-4F, which show plots summarizing concentration of imatinib in lung versus plasma over time after IT administration of imatinib free base/cyclodextrin formulation or IV administration of imatinib mesylate formulation.

### Example 10: Single-Dose Pharmacokinetics Study of Exemplary Formulation in Rats

This example illustrates and compares pharmacokinetics and distribution of an exemplary imatinib free base/cyclodextrin formulation in rats.

Test article: the test article will be imatinib free base/cyclodextrin formulation as shown in Table 9.

TABLE 9
Test Article Formulation T1

| | |
|---|---|
| Identity: | Imatinib free base |
| Description: | 100 mg/mL or 80 mg/mLimatinib free |
| | base, 15% w/v sulfobutylether-β- |
| | cyclodextrin, 50 mM phosphate |
| | buffer, pH 5.0 solution |

Test System: 120 Sprague Dawley rats with 11-12 weeks of age at initiation.

Experimental Design and Execution: After being transferred to study, all animals will be randomly assigned to treatment groups (different dose level and dose route) per the Experimental Design (Table 10) shown below. Animals will be treated with formulation T1 of the test article either via intravenous (IV) administration, via inhalation administration (IH), or via oral P.O. After the completion of administration, blood will be collected for test and evaluation according to Table 10.

TABLE 10
Experimental Design

| | | Route | Dose | |
|---|---|---|---|---|
| | | of | Level | |
| Group | Animals | Administration | (mg/kg) | Collections |
| 1 | 24 Male Rats | Inhalation | 0.3 | Blood collected from 3 animals |
| 2 | 24 Male Rats | Inhalation | 1 | from each group via jugular |
| 3 | 24 Male Rats | Inhalation | 3 | venipuncture at each of the time |
| | | | | points at 0.083, 0.25, 0.5, 2, and 6 |
| | | | | hours post-dose. |
| | | | | In addition, for each group, 3 |
| | | | | animals will be sacrificed following |
| | | | | appropriate anesthetization at each |
| | | | | of the time points at 1, 4, 8, 24, 48, |
| | | | | 72, and 96 hours post-dose to collect |
| | | | | blood (as much as possible) and |
| | | | | lungs from each animal as terminal |
| | | | | collections. |
| 4 | 24 Male Rats | Intravenous | 1 | Blood collected from 3 animals at |
| | | | | each of the time points via jugular |
| | | | | venipuncture at 0.083, 0.5, 1, 2, 6, 8, |
| | | | | and 24 hours post-dose. |
| | | | | In addition, for each group, 3 |
| | | | | animals will be sacrificed following |
| | | | | appropriate anesthetization at each |
| | | | | of the time points at 1, 4, 8, 24, 48, |
| | | | | 72, and 96 hours post-dose to collect |
| | | | | blood (as much as possible) and |
| | | | | lungs from each animal as terminal |
| | | | | collections. |
| 5 | 24 Male Rats | Oral P.O. | 1 | Blood collected from 3 animals |
| | | | | from each group via jugular |
| | | | | venipuncture at each of the time |
| | | | | points at 0.083, 0.25, 0.5, 2, and 6 |
| | | | | hours post-dose. |
| | | | | In addition, for each group, 3 |
| | | | | animals will be sacrificed following |
| | | | | appropriate anesthetization at each |
| | | | | of the time points at 1, 4, 8, 24, 48, |
| | | | | 72, and 96 hours post-dose to collect |
| | | | | blood (as much as possible) and |
| | | | | lungs from each animal as terminal |
| | | | | collections. |

Blood will be centrifuged to obtain plasma. Plasma and lung concentration vs. time data will be analyzed using JMP version 15.2.0. Averages for each time point will be reported for each group at each time point. The following PK parameters will be estimated using the non-linear model fit platform in JMP version 15.2.0: maximum observed concentration (Cₘₐₓ), time at which Cₘₐₓ is observed (Tₘₐₓ), half-life (t½), area under the plasma concentration vs. time curve from time zero through the last measured concentration (AUC₀₋ₜ), area under the plasma concentration vs. time curve from time zero to infinity (AUC_{0-∞}), and area under the plasma concentration vs. time curve from time zero to 24 hours after administration (AUC_{0-24 hr}). AUC₀₋ₜ will be calculated from the modeled curves using the trapezoidal integration method.

Direct delivery of imatinib to the lungs via IH administration of the imatinib free base/cyclodextrin formulation will presents a 2 to 200 fold therapeutic advantage compared to IV administration. Therapeutic advantage can be calculated following the formula in Example 9, where AUC₀₋ₜ or AUC_{0-24 hr} is used for the calculation.

### Example 11: Multiple-dose Pharmacokinetics Study of Exemplary Formulation in Rats

This example illustrates and compares pharmacokinetics and distribution of an exemplary imatinib free base/cyclodextrin formulation in rats after multiple-dose administrations.

Test article: the test article is imatinib free base/cyclodextrin formulation as shown in Table 9. The rats will be administered with test article once a day for 10, 20, or 30 consecutive days.

Test System: 96 Sprague Dawley rats with 11-12 weeks of age at initiation.

Experimental Design and Execution: After being transferred to study, all animals will be randomly assigned to treatment groups (different dose levels and different routes of administration) per the Experimental Design (Table 11) shown below. Animals will be treated with multiple-dose administrations for formulation T1 of the test article. After the completion of the administration of the last dose, blood will be collected for test and evaluation according to Table 11.

TABLE 11
Experimental Design

| | | Route | Dose | |
|---|---|---|---|---|
| | | of | Level | |
| Group | Animals | Administration | (mg/kg) | Collections |
| 1 | 24 Male Rats | Inhalation | 0.3 | Blood collected from 3 animals |
| 2 | 24 Male Rats | Inhalation | 1 | from each group via jugular |
| 3 | 24 Male Rats | Inhalation | 3 | venipuncture at each of the time |
| | | | | points at 0.083, 0.25, 0.5, 2, and 6 |
| | | | | hours post-dose. |
| | | | | In addition, for each group, 3 |
| | | | | animals will be sacrificed following |
| | | | | appropriate anesthetization at each |
| | | | | of the time points at 1, 4, 8, 24, 48, |
| | | | | 72, and 96 hours post-dose to collect |
| | | | | blood (as much as possible) and |
| | | | | lungs from each animal as terminal |
| | | | | collections. |
| 4 | 24 Male Rats | Intravenous | 1 | Blood collected from 3 animals at |
| | | | | each of the time points via jugular |
| | | | | venipuncture at 0.083, 0.5, 1, 2, 6, 8, |
| | | | | and 24 hours post-dose. |
| | | | | In addition, for each group, 3 |
| | | | | animals will be sacrificed following |
| | | | | appropriate anesthetization at each |
| | | | | of the time points at 1, 4, 8, 24, 48, |
| | | | | 72, and 96 hours post-dose to collect |
| | | | | blood (as much as possible) and |
| | | | | lungs from each animal as terminal |
| | | | | collections. |

Blood will be centrifuged to obtain plasma. Plasma and lung concentration vs. time data will be analyzed using JMP version 15.2.0. Averages for each time point will be reported for each group at each time point. The following PK parameters will be estimated using the non-linear model fit platform in JMP version 15.2.0: Cₘₐₓ, Tₘₐₓ, t½, AUC₀₋₂₄, and AUC_{0-∞}.

### Example 12: Radiolabeled Studies by Microautoradiography (MARG)

This example illustrates and compares distribution of an exemplary GLP Carbon-14 Labeled Imatinib free base/cyclodextrin formulation in rats using scintigraphy.

Test article: GLP Carbon-14 Labeled Imatinib free base (C14-Imatinib)/cyclodextrin formulation T2 as shown in Table 12.

TABLE 12
Test Article Formulation T2

| | |
|---|---|
| Identity: | C14-Imatinib free base |
| Description: | 80 mg/mL C14-Imatinib free base, |
| | 15% w/v sulfobutylether-b- |
| | cyclodextrin, 50 mM phosphate |
| | buffer, pH 5.0 solution |

Test System: 24 Sprague Dawley rats with 11-12 weeks of age at initiation.

Experimental Design and Execution: After being transferred to study, all animals will be treated via inhalation administration (IH) for formulation T2 at a dose level of 1 mg/kg, 1.5 mg/kg, 2 mg/kg, 2.5 mg/kg, or 3 mg/kg. Three of the animals will then be euthanized at one of the 8 time points (0.25, 0.5, 1, 2, 4, 6, 12 and 24 hours) post-dose for sample collection. Lung vasculature and pulmonary arterioles will be collected for MARG. The tissue samples will be frozen, sectioned at 5 m under darkroom conditions, and transferred to microscope slides pre-coated with nuclear emulsion (maintained at room temperature). Slides will be placed in light tight cassettes and stored at −20° C. to expose. Following exposure, all slides will be photographically processed, and the tissue sections will be histologically stained with haematoxylin and eosin. Slides will then be examined under a light microscope with objectives up to ×100 to assess the distribution of silver grains which relate to radioactivity. The distribution of C14-Imatinib in the frozen tissue will be compared among samples collected at different time points to illustrate the distribution profile of C14-Imatinib in lung vasculature and pulmonary arterioles post-dose. The intensity of the C14 signal can be used to calculate an imatinib concentration. Whole body autoradiography (QWBA) will also be performed to provide longitudinal and transverse views of whole body and the lungs.

High C14 signal can be detected in the lung vasculature and pulmonary arterioles samples from the animals that received the inhalational administration of T2, and higher C14 signal can be seen in the lungs as compared to another organ when examined using QWBA. Imatinib concentration vs. time curve will be plotted to examine the change of imatinib in the lung tissue and pulmonary arterioles post-dose.

### Example 13: Clinical Study of Exemplary Imatinib Formulation

This example illustrates pharmacokinetic and pharmacodynamic studies of exemplary imatinib formulation according to some embodiments of the present disclosure in human subjects.

### Study Design

In one instance, a randomized, double-blind, placebo-controlled, dose escalation study will be conducted to evaluate the safety, tolerability, and pharmacokinetics (PK) of single and multiple inhaled doses of test imatinib formulation in healthy adult volunteers. There 3 parts in this study:

Part A: double-blind, placebo-controlled, single ascending dose (SAD).

Part B: double-blind, placebo-controlled, multiple ascending dose (MAD).

Part C: double-blind, placebo-controlled, 20 mg single dose (SD) followed by a twice daily (BID) 20 mg dose (total daily dose of 40 mg) of exemplary imatinib formulation vs. placebo.

Test imatinib formulation assessed in the study is an exemplary imatinib formulation according to some embodiments of the present disclosure, containing 100 mg/mL imatinib free base, 15% w/v SBEβCD, 50 mM phosphate buffer, pH 5.0; or 50 mg/mL imatinib free base, 10% SBEβCD, pH 5.1.

The study will be monitored by a committee composed of Sponsor, Medical Investigator, and Independent Monitor. The decision to progress at each step will be based on overall assessment of the safety and tolerability data, with or without PK data.

All parts of the study (A, B, and C) will include a 28-day Screening period, a Treatment period, and a Follow-up period.

### Dose

The test imatinib formulation is administered to the subjects by inhalation via a vibrating mesh nebulizer once.

In Part A of the study, subjects will be enrolled into 1 of up to 4 sequential cohorts (A1 to A5). Inhalation of nominal doses (estimated doses delivered to mouth) of 10, 20, 40, and 50 mg are planned.

In Part B, subjects will be enrolled into 1 of up to 3 cohorts (B1 to B3).

Cohorts B2 and B3 will commence following Part C of the study and following committee review of safety, tolerability, and PK data.

In Part C of the study, a three-segment design (Segment 1: treatment, Segment 2: washout, Segment 3: treatment) will be used, and placebo will be compared to a single-dose of test imatinib formulation (20 mg nominal dose) followed by two doses of the same.

Each cohort will consist of 7 participants (5 receiving the test imatinib formulation and 2 receiving placebo).

### Measurements

Safety is assessed during the study by documentation of AEs, clinical laboratory tests, physical examination, vital sign measurements, electrocardiograms (ECGs), spirometry, and other relevant procedures.

Number of Participants with Treatment Emergent Adverse Events (TEAEs) will be summarized by treatment group using frequency and percent.

Number of Participants with Serious Adverse Events (SAEs) will also be summarized.

Systemic exposure to imatinib (in plasma) after a single administration of test imatinib formulation [Time Frame: Pre-dose through 72 hours post dose] will be determined in Part A. Plasma will be collected pre-dose, 0.5 h, 1 h, 2 h, 3 h, 4 h, 6 h, 9 h, 12 h, 24 h, 48 h, and 72 h post dose. As a result, Cₘₐₓ and AUC_{inhalation,plasma,0-24} will be calculated for single dose of imatinib.

Systemic exposure to imatinib (in plasma) after multiple administrations of test imatinib formulation [Time Frame: Pre-dose through 96 hours post last dose] will be determined in Part B. Plasma will be collected at Days 1, 3, 5, and 7 at pre-dose, 0.5 h, 1 h, 2 h, 3 h, 4 h, 6 h, 9 h, 12 h (and 24 h, 48 h, 72 h, and 96 h post last dose). As a result, Cₘₐₓ and AUC_{inhalation,plasma,0-24} will be calculated for multiple doses of imatinib.

### Example 14: Clinical Study of Exemplary Imatinib Formulation

This example showcases other pharmacokinetic and pharmacodynamic studies of exemplary imatinib formulation according to some embodiments of the present disclosure in human subjects.

### Study Design

In one instance, a randomized, double-blind, placebo-controlled, dose escalation study will be conducted to evaluate the safety, tolerability, and pharmacokinetics (PK) of single and multiple inhaled doses of test imatinib formulation in healthy adult volunteers. There 3 parts in this study:

Part A: double-blind, placebo-controlled, single ascending dose (SAD).

Part B: double-blind, placebo-controlled, multiple ascending dose (MAD).

Part C: double-blind, placebo-controlled, 75 mg single dose (SD) followed by a twice daily (BID) 75 mg dose (total daily dose of 150 mg) of exemplary imatinib formulation vs. placebo.

Test imatinib formulation assessed in the study is an exemplary imatinib formulation according to some embodiments of the present disclosure, containing 100 mg/mL imatinib free base, 15% w/v SBEβCD, 50 mM phosphate buffer, pH 5.0; or 50 mg/mL imatinib free base, 10% SBEβCD, pH 5.1.

The study will be monitored by a committee composed of Sponsor, Medical Investigator, and Independent Monitor. The decision to progress at each step will be based on overall assessment of the safety and tolerability data, with or without PK data.

All parts of the study (A, B, and C) will include a 28-day Screening period, a Treatment period, and a Follow-up period.

### Dose

The test imatinib formulation is administered to the subjects by inhalation via a vibrating mesh nebulizer once.

In Part A of the study, subjects will be enrolled into 1 of up to 4 sequential cohorts (A1 to A5). Inhalation of nominal doses (estimated doses delivered to mouth) of 60, 80, 100, and 120 mg are planned.

In Part B, subjects will be enrolled into 1 of up to 3 cohorts (B1 to B3).

Cohorts B2 and B3 will commence following Part C of the study and following committee review of safety, tolerability, and PK data.

In Part C of the study, a three-segment design (Segment 1: treatment, Segment 2: washout, Segment 3: treatment) will be used, and placebo will be compared to a single-dose of test imatinib formulation (75 mg nominal dose) followed by two doses of the same.

Each cohort will consist of 7 participants (5 receiving the test imatinib formulation and 2 receiving placebo).

### Measurements

Safety is assessed during the study by documentation of AEs, clinical laboratory tests, physical examination, vital sign measurements, electrocardiograms (ECGs), spirometry, and other relevant procedures.

Number of Participants with Treatment Emergent Adverse Events (TEAEs) will be summarized by treatment group using frequency and percent.

Number of Participants with Serious Adverse Events (SAEs) will also be summarized.

Systemic exposure to imatinib (in plasma) after a single administration of test imatinib formulation [Time Frame: Pre-dose through 72 hours post dose] will be determined in Part A. Plasma will be collected pre-dose, 0.5 h, 1 h, 2 h, 3 h, 4 h, 6 h, 9 h, 12 h, 24 h, 48 h, and 72 h post dose. As a result, Cₘₐₓ and AUC_{inhalation,plasma,0-24} will be calculated for single dose of imatinib.

Systemic exposure to imatinib (in plasma) after multiple administrations of test imatinib formulation [Time Frame: Pre-dose through 96 hours post last dose] will be determined in Part B. Plasma will be collected at Days 1, 3, 5, and 7 at pre-dose, 0.5 h, 1 h, 2 h, 3 h, 4 h, 6 h, 9 h, 12 h (and 24 h, 48 h, 72 h, and 96 h post last dose). As a result, Cₘₐₓ and AUC_{inhalation,plasma,0-24} will be calculated for multiple doses of imatinib.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A method of treating a subject in need thereof, comprising administering to said subject a pharmaceutical composition,
wherein said administering comprises delivering said pharmaceutical composition via inhalation,
wherein said pharmaceutical composition comprises an aqueous solution or suspension that comprises imatinib or a derivative thereof and cyclodextrin,
wherein said pharmaceutical composition provides a pharmacokinetic profile when measured in a human clinical trial in which human subjects receive said administering of a single dose of said pharmaceutical composition, and
wherein said pharmacokinetic profile is characterized by:
(a) a maximum measured plasma concentration of said imatinib or derivative thereof (Cₘₐₓ) ranging from about 50 ng/mL to about 1900 ng/mL;
(b) an area under the plasma concentration of said imatinib or derivative thereof-time curve from 0 to 24 hour (AUC₀₋₂₄) ranging from about 1 μg*h/mL to about 24 μg*h/mL;
(c) an area under the plasma concentration of said imatinib or derivative thereof-time curve from 0 to infinity (AUC_{0-∞}) ranging from about 1 μg*h/mL to about 38 μg*h/mL; or
(d) any combination of (a)-(c).

2. The method of claim 1, wherein said Cₘₐₓ is from about 50 ng/mL to about 1900 ng/mL.

3. The method of claim 1, wherein said Cₘₐₓ is from about 100 ng/mL to about 1500 ng/mL.

4. The method of claim 1, wherein said AUC₀₋₂₄ is from about 1 μg*h/mL to about 24 μg*h/mL.

5. The method of claim 1, wherein said AUC₀₋₂₄ is from about 1 μg*h/mL to about 17 μg*h/mL.

6. The method of claim 1, wherein said AUC_{0-∞} is from about 1 μg*h/mL to about 38 μg*h/mL.

7. The method of claim 1, wherein said AUC_{0-∞} is from about 1 μg*h/mL to about 23 μg*h/mL.

8. A method of treating a subject in need thereof, comprising administering to said subject a pharmaceutical composition,
wherein said administering comprises delivering said pharmaceutical composition via inhalation,
wherein said pharmaceutical composition comprises an aqueous solution or suspension that comprises imatinib or a derivative thereof and cyclodextrin,
wherein said pharmaceutical composition provides a pharmacokinetic profile when measured in a human clinical trial in which human subjects receive said administering of a multiple-dose regimen of said pharmaceutical composition, wherein said multiple-dose regimen comprises at least one dose of said pharmaceutical composition every day for at least 3 consecutive days, and
wherein said pharmacokinetic profile is characterized by:
(1) a maximum measured plasma concentration of said imatinib or derivative thereof (Cₘₐₓ) ranging from about 50 ng/mL to about 1900 ng/mL;
(2) an area under the plasma concentration of said imatinib or derivative thereof-time curve from 0 to 24 hour (AUC₀₋₂₄) ranging from about 1 μg*h/mL to about 24 μg*h/mL;
(3) an area under the plasma concentration of said imatinib or derivative thereof-time curve from 0 to infinity (AUC_{0-∞}) ranging from about 1 μg*h/mL to about 38 μg*h/mL; or
(4) any combination of (1)-(3).

9. The method of claim 8, wherein said Cₘₐₓ is from about 50 ng/mL to about 2500 ng/mL.

10. The method of claim 8, wherein said Cₘₐₓ is from about 100 ng/mL to about 1800 ng/mL.

11. The method of claim 8, wherein said AUC₀₋₂₄ is from about 1 μg*h/mL to about 40 μg*h/mL.

12. The method of claim 8, wherein said AUC₀₋₂₄ is from about 1 μg*h/mL to about 24 μg*h/mL.

13. The method of claim 8, wherein said AUC_{0-∞} is from about 1 μg*h/mL to about 80 μg*h/mL.

14. The method of claim 8, wherein said AUC_{0-∞} is from about 1 μg*h/mL to about 37 μg*h/mL.

15. The method of claim 8, wherein said multiple-dose regimen comprises two doses of said pharmaceutical composition every day for at least 3 consecutive days, optionally for 3, 4, 5, 7, 10, 12, 14, 20, 21, 25, 28, or 35 consecutive days.

16. The method of claim 8, wherein said multiple-dose regimen comprises three doses of said pharmaceutical composition every day for at least 3 consecutive days, optionally for 3, 4, 5, 7, 10, 12, 14, 20, 21, 25, 28, or 35 consecutive days.

17. A method of treating a subject in need thereof, comprising administering to said subject in need thereof a pharmaceutical composition,
wherein said administering comprises delivering said pharmaceutical composition via inhalation,
wherein said pharmaceutical composition comprises an aqueous solution or suspension that comprises imatinib or a derivative thereof and cyclodextrin,
wherein said pharmaceutical composition provides a pharmacokinetic profile when measured in an animal study in which mammalian animal subjects receive a single dose of said pharmaceutical composition or a diluted version thereof via inhalational administration or oral administration, and
wherein said pharmacokinetic profile is characterized by:
(i) a ratio of maximum measured lung concentration of said imatinib or derivative thereof post said inhalational administration (C_{max,inhalation,lung}) to maximum plasma concentration of said imatinib or derivative thereof said inhalational administration (C_{max,inhalation,plasma}) ranging from about 20 to about 1000; or
(ii) a ratio of maximum measured lung concentration of said imatinib or derivative thereof post said inhalational administration (C_{max,inhalation,lung}) to maximum measured lung concentration of said imatinib or derivative thereof post said oral administration (C_{max,po,lung}) ranging from about 20 to about 1000; or
(iii) a ratio of area under lung concentration of said imatinib or derivative thereof-time curve from 0 to 24 hours post said inhalational administration (AUC_{inhalation,lung,0-24}) to area under plasma concentration of said imatinib or derivative thereof-time curve from 0 to 24 hours post said inhalational administration (AUC_{inhalation,plasma,0-24}) ranging from about 10 to about 1000; or
(iv) a ratio of area under lung concentration of said imatinib or derivative thereof-time curve from 0 to 24 hours post said inhalational administration (AUC_{inhalation,lung,0-24}) to area under lung concentration of said imatinib or derivative thereof-time curve from 0 to 24 hours post said oral administration (AUC_{po,lung,0-24}) ranging from about 10 to about 1000; or
(v) a therapeutic advantage (Rd) of inhalational administration compared to oral administration ranging from about 2 to about 200, wherein Rd is calculated according to the following equation:
$$Rd = \frac{( {AUC}_{inhalation , lung , 0 - 24} / {AUC}_{inhalation , plasma , 0 - 24} )}{( {AUC}_{po , lung , 0 - 24} / {AUC}_{po , plasma , 0 - 24} )} ,$$
and
wherein AUC_{po,plasma,0-24} is area under plasma concentration of said imatinib or derivative thereof-time curve from 0 to 24 hours post said oral administration; or
(vi) any combination of (i)-(v).

18. The method of claim 17, wherein in said animal study, said mammalian animal subjects receive a single dose of a diluted version of said pharmaceutical composition via inhalational administration or oral administration.

19. The method of claim 18, wherein said diluted version of said pharmaceutical composition is diluted by at least 2 times with water as compared to said pharmaceutical composition.

20. The method of claim 18, wherein said diluted version of said pharmaceutical composition is diluted by 2 to 20 times with water as compared to said pharmaceutical composition.

21. The method of claim 18, wherein said diluted version of said pharmaceutical composition is diluted by about 2, 3, 4, 5, 6, 7, 8, 9, or 10 times with water as compared to said pharmaceutical composition.

22. The method of claim 18, wherein said diluted version of said pharmaceutical composition is diluted by about 5 times with water as compared to said pharmaceutical composition.

23. The method of claim 17, wherein said mammalian subjects are rats.

24. The method of claim 17, wherein said mammalian subjects are dogs or pigs.

25. The method of claim 17, wherein said mammalian subjects receive administration of 0.1 mg/kg to 10 mg/kg of said imatinib or derivative thereof for said animal study, optionally 0.3 mg/kg, 1 mg/kg, or 3 mg/kg of said imatinib or derivative thereof.

26. The method of claim 1, 8, or 17, wherein said pharmaceutical composition comprises from about 10 mg to about 250 mg of said imatinib or derivative thereof.

27. The method of claim 1, 8, or 17, wherein said pharmaceutical composition comprises about 20 mg of said imatinib or derivative thereof.

28. The method of claim 1, 8, or 17, wherein said pharmaceutical composition comprises about 60 mg of said imatinib or derivative thereof.

29. The method of claim 1, 8, or 17, wherein said pharmaceutical composition comprises about 80 mg of said imatinib or derivative thereof.

30. The method of claim 1, 8, or 17, wherein said pharmaceutical composition comprises about 100 mg of said imatinib or derivative thereof.

31. The method of claim 1, 8, or 17, wherein said pharmaceutical composition comprises about 120 mg of said imatinib or derivative thereof.

32. The method of claim 1, 8, or 17, wherein said pharmaceutical composition comprises about 160 mg of said imatinib or derivative thereof.

33. The method of claim 1, 8, or 17, wherein said pharmaceutical composition comprises about 200 mg of said imatinib or derivative thereof.

34. The method of claim 1, 8, or 17, wherein said pharmaceutical composition comprises about 240 mg of said imatinib or derivative thereof.

35. The method of claim 1, 8, or 17, wherein the method results in a central lung dose of said imatinib or derivative thereof ranging from about 10 mg to 100 mg, wherein said central lung dose measures the amount of said imatinib or derivative thereof delivered to the conducting zone of the subject's lungs.

36. The method of claim 35, wherein said central lung dose is from about 15 mg to 80 mg.

37. The method of claim 35, wherein said central lung dose is from about 30 mg to 60 mg.

38. The method of claim 35, wherein said central lung dose is about 15 mg.

39. The method of claim 35, wherein said central lung dose is about 20 mg.

40. The method of claim 35, wherein said central lung dose is about 30 mg.

41. The method of claim 35, wherein said central lung dose is about 45 mg.

42. The method of claim 35, wherein said central lung dose is about 60 mg.

43. The method of claim 1, 8, or 17, wherein the method results in a peripheral lung dose of said imatinib or derivative thereof ranging from about 15 mg to 120 mg, wherein said peripheral lung dose measures the amount of said imatinib or derivative thereof delivered to respiratory zone of the subject's lungs.

44. The method of claim 43, wherein said peripheral lung dose is from about 20 mg to 100 mg.

45. The method of claim 43, wherein said peripheral lung dose is from about 25 mg to 90 mg.

46. The method of claim 43, wherein said peripheral lung dose is about 20 mg.

47. The method of claim 43, wherein said peripheral lung dose is about 30 mg.

48. The method of claim 43, wherein said peripheral lung dose is about 45 mg.

49. The method of claim 43, wherein said peripheral lung dose is about 50 mg.

50. The method of claim 43, wherein said peripheral lung dose is about 65 mg.

51. The method of claim 43, wherein said peripheral lung dose is about 80 mg.

52. The method of claim 43, wherein said peripheral lung dose is about 90 mg.

53. The method of claim 43, wherein said peripheral lung dose is from about 15 mg to 35 mg.

54. The method of claim 43, wherein said peripheral lung dose is from about 20 mg to 30 mg.

55. The method of claim 43, wherein said peripheral lung dose is about 12 mg.

56. The method of claim 43, wherein said peripheral lung dose is about 16 mg.

57. The method of claim 43, wherein said peripheral lung dose is about 20 mg.

58. The method of claim 43, wherein said peripheral lung dose is about 24 mg.

59. The method of claim 43, wherein said peripheral lung dose is about 28 mg.

60. The method of claim 43, wherein said peripheral lung dose is about 32 mg.

61. The method of claim 43, wherein said peripheral lung dose is about 36 mg.

62. The method of claim 1, 8, or 17, wherein said subject suffers from a pulmonary disease.

63. The method of claim 62, wherein said pulmonary disease comprises lung fibrosis, lung cancer, or pulmonary hypertension.

64. The method of claim 62, wherein said pulmonary disease comprises pulmonary arterial hypertension.

65. The method of claim 1, 8, or 17, comprising administering to said subject a dose of said pharmaceutical composition at least once per day.

66. The method of claim 1, 8, or 17, comprising administering to said subject a dose of said pharmaceutical composition 2, 3, 4, or 5 times per day.

67. The method of claim 1, 8, or 17, comprising administering to said subject at least one dose of said pharmaceutical composition daily for a period of at least 5, 10, 20, 30, 60, 100, or 300 days, at least 1, 2, 3, 4, or 5 years.

68. The method of claim 1, 8, or 17, wherein said administering is performed using a nebulizer.

69. The method of claim 68, wherein said nebulizer is a jet nebulizer, a vibrating mesh nebulizer, an ultrasonic nebulizer, an electrospray nebulizer, or a hydraulic atomization device.

70. The method of claim 68, wherein said nebulizer is a vibrating mesh nebulizer.

71. The method of claim 68, wherein said nebulizer is a hydraulic atomization device.

72. The method of claim 68, wherein said nebulizer is an electrospray nebulizer.

73. The method of claim 1, 8, or 17, wherein said administering of a single dose of said pharmaceutical composition takes place within 30 minutes.

74. The method of claim 1, 8, or 17, wherein said administering of a single dose of said pharmaceutical composition takes place within 15 minutes, 10 minutes, or 5 minutes.

75. The method of claim 1, 8, or 17, wherein said administering of a single dose of said pharmaceutical composition takes place for about 2 minutes, 4 minutes, 6 minutes, or 8 minutes.

76. The method of claim 1, 8, or 17, wherein said aqueous solution or suspension has said cyclodextrin at a concentration of from about 1% (w/v) to about 80% (w/v).

77. The method of claim 1, 8, or 17, wherein said cyclodextrin is selected from the group consisting of: α-cyclodextrin, β-cyclodextrin, γ-cyclodextrin, hydroxypropyl-β-cyclodextrin, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin dimaltosyl-β-cyclodextrin, methyl-β-cyclodextrin, 6A-amino-6A-deoxy-N-(3-hydroxypropyl)-β-cyclodextrin, succinyl-α-cyclodextrin, succinyl-β-cyclodextrin, succinyl-γ-cyclodextrin, sulfobutylether-α-cyclodextrin, sulfobutylether-β-cyclodextrin, sulfobutylether-γ-cyclodextrin, carboxymethyl-α-cyclodextrin, carboxymethyl-β-cyclodextrin, carboxymethyl-γ-cyclodextrin, 2-carboxyethyl-α-cyclodextrin, 2-carboxyethyl-β-cyclodextrin, 2-carboxyethyl-γ-cyclodextrin, phosphate-α-cyclodextrin, phosphate-β-cyclodextrin, phosphate-γ-cyclodextrin, sulfoalkylether-β-cyclodextrins, and sulfoalkylether-γ-cyclodextrins.

78. The method of claim 1, 8, or 17, wherein said cyclodextrin comprises succinyl-α-cyclodextrin, succinyl-β-cyclodextrin, succinyl-γ-cyclodextrin, sulfobutylether-α-cyclodextrin, sulfobutylether-β-cyclodextrin, sulfobutylether-γ-cyclodextrin, carboxymethyl-α-cyclodextrin, carboxymethyl-β-cyclodextrin, carboxymethyl-γ-cyclodextrin, 2-carboxyethyl-α-cyclodextrin, 2-carboxyethyl-β-cyclodextrin, 2-carboxyethyl-γ-cyclodextrin, phosphate-α-cyclodextrin, phosphate-β-cyclodextrin, or phosphate-γ-cyclodextrin.

79. The method of claim 1, 8, or 17, wherein said cyclodextrin comprises an anionic cyclodextrin.

80. The method of claim 1, 8, or 17, wherein said aqueous solution or suspension comprises a salt of said cyclodextrin.

81. The composition of claim 80, wherein said salt of said cyclodextrin is a salt selected from the group consisting of: sodium salt, calcium salt, magnesium salt, iron salt, chromium salt, copper salt, zinc salt, lysine salt, arginine salt, and histidine salt.

82. The method of claim 1, 8, or 17, wherein said cyclodextrin comprises sulfobutylether-β-cyclodextrin.

83. The method of claim 1, 8, or 17, wherein said aqueous solution or suspension comprises sulfobutylether-β-cyclodextrin sodium.

84. The method of claim 1, 8, or 17, wherein said cyclodextrin comprises hydroxypropyl-β-cyclodextrin.

85. The method of claim 1, 8, or 17, wherein said aqueous solution or suspension further comprises a pH buffer.

86. The method of claim 85, wherein said pH buffer comprises an organic acid salt of citric acid, lactic acid, ascorbic acid, gluconic acid, carbonic acid, tartaric acid, succinic acid, acetic acid, or phthalic acid, Tris, tromethamine hydrochloride, or a phosphate buffer.

87. The method of claim 85, wherein said pH buffer comprises a phosphate buffer.

88. The method of claim 1, 8, or 17, wherein said aqueous solution or suspension further comprises an artificial sweetener.

89. The method of claim 88, wherein said artificial sweetener is selected from the group consisting of: acesulfame potassium, aspartame, cyclamate, mogrosides, saccharin, stevia, sucralose, neotame, and sugar alcohols, and combinations thereof.

90. The method of claim 1, 8, or 17, wherein said aqueous solution or suspension has a viscosity of at most 10 centipoise.

91. The method of claim 1, 8, or 17, wherein said aqueous solution or suspension has a viscosity of at most 9.5 centipoise, at most 9.0 centipoise, at most 8.5 centipoise, at most 8.0 centipoise, at most 7.6 centipoise, at most 7.4 centipoise, at most 7.2 centipoise, at most 7.0 centipoise, at most 6.8 centipoise, at most 6.6 centipoise, at most 6.4 centipoise, at most 6.2 centipoise, at most 6.0 centipoise, at most 5.8 centipoise, at most 5.6 centipoise, at most 5.4 centipoise, at most 5.2 centipoise, at most 5.0 centipoise, at most 4.8 centipoise, at most 4.6 centipoise, at most 4.4 centipoise, at most 4.2 centipoise, at most 4.0 centipoise, at most 3.8 centipoise, at most 3.6 centipoise, at most 3.4 centipoise, at most 3.2 centipoise, at most 3.0 centipoise, at most 2.8 centipoise, at most 2.6 centipoise, at most 2.4 centipoise, at most 2.2 centipoise, at most 2.0 centipoise, at most 1.8 centipoise, at most 1.6 centipoise, at most 1.4 centipoise, at most 1.2 centipoise, at most 1.0 centipoise, at most 0.8 centipoise, at most 0.6 centipoise, at most 0.4 centipoise, or at most 0.2 centipoise.

92. The method of claim 1, 8, or 17, wherein said aqueous solution or suspension has a viscosity of about 0.1 centipoise, 0.2 centipoise, 0.3 centipoise, 0.4 centipoise, 0.5 centipoise, 0.6 centipoise, 0.7 centipoise, 0.8 centipoise, 0.9 centipoise, 1.0 centipoise, 1.1 centipoise, 1.2 centipoise, 1.3 centipoise, 1.4 centipoise, 1.5 centipoise, 1.6 centipoise, 1.7 centipoise, 1.8 centipoise, 1.9 centipoise, 2.0 centipoise, 2.1 centipoise, 2.2 centipoise, 2.3 centipoise, 2.4 centipoise, 2.5 centipoise, 2.6 centipoise, 2.8 centipoise, 3.0 centipoise, 3.2 centipoise, 3.5 centipoise, 3.8 centipoise, 4.0 centipoise, 4.2 centipoise, 4.5 centipoise, 4.8 centipoise, 5.0 centipoise, 5.5 centipoise, 6.0 centipoise, 6.5 centipoise, 7.0 centipoise, 7.5 centipoise, 8.0 centipoise, or 8.5 centipoise.

93. The method of claim 1, 8, or 17, wherein said aqueous solution or suspension has from 1 to 250 mg/mL of said imatinib or derivative thereof.

94. The method of claim 1, 8, or 17, wherein said aqueous solution or suspension has from 60 mg/mL to 120 mg/mL of said imatinib or derivative thereof.

95. The method of claim 1, 8, or 17, wherein said aqueous solution or suspension has about 60 mg/mL of said imatinib or derivative thereof.

96. The method of claim 1, 8, or 17, wherein said aqueous solution or suspension has about 120 mg/mL of said imatinib or derivative thereof.

97. The method of claim 1, 8, or 17, wherein said aqueous solution or suspension has a pH of about 3 to about 8.

98. The method of claim 97, wherein said pH of said aqueous solution or suspension is from about 4 to about 8.

99. The method of claim 97, wherein said pH of said aqueous solution or suspension is from about 4 to about 6.

100. The method of claim 97, wherein said pH of said aqueous solution or suspension is about 5.

101. The method of claim 1, 8, or 17, wherein said aqueous solution or suspension has said cyclodextrin at a concentration of from about 2% (w/v) to about 70% (w/v), from about 2% (w/v) to about 60% (w/v), from about 2% (w/v) to about 50% (w/v), from about 2% (w/v) to about 40% (w/v), from about 2% (w/v) to about 30% (w/v), from about 2% (w/v) to about 20% (w/v), from about 2% (w/v) to about 15% (w/v), from about 2% (w/v) to about 10% (w/v), from about 2% (w/v) to about 8% (w/v), from about 2% (w/v) to about 5% (w/v), from about 5% (w/v) to about 80% (w/v), from about 5% (w/v) to about 70% (w/v), from about 5% (w/v) to about 60% (w/v), from about 5% (w/v) to about 50% (w/v), from about 5% (w/v) to about 40% (w/v), from about 5% (w/v) to about 30% (w/v), from about 5% (w/v) to about 20% (w/v), from about 5% (w/v) to about 15% (w/v), from about 5% (w/v) to about 12% (w/v), from about 5% (w/v) to about 10% (w/v), from about 10% (w/v) to about 60% (w/v), from about 10% (w/v) to about 50% (w/v), from about 10% (w/v) to about 40% (w/v), from about 10% (w/v) to about 30% (w/v), from about 20% (w/v) to about 30% (w/v), from about 10% (w/v) to about 25% (w/v), from about 19% (w/v) to about 25% (w/v), from about 19.5% (w/v) to about 25% (w/v), from about 20% (w/v) to about 25% (w/v), from about 20.5% (w/v) to about 25% (w/v), from about 21% (w/v) to about 25% (w/v), from about 21.5% (w/v) to about 25% (w/v), from about 22% (w/v) to about 25% (w/v), from about 22.5% (w/v) to about 25% (w/v), from about 23% (w/v) to about 25% (w/v), from about 10% (w/v) to about 20% (w/v), or from about 10% (w/v) to about 15% (w/v).

102. The method of claim 1, 8, or 17, wherein said aqueous solution or suspension has said cyclodextrin at a concentration of from 5% (w/v) to 40% (w/v).

103. The method of claim 1, 8, or 17, wherein said aqueous solution or suspension has said cyclodextrin at a concentration of from 10% (w/v) to 20% (w/v).

104. The method of claim 1, 8, or 17, wherein said aqueous solution or suspension has said cyclodextrin at a concentration of from 25% (w/v) to 40% (w/v).

105. The method of claim 1, 8, or 17, wherein said aqueous solution or suspension has said cyclodextrin at a concentration of about 10% (w/v), about 12% (w/v), about 14% (w/v), about 15% (w/v), about 16% (w/v), about 18% (w/v), or about 20% (w/v).

106. The method of claim 1, 8, or 17, wherein said aqueous solution or suspension has said cyclodextrin at a concentration of about 22% (w/v), about 24% (w/v), about 26% (w/v), about 28% (w/v), about 30% (w/v), about 32% (w/v), about 34% (w/v), about 36% (w/v), about 38% (w/v), or about 40% (w/v).

107. The method of claim 1, 8, or 17, wherein said composition comprises said aqueous solution.

108. The composition of claim 107, wherein solubility of the imatinib or derivative thereof in the aqueous solution is negatively correlated with the pH of the aqueous solution.

109. The composition of claim 107, wherein solubility of the imatinib or derivative thereof in the aqueous solution is positively correlated with concentration of the cyclodextrin in the aqueous solution.

110. The method of claim 1, 8, or 17, wherein said pharmaceutical composition comprises said aqueous suspension.

111. The method of claim 1, 8, or 17, wherein said pharmaceutical composition comprises less than 1 mg/mL, less than 0.5 mg/mL, less than 0.1 mg/mL, less than 0.05 mg/mL, less than 0.01 mg/mL, less than 0.005 mg/mL, less than 0.001 mg/mL, or less than 0.0001 mg/mL imatinib mesylate.

112. The method of claim 1, 8, or 17, wherein said pharmaceutical composition does not comprise imatinib mesylate.

113. The method of claim 1, 8, or 17, wherein said imatinib or derivative thereof comprises imatinib free base.

114. The method of claim 1, 8, or 17, wherein said imatinib or derivative thereof is imatinib free base.

115. The method of claim 1, 8, or 17, wherein said pharmaceutical composition comprises a salt of said imatinib or derivative thereof selected from the group consisting of: acetate salt, formate salt, citrate salt, phosphate salt, maleate salt, fumarate salt, tartrate salt, malonate salt, lactic salt, and succinate salt.

116. The method of claim 1, 8, or 17, wherein said pharmaceutical composition further comprises a pharmaceutically acceptable excipient.

117. The method of claim 116, wherein said pharmaceutically acceptable excipient comprises a surfactant.

118. The method of claim 117, wherein said surfactant comprises Tween, sodium lauryl sulfate (SLS), or dipalmitoylphosphatidylcholine (DPPC).

119. The method of claim 116, wherein said pharmaceutically acceptable excipient comprises a lipid.

120. The method of claim 119, wherein said lipid comprises a polymeric lipid, a sulfonated poly saccharide, or a fatty acid.

121. The method of claim 119, wherein said lipid comprises a polymeric lipid, a sulfonated poly saccharide, or a fatty acid.

122. The method of claim 1, 8, or 17, wherein said pharmaceutical composition is organoleptically tolerated when inhaled by a human subject.

123. The method of claim 1, 8, or 17, wherein said pharmaceutical composition does not induce cough reflex when inhaled by a human subject.

124. The method of claim 1, 8, or 17, wherein said pharmaceutical composition is not or minimally irritative to mouth or throat when inhaled by a human subject.
